SEQUENCE LISTING (455 sequences)

SEQ ID NO: 1 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSS

SEQ ID NO: 2 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCEQYEDYPLTFGGGTKVEIK

SEQ ID NO: 3 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSS

SEQ ID NO: 4 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCKQYEDYPLTFGGGTKVEIK

SEQ ID NO: 5 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSS

SEQ ID NO: 6 - protein, 107 aa, synthetic construct; identical to SEQ ID NO: 4

SEQ ID NO: 7 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSS

SEQ ID NO: 8 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCHQYEDYPLTFGGGTKVEIK

SEQ ID NO: 9 - protein, 118 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSS

SEQ ID NO: 10 - protein, 107 aa, synthetic construct; identical to SEQ ID NO: 8

SEQ ID NO: 11 - protein, 118 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSS

SEQ ID NO: 12 - protein, 107 aa, synthetic construct; identical to SEQ ID NO: 8

SEQ ID NO: 13 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSS

SEQ ID NO: 14 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDYPLVFGGGTKVEIK

SEQ ID NO: 15 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSS

SEQ ID NO: 16 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQHEDYPLTFGGGTKVEIK

SEQ ID NO: 17 - protein, 117 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSS

SEQ ID NO: 18 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHYEDYPLTFGGGTKVEIK

SEQ ID NO: 19 - protein, 118 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSS

SEQ ID NO: 20 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDLIPTFGGGTKVEIK

SEQ ID NO: 21 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSS

SEQ ID NO: 22 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQSYNSYVWTFGQGTKVEIK

SEQ ID NO: 23 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSS

SEQ ID NO: 24 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVNSYDWTFGQGTKVEIK

SEQ ID NO: 25 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSS

SEQ ID NO: 26 - protein, 107 aa, synthetic construct; identical to SEQ ID NO: 24

SEQ ID NO: 27 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSS

SEQ ID NO: 28 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSYAWTFGQGTKVEIK

SEQ ID NO: 29 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSS

SEQ ID NO: 30 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQARSYSWTFGQGTKVEIK

SEQ ID NO: 31 - protein, 119 aa, synthetic construct; identical to SEQ ID NO: 21

SEQ ID NO: 32 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQHNSYRWTFGQGTKVEIK

SEQ ID NO: 33 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSS

SEQ ID NO: 34 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCVQYPSYSWTFGQGTKVEIK

SEQ ID NO: 35 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSS

SEQ ID NO: 36 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYKLTFGQGTKVEIK

SEQ ID NO: 37 - protein, 119 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSS

SEQ ID NO: 38 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSPSWLFGQGTKVEIK

SEQ ID NO: 39 - protein, 119 aa, synthetic construct; identical to SEQ ID NO: 37

SEQ ID NO: 40 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVNSLSWTFGQGTKVEIK

SEQ ID NO: 41 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSS

SEQ ID NO: 42 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCFQYNSYPLGFGGGTKVEIK

SEQ ID NO: 43 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSS

SEQ ID NO: 44 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYRSHPLTFGGGTKVEIK

SEQ ID NO: 45 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSS

SEQ ID NO: 46 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNQVPLTFGGGTKVEIK

SEQ ID NO: 47 - protein, 125 aa, synthetic construct; identical to SEQ ID NO: 41

SEQ ID NO: 48 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQTNIYPLTFGGGTKVEIK

SEQ ID NO: 49 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSS

SEQ ID NO: 50 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYKQYPLTFGGGTKVEIK

SEQ ID NO: 51 - protein, 125 aa, synthetic construct
EVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSS

SEQ ID NO: 52 - protein, 107 aa, synthetic construct; identical to SEQ ID NO: 48

SEQ ID NO: 53 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSS

SEQ ID NO: 54 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNSYPLAFGGGTKVEIK

SEQ ID NO: 55 - protein, 125 aa, synthetic construct; identical to SEQ ID NO: 51

SEQ ID NO: 56 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYHSYPLRFGGGTKVEIK

SEQ ID NO: 57 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSS

SEQ ID NO: 58 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYASYPLKFGGGTKVEIK

SEQ ID NO: 59 - protein, 125 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSS

SEQ ID NO: 60 - protein, 107 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNAYPLIFGGGTKVEIK

SEQ ID NO: 61 - protein, 5 aa, synthetic construct
DYYMS

SEQ ID NO: 62 - protein, 17 aa, synthetic construct
YISRSGSTKYYADSVKG

SEQ ID NO: 63 - protein, 9 aa, synthetic construct
DQEDYALDY

SEQ ID NO: 64 - protein, 11 aa, synthetic construct
QASQDISNYLN

SEQ ID NO: 65 - protein, 7 aa, synthetic construct
DASNLET

SEQ ID NO: 66 - protein, 9 aa, synthetic construct
EQYEDYPLT

SEQ ID NO: 67 - protein, 9 aa, synthetic construct
DETDYGWDY

SEQ ID NO: 68 - protein, 9 aa, synthetic construct
KQYEDYPLT

SEQ ID NO: 69 - protein, 9 aa, synthetic construct
DETDYAFDE

SEQ ID NO: 70 - protein, 9 aa, synthetic construct
DESQYALDY

SEQ ID NO: 71 - protein, 9 aa, synthetic construct
HQYEDYPLT

SEQ ID NO: 72 - protein, 9 aa, synthetic construct
DNTDYALDL

SEQ ID NO: 73 - protein, 9 aa, synthetic construct
DETDYAYDE

SEQ ID NO: 74 - protein, 9 aa, synthetic construct
DETDYAYDN

SEQ ID NO: 75 - protein, 9 aa, synthetic construct
QQYEDYPLV

SEQ ID NO: 76 - protein, 9 aa, synthetic construct
DESDYAYDY

SEQ ID NO: 77 - protein, 8 aa, synthetic construct
QQHEDYPL

SEQ ID NO: 78 - protein, 8 aa, synthetic construct
DENYALDW

SEQ ID NO: 79 - protein, 8 aa, synthetic construct
QHYEDYPL

SEQ ID NO: 80 - protein, 9 aa, synthetic construct
DESDYALDF

SEQ ID NO: 81 - protein, 9 aa, synthetic construct
QQYEDLIPT

SEQ ID NO: 82 - protein, 5 aa, synthetic construct
ELSMH

SEQ ID NO: 83 - protein, 17 aa, synthetic construct
TFDPEEGETIYAQKFQG

SEQ ID NO: 84 - protein, 10 aa, synthetic construct
EGLAGVPFDL

SEQ ID NO: 85 - protein, 11 aa, synthetic construct
RASQSISSWLA

SEQ ID NO: 86 - protein, 7 aa, synthetic construct
KASSLES

SEQ ID NO: 87 - protein, 9 aa, synthetic construct
QSYNSYVWT

SEQ ID NO: 88 - protein, 10 aa, synthetic construct
EGLGGRPFDH

SEQ ID NO: 89 - protein, 9 aa, synthetic construct
QQVNSYDWT

SEQ ID NO: 90 - protein, 10 aa, synthetic construct
EGLAGFPFDI

SEQ ID NO: 91 - protein, 10 aa, synthetic construct
EGLAWRPTDS

SEQ ID NO: 92 - protein, 9 aa, synthetic construct
QQYSSYAWT

SEQ ID NO: 93 - protein, 10 aa, synthetic construct
KGLAWLPYYS

SEQ ID NO: 94 - protein, 9 aa, synthetic construct
QQARSYSWT

SEQ ID NO: 95 - protein, 9 aa, synthetic construct
QQHNSYRWT

SEQ ID NO: 96 - protein, 10 aa, synthetic construct
EGEAGRPFDA

SEQ ID NO: 97 - protein, 9 aa, synthetic construct
VQYPSYSWT

SEQ ID NO: 98 - protein, 10 aa, synthetic construct
EGLAGRPYDV

SEQ ID NO: 99 - protein, 9 aa, synthetic construct
QQYNSYKLT

SEQ ID NO: 100 - protein, 11 aa, synthetic construct
EGLAGIPFDSW

SEQ ID NO: 101 - protein, 9 aa, synthetic construct
QQYNSPSWL

SEQ ID NO: 102 - protein, 9 aa, synthetic construct
QQVNSLSWT

SEQ ID NO: 103 - protein, 5 aa, synthetic construct
DYGMS

SEQ ID NO: 104 - protein, 17 aa, synthetic construct
GINWEGGSTGYADSVKG

SEQ ID NO: 105 - protein, 16 aa, synthetic construct
DEQLGGTTYYYYYMDV

SEQ ID NO: 106 - protein, 11 aa, synthetic construct
RASQGIRNDLG

SEQ ID NO: 107 - protein, 7 aa, synthetic construct
TASNLQS

SEQ ID NO: 108 - protein, 9 aa, synthetic construct
FQYNSYPLG

SEQ ID NO: 109 - protein, 16 aa, synthetic construct
DEQLGGKYYYYYYMDV

SEQ ID NO: 110 - protein, 9 aa, synthetic construct
LQYRSHPLT

SEQ ID NO: 111 - protein, 16 aa, synthetic construct
DEQLGGQTYYYYYMDV

SEQ ID NO: 112 - protein, 9 aa, synthetic construct
LQYNQVPLT

SEQ ID NO: 113 - protein, 9 aa, synthetic construct
LQTNIYPLT

SEQ ID NO: 114 - protein, 16 aa, synthetic construct
DEQLGGIKYYYYYMDV

SEQ ID NO: 115 - protein, 9 aa, synthetic construct
LQYKQYPLT

SEQ ID NO: 116 - protein, 16 aa, synthetic construct
DEQLGGLKYYYYYMDV

SEQ ID NO: 117 - protein, 16 aa, synthetic construct
DEQLGGKVYYYYYMDV

SEQ ID NO: 118 - protein, 9 aa, synthetic construct
LQYNSYPLA

SEQ ID NO: 119 - protein, 9 aa, synthetic construct
LQYHSYPLR

SEQ ID NO: 120 - protein, 16 aa, synthetic construct
DEQLGGNHYYYYYMDA

SEQ ID NO: 121 - protein, 9 aa, synthetic construct
LQYASYPLK

SEQ ID NO: 122 - protein, 16 aa, synthetic construct
DEQLGGRHYYYYYMDV

SEQ ID NO: 123 - protein, 9 aa, synthetic construct
LQYNAYPLI

SEQ ID NO: 124 - protein, 7 aa, synthetic construct
GFTFSDY

SEQ ID NO: 125 - protein, 6 aa, synthetic construct
SRSGST

SEQ ID NO: 126 - protein, 7 aa, synthetic construct
GDTLTEL

SEQ ID NO: 127 - protein, 6 aa, synthetic construct
DPEEGE

SEQ ID NO: 128 - protein, 7 aa, synthetic construct
GFTFDDY

SEQ ID NO: 129 - protein, 6 aa, synthetic construct
NWEGGS

SEQ ID NO: 130 - protein, 8 aa, synthetic construct
GFTFSDYY

SEQ ID NO: 131 - protein, 8 aa, synthetic construct
ISRSGSTK

SEQ ID NO: 132 - protein, 11 aa, synthetic construct
ARDQEDYALDY

SEQ ID NO: 133 - protein, 6 aa, synthetic construct
QDISNY

SEQ ID NO: 134 - skipped

SEQ ID NO: 135 - protein, 11 aa, synthetic construct
ARDETDYGWDY

SEQ ID NO: 136 - protein, 11 aa, synthetic construct
ARDETDYAFDE

SEQ ID NO: 137 - protein, 11 aa, synthetic construct
ARDESQYALDY

SEQ ID NO: 138 - protein, 11 aa, synthetic construct
ARDNTDYALDL

SEQ ID NO: 139 - protein, 11 aa, synthetic construct
ARDETDYAYDE

SEQ ID NO: 140 - protein, 11 aa, synthetic construct
ARDETDYAYDN

SEQ ID NO: 141 - protein, 11 aa, synthetic construct
ARDESDYAYDY

SEQ ID NO: 142 - protein, 10 aa, synthetic construct
ARDENYALDW

SEQ ID NO: 143 - protein, 11 aa, synthetic construct
ARDESDYALDF

SEQ ID NO: 144 - protein, 8 aa, synthetic construct
GDTLTELS

SEQ ID NO: 145 - protein, 8 aa, synthetic construct
FDPEEGET

SEQ ID NO: 146 - protein, 12 aa, synthetic construct
VTEGLAGVPFDL

SEQ ID NO: 147 - protein, 6 aa, synthetic construct
QSISSW

SEQ ID NO: 148 - skipped

SEQ ID NO: 149 - protein, 12 aa, synthetic construct
VTEGLGGRPFDH

SEQ ID NO: 150 - protein, 12 aa, synthetic construct
VTEGLAGFPFDI

SEQ ID NO: 151 - protein, 12 aa, synthetic construct
VTEGLAWRPTDS

SEQ ID NO: 152 - protein, 12 aa, synthetic construct
VTKGLAWLPYYS

SEQ ID NO: 153 - protein, 12 aa, synthetic construct
VTEGEAGRPFDA

SEQ ID NO: 154 - protein, 12 aa, synthetic construct
VTEGLAGRPYDV

SEQ ID NO: 155 - protein, 13 aa, synthetic construct
VTEGLAGIPFDSW

SEQ ID NO: 156 - protein, 8 aa, synthetic construct
GFTFDDYG

SEQ ID NO: 157 - protein, 8 aa, synthetic construct
INWEGGST

SEQ ID NO: 158 - protein, 18 aa, synthetic construct
ARDEQLGGTTYYYYYMDV

SEQ ID NO: 159 - protein, 6 aa, synthetic construct
QGIRND

SEQ ID NO: 160 - skipped

SEQ ID NO: 161 - protein, 17 aa, synthetic construct
ARDEQLGGKYYYYYMDV

SEQ ID NO: 162 - protein, 18 aa, synthetic construct
ARDEQLGGQTYYYYYMDV

SEQ ID NO: 163 - protein, 18 aa, synthetic construct
ARDEQLGGIKYYYYYMDV

SEQ ID NO: 164 - protein, 18 aa, synthetic construct
ARDEQLGGLKYYYYYMDV

SEQ ID NO: 165 - protein, 18 aa, synthetic construct
ARDEQLGGKVYYYYYMDV

SEQ ID NO: 166 - protein, 18 aa, synthetic construct
ARDEQLGGNHYYYYYMDA

SEQ ID NO: 167 - protein, 18 aa, synthetic construct
ARDEQLGGRHYYYYYMDV

SEQ ID NO: 168 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 169 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 170 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 171 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 172 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 173 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 174 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 175 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 176 - protein, 444 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 177 - protein, 444 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 178 - protein, 444 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 179 - protein, 444 aa, synthetic construct
QVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 180 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 181 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 182 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 183 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 184 - protein, 443 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 185 - protein, 443 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 186 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 187 - protein, 444 aa, synthetic construct
QVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 188 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 189 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 190 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 191 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 192 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 193 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 194 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 195 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 196 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 197 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 198 - protein, 445 aa, synthetic construct; identical to SEQ ID NO: 188

SEQ ID NO: 199 - protein, 445 aa, synthetic construct; identical to SEQ ID NO: 189

SEQ ID NO: 200 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 201 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 202 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 203 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 204 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 205 - protein, 445 aa, synthetic construct
QVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 206 - protein, 445 aa, synthetic construct; identical to SEQ ID NO: 204

SEQ ID NO: 207 - protein, 445 aa, synthetic construct; identical to SEQ ID NO: 205

SEQ ID NO: 208 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 209 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 210 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 211 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 212 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 213 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 214 - protein, 451 aa, synthetic construct; identical to SEQ ID NO: 208

SEQ ID NO: 215 - protein, 451 aa, synthetic construct; identical to SEQ ID NO: 209

SEQ ID NO: 216 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 217 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 218 - protein, 451 aa, synthetic construct
EVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 219 - protein, 451 aa, synthetic construct
EVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 220 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 221 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 222 - protein, 451 aa, synthetic construct; identical to SEQ ID NO: 218

SEQ ID NO: 223 - protein, 451 aa, synthetic construct; identical to SEQ ID NO: 219

SEQ ID NO: 224 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 225 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 226 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 227 - protein, 451 aa, synthetic construct
EVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 228 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCEQYEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCKQYEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 214 aa, synthetic construct; identical to SEQ ID NO: 229

SEQ ID NO: 231 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCHQYEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 214 aa, synthetic construct; identical to SEQ ID NO: 231

SEQ ID NO: 233 - protein, 214 aa, synthetic construct; identical to SEQ ID NO: 231

SEQ ID NO: 234 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDYPLVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQHEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQHYEDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEDLIPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQSYNSYVWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVNSYDWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 214 aa, synthetic construct; identical to SEQ ID NO: 239

SEQ ID NO: 241 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSYAWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQARSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQHNSYRWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCVQYPSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYKLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSPSWLFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQVNSLSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCFQYNSYPLGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYRSHPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNQVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQTNIYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYKQYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 253 - protein, 214 aa, synthetic construct; identical to SEQ ID NO: 251

SEQ ID NO: 254 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNSYPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYHSYPLRFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYASYPLKFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 214 aa, synthetic construct
DIQMTQSPSSLSASVGDRVTITCRASQGIRNDLGWYQQKPGKAPKRLIYTASNLQSGVPSRFSGSGSGTEFTLTISSLQPEDFATYYCLQYNAYPLIFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 259 - protein, 15 aa, synthetic construct
MWCIVLFSLLAWVYA

SEQ ID NO: 260 - protein, 673 aa, Homo sapiens
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 261 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 262 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 263 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 264 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 265 - protein, 107 aa, Homo sapiens [sequence omitted]

SEQ ID NO: 266 - protein, 5 aa, synthetic construct
  REPEAT 1..5: note = The sequence can be repeated 1 time, 2 times, 3 times, 4 times, or 5 times
GGGGS

SEQ ID NO: 267 - protein, 5 aa, synthetic construct
  REPEAT 1..5: note = The sequence can be repeated 1 time, 2 times, 3 times, 4 times, or 5 times
GGGGA

SEQ ID NO: 268 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagaccaggaagattacgctcttgactactggggccaaggtacgctggttacggtc

SEQ ID NO: 269 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtgaacagtatgaggactaccctcttaccttcggcggtggaacgaaagttgagattaagcgaacc

SEQ ID NO: 270 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacggctgggactactggggccaaggtacgctggttacggtc

SEQ ID NO: 271 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtaagcagtatgaggactaccctcttaccttcggcggtggaacgaaagttgagattaagcgaacc

SEQ ID NO: 272 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacgctttcgacgaatggggccaaggtacgctggttacggtc

SEQ ID NO: 273 - DNA, 327 bp, synthetic construct; identical to SEQ ID NO: 271
  source 1..327: mol_type = other DNA

SEQ ID NO: 274 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtcagtacgctcttgactactggggccaaggtacgctggttacggtc

SEQ ID NO: 275 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcaccagtatgaggactaccctcttaccttcggcggtggaacgaaagttgagattaagcgaacc

SEQ ID NO: 276 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcacattcagtgaagggcaggtttacgattagccgagacaacgcaaagaactctctttatctgcagatgaattcactgagagcagaagataccgctgtctattattgtgccagagacaacaccgattacgctcttgacctgtggggccaaggtacgctggttacggtc

SEQ ID NO: 277 - DNA, 327 bp, synthetic construct; identical to SEQ ID NO: 275
  source 1..327: mol_type = other DNA

SEQ ID NO: 278 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcacattcagtgaagggcaggtttacgattagccgagacaacgcaaagaactctctttatctgcagatgaattcactgagagcagaagataccgctgtctattattgtgccagagacgagaccgattacgcttacgacgaatggggccaaggtacgctggttacggtc

SEQ ID NO: 279 - DNA, 327 bp, synthetic construct; identical to SEQ ID NO: 275
  source 1..327: mol_type = other DNA

SEQ ID NO: 280 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacgcttacgacaactggggccaaggtacgctggttacggtc

SEQ ID NO: 281 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcagcagtatgaggactaccctcttgtgttcggcggtggaacgaaagttgagattaagcgaacc

SEQ ID NO: 282 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtgattacgcttacgactactggggccaaggtacgctggttacggtc

SEQ ID NO: 283 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcagcagcacgaggactaccctcttaccttcggcggtggaacgaaagttgagattaagcgaacc

SEQ ID NO: 284 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaattacgctcttgactggtggggccaaggtacgctggttacggtctcg

SEQ ID NO: 285 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcagcactatgaggactaccctcttaccttcggcggtggaacgaaagttgagattaagcgaacc

SEQ ID NO: 286 - DNA, 348 bp, synthetic construct
  source 1..348: mol_type = other DNA
caggtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtgattacgctcttgacttctggggccaaggtacgctggttacggtc

SEQ ID NO: 287 - DNA, 327 bp, synthetic construct
  source 1..327: mol_type = other DNA
gacattcagatgacgcaaagcccctctagcttgtccgctagtgtgggtgacagggtcaccattacctgccaggcttcacaagacatcagtaattacctcaactggtatcagcagaaacctgggaaggcccctaagctgttgatttacgatgctagtaacttggagaccggggtacccagcagatttagcgggagcggaagtggtacggattttacgtttaccattagcagcttgcagcccgaggacatcgctacatattactgtcagcagtatgaggacctcatccccaccttcggcggtggaacgaaagttgagattaagcgaacc

SEQ ID NO: 288 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccggggtgccgtttgatctgtgggggcaggggacgctggtaacggtctcgagt

SEQ ID NO: 289 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagtggatcaggatctggtactgaattcactctgaccatttcaagtcttcagcctgatgactttgcgacctattattgccagagctacaatagctacgtgtggacgttcgggcagggtactaaagtcgagattaaacgaacc

SEQ ID NO: 290 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttgggcgggcgcccgtttgatcactgggggcaggggacgctggtaacggtctcgagt

SEQ ID NO: 291 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagtggatcaggatctggtactgaattcactctgaccatttcaagtcttcagcctgatgactttgcgacctattattgccagcaggtgaatagctacgactggacgttcgggcagggtactaaagtcgagattaaacgaacc

SEQ ID NO: 292 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccgggttcccgtttgatatctgggggcaggggacgctggtaacggtctcgagt

SEQ ID NO: 293 - DNA, 327 bp, synthetic construct; identical to SEQ ID NO: 291
  source 1..327: mol_type = other DNA

SEQ ID NO: 294 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggcctggcgcccgaccgattcatgggggcaggggacgctggtaacggtctcgagt

SEQ ID NO: 295 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcagtacagcagctacgcctggacgttcgggcagggtactaaagtcgagattaaacgaacc

SEQ ID NO: 296 - DNA, 357 bp, synthetic construct
  source 1..357: mol_type = other DNA
caagttcaactggttcaaagcggggccgaggtaaagaagtcaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccaagggtttggccgggctgccgtactactcatgggggcaggggacgctggtaacggtctcgagt

SEQ ID NO: 297 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcaggcccgcagctactcatggacgttcgggcagggtactaaagtcgagattaaacgaacc

SEQ ID NO: 298 - DNA, 357 bp, synthetic construct; identical to SEQ ID NO: 288
  source 1..357: mol_type = other DNA

SEQ ID NO: 299 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcagcacaatagctaccgctggacgttcgggcagggtactaaagtcgagattaaacgaacc

SEQ ID NO: 300 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtgaggccgggcgcccgtttgatgcctgggggcaggggacgctggtaacggtctcgagt

SEQ ID NO: 301 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgcgtgcagtaccccagctactcatggacgttcgggcagggtactaaagtcgagattaaacgaacc

SEQ ID NO: 302 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccgggcgcccgtacgatgtgtgggggcaggggacgctggtaacggtctcgagt

SEQ ID NO: 303 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcagtacaatagctacaagctgacgttcgggcagggtactaaagtcgagattaaacgaacc

SEQ ID NO: 304 - DNA, 357 bp, synthetic construct
  source 1..357: mol_type = other DNA
caagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctacctcagaagtgaagatactgccgtctactactgtgtgaccgagggtttggccgggatcccgtttgattcatgggggcaggggacgctggtaacggtctcgagt

SEQ ID NO: 305 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcagtacaatagcccctcatggctgttcgggcagggtactaaagtcgagattaaacgaacc

SEQ ID NO: 306 - DNA, 357 bp, synthetic construct; identical to SEQ ID NO: 304
  source 1..357: mol_type = other DNA

SEQ ID NO: 307 - DNA, 327 bp, synthetic construct
  source 1..327: mol_type = other DNA
gacatacaaatgactcaatcaccctctacactttccgcctctgtcggcgacagggtaacgataacttgccgggcaagtcaatcaatcagctcctggcttgcttggtatcaacaaaaaccaggcaaggcacctaagctcctcatttacaaggcgtcatcacttgaatcaggggtgccttcacgattcagcggatcaggatctggtactgaattcactctgaccatttcaagtcttcagcctgatgactttgcgacctattattgccagcaggtgaatagcctgtcatggacgttcgggcagggtactaaagtcgagattaaacgaacc

SEQ ID NO: 308 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggactacttattattactattacatggacgtgtggggtaaaggaacgaccgtcacagtc

SEQ ID NO: 309 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttccagtataatagttatcctctcggctttggcggtggcaccaaagtggagattaagcgaacc

SEQ ID NO: 310 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaagtattactattactattacatggacgtgtggggtaaaggaacgaccgtcacagtc

SEQ ID NO: 311 - DNA, 330 bp, synthetic construct
  source 1..330: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtaccgcagtcactatcctctcacttttggcggtggcaccaaagtggagattaagcgaacc

SEQ ID NO: 312 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggcagacttattattactattacatggacgtgtggggtaaaggaacgaccgtcacagtc

SEQ ID NO: 313 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtataatcaggtgcctctcacttttggcggtggcaccaaagtggagattaagcgaacc

SEQ ID NO: 314 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaccacctattattactattacatggacgtgtggggtaaaggaacgaccgtcacagtc

SEQ ID NO: 315 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagaccaatatctatcctctcacttttggcggtggcaccaaagtggagattaagcgaacc

SEQ ID NO: 316 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggatcaagtattattactattacatggacgtgtggggtaaaggaacgaccgtcacagtc

SEQ ID NO: 317 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtataagcagtatcctctcacttttggcggtggcaccaaagtggagattaagcgaacc

SEQ ID NO: 318 - DNA, 369 bp, synthetic construct
  source 1..369: mol_type = other DNA
gaagtccagcttgttgagtctggcggaggggtggttaggccaggaggttcccttaggctttcctgtgcggcatcaggatttactttcgatgactacggaatgtcatgggtacgacaagctcccggcaaaggccttgaatgggtttctggcatcaactgggagggcggttccactggctacgcggactcagttaagggtaggttcagcataagcagggacaatgcgaagaactcactgtatctccaaatgaacagtctccgcgccgaggatacagccctttactattgtgcacgagatgagcaattgggggggctgaagtattattactattacatggacgtgtggggtaaaggaacgaccgtcacagtc

SEQ ID NO: 319 - DNA, 327 bp, synthetic construct; identical to SEQ ID NO: 315
  source 1..327: mol_type = other DNA

SEQ ID NO: 320 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaaggtgtattattactattacatggacgtgtggggtaaaggaacgaccgtcacagtc

SEQ ID NO: 321 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtataatagttatcctctcgcctttggcggtggcaccaaagtggagattaagcgaacc

SEQ ID NO: 322 - DNA, 369 bp, synthetic construct; identical to SEQ ID NO: 318
  source 1..369: mol_type = other DNA

SEQ ID NO: 323 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtatcacagttatcctctccgctttggcggtggcaccaaagtggagattaagcgaacc

SEQ ID NO: 324 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaatcactattattactattacatggacgcttggggtaaaggaacgaccgtcacagtc

SEQ ID NO: 325 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtatgccagttatcctctcaagtttggcggtggcaccaaagtggagattaagcgaacc

SEQ ID NO: 326 - DNA, 369 bp, synthetic construct
  source 1..369: mol_type = other DNA
gaagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggcgccactattattactattacatggacgtgtggggtaaaggaacgaccgtcacagtc

SEQ ID NO: 327 - DNA, 327 bp, synthetic construct
  source 1..327: mol_type = other DNA
gacatacagatgactcagtccccaagtagtctgagcgcctctgtcggcgaccgcgttaccattacttgccgggcgagtcaagggatacgcaacgatttgggttggtatcagcaaaagcccggaaaggccccaaagcggctgatatacaccgcctcaaacctccagtctggagttccatcaaggtttagtggatctggatctggcactgagtttactctcactataagctcccttcagcctgaagacttcgcaacttattattgcttgcagtataatgcctatcctctcatctttggcggtggcaccaaagtggagattaagcgaacc

SEQ ID NO: 328-332 - skipped (5 entries)

SEQ ID NO: 333 - protein, 16 aa, synthetic construct
  VARIANT 7: note = X can be N, T, I, Q, L, K, or R
  VARIANT 8: note = X can be Y, T, K, H, or V
  VARIANT 16: note = X can be V or A
DEQLGGXXYYYYYMDX

SEQ ID NO: 334 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSS

SEQ ID NO: 335 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSS

SEQ ID NO: 336 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSS

SEQ ID NO: 337 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSS

SEQ ID NO: 338 - protein, 118 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSS

SEQ ID NO: 339 - protein, 118 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSS

SEQ ID NO: 340 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSS

SEQ ID NO: 341 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSS

SEQ ID NO: 342 - protein, 117 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSS

SEQ ID NO: 343 - protein, 118 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSS

SEQ ID NO: 344 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSS

SEQ ID NO: 345 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSS

SEQ ID NO: 346 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSS

SEQ ID NO: 347 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSS

SEQ ID NO: 348 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSS

SEQ ID NO: 349 - protein, 119 aa, synthetic construct; identical to SEQ ID NO: 344

SEQ ID NO: 350 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSS

SEQ ID NO: 351 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSS

SEQ ID NO: 352 - protein, 119 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSS

SEQ ID NO: 353 - protein, 119 aa, synthetic construct; identical to SEQ ID NO: 352

SEQ ID NO: 354 - protein, 125 aa, synthetic construct; identical to SEQ ID NO: 41

SEQ ID NO: 355 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSS

SEQ ID NO: 356 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSS

SEQ ID NO: 357 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSS

SEQ ID NO: 358 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSS

SEQ ID NO: 359 - protein, 125 aa, synthetic construct
QVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSS

SEQ ID NO: 360 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSS

SEQ ID NO: 361 - protein, 125 aa, synthetic construct; identical to SEQ ID NO: 359

SEQ ID NO: 362 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSS

SEQ ID NO: 363 - protein, 125 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSS

SEQ ID NO: 364 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 365 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDQEDYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 366 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 367 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYGWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 368 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 369 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAFDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 370 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 371 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESQYALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 372 - protein, 444 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 373 - protein, 444 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDNTDYALDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 374 - protein, 444 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 375 - protein, 444 aa, synthetic construct
EVQLVESGGGLVKPGGSLRLSCAASGFTFSDYYMSWIRQAPGKGLEWVSYISRSGSTKYYAHSVKGRFTISRDNAKNSLYLQMNSLRAEDTAVYYCARDETDYAYDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 376 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 377 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDETDYAYDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 378 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 379 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYAYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 380 - protein, 443 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 381 - protein, 443 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 382 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 383 - protein, 444 aa, synthetic construct
EVQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDESDYALDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 384 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 385 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGVPFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 386 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 387 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLGGRPFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 388 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 389 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGFPFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 390 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 391 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAWRPTDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 392 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 393 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTKGLAWLPYYSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 394 - protein, 445 aa, synthetic construct; identical to SEQ ID NO: 384

SEQ ID NO: 395 - protein, 445 aa, synthetic construct; identical to SEQ ID NO: 385

SEQ ID NO: 396 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 397 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGEAGRPFDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 398 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 399 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSSLRSEDTAVYYCVTEGLAGRPYDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 400 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 401 - protein, 445 aa, synthetic construct
EVQLVQSGAEVKKPGASVKVSCKVSGDTLTELSMHWVRQAPGKGLEWMGTFDPEEGETIYAQKFQGRVTMTEDTSTDTAYMELSYLRSEDTAVYYCVTEGLAGIPFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 402 - protein, 445 aa, synthetic construct; identical to SEQ ID NO: 400

SEQ ID NO: 403 - protein, 445 aa, synthetic construct; identical to SEQ ID NO: 401

SEQ ID NO: 404 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 405 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGTTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 406 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 407 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKYYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 408 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 409 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGQTYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 410 - protein, 451 aa, synthetic construct; identical to SEQ ID NO: 404

SEQ ID NO: 411 - protein, 451 aa, synthetic construct; identical to SEQ ID NO: 405

SEQ ID NO: 412 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 413 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGIKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 414 - protein, 451 aa, synthetic construct
QVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 415 - protein, 451 aa, synthetic construct
QVQLVESGGGVVRPGGSLRLSCAASGFTFDDYGMSWVRQAPGKGLEWVSGINWEGGSTGYADSVKGRFSISRDNAKNSLYLQMNSLRAEDTALYYCARDEQLGGLKYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 416 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 417 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGKVYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 418 - protein, 451 aa, synthetic construct; identical to SEQ ID NO: 414

SEQ ID NO: 419 - protein, 451 aa, synthetic construct; identical to SEQ ID NO: 415

SEQ ID NO: 420 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 421 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGNHYYYYYMDAWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 422 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 423 - protein, 451 aa, synthetic construct
QVQLVESGGGVVRPGGSLRLSCAASGFTFDDYGMSWVRQAPGKGLEWVSGINWEGGSTGYADSVKGRFTISRDNAKNSLYLQMNSLRAEDTALYYCARDEQLGGRHYYYYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 424 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagaccaggaagattacgctcttgactactggggccaaggtacgctggttacggtc

SEQ ID NO: 425 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacggctgggactactggggccaaggtacgctggttacggtc

SEQ ID NO: 426 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacgctttcgacgaatggggccaaggtacgctggttacggtc

SEQ ID NO: 427 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtcagtacgctcttgactactggggccaaggtacgctggttacggtc

SEQ ID NO: 428 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcacattcagtgaagggcaggtttacgattagccgagacaacgcaaagaactctctttatctgcagatgaattcactgagagcagaagataccgctgtctattattgtgccagagacaacaccgattacgctcttgacctgtggggccaaggtacgctggttacggtc

SEQ ID NO: 429 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcacattcagtgaagggcaggtttacgattagccgagacaacgcaaagaactctctttatctgcagatgaattcactgagagcagaagataccgctgtctattattgtgccagagacgagaccgattacgcttacgacgaatggggccaaggtacgctggttacggtc

SEQ ID NO: 430 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaccgattacgcttacgacaactggggccaaggtacgctggttacggtc

SEQ ID NO: 431 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtgattacgcttacgactactggggccaaggtacgctggttacggtc

SEQ ID NO: 432 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagaattacgctcttgactggtggggccaaggtacgctggttacggtctcg

SEQ ID NO: 433 - DNA, 348 bp, synthetic construct
  source 1..348: mol_type = other DNA
gaagtgcagcttgttgaaagtggtgggggtttggttaaacctggcggttcccttcgacttagctgcgcggcgtcagggttcacattctcagattattacatgtcttggattcggcaagcccctggtaagggactggagtgggtaagctacatatctcggtcaggaagtacaaagtactatgcagattcagtgaagggcaggtttacgattagccgagacaacgcaaagaactctctttatctgcagatgaattcactgagagcagaagataccgctgtctattattgtgccagagacgagagtgattacgctcttgacttctggggccaaggtacgctggttacggtc

SEQ ID NO: 434 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccggggtgccgtttgatctgtgggggcaggggacgctggtaacggtctcgagt

SEQ ID NO: 435 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttgggcgggcgcccgtttgatcactgggggcaggggacgctggtaacggtctcgagt

SEQ ID NO: 436 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccgggttcccgtttgatatctgggggcaggggacgctggtaacggtctcgagt

SEQ ID NO: 437 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggcctggcgcccgaccgattcatgggggcaggggacgctggtaacggtctcgagt

SEQ ID NO: 438 - DNA, 357 bp, synthetic construct
  source 1..357: mol_type = other DNA
gaagttcaactggttcaaagcggggccgaggtaaagaagtcaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccaagggtttggccgggctgccgtactactcatgggggcaggggacgctggtaacggtctcgagt

SEQ ID NO: 439 - DNA, 357 bp, synthetic construct; identical to SEQ ID NO: 434
  source 1..357: mol_type = other DNA

SEQ ID NO: 440 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtgaggccgggcgcccgtttgatgcctgggggcaggggacgctggtaacggtctcgagt

SEQ ID NO: 441 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctccctcagaagtgaagatactgccgtctactactgtgtgaccgagggtttggccgggcgcccgtacgatgtgtgggggcaggggacgctggtaacggtctcgagt

SEQ ID NO: 442 - DNA, 357 bp, synthetic construct
  source 1..357: mol_type = other DNA
gaagttcaactggttcaaagcggggccgaggtaaagaagccaggcgcttctgtgaaagtcagttgcaaggtgagtggggatacattgaccgagctgagtatgcactgggtccggcaagctcctggcaagggtttggaatggatgggtacttttgacccggaggagggagagaccatctacgcgcaaaaattccaaggtagggtgaccatgacggaggataccagcacggatactgcttacatggagctcagctacctcagaagtgaagatactgccgtctactactgtgtgaccgagggtttggccgggatcccgtttgattcatgggggcaggggacgctggtaacggtctcgagt

SEQ ID NO: 443 - DNA, 357 bp, synthetic construct; identical to SEQ ID NO: 442
  source 1..357: mol_type = other DNA

SEQ ID NO: 444 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggactacttattattactattacatggacgtgtggggtaaaggaacgaccgtcacagtc

SEQ ID NO: 445 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaagtattactattactattacatggacgtgtggggtaaaggaacgaccgtcacagtc

SEQ ID NO: 446 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggcagacttattattactattacatggacgtgtggggtaaaggaacgaccgtcacagtc

SEQ ID NO: 447 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaccacctattattactattacatggacgtgtggggtaaaggaacgaccgtcacagtc

SEQ ID NO: 448 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggatcaagtattattactattacatggacgtgtggggtaaaggaacgaccgtcacagtc

SEQ ID NO: 449 - DNA, 369 bp, synthetic construct
  source 1..369: mol_type = other DNA
caagtccagcttgttgagtctggcggaggggtggttaggccaggaggttcccttaggctttcctgtgcggcatcaggatttactttcgatgactacggaatgtcatgggtacgacaagctcccggcaaaggccttgaatgggtttctggcatcaactgggagggcggttccactggctacgcggactcagttaagggtaggttcagcataagcagggacaatgcgaagaactcactgtatctccaaatgaacagtctccgcgccgaggatacagccctttactattgtgcacgagatgagcaattgggggggctgaagtattattactattacatggacgtgtggggtaaaggaacgaccgtcacagtc

SEQ ID NO: 450 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaaggtgtattattactattacatggacgtgtggggtaaaggaacgaccgtcacagtc

SEQ ID NO: 451 - DNA, 369 bp, synthetic construct; identical to SEQ ID NO: 449
  source 1..369: mol_type = other DNA

SEQ ID NO: 452 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggaatcactattattactattacatggacgcttggggtaaaggaacgaccgtcacagtc

SEQ ID NO: 453 - DNA, 369 bp, synthetic construct
  source 1..369: mol_type = other DNA
caagtccagcttgttgagtctggcggaggggtggttaggccaggaggttcccttaggctttcctgtgcggcatcaggatttaccttcgatgactacggaatgtcatgggtacgacaagctcccggcaaaggcctcgaatgggtttctggcatcaactgggagggcggttccactggctacgcggactcagttaagggtaggttcaccataagcagggacaatgcgaagaactcactgtatctccaaatgaacagtctccgcgccgaggatacagccctttactattgtgcacgagatgagcaattgggggggcgccactattattactattacatggacgtgtggggtaaaggaacgaccgtcacagtc

SEQ ID NO: 454 - protein, 116 aa, synthetic construct
VQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSS

SEQ ID NO: 455 - protein, 442 aa, synthetic construct
VQLVESGGGLVKPGGSLRLSCAASGFTFSDYYMSWIRQAPGKGLEWVSYISRSGSTKYYADSVKGRFTISRDNAKNSLYLQMNSLRAEDTAVYYCARDENYALDWWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2025/0206814 A1
(43) Pub. Date: Jun. 26, 2025

(54) PHARMACEUTICAL COMPOSITIONS COMPRISING ANTIBODIES FOR TREATMENT OF C1S MEDIATED DISORDERS AND METHODS OF USING THE SAME

(71) Applicant: DIANTHUS THERAPEUTICS OPCO, INC., New York, NY (US)

(72) Inventors: Taotao Chen, New York, NY (US); Robert McGarr, New York, NY (US); Peipei Li, Shanghai (CN); Zhixin Zhao, Shanghai (CN)

(21) Appl. No.: 18/990,144

(22) Filed: Dec. 20, 2024

Related U.S. Application Data
(63) Continuation-in-part of application No. PCT/CN2023/140449, filed on Dec. 20, 2023.

(30) Foreign Application Priority Data
Dec. 13, 2024 (CN) CN202411844428

(51) Int. Cl. C07K 16/18; A61K 39/00; A61K 47/02; A61K 47/10; A61K 47/18; A61K 47/26

(52) U.S. Cl. CPC C07K 16/18; A61K 47/02; A61K 47/10; A61K 47/183; A61K 47/26; A61K 2039/54; C07K 2317/565

(57) ABSTRACT

Provided for herein are pharmaceutical compositions comprising a therapeutic antibody and uses thereof for the treatment of C1s mediated disorders.

### CROSS-REFERENCE TO RELATED APPLICATIONS

This application is a continuation-in-part of, and claims priority to, International Application No. PCT/CN2023/140449, filed Dec. 20, 2023, and this application claims priority to Chinese Application No. 202411844428.4, filed Dec. 13, 2024, each of which is hereby incorporated by reference in its entirety.

REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Oct. 31, 2024, is named “DIN-003WO2 Sequence listings.xml” and is 494,660 bytes in size.

### FIELD

Embodiments provided herein related to therapeutic antibodies, and pharmaceutical compositions comprising the same for the treatment of C1s mediated disorders.

### BACKGROUND

The complement system is a well-known effector mechanism of the innate immune response, providing not only protection against pathogens and other harmful agents but also recovery from injury. Complement activation due to autoantibodies and alloantibodies can lead to damage to normal cells or rejection of transplanted tissue. The complement pathway comprises a number of proteins that typically exist in the body in inactive form. The classical complement pathway is triggered by activation of the first component of complement, referred to as the C1 complex, which consists of C1q, C1r, and C1s proteins. Upon binding of C1 to an immune complex or other activator, the C1s component, a diisopropyl fluorophosphate (DFP)-sensitive serine protease, cleaves complement components C4 and C2 to initiate activation of the classical complement pathway. The classical complement pathway appears to play a role in many diseases and disorders. For example, sutimlimab (TNT009), marketed as EnJaymo®, is an antibody that inhibits C1s for treatment of hemolysis in adults with cold agglutinin disease. However, sutimlimab binds to both the active form of C1s, and the inactive zymogen proC1s, and due to the lack of specificity of sutimlimab for either form of C1s, a very high dose must be administered to overcome circulating levels of proC1s, and may limit the clinical use of sutimlimab. Therefore, there is a need in the art for compounds that treat a complement classical pathway-mediated disease or disorder with specificity for active forms of the complement proteins and pharmaceutically acceptable formulations to safely and effectively store and administer these compounds in a therapeutic setting. The embodiments provided for herein satisfy these needs as well as others.

### SUMMARY

Disclosed herein are pharmaceutical compositions for treating C1s mediated disorders and dosage forms, kits, and administration methods thereof. Embodiments disclosed herein are incorporated by reference into this section.

In some embodiments, an antibody, or antigen binding fragment thereof, is provided that binds to the active form of C1s. In some embodiments, an antibody, or antigen binding fragment thereof, is provided that specifically binds to the active form of C1s. In some embodiments, the antibody, or antigen binding fragment thereof, is as provided herein.

### DETAILED DESCRIPTION

Provided herein are binding proteins, e.g., antibodies, or fragments thereof, that selectively bind to C1s and have low binding to the zymogen proC1s. In some embodiments, the antibodies inhibit activation of the classical complement pathway and can be used in methods to treat complement mediated disorders, such as, but not limited to those provided for herein. In some embodiments, the selectivity for Cls over proC1s can be used to reduce or prevent target mediated clearance of the therapeutic antibody, thus requiring lower doses and frequency of administration of the antibody.

Also provided herein are pharmaceutical compositions of antibodies that bind and modulate the activity of C1s. The antibodies can be used, for example, to treat C1s mediated disorders.

It is to be understood that the embodiments described herein are not limited to particular formulations, compositions and experimental conditions disclosed, as such formulations, compositions, and experimental conditions may vary. It is also to be understood that the terminology used herein is only for the purpose of describing particular embodiments, and it is not intended to be limiting.

The following explanations of terms and methods are provided to better describe the present disclosure and to guide those of ordinary skill in the art in the practice of the present disclosure.

Those of ordinary skill in the art will understand that given the amino acid sequence of a variable region of the binding molecules (e.g., antibodies) set forth herein can determine the complementarity determining region (CDR) sequences therein by these or any other conventions used to define CDR, such as, for example, IMGT, Kabat, Chothia, or Contact using web-based tools are available for determining the CDRs in such variable regions based on any known convention. Such tools include those found at www.abysis.org/abysis/sequence input/key_annotation/key_annotation.cgi and at www.novoprolabs.com/tools/cdr. Accordingly, the disclosure herein of a set of three CDRs in a heavy or light chain variable region based upon one CDR convention is considered the equivalent of that same set of CDRs as determined by any other convention, which can be referred to as “convention equivalents” or “convention equivalent CDRs”.

Furthermore, the formulations, compositions, and experimental conditions described herein, unless otherwise indicated, use conventional molecular and cellular biological and immunological techniques known within the skill of the art. Such techniques are well known to the skilled worker, and are explained fully in the literature. (see, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition), by MR Green and J. Sambrook and Harlow et al., Antibodies: A Laboratory Manual, Chapter 14, Cold Spring Harbor Laboratory, Cold Spring Harbor (2013, 2^{nd} edition)).

Unless otherwise defined, scientific and technical terms used herein have the meanings that are commonly understood by those of ordinary skill in the art. In the event of any latent ambiguity, definitions provided herein take precedent over any dictionary or extrinsic definition.

Unless otherwise required by context, singular terms shall include pluralities and plural terms shall include the singular.

The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions are performed according to manufacturer's specifications, as commonly accomplished in the art or as described herein. The nomenclatures used in connection with the laboratory procedures and techniques of analytical chemistry, synthetic organic chemistry, and medicinal and pharmaceutical chemistry described herein are those well-known and commonly used in the art.

As used herein, the terms “a” or “and” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, where a phrase recites “about x to y”, the term “about” modifies both x and y and can be used interchangeably with the phrase “about x to about y” unless context dictates differently.

As used herein, the terms “comprising” (and any form of comprising, such as “comprise”, “comprises” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists of”. Further, the singular forms “a” or “an” or “the” include plural references unless the context clearly dictates otherwise. For example, reference to “comprising a therapeutic agent” includes one or a plurality of such therapeutic agents. The term “or” refers to a single element of stated alternative elements, unless the context clearly indicates otherwise. For example, the phrase “A or B” refers to A alone or B alone. The phrase “A, B, or a combination thereof” refers to A alone, B alone, or a combination of A and B. Similarly, “one or more of A and B” refers to A, B, or a combination of both A and B. The phrase “A and B” refers to a combination of A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples.

A “disease” in an animal is a state of health wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Parenteral” administration of a composition includes, e.g., subcutaneous (s.c.), intravenous (i.v.), intramuscular (i.m.), or intracisternal injection, intrathecal, or infusion techniques.

The term “therapeutic” as used herein means a treatment and/or prophylaxis. A therapeutic effect is obtained by suppression, remission, or eradication of a disease state.

Throughout this disclosure, various aspects of the embodiments can be presented in a range format. It should be understood that the description in the range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as the individual numbers within that range, for example, 1, 2, 2.7, 3, 4, 5, and 6. This applies regardless of the breadth of the range. Unless otherwise explicitly stated to the contrary, a range that is disclosed also includes the endpoints of the range.

The term “composition” as used herein means a product which results from the mixing or combining of more than one element or ingredient.

As used herein, the term “pharmaceutical composition” refers to a medicinal or pharmaceutical formulation that contains an active ingredient as well as one or more excipients and diluents to enable the active ingredient suitable for the method of administration.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer.

The phrase “pharmaceutically acceptable” is employed herein to refer to those agents of interest, compounds, salts, compositions, pharmaceutical dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some embodiments, pharmaceutical acceptable means approved by a regulatory agency of the federal or a state government, or listed in the U.S. Pharmacopeia or other generally recognized pharmacopeia for the use in animals (e.g., mammals), and more particularly, in humans.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. The nature of the carrier differs with the mode of administration. For example, for intravenous administration, an aqueous solution carrier is generally used; for oral administration, a solid carrier is generally used.

As used herein, “stable” a “stable composition” or a “stable pharmaceutical composition” refers to a composition or pharmaceutical composition that maintains one or more of the characteristics of the composition within a defined margin when subject to various stressors such as, but not limited to, heat, agitation, light, temperature, humidity, repeated freeze thaw cycles, and extended storage. Such characteristics include, but are not limited to, pH, presence of aggregates, functional titer, or concentration of protein excipient. In some embodiments, the defined margins for pH, presence of aggregates, functional titer, or concentration of protein excipient are as provided for herein.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. An antibody consists of four polypeptide chains; two heavy chains (C_{H}1 and C_{H}2) and two light chains (C_{L}1 and C_{L}2) connected by disulfide bonds. Each chain is a series of domains: light chains consist of one variable domain (V_{L}) and one constant domain (C_{L}), while heavy chains contain one variable domain (V_{H}) and three to four constant domains (C_{H}1, C_{H}2, C_{H}3, C_{H}4). “Parental antibodies” are antibodies obtained by exposure of an immune system to an antigen prior to modification of the antibodies for an intended use, such as humanization of an antibody for use as a human therapeutic antibody.

As used herein, unless otherwise indicated, “antibody fragment” or “antigen binding fragment” refers to antigen binding fragments of antibodies, i.e., antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, Fv fragments, diabodies, linear antibodies, single-chain antibody molecules, e.g., sc-Fv, nanobodies and multispecific antibodies formed from antibody fragments.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds, and by hydrophobic interactions of the C_{H}3 domains.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as a glycine/serine, glycine/alanine linker, or other types of peptide linkers generally known to link the two domains together. Two peptide sequences are linked directly if they are directly connected to one another, or indirectly if there is a linker or other structure that links the two regions. A linker can be directly linked to two different peptide sequences or domains.

A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315; International Patent Application Publication No. WO 88/01649; and U.S. Pat. Nos. 4,946,778 and 5,260,203.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

A “single-domain antibody” is an immunologically functional immunoglobulin fragment containing only the variable region of a heavy chain or the variable region of a light chain. In some instances, two or more V_{H} regions are covalently joined with a peptide linker to create a bivalent domain antibody.

In some embodiments, as provided for herein, antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

In some embodiments, monoclonal antibodies herein also include camelized single domain antibodies (see, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079)). In some embodiments, the present disclosure provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

Typically, a variant antibody or antigen binding fragment of the antibodies provided herein retain at least 10% of its C1s binding activity (when compared to a parental antibody that is modified) when that activity is expressed on a molar basis. In some embodiments, a variant antibody, or antigen fragment thereof, or antigen binding fragment of an antibody provided herein, retains at least 20%, 50%, 70%, 80%, 90%, 95% or 100% or more of the C1s binding affinity as the parental antibody. As described herein, it is also intended that an antibody or antigen binding fragment of the disclosure can include conservative or non-conservative amino acid substitutions, which can also be referred to as “conservative variants” or “function conserved variants” of the antibody, that do not substantially alter its biologic activity.

“Isolated antibody” refers to the purification status of a binding compound and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to population of substantially homogeneous antibodies, i.e., the antibody molecules comprising the population are identical in amino acid sequence except for possible naturally occurring mutations and/or post-translational modifications that may be present in minor amounts. In contrast, conventional (polyclonal) antibody preparations typically include a multitude of different antibodies having different amino acid sequences in their variable domains, particularly their CDRs, that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991)Nature 352: 624-628 and Marks et al. (1991) J Mol. Biol. 222: 581-597, for example. See also Presta (2005) J. Allergy Clin. Immunol. 116:731.

As used herein, a “chimeric antibody” is an antibody having the variable domain from a first antibody and constant domain from a second antibody, where the first and second antibodies are from different species. (U.S. Pat. No. 4,816,567; and Morrison et al., (1984) Proc. Natl. Acad. Sci. USA 81: 6851-6855). Typically, the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g. rodent) antibody.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

The term “fully human antibody” refers to an antibody that comprises human immunoglobulin protein sequences only. A fully human antibody may contain murine carbohydrate chains if produced in a mouse, a mouse cell, or a hybridoma derived from a mouse cell. Similarly, “mouse antibody” refers to an antibody that comprises mouse immunoglobulin sequences only. Alternatively, a fully human antibody may contain rat carbohydrate chains if produced in a rat, a rat cell, or a hybridoma derived from a rat cell. Similarly, “rat antibody” refers to an antibody that comprises rat immunoglobulin sequences only.

In some embodiments, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. However, in bifunctional or bispecific antibodies, the two binding sites are, in general, not the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain; Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (CDRL1), 50-52 (CDRL2) and 91-96 (CDRL3) in the light chain variable domain and 26-32 (CDRH1), 53-55 (CDRH2) and 96-101 (CDRH3) in the heavy chain variable domain (Chothia and Lesk (1987) J. Mol. Biol. 196: 901-917)). The CDRs can also be referenced according to the IMGT system for the identification of CDRs, which is described in Lefranc MP. Unique database numbering system for immunogenetic analysis. (Immunol Today (1997) 18:509).

As used herein, the term “framework” or “FR” residues refers to those variable domain residues other than the hypervariable region residues defined herein as CDR residues. CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen binding specificity and/or neutralizing ability as the donor antibody from which they were derived.

As used herein, “specific binding” or “immunospecific binding” or “binds immunospecifically” refer to antibody binding to a predetermined antigen at a much higher affinity than for another antigen(s) (e.g. selectively binds the active form of complement component C1s as compared to inactive C1s, which can also be referred to as proC1s zymogen). In some embodiments, the antibody binds the predetermined antigen with a dissociation constant (K_{D}) of 10⁻⁷ M or less, and such K_{D} is at least two-fold less than its K_{D} for binding to a non-specific antigen (e.g., BSA, casein, or another non-specific polypeptide).

The phrases “an antibody recognizing C1s” and “an antibody specific for C1s” are used interchangeably herein with the term “an antibody which binds immunospecifically to C1s”. In some embodiments, the antibody binds specifically or preferentially to C1s, such as the active form of C1s over other proteins, such as, but not limited to, the inactive form of C1s (proC1s). The degree of specificity necessary for an anti-C1s antibody may depend on the intended use of the antibody, and at any rate is defined by its suitability for use for an intended purpose. In some embodiments, the antibody, or binding compound derived from the antigen-binding site of an antibody, of the contemplated method binds to its antigen (active form of C1s), with an affinity that is at least two-fold greater, at least ten times greater, at least 20-times greater, or at least 100-times greater than the affinity with any other antigen, including, but not limited to inactive C1s.

Methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589 601 (1983), which references are entirely incorporated herein by reference.

The term “homolog” means protein sequences having between 40% and 100% sequence identity to a reference sequence. Percent identity between two peptide chains can be determined by pair wise alignment using the default settings of the AlignX module of Vector NTI v.9.0.0 (Invitrogen Corp., Carlsbad, Calif.) or other suitable alignment software, such as BLAST. In some embodiments, the antibody, or antigen binding fragment thereof has, at least 50, 60, 70, 80, 90, 91, 92, 93, 94, 95, 96, 97, 98, or 99% homology or identity to a sequence described herein. In some embodiments, the antibody has conservative substitutions as compared to a sequence described herein. Exemplary conservative substitutions are illustrated in Table 1 and are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in the table below.

TABLE 1
Exemplary Conservative Substitutions

| Original | Exemplary Conservative |
|---|---|
| Residue | Substitutions |
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, a variant comprises a substitution, deletions, or insertion. In some embodiments, the variant comprises 1, 2, 3, 4, 5, 6, 7, 8, 9, or 10 (e.g., 1-10) substitutions. As described herein, the substitutions can be conservative substitutions. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein.

The term “in combination with” as used herein means that the described agents can be administered to an animal or subject together in a mixture, concurrently as single agents or sequentially as single agents in any order.

The term “epitope” is meant to refer to that portion of any molecule capable of being recognized by and bound by an antibody at one or more of the Ab's antigen binding regions. Epitopes usually consist of chemically active surface groupings of molecules such as amino acids or sugar side chains and have specific three-dimensional structural characteristics as well as specific charge characteristics.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

In some embodiments, the antibody is a monoclonal antibody which binds to CIs. The sequence of active CIs is as follows (SEQ ID NO: 258), and residues 1-15 (SEQ ID NO: 259) constitute the signal peptide which is cleaved during translation, while residues 16-688 (SEQ ID NO: 260) constitute the mature protein:

(SEQ ID NO: 258)
MWCIVLFSLLAWVYAEPTMYGEILSPNYPQAYPSEVEKSWDIEVPEGYGI
HLYFTHLDIELSENCAYDSVQIISGDTEEGRLCGQRSSNNPHSPIVEEFQ
VPYNKLQVIFKSDFSNEERFTGFAAYYVATDINECTDFVDVPCSHFCNNF
IGGYFCSCPPEYFLHDDMKNCGVNCSGDVFTALIGEIASPNYPKPYPENS
RCEYQIRLEKGFQVVVTLRREDFDVEAADSAGNCLDSLVFVAGDRQFGPY
CGHGFPGPLNIETKSNALDIIFQTDLTGQKKGWKLRYHGDPMPCPKEDTP
NSVWEPAKAKYVFRDVVQITCLDGFEVVEGRVGATSFYSTCQSNGKWSNS
KLKCQPVDCGIPESIENGKVEDPESTLFGSVIRYTCEEPYYYMENGGGGE
YHCAGNGSWVNEVLGPELPKCVPVCGVPREPFEEKQRIIGGSDADIKNFP
WQVFFDNPWAGGALINEYWVLTAAHVVEGNREPTMYVGSTSVQTSRLAKS
KMLTPEHVFIHPGWKLLEVPEGRTNFDNDIALVRLKDPVKMGPTVSPICL
PGTSSDYNLMDGDLGLISGWGRTEKRDRAVRLKAARLPVAPLRKCKEVKV
EKPTADAEAYVFTPNMICAGGEKGMDSCKGDSGGAFAVQDPNDKTKFYAA
GLVSWGPQCGTYGLYTRVKNYVDWIMKTMQENSTPRED;
(SEQ ID NO: 259)
MWCIVLFSLLAWVYA;
(SEQ ID NO: 260)
EPTMYGEILSPNYPQAYPSEVEKSWDIEVPEGYGIHLYFTHLDIELSENC
AYDSVQIISGDTEEGRLCGQRSSNNPHSPIVEEFQVPYNKLQVIFKSDFS
NEERFTGFAAYYVATDINECTDFVDVPCSHFCNNFIGGYFCSCPPEYFLH
DDMKNCGVNCSGDVFTALIGEIASPNYPKPYPENSRCEYQIRLEKGFQVV
VTLRREDFDVEAADSAGNCLDSLVFVAGDRQFGPYCGHGFPGPLNIETKS
NALDIIFQTDLTGQKKGWKLRYHGDPMPCPKEDTPNSVWEPAKAKYVFRD
VVQITCLDGFEVVEGRVGATSFYSTCQSNGKWSNSKLKCQPVDCGIPESI
ENGKVEDPESTLFGSVIRYTCEEPYYYMENGGGGEYHCAGNGSWVNEVLG
PELPKCVPVCGVPREPFEEKQRIIGGSDADIKNFPWQVFFDNPWAGGALI
NEYWVLTAAHVVEGNREPTMYVGSTSVQTSRLAKSKMLTPEHVFIHPGWK
LLEVPEGRTNFDNDIALVRLKDPVKMGPTVSPICLPGTSSDYNLMDGDLG
LISGWGRTEKRDRAVRLKAARLPVAPLRKCKEVKVEKPTADAEAYVFTPN
MICAGGEKGMDSCKGDSGGAFAVQDPNDKTKFYAAGLVSWGPQCGTYGLY
TRVKNYVDWIMKTMQENSTPRED.

The difference between inactive C1s and active C1s is that inactive proC1s is cleaved at the peptide bond between R437 and 1438 and undergoes a conformational change. The two fragments generated by this cleavage remain associated by a disulfide bond. Without wishing to be bound by a particular theory, proC1s is a single chain 86,000 Da protein that is the native form of C1s proteins (e.g., serine protease). Cls is a subunit of the C1 complex which is the first complement component in the cascade referred to as the classical pathway of complement. ProC1s is an inactive zymogen until C1 is activated. C1 complex binds to and is activated by antigen-antibody complexes (immune complexes) yielding C1r enzyme. Cir enzyme in the C1 complex activates proC1s generating C1s enzyme. C1 complex is a non-covalent calcium-dependent complex of one C1q, two C1r and two C1s molecules. C1q binds through two or more of its six arms to the Fc domains of IgG or IgM. The binding of multiple arms to immune complexes causes the two C1r proteins in the complex (protease zymogens) to activate producing two proteases that cleave and activate the two proC1s in the complex (Morikis, D. and Lambris, J. D. (2005)). This activation of proC1s is caused by cleavage into the two chain C1s enzyme with 58,000 and 28,000 Dalton fragments.

In some embodiments, antibodies (e.g. an anti-C1s antibody) are provided herein. In some embodiments, the antibody is a recombinant antibody that binds to C1s. In some embodiments, the antibody binds to the active form of C1s. In some embodiments, the antibody binds to active form preferentially over the inactive form of C1s. In some embodiments, the antibody binds to the active form with an affinity that is at least 10, 20, 30, 40, 50, 60, 70, 80, 90, 100, or 200% higher as compared to its affinity for the inactive form of C1s. In some embodiments, the C1s protein is a human C1s protein, such as the active form of C1s. In some embodiments, the antibody does not specifically bind to the inactive form of the C1s protein. As used herein, the term “recombinant antibody” refers to an antibody that is not naturally occurring. In some embodiments, the term “recombinant antibody” refers to an antibody that is not isolated from a human subject. In some embodiments, the antibody binds with at least 100× more affinity to the active form of C1s as compared to proC1s.

In some embodiments, an antibody, or antigen binding fragment thereof is provided, wherein the antibody or antibody fragment comprises a peptide selected from the following table, which illustrate the CDRs based on Kabat numbering.

Kabat CDRs

| Ab ID No | HCDR1 | HCDR2 | HCDR3 | LCDR1 | LCDR2 | LCDR3 |
|---|---|---|---|---|---|---|
| MAB1 | DYYMS | YISRSGSTKY | DQEDYALDY | QASQDISNY | DASNLET (SEQ ID | EQYEDYPLT |
| | (SEQ ID | YADSVKG | (SEQ ID NO: | LN (SEQ ID | NO: 65) | (SEQ ID NO: |
| | NO: 61) | (SEQ ID NO: | 63) | NO: 64) | | 66) |
| | | 62) | | | | |
| MAB2 | DYYMS | YISRSGSTKY | DETDYGWDY | QASQDISNY | DASNLET (SEQ ID | KQYEDYPLT |
| | (SEQ ID | YADSVKG | (SEQ ID NO: | LN (SEQ ID | NO: 65) | (SEQ ID NO: |
| | NO: 61) | (SEQ ID NO: | 67) | NO: 64) | | 68) |
| | | 62) | | | | |
| MAB3 | DYYMS | YISRSGSTKY | DETDYAFDE | QASQDISNY | DASNLET (SEQ ID | KQYEDYPLT |
| | (SEQ ID | YADSVKG | (SEQ ID NO: | LN (SEQ ID | NO: 65) | (SEQ ID NO: |
| | NO: 61) | (SEQ ID NO: | 69) | NO: 64) | | 68) |
| | | 62) | | | | |
| MAB4 | DYYMS | YISRSGSTKY | DESQYALDY | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 70) | NO: 64) | | 71) |
| | | 62) | | | | |
| MAB5 | DYYMS | YISRSGSTKY | DNTDYALDL | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 72) | NO: 64) | | 71) |
| | | 62) | | | | |
| MAB6 | DYYMS | YISRSGSTKY | DETDYAYDE | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 73) | NO: 64) | | 71) |
| | | 62) | | | | |
| MAB7 | DYYMS | YISRSGSTKY | DETDYAYDN | QASQDISNY | DASNLET (SEQ ID | QQYEDYPLV |
| | (SEQ ID | YADSVKG | (SEQ ID NO: | LN (SEQ ID | NO: 65) | (SEQ ID NO: |
| | NO: 61) | (SEQ ID NO: | 74) | NO: 64) | | 75) |
| | | 62) | | | | |
| MAB8 | DYYMS | YISRSGSTKY | DESDYAYDY | QASQDISNY | DASNLET (SEQ ID | QQHEDYPL |
| | (SEQ ID | YADSVKG | (SEQ ID NO: | LN (SEQ ID | NO: 65) | (SEQ ID NO: |
| | NO: 61) | (SEQ ID NO: | 76) | NO: 64) | | 77) |
| | | 62) | | | | |
| MAB9 | DYYMS | YISRSGSTKY | DENYALDW | QASQDISNY | DASNLET (SEQ ID | QHYEDYPL |
| | (SEQ ID | YADSVKG | (SEQ ID NO: | LN (SEQ ID | NO: 65) | (SEQ ID NO: |
| | NO: 61) | (SEQ ID NO: | 78) | NO: 64) | | 79) |
| | | 62) | | | | |
| MAB10 | DYYMS | YISRSGSTKY | DESDYALDF | QASQDISNY | DASNLET (SEQ ID | QQYEDLIPT |
| | (SEQ ID | YADSVKG | (SEQ ID NO: | LN (SEQ ID | NO: 65) | (SEQ ID NO: |
| | NO: 61) | (SEQ ID NO: | 80) | NO: 64) | | 81) |
| | | 62) | | | | |
| MAB11 | ELSMH | TFDPEEGETI | EGLAGVPFDL | RASQSISSW | KASSLES (SEQ ID | QSYNSYVWT |
| | (SEQ ID | YAQKFQG | (SEQ ID NO: | LA (SEQ ID | NO: 86) | (SEQ ID NO: |
| | NO: 82) | (SEQ ID NO: | 84) | NO: 85) | | 87) |
| | | 83) | | | | |
| MAB12 | ELSMH | TFDPEEGETI | EGLGGRPFDH | RASQSISSW | KASSLES (SEQ ID | QQVNSYDWT |
| | (SEQ ID | YAQKFQG | (SEQ ID NO: | LA (SEQ ID | NO: 86) | (SEQ ID NO |
| | NO: 82) | (SEQ ID NO: | 88) | NO: 85) | | 89) |
| | | 83 | | | | |
| MAB13 | ELSMH | TFDPEEGETI | EGLAGFPFDI | RASQSISSW | KASSLES (SEQ ID | QQVNSYDWT |
| | (SEQ ID | YAQKFQG | (SEQ ID NO: | LA (SEQ ID | NO: 86) | (SEQ ID NO |
| | NO: 82) | (SEQ ID NO: | 90) | NO: 85) | | 89) |
| | | 83 | | | | |
| MAB14 | ELSMH | TFDPEEGETI | EGLAWRPTDS | RASQSISSW | KASSLES (SEQ ID | QQYSSYAWT |
| | (SEQ ID | YAQKFQG | (SEQ ID NO: | LA (SEQ ID | NO: 86) | (SEQ ID NO: |
| | NO: 82) | (SEQ ID NO: | 91) | NO: 85) | | 92) |
| | | 83 | | | | |
| MAB15 | ELSMH | TFDPEEGETI | KGLAWLPYY | RASQSISSW | KASSLES (SEQ ID | QQARSYSWT |
| | (SEQ ID | YAQKFQG | S (SEQ ID NO: | LA (SEQ ID | NO: 86) | (SEQ ID NO: |
| | NO: 82) | (SEQ ID NO: | 93) | NO: 85) | | 94) |
| | | 83 | | | | |
| MAB16 | ELSMH | TFDPEEGETI | EGLAGVPFDL | RASQSISSW | KASSLES (SEQ ID | QQHNSYRWT |
| | (SEQ ID | YAQKFQG | (SEQ ID NO: | LA (SEQ ID | NO: 86) | (SEQ ID NO: |
| | NO: 82) | (SEQ ID NO: | 84) | NO: 85) | | 95) |
| | | 83 | | | | |
| MAB17 | ELSMH | TFDPEEGETI | EGEAGRPFDA | RASQSISSW | KASSLES (SEQ ID | VQYPSYSWT |
| | (SEQ ID | YAQKFQG | (SEQ ID NO: | LA (SEQ ID | NO: 86) | (SEQ ID NO: |
| | NO: 82) | (SEQ ID NO: | 96) | NO: 85) | | 97) |
| | | 83 | | | | |
| MAB18 | ELSMH | TFDPEEGETI | EGLAGRPYDV | RASQSISSW | KASSLES (SEQ ID | QQYNSYKLT |
| | (SEQ ID | YAQKFQG | (SEQ ID NO: | LA (SEQ ID | NO: 86) | (SEQ ID NO: |
| | NO: 82) | (SEQ ID NO: | 98) | NO: 85) | | 99) |
| | | 83 | | | | |
| MAB19 | ELSMH | TFDPEEGETI | EGLAGIPFDS | RASQSISSW | KASSLES (SEQ ID | QQYNSPSWL |
| | (SEQ ID | YAQKFQG | W (SEQ ID NO: | LA (SEQ ID | NO: 86) | (SEQ ID NO: |
| | NO: 82) | (SEQ ID NO: | 100) | NO: 85) | | 101) |
| | | 83 | | | | |
| MAB20 | ELSMH | TFDPEEGETI | EGLAGIPFDS | RASQSISSW | KASSLES (SEQ ID | QQVNSLSWT |
| | (SEQ ID | YAQKFQG | W (SEQ ID NO: | LA (SEQ ID | NO: 86) | (SEQ ID NO: |
| | NO: 82) | (SEQ ID NO: | 100) | NO: 85) | | 102) |
| | | 83 | | | | |
| MAB21 | DYGMS | GINWEGGST | DEQLGGTTYY | RASQGIRN | TASNLQS (SEQ ID | FQYNSYPLG |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 108) |
| | | 104) | 105) | | | |
| MAB22 | DYGMS | GINWEGGST | DEQLGGKYY | RASQGIRN | TASNLQS (SEQ ID | LQYRSHPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 110) |
| | | 104) | 109) | | | |
| MAB23 | DYGMS | GINWEGGST | DEQLGGQTY | RASQGIRN | TASNLQS (SEQ ID | LQYNQVPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 112) |
| | | 104) | 111) | | | |
| MAB24 | DYGMS | GINWEGGST | DEQLGGTTYY | RASQGIRN | TASNLQS (SEQ ID | LQTNIYPLT |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 113) |
| | | 104) | 105) | | | |
| MAB25 | DYGMS | GINWEGGST | DEQLGGIKYY | RASQGIRN | TASNLQS (SEQ ID | LQYKQYPLT |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 115) |
| | | 104) | 114) | | | |
| MAB26 | DYGMS | GINWEGGST | DEQLGGLKY | RASQGIRN | TASNLQS (SEQ ID | LQTNIYPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 113) |
| | | 104) | 116) | | | |
| MAB27 | DYGMS | GINWEGGST | DEQLGGKVY | RASQGIRN | TASNLQS (SEQ ID | LQYNSYPLA |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 118) |
| | | 104) | 117) | | | |
| MAB28 | DYGMS | GINWEGGST | DEQLGGLKY | RASQGIRN | TASNLQS (SEQ ID | LQYHSYPLR |
| | (SEQ ID | GYADSVKG | YYYYMDV | DLG (SEQ | NO: 107) | (SEQ ID NO: |
| | NO: 103) | (SEQ ID NO: | (SEQ ID NO | ID NO: 106) | | 119) |
| | | 104) | 116) | | | |
| MAB29 | DYGMS | GINWEGGST | DEQLGGNHY | RASQGIRN | TASNLQS (SEQ ID | LQYASYPLK |
| | (SEQ ID | GYADSVKG | YYYYMDA | DLG (SEQ | NO: 107) | (SEQ ID NO: |
| | NO: 103) | (SEQ ID NO: | (SEQ ID NO: | ID NO: 106) | | 121) |
| | | 104) | 120) | | | |
| MAB30 | DYGMS | GINWEGGST | DEQLGGRHY | RASQGIRN | TASNLQS (SEQ ID | LQYNAYPLI |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 123) |
| | | 104) | 122) | | | |
| MAB31 | DYYMS | YISRSGSTKY | DQEDYALDY | QASQDISNY | DASNLET (SEQ ID | EQYEDYPLT |
| | (SEQ ID | YADSVKG | (SEQ ID NO: | LN (SEQ ID | NO: 65) | (SEQ ID NO: |
| | NO: 61) | (SEQ ID NO: | 63) | NO: 64) | | 66) |
| | | 62) | | | | |
| MAB32 | DYYMS | YISRSGSTKY | DETDYGWDY | QASQDISNY | DASNLET (SEQ ID | KQYEDYPLT |
| | (SEQ ID | YADSVKG | (SEQ ID NO: | LN (SEQ ID | NO: 65) | (SEQ ID NO: |
| | NO: 61) | (SEQ ID NO: | 67) | NO: 64) | | 68) |
| | | 62) | | | | |
| MAB33 | DYYMS | YISRSGSTKY | DETDYAFDE | QASQDISNY | DASNLET (SEQ ID | KQYEDYPLT |
| | (SEQ ID | YADSVKG | (SEQ ID NO: | LN (SEQ ID | NO: 65) | (SEQ ID NO: |
| | NO: 61) | (SEQ ID NO: | 69) | NO: 64) | | 68) |
| | | 62) | | | | |
| MAB34 | DYYMS | YISRSGSTKY | DESQYALDY | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 70) | NO: 64) | | 71) |
| | | 62) | | | | |
| MAB35 | DYYMS | YISRSGSTKY | DNTDYALDL | QASQDISNY | DASNLET (SEQ ID | HQYEDYPLT |
| | (SEQ ID | YADSVKG | (SEQ ID NO: | LN (SEQ ID | NO: 65) | (SEQ ID NO: |
| | NO: 61) | (SEQ ID NO: | 72) | NO: 64) | | 71) |
| | | 62) | | | | |
| MAB36 | DYYMS | YISRSGSTKY | DETDYAYDE | QASQDISNY | DASNLET (SEQ ID | HQYEDYPLT |
| | (SEQ ID | YADSVKG | (SEQ ID NO: | LN (SEQ ID | NO: 65) | (SEQ ID NO |
| | NO: 61) | (SEQ ID NO: | 73) | NO: 64) | | 71) |
| | | 62) | | | | |
| MAB37 | DYYMS | YISRSGSTKY | DETDYAYDN | QASQDISNY | DASNLET (SEQ ID | QQYEDYPLV |
| | (SEQ ID | YADSVKG | (SEQ ID NO: | LN (SEQ ID | NO: 65) | (SEQ ID NO: |
| | NO: 61) | (SEQ ID NO: | 74) | NO: 64) | | 75) |
| | | 62) | | | | |
| MAB38 | DYYMS | YISRSGSTKY | DESDYAYDY | QASQDISNY | DASNLET (SEQ ID | QQHEDYPL |
| | (SEQ ID | YADSVKG | (SEQ ID NO: | LN (SEQ ID | NO: 65) | (SEQ ID NO: |
| | NO: 61) | (SEQ ID NO: | 76) | NO: 64) | | 77) |
| | | 62) | | | | |
| MAB39 | DYYMS | YISRSGSTKY | DENYALDW | QASQDISNY | DASNLET (SEQ ID | QHYEDYPL |
| | (SEQ ID | YADSVKG | (SEQ ID NO: | LN (SEQ ID | NO: 65) | (SEQ ID NO: |
| | NO: 61) | (SEQ ID NO: | 78) | NO: 64) | | 79) |
| | | 62) | | | | |
| MAB40 | DYYMS | YISRSGSTKY | DESDYALDF | QASQDISNY | DASNLET (SEQ ID | QQYEDLIPT |
| | (SEQ ID | YADSVKG | (SEQ ID NO: | LN (SEQ ID | NO: 65) | (SEQ ID NO: |
| | NO: 61) | (SEQ ID NO: | 80) | NO: 64) | | 81) |
| | | 62) | | | | |
| MAB41 | ELSMH | TFDPEEGETI | EGLAGVPFDL | RASQSISSW | KASSLES (SEQ ID | QSYNSYVWT |
| | (SEQ ID | YAQKFQG | (SEQ ID NO: | LA (SEQ ID | NO: 86) | (SEQ ID NO: |
| | NO: 82) | (SEQ ID NO: | 84) | NO: 85) | | 87) |
| | | 83) | | | | |
| MAB42 | ELSMH | TFDPEEGETI | EGLGGRPFDH | RASQSISSW | KASSLES (SEQ ID | QQVNSYDWT |
| | (SEQ ID | YAQKFQG | (SEQ ID NO: | LA (SEQ ID | NO: 86) | (SEQ ID NO |
| | NO: 82) | (SEQ ID NO: | 88) | NO: 85) | | 89) |
| | | 83 | | | | |
| MAB43 | ELSMH | TFDPEEGETI | EGLAGFPFDI | RASQSISSW | KASSLES (SEQ ID | QQVNSYDWT |
| | (SEQ ID | YAQKFQG | (SEQ ID NO: | LA (SEQ ID | NO: 86) | (SEQ ID NO |
| | NO: 82) | (SEQ ID NO: | 90) | NO: 85) | | 89) |
| | | 83 | | | | |
| MAB44 | ELSMH | TFDPEEGETI | EGLAWRPTDS | RASQSISSW | KASSLES (SEQ ID | QQYSSYAWT |
| | (SEQ ID | YAQKFQG | (SEQ ID NO: | LA (SEQ ID | NO: 86) | (SEQ ID NO: |
| | NO: 82) | (SEQ ID NO: | 91) | NO: 85) | | 92) |
| | | 83 | | | | |
| MAB45 | ELSMH | TFDPEEGETI | KGLAWLPYY | RASQSISSW | KASSLES (SEQ ID | QQARSYSWT |
| | (SEQ ID | YAQKFQG | S (SEQ ID NO: | LA (SEQ ID | NO: 86) | (SEQ ID NO: |
| | NO: 82) | (SEQ ID NO: | 93) | NO: 85) | | 94) |
| | | 83 | | | | |
| MAB46 | ELSMH | TFDPEEGETI | EGLAGVPFDL | RASQSISSW | KASSLES (SEQ ID | QQHNSYRWT |
| | (SEQ ID | YAQKFQG | (SEQ ID NO: | LA (SEQ ID | NO: 86) | (SEQ ID NO: |
| | NO: 82) | (SEQ ID NO: | 84) | NO: 85) | | 95) |
| | | 83 | | | | |
| MAB47 | ELSMH | TFDPEEGETI | EGEAGRPFDA | RASQSISSW | KASSLES (SEQ ID | VQYPSYSWT |
| | (SEQ ID | YAQKFQG | (SEQ ID NO: | LA (SEQ ID | NO: 86) | (SEQ ID NO: |
| | NO: 82) | (SEQ ID NO: | 96) | NO: 85) | | 97) |
| | | 83 | | | | |
| MAB48 | ELSMH | TFDPEEGETI | EGLAGRPYDV | RASQSISSW | KASSLES (SEQ ID | QQYNSYKLT |
| | (SEQ ID | YAQKFQG | (SEQ ID NO: | LA (SEQ ID | NO: 86) | (SEQ ID NO: |
| | NO: 82) | (SEQ ID NO: | 98) | NO: 85) | | 99) |
| | | 83 | | | | |
| MAB49 | ELSMH | TFDPEEGETI | EGLAGIPFDS | RASQSISSW | KASSLES (SEQ ID | QQYNSPSWL |
| | (SEQ ID | YAQKFQG | W (SEQ ID NO: | LA (SEQ ID | NO: 86) | (SEQ ID NO: |
| | NO: 82) | (SEQ ID NO: | 100) | NO: 85) | | 101) |
| | | 83 | | | | |
| MAB50 | ELSMH | TFDPEEGETI | EGLAGIPFDS | RASQSISSW | KASSLES (SEQ ID | QQVNSLSWT |
| | (SEQ ID | YAQKFQG | W (SEQ ID NO: | LA (SEQ ID | NO: 86) | (SEQ ID NO: |
| | NO: 82) | (SEQ ID NO: | 100) | NO: 85) | | 102) |
| | | 83 | | | | |
| MAB51 | DYGMS | GINWEGGST | DEQLGGTTYY | RASQGIRN | TASNLQS (SEQ ID | FQYNSYPLG |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 108) |
| | | 104) | 105) | | | |
| MAB52 | DYGMS | GINWEGGST | DEQLGGKYY | RASQGIRN | TASNLQS (SEQ ID | LQYRSHPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 110) |
| | | 104) | 109) | | | |
| MAB53 | DYGMS | GINWEGGST | DEQLGGQTY | RASQGIRN | TASNLQS (SEQ ID | LQYNQVPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 112) |
| | | 104) | 111) | | | |
| MAB54 | DYGMS | GINWEGGST | DEQLGGTTYY | RASQGIRN | TASNLQS (SEQ ID | LQTNIYPLT |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 113) |
| | | 104) | 105) | | | |
| MAB55 | DYGMS | GINWEGGST | DEQLGGIKYY | RASQGIRN | TASNLQS (SEQ ID | LQYKQYPLT |
| | (SEQ ID | GYADSVKG | YYYMDV | DLG (SEQ | NO: 107) | (SEQ ID NO: |
| | NO: 103) | (SEQ ID NO: | (SEQ ID NO: | ID NO: 106) | | 115) |
| | | 104) | 114) | | | |
| MAB56 | DYGMS | GINWEGGST | DEQLGGLKY | RASQGIRN | TASNLQS (SEQ ID | LQTNIYPLT |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 113) |
| | | 104) | 116) | | | |
| MAB57 | DYGMS | GINWEGGST | DEQLGGKVY | RASQGIRN | TASNLQS (SEQ ID | LQYNSYPLA |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 118) |
| | | 104) | 117) | | | |
| MAB58 | DYGMS | GINWEGGST | DEQLGGLKY | RASQGIRN | TASNLQS (SEQ ID | LQYHSYPLR |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 119) |
| | | 104) | 116) | | | |
| MAB59 | DYGMS | GINWEGGST | DEQLGGNHY | RASQGIRN | TASNLQS (SEQ ID | LQYASYPLK |
| | (SEQ ID | GYADSVKG | YYYYMDA | DLG (SEQ | NO: 107) | (SEQ ID NO: |
| | NO: 103) | (SEQ ID NO: | (SEQ ID NO: | ID NO: 106) | | 121) |
| | | 104) | 120) | | | |
| MAB60 | DYGMS | GINWEGGST | DEQLGGRHY | RASQGIRN | TASNLQS (SEQ ID | LQYNAYPLI |
| | (SEQ ID | GYADSVKG | YYYYMDV | DLG (SEQ | NO: 107) | (SEQ ID NO: |
| | NO: 103) | (SEQ ID NO: | (SEQ ID NO: | ID NO: 106) | | 123) |
| | | 104) | 122) | | | |

The CDRs in the above-identified table can also be referred to according to Chothia CDRs or IMGT CDRs. These are illustrated in the following tables for the different antibodies.

CHOTHIA CDRS

| Ab ID No | HCDR1 | HCDR2 | HCDR3 | LCDR1 | LCDR2 | LCDR3 |
|---|---|---|---|---|---|---|
| MAB1 | GFTFSDY | SRSGST | DQEDYALDY | QASQDISNY | DASNLET (SEQ ID | EQYEDYPLT |
| | (SEQ ID NO: | (SEQ ID | (SEQ ID NO: | LN (SEQ ID | NO: 65) | (SEQ ID NO: |
| | 124) | NO: 125) | 63) | NO: 64) | | 66) |
| MAB2 | GFTFSDY | SRSGST | DETDYGWDY | QASQDISNY | DASNLET (SEQ ID | KQYEDYPLT |
| | (SEQ ID NO: | (SEQ ID | (SEQ ID NO: | LN (SEQ ID | NO: 65) | (SEQ ID NO: |
| | 124) | NO: 125) | 67) | NO: 64) | | 68) |
| MAB3 | GFTFSDY | SRSGST | DETDYAFDE | QASQDISNY | DASNLET (SEQ ID | KQYEDYPLT |
| | (SEQ ID NO: | (SEQ ID | (SEQ ID NO: | LN (SEQ ID | NO: 65) | (SEQ ID NO: |
| | 124) | NO: 125) | 69) | NO: 64) | | 68) |
| MAB4 | GFTFSDY | SRSGST | DESQYALDY | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 70) | NO: 64) | | 71) |
| MAB5 | GFTFSDY | SRSGST | DNTDYALDL | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 72) | NO: 64) | | 71) |
| MAB6 | GFTFSDY | SRSGST | DETDYAYDE | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 73) | NO: 64) | | 71) |
| MAB7 | GFTFSDY | SRSGST | DETDYAYDN | QASQDISNY | DASNLET (SEQ ID | QQYEDYPLV |
| | (SEQ ID NO: | (SEQ ID | (SEQ ID NO: | LN (SEQ ID | NO: 65) | (SEQ ID NO: |
| | 124) | NO: 125) | 74) | NO: 64) | | 75) |
| MAB8 | GFTFSDY | SRSGST | DESDYAYDY | QASQDISNY | DASNLET (SEQ ID | QQHEDYPL |
| | (SEQ ID NO: | (SEQ ID | (SEQ ID NO: | LN (SEQ ID | NO: 65) | (SEQ ID NO: |
| | 124) | NO: 125) | 76) | NO: 64) | | 77) |
| MAB9 | GFTFSDY | SRSGST | DENYALDW | QASQDISNY | DASNLET (SEQ ID | QHYEDYPL |
| | (SEQ ID NO: | (SEQ ID | (SEQ ID NO: | LN (SEQ ID | NO: 65) | (SEQ ID NO: |
| | 124) | NO: 125) | 78) | NO: 64) | | 79) |
| MAB10 | GFTFSDY | SRSGST | DESDYALDF | QASQDISNY | DASNLET (SEQ ID | QQYEDLIPT |
| | (SEQ ID NO: | (SEQ ID | (SEQ ID NO: | LN (SEQ ID | NO: 65) | (SEQ ID NO: |
| | 124) | NO: 125) | 80) | NO: 64) | | 81) |
| MAB11 | GDTLTEL | DPEEGE | EGLAGVPFDL | RASQSISSW | KASSLES (SEQ ID | QSYNSYVWT |
| | (SEQ ID NO: | (SEQ ID | (SEQ ID NO: | LA (SEQ ID | NO: 86) | (SEQ ID NO: |
| | 126) | NO: 127) | 84) | NO: 85) | | 87) |
| MAB12 | GDTLTEL | DPEEGE | EGLGGRPFDH | RASQSISSW | KASSLES (SEQ ID | QQVNSYDWT |
| | (SEQ ID NO: | (SEQ ID | (SEQ ID NO: | LA (SEQ ID | NO: 86) | (SEQ ID NO |
| | 126) | NO: 127) | 88) | NO: 85) | | 89) |
| MAB13 | GDTLTEL | DPEEGE | EGLAGFPFDI | RASQSISSW | KASSLES (SEQ ID | QQVNSYDWT |
| | (SEQ ID NO: | (SEQ ID | (SEQ ID NO: | LA (SEQ ID | NO: 86) | (SEQ ID NO |
| | 126) | NO: 127) | 90) | NO: 85) | | 89) |
| MAB14 | GDTLTEL | DPEEGE | EGLAWRPTDS | RASQSISSW | KASSLES (SEQ ID | QQYSSYAWT |
| | (SEQ ID NO: | (SEQ ID | (SEQ ID NO: | LA (SEQ ID | NO: 86) | (SEQ ID NO: |
| | 126) | NO: 127) | 91) | NO: 85) | | 92) |
| MAB15 | GDTLTEL | DPEEGE | KGLAWLPYY | RASQSISSW | KASSLES (SEQ ID | QQARSYSWT |
| | (SEQ ID NO: | (SEQ ID | S (SEQ ID NO: | LA (SEQ ID | NO: 86) | (SEQ ID NO: |
| | 126) | NO: 127) | 93) | NO: 85) | | 94) |
| MAB16 | GDTLTEL | DPEEGE | EGLAGVPFDL | RASQSISSW | KASSLES (SEQ ID | QQHNSYRWT |
| | (SEQ ID NO: | (SEQ ID | (SEQ ID NO | LA (SEQ ID | NO: 86) | (SEQ ID NO: |
| | 126) | NO: 127) | 84) | NO: 85) | | 95) |
| MAB17 | GDTLTEL | DPEEGE | EGEAGRPFDA | RASQSISSW | KASSLES (SEQ ID | VQYPSYSWT |
| | (SEQ ID NO: | (SEQ ID | (SEQ ID NO: | LA (SEQ ID | NO: 86) | (SEQ ID NO: |
| | 126) | NO: 127) | 96) | NO: 85) | | 97) |
| MAB18 | GDTLTEL | DPEEGE | EGLAGRPYDV | RASQSISSW | KASSLES (SEQ ID | QQYNSYKLT |
| | (SEQ ID NO: | (SEQ ID | (SEQ ID NO: | LA (SEQ ID | NO: 86) | (SEQ ID NO: |
| | 126) | NO: 127) | 98) | NO: 85) | | 99) |
| MAB19 | GDTLTEL | DPEEGE | EGLA GIPFDS | RASQSISSW | KASSLES (SEQ ID | QQYNSPSWL |
| | (SEQ ID NO: | (SEQ ID | W (SEQ ID NO: | LA (SEQ ID | NO: 86) | (SEQ ID NO: |
| | 126) | NO: 127) | 100) | NO: 85) | | 101) |
| MAB20 | GDTLTEL | DPEEGE | EGLAGIPFDS | RASQSISSW | KASSLES (SEQ ID | QQVNSLSWT |
| | (SEQ ID NO: | (SEQ ID | W (SEQ ID NO: | LA (SEQ ID | NO: 86) | (SEQ ID NO: |
| | 126) | NO: 127) | 100) | NO: 85) | | 102) |
| MAB21 | GFTFDDY | NWEGGS | DEQLGGTTYY | RASQGIRN | TASNLQS (SEQ ID | FQYNSYPLG |
| | (SEQ ID | (SEQ ID | YYYMDV | DLG (SEQ | NO: 107) | (SEQ ID NO: |
| | NO: 128) | NO: 129) | (SEQ ID NO: | ID NO: 106) | | 108) |
| | | | 105) | | | |
| MAB22 | GFTFDDY | NWEGGS | DEQLGGKYY | RASQGIRN | TASNLQS (SEQ ID | LQYRSHPLT |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 110) |
| | | | 109) | | | |
| MAB23 | GFTFDDY | NWEGGS | DEQLGGQTY | RASQGIRN | TASNLQS (SEQ ID | LQYNQVPLT |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 112) |
| | | | 111) | | | |
| MAB24 | GFTFDDY | NWEGGS | DEQLGGTTYY | RASQGIRN | TASNLQS (SEQ ID | LQTNIYPLT |
| | (SEQ ID | (SEQ ID | YYYMDV | DLG (SEQ | NO: 107) | (SEQ ID NO: |
| | NO: 128) | NO: 129) | (SEQ ID NO: | ID NO: 106) | | 113) |
| | | | 105) | | | |
| MAB25 | GFTFDDY | NWEGGS | DEQLGGIKYY | RASQGIRN | TASNLQS (SEQ ID | LQYKQYPLT |
| | (SEQ ID | (SEQ ID | YYYMDV | DLG (SEQ | NO: 107) | (SEQ ID NO: |
| | NO: 128) | NO: 129) | (SEQ ID NO: | ID NO: 106) | | 115) |
| | | | 114) | | | |
| MAB26 | GFTFDDY | NWEGGS | DEQLGGLKY | RASQGIRN | TASNLQS (SEQ ID | LQTNIYPLT |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 113) |
| | | | 116) | | | |
| MAB27 | GFTFDDY | NWEGGS | DEQLGGKVY | RASQGIRN | TASNLQS (SEQ ID | LQYNSYPLA |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 118) |
| | | | 117) | | | |
| MAB28 | GFTFDDY | NWEGGS | DEQLGGLKY | RASQGIRN | TASNLQS (SEQ ID | LQYHSYPLR |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 119) |
| | | | 116) | | | |
| MAB29 | GFTFDDY | NWEGGS | DEQLGGNHY | RASQGIRN | TASNLQS (SEQ ID | LQYASYPLK |
| | (SEQ ID | (SEQ ID | YYYYMDA | DLG (SEQ | NO: 107) | (SEQ ID NO: |
| | NO: 128) | NO: 129) | (SEQ ID NO: | ID NO: 106) | | 121) |
| | | | 120) | | | |
| MAB30 | GFTFDDY | NWEGGS | DEQLGGRHY | RASQGIRN | TASNLQS (SEQ ID | LQYNAYPLI |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 123) |
| | | | 122) | | | |
| MAB31 | GFTFSDY | SRSGST | DQEDYALDY | QASQDISNY | DASNLET (SEQ ID | EQYEDYPLT |
| | (SEQ ID NO: | (SEQ ID | (SEQ ID NO | LN (SEQ ID | NO: 65) | (SEQ ID NO: |
| | 124) | NO: 125) | 63) | NO: 64) | | 66) |
| MAB32 | GFTFSDY | SRSGST | DETDYGWDY | QASQDISNY | DASNLET (SEQ ID | KQYEDYPLT |
| | (SEQ ID NO: | (SEQ ID | (SEQ ID NO: | LN (SEQ ID | NO: 65) | (SEQ ID NO: |
| | 124) | NO: 125) | 67) | NO: 64) | | 68) |
| MAB33 | GFTFSDY | SRSGST | DETDYAFDE | QASQDISNY | DASNLET (SEQ ID | KQYEDYPLT |
| | (SEQ ID NO: | (SEQ ID | (SEQ ID NO | LN (SEQ ID | NO: 65) | (SEQ ID NO: |
| | 124) | NO: 125) | 69) | NO: 64) | | 68) |
| MAB34 | GFTFSDY | SRSGST | DESQYALDY | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 70) | NO: 64) | | 71) |
| MAB35 | GFTFSDY | SRSGST | DNTDYALDL | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 72) | NO: 64) | | 71) |
| MAB36 | GFTFSDY | SRSGST | DETDYAYDE | QASQDISNY | DASNLET (SEQ ID | HQYEDYPLT |
| | (SEQ ID NO: | (SEQ ID | (SEQ ID NO: | LN (SEQ ID | NO: 65) | (SEQ ID NO: |
| | 124) | NO: 125) | 73) | NO: 64) | | 71) |
| MAB37 | GFTFSDY | SRSGST | DETDYAYDN | QASQDISNY | DASNLET (SEQ ID | QQYEDYPLV |
| | (SEQ ID NO: | (SEQ ID | (SEQ ID NO: | LN (SEQ ID | NO: 65) | (SEQ ID NO: |
| | 124) | NO: 125) | 74) | NO: 64) | | 75) |
| MAB38 | GFTFSDY | SRSGST | DESDYAYDY | QASQDISNY | DASNLET (SEQ ID | QQHEDYPL |
| | (SEQ ID NO: | (SEQ ID | (SEQ ID NO: | LN (SEQ ID | NO: 65) | (SEQ ID NO: |
| | 124) | NO: 125) | 76) | NO: 64) | | 77) |
| MAB39 | GFTFSDY | SRSGST | DENYALDW | QASQDISNY | DASNLET (SEQ ID | QHYEDYPL |
| | (SEQ ID NO: | (SEQ ID | (SEQ ID NO: | LN (SEQ ID | NO: 65) | (SEQ ID NO: |
| | 124) | NO: 125) | 78) | NO: 64) | | 79) |
| MAB40 | GFTFSDY | SRSGST | DESDYALDF | QASQDISNY | DASNLET (SEQ ID | QQYEDLIPT |
| | (SEQ ID NO: | (SEQ ID | (SEQ ID NO: | LN (SEQ ID | NO: 65) | (SEQ ID NO: |
| | 124) | NO: 125) | 80) | NO: 64) | | 81) |
| MAB41 | GDTLTEL | DPEEGE | EGLAGVPFDL | RASQSISSW | KASSLES (SEQ ID | QSYNSYVWT |
| | (SEQ ID NO: | (SEQ ID | (SEQ ID NO: | LA (SEQ ID | NO: 86) | (SEQ ID NO: |
| | 126) | NO: 127) | 84) | NO: 85) | | 87) |
| MAB42 | GDTLTEL | DPEEGE | EGLGGRPFDH | RASQSISSW | KASSLES (SEQ ID | QQVNSYDWT |
| | (SEQ ID NO: | (SEQ ID | (SEQ ID NO: | LA (SEQ ID | NO: 86) | (SEQ ID NO |
| | 126) | NO: 127) | 88) | NO: 85) | | 89) |
| MAB43 | GDTLTEL | DPEEGE | EGLAGFPFDI | RASQSISSW | KASSLES (SEQ ID | QQVNSYDWT |
| | (SEQ ID NO: | (SEQ ID | (SEQ ID NO: | LA (SEQ ID | NO: 86) | (SEQ ID NO |
| | 126) | NO: 127) | 90) | NO: 85) | | 89) |
| MAB44 | GDTLTEL | DPEEGE | EGLAWRPTDS | RASQSISSW | KASSLES (SEQ ID | QQYSSYAWT |
| | (SEQ ID NO: | (SEQ ID | (SEQ ID NO: | LA (SEQ ID | NO: 86) | (SEQ ID NO: |
| | 126) | NO: 127) | 91) | NO: 85) | | 92) |
| MAB45 | GDTLTEL | DPEEGE | KGLAWLPYY | RASQSISSW | KASSLES (SEQ ID | QQARSYSWT |
| | (SEQ ID NO: | (SEQ ID | S (SEQ ID NO: | LA (SEQ ID | NO: 86) | (SEQ ID NO: |
| | 126) | NO: 127) | 93) | NO: 85) | | 94) |
| MAB46 | GDTLTEL | DPEEGE | EGLAGVPFDL | RASQSISSW | KASSLES (SEQ ID | QQHNSYRWT |
| | (SEQ ID NO: | (SEQ ID | (SEQ ID NO: | LA (SEQ ID | NO: 86) | (SEQ ID NO: |
| | 126) | NO: 127) | 84) | NO: 85) | | 95) |
| MAB47 | GDTLTEL | DPEEGE | EGEAGRPFDA | RASQSISSW | KASSLES (SEQ ID | VQYPSYSWT |
| | (SEQ ID NO: | (SEQ ID | (SEQ ID NO: | LA (SEQ ID | NO: 86) | (SEQ ID NO: |
| | 126) | NO: 127) | 96) | NO: 85) | | 97) |
| MAB48 | GDTLTEL | DPEEGE | EGLAGRPYDV | RASQSISSW | KASSLES (SEQ ID | QQYNSYKLT |
| | (SEQ ID NO: | (SEQ ID | (SEQ ID NO: | LA (SEQ ID | NO: 86) | (SEQ ID NO: |
| | 126) | NO: 127) | 98) | NO: 85) | | 99) |
| MAB49 | GDTLTEL | DPEEGE | EGLAGIPFDS | RASQSISSW | KASSLES (SEQ ID | QQYNSPSWL |
| | (SEQ ID NO: | (SEQ ID | W (SEQ ID NO: | LA (SEQ ID | NO: 86) | (SEQ ID NO: |
| | 126) | NO: 127) | 100) | NO: 85) | | 101) |
| MAB50 | GDTLTEL | DPEEGE | EGLAGIPFDS | RASQSISSW | KASSLES (SEQ ID | QQVNSLSWT |
| | (SEQ ID NO: | (SEQ ID | W (SEQ ID NO: | LA (SEQ ID | NO: 86) | (SEQ ID NO: |
| | 126) | NO: 127) | 100) | NO: 85) | | 102) |
| MAB51 | GFTFDDY | NWEGGS | DEQLGGTTYY | RASQGIRN | TASNLQS (SEQ ID | FQYNSYPLG |
| | (SEQ ID | (SEQ ID | YYYMDV | DLG (SEQ | NO: 107) | (SEQ ID NO: |
| | NO: 128) | NO: 129) | (SEQ ID NO: | ID NO: 106) | | 108) |
| | | | 105) | | | |
| MAB52 | GFTFDDY | NWEGGS | DEQLGGKYY | RASQGIRN | TASNLQS (SEQ ID | LQYRSHPLT |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 110) |
| | | | 109) | | | |
| MAB53 | GFTFDDY | NWEGGS | DEQLGGQTY | RASQGIRN | TASNLQS (SEQ ID | LQYNQVPLT |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 112) |
| | | | 111) | | | |
| MAB54 | GFTFDDY | NWEGGS | DEQLGGTTYY | RASQGIRN | TASNLQS (SEQ ID | LQTNIYPLT |
| | (SEQ ID | (SEQ ID | YYYMDV | DLG (SEQ | | (SEQ ID NO: |
| | NO: 128) | NO: 129) | (SEQ ID NO: | ID NO: 106) | NO: 107) | 113) |
| | | | 105) | | | |
| MAB55 | GFTFDDY | NWEGGS | DEQLGGIKYY | RASQGIRN | TASNLQS (SEQ ID | LQYKQYPLT |
| | (SEQ ID | (SEQ ID | YYYMDV | DLG (SEQ | NO: 107) | (SEQ ID NO: |
| | NO: 128) | NO: 129) | (SEQ ID NO: | ID NO: 106) | | 115) |
| | | | 114) | | | |
| MAB56 | GFTFDDY | NWEGGS | DEQLGGLKY | RASQGIRN | TASNLQS (SEQ ID | LQTNIYPLT |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 113) |
| | | | 116) | | | |
| MAB57 | GFTFDDY | NWEGGS | DEQLGGKVY | RASQGIRN | TASNLQS (SEQ ID | LQYNSYPLA |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 118) |
| | | | 117) | | | |
| MAB58 | GFTFDDY | NWEGGS | DEQLGGLKY | RASQGIRN | TASNLQS (SEQ ID | LQYHSYPLR |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 119) |
| | | | 116) | | | |
| MAB59 | GFTFDDY | NWEGGS | DEQLGGNHY | RASQGIRN | TASNLQS (SEQ ID | LQYASYPLK |
| | (SEQ ID | (SEQ ID | YYYYMDA | DLG (SEQ | NO: 107) | (SEQ ID NO: |
| | NO: 128) | NO: 129) | (SEQ ID NO: | ID NO: 106) | | 121) |
| | | | 120) | | | |
| MAB60 | GFTFDDY | NWEGGS | DEQLGGRHY | RASQGIRN | TASNLQS (SEQ ID | LQYNAYPLI |
| | (SEQ ID | (SEQ ID | YYYYMDV | DLG (SEQ | NO: 107) | (SEQ ID NO: |
| | NO: 128) | NO: 129) | (SEQ ID NO: | ID NO: 106) | | 123) |
| | | | 122) | | | |

IMGT CDRS

| Ab ID No | HCDR1 | HCDR2 | HCDR3 | LCDR1 | LCDR2 | LCDR3 |
|---|---|---|---|---|---|---|
| MAB1 | GFTFSDY | ISRSGSTK | ARDQEDYAL | QDISNY | DAS (SEQ ID NO: | EQYEDYPLT |
| | Y (SEQ ID | (SEQ ID NO: | DY (SEQ ID | (SEQ ID NO: | 134) | (SEQ ID NO: |
| | NO: 130) | 131) | NO: 132) | 133) | | 66) |
| MAB2 | GFTFSDY | ISRSGSTK | ARDETDYGW | QDISNY | DAS (SEQ ID NO: | KQYEDYPLT |
| | Y (SEQ ID | (SEQ ID NO: | DY (SEQ ID | (SEQ ID NO: | 134) | (SEQ ID NO: |
| | NO: 130) | 131) | NO: 135) | 133) | | 68) |
| MAB3 | GFTFSDY | ISRSGSTK | ARDETDYAFD | QDISNY | DAS (SEQ ID NO: | KQYEDYPLT |
| | Y (SEQ ID | (SEQ ID NO: | E (SEQ ID NO: | (SEQ ID NO: | 134) | (SEQ ID NO: |
| | NO: 130) | 131) | 136) | 133) | | 68) |
| MAB4 | GFTFSDY | ISRSGSTK | ARDESQYALD | QDISNY | DAS (SEQ ID NO: | HQYEDYPLT |
| | Y (SEQ ID | (SEQ ID NO: | Y (SEQ ID NO: | (SEQ ID NO: | 134) | (SEQ ID NO: |
| | NO: 130) | 131) | 137) | 133) | | 71) |
| MAB5 | GFTFSDY | ISRSGSTK | ARDNTDYAL | QDISNY | DAS (SEQ ID NO: | HQYEDYPLT |
| | Y (SEQ ID | (SEQ ID NO: | DL (SEQ ID | (SEQ ID NO: | 134) | (SEQ ID NO: |
| | NO: 130) | 131) | NO: 138) | 133) | | 71) |
| MAB6 | GFTFSDY | ISRSGSTK | ARDETDYAY | QDISNY | DAS (SEQ ID NO: | HQYEDYPLT |
| | Y (SEQ ID | (SEQ ID NO: | DE (SEQ ID | (SEQ ID NO: | 134) | (SEQ ID NO: |
| | NO: 130) | 131) | NO: 139) | 133) | | 71) |
| MAB7 | GFTFSDY | ISRSGSTK | ARDETDYAY | QDISNY | DAS (SEQ ID NO: | QQYEDYPLV |
| | Y (SEQ ID | (SEQ ID NO: | DN (SEQ ID | (SEQ ID NO: | 134) | (SEQ ID NO: |
| | NO: 130) | 131) | NO: 140) | 133) | | 75) |
| MAB8 | GFTFSDY | ISRSGSTK | ARDESDYAY | QDISNY | DAS (SEQ ID NO: | QQHEDYPL |
| | Y (SEQ ID | (SEQ ID NO: | DY (SEQ ID | (SEQ ID NO: | 134) | (SEQ ID NO: |
| | NO: 130) | 131) | NO: 141) | 133) | | 77) |
| MAB9 | GFTFSDY | ISRSGSTK | ARDENYALD | QDISNY | DAS (SEQ ID NO: | QHYEDYPL |
| | Y (SEQ ID | (SEQ ID NO: | W (SEQ ID NO: | (SEQ ID NO: | 134) | (SEQ ID NO: |
| | NO: 130) | 131) | 142) | 133) | | 79) |
| MAB10 | GFTFSDY | ISRSGSTK | ARDESDYALD | QDISNY | DAS (SEQ ID NO: | QQYEDLIPT |
| | Y (SEQ ID | (SEQ ID NO: | F (SEQ ID NO: | (SEQ ID NO: | 134) | (SEQ ID NO: |
| | NO: 130) | 131) | 143) | 133) | | 81) |
| MAB11 | GDTLTEL | FDPEEGET | VTEGLAGVPF | QSISSW | KAS (SEQ ID NO: | QSYNSYVWT |
| | S (SEQ ID | (SEQ ID NO: | DL (SEQ ID | (SEQ ID NO: | 148) | (SEQ ID NO: |
| | NO: 144) | 145) | NO: 146) | 147) | | 87) |
| MAB12 | GDTLTEL | FDPEEGET | VTEGLGGRPF | QSISSW | KAS (SEQ ID NO: | QQVNSYDWT |
| | S (SEQ ID | (SEQ ID NO: | DH (SEQ ID | (SEQ ID NO: | 148) | (SEQ ID NO |
| | NO: 144) | 145) | NO: 149) | 147) | | 89) |
| MAB13 | GDTLTEL | FDPEEGET | VTEGLAGFPF | QSISSW | KAS (SEQ ID NO: | QQVNSYDWT |
| | S (SEQ ID | (SEQ ID NO: | DI (SEQ ID | (SEQ ID NO: | 148) | (SEQ ID NO |
| | NO: 144) | 145) | NO: 150) | 147) | | 89) |
| MAB14 | GDTLTEL | FDPEEGET | VTEGLAWRPT | QSISSW | KAS (SEQ ID NO: | QQYSSYAWT |
| | S (SEQ ID | (SEQ ID NO: | DS (SEQ ID | (SEQ ID NO: | 148) | (SEQ ID NO: |
| | NO: 144) | 145) | NO: 151) | 147) | | 92) |
| MAB15 | GDTLTEL | FDPEEGET | VTKGLAWLP | QSISSW | KAS (SEQ ID NO: | QQARSYSWT |
| | S (SEQ ID | (SEQ ID NO: | YYS (SEQ ID | (SEQ ID NO: | 148) | (SEQ ID NO: |
| | NO: 144) | 145) | NO: 152) | 147) | | 94) |
| MAB16 | GDTLTEL | FDPEEGET | VTEGLAGVPF | QSISSW | KAS (SEQ ID NO: | QQHNSYRWT |
| | S (SEQ ID | (SEQ ID NO: | DL (SEQ ID | (SEQ ID NO: | 148) | (SEQ ID NO: |
| | NO: 144) | 145) | NO: 146) | 147) | | 95) |
| MAB17 | GDTLTEL | FDPEEGET | VTEGEAGRPF | QSISSW | KAS (SEQ ID NO: | VQYPSYSWT |
| | S (SEQ ID | (SEQ ID NO: | DA (SEQ ID | (SEQ ID NO: | 148) | (SEQ ID NO: |
| | NO: 144) | 145) | NO: 153) | 147) | | 97) |
| MAB18 | GDTLTEL | FDPEEGET | VTEGLAGRPY | QSISSW | KAS (SEQ ID NO: | QQYNSYKLT |
| | S (SEQ ID | (SEQ ID NO: | DV (SEQ ID | (SEQ ID NO: | 148) | (SEQ ID NO: |
| | NO: 144) | 145) | NO: 154) | 147) | | 99) |
| MAB19 | GDTLTEL | FDPEEGET | VTEGLAGIPF | QSISSW | KAS (SEQ ID NO: | QQYNSPSWL |
| | S (SEQ ID | (SEQ ID NO: | DSW (SEQ ID | (SEQ ID NO: | 148) | (SEQ ID NO: |
| | NO: 144) | 145) | NO: 155) | 147) | | 101) |
| MAB20 | GDTLTEL | FDPEEGET | VTEGLAGIPF | QSISSW | KAS (SEQ ID NO: | QQVNSLSWT |
| | S (SEQ ID | (SEQ ID NO: | DSW (SEQ ID | (SEQ ID NO: | 148) | (SEQ ID NO: |
| | NO: 144) | 145) | NO: 155) | 147) | | 102) |
| MAB21 | GFTFDDY | INWEGGST | ARDEQLGGTT | QGIRND | TAS (SEQ ID NO: 160) | FQYNSYPLG |
| | G (SEQ ID | (SEQ ID NO: | YYYYYMDV | (SEQ ID NO: | | (SEQ ID NO: |
| | NO: 156) | 157) | (SEQ ID NO: | 159) | | 108) |
| | | | 158) | | | |
| MAB22 | GFTFDDY | INWEGGST | ARDEQLGGK | QGIRND | TAS (SEQ ID NO: 160) | LQYRSHPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 110) |
| | | | 161) | | | |
| MAB23 | GFTFDDY | INWEGGST | ARDEQLGGQ | QGIRND | TAS (SEQ ID NO: 160) | LQYNQVPLT |
| | G (SEQ ID | (SEQ ID NO: | TYYYYYMDV | (SEQ ID NO: | | (SEQ ID NO: |
| | NO: 156) | 157) | (SEQ ID NO: | 159) | | 112) |
| | | | 162) | | | |
| MAB24 | GFTFDDY | INWEGGST | ARDEQLGGTT | QGIRND | TAS (SEQ ID NO: 160) | LQTNIYPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 113) |
| | | | 158) | | | |
| MAB25 | GFTFDDY | INWEGGST | ARDEQLGGIK | QGIRND | TAS (SEQ ID NO: 160) | LQYKQYPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 115) |
| | | | 163) | | | |
| MAB26 | GFTFDDY | INWEGGST | ARDEQLGGL | QGIRND | TAS (SEQ ID NO: 160) | LQTNIYPLT |
| | G (SEQ ID | (SEQ ID NO: | KYYYYYMDV | (SEQ ID NO: | | (SEQ ID NO: |
| | NO: 156) | 157) | (SEQ ID NO: | 159) | | 113) |
| | | | 164) | | | |
| MAB27 | GFTFDDY | INWEGGST | ARDEQLGGK | QGIRND | TAS (SEQ ID NO: 160) | LQYNSYPLA |
| | G (SEQ ID | (SEQ ID NO: | VYYYYYMDV | (SEQ ID NO: | | (SEQ ID NO: |
| | NO: 156) | 157) | (SEQ ID NO: | 159) | | 118) |
| | | | 165) | | | |
| MAB28 | GFTFDDY | INWEGGST | ARDEQLGGL | QGIRND | TAS (SEQ ID NO: 160) | LQYHSYPLR |
| | G (SEQ ID | (SEQ ID NO: | KYYYYYMDV | (SEQ ID NO: | | (SEQ ID NO: |
| | NO: 156) | 157) | (SEQ ID NO: | 159) | | 119) |
| | | | 164) | | | |
| MAB29 | GFTFDDY | INWEGGST | ARDEQLGGN | QGIRND | TAS (SEQ ID NO: 160) | LQYASYPLK |
| | G (SEQ ID | (SEQ ID NO: | HYYYYYMDA | (SEQ ID NO: | | (SEQ ID NO: |
| | NO: 156) | 157) | (SEQ ID NO: | 159) | | 121) |
| | | | 166) | | | |
| MAB30 | GFTFDDY | INWEGGST | ARDEQLGGR | QGIRND | TAS (SEQ ID NO: 160) | LQYNAYPLI |
| | G (SEQ ID | (SEQ ID NO: | HYYYYYMDV | (SEQ ID NO: | | (SEQ ID NO: |
| | NO: 156) | 157) | (SEQ ID NO | 159) | | 123) |
| | | | 167) | | | |
| MAB31 | GFTFSDY | ISRSGSTK | ARDQEDYAL | QDISNY | DAS (SEQ ID NO: | EQYEDYPLT |
| | Y (SEQ ID | (SEQ ID NO: | DY (SEQ ID | (SEQ ID NO: | 134) | (SEQ ID NO: |
| | NO: 130) | 131) | NO: 132) | 133) | | 66) |
| MAB32 | GFTFSDY | ISRSGSTK | ARDETDYGW | QDISNY | DAS (SEQ ID NO: | KQYEDYPLT |
| | Y (SEQ ID | (SEQ ID NO: | DY (SEQ ID | (SEQ ID NO: | 134) | (SEQ ID NO: |
| | NO: 130) | 131) | NO: 135) | 133) | | 68) |
| MAB33 | GFTFSDY | ISRSGSTK | ARDETDYAFD | QDISNY | DAS (SEQ ID NO: | KQYEDYPLT |
| | Y (SEQ ID | (SEQ ID NO: | E (SEQ ID NO: | (SEQ ID NO: | 134) | (SEQ ID NO: |
| | NO: 130) | 131) | 136) | 133) | | 68) |
| MAB34 | GFTFSDY | ISRSGSTK | ARDESQYALD | QDISNY | DAS (SEQ ID NO: | HQYEDYPLT |
| | Y (SEQ ID | (SEQ ID NO | Y (SEQ ID NO: | (SEQ ID NO: | 134) | (SEQ ID NO: |
| | NO: 130) | 131) | 137) | 133) | | 71) |
| MAB35 | GFTFSDY | ISRSGSTK | ARDNTDYAL | QDISNY | DAS (SEQ ID NO: | HQYEDYPLT |
| | Y (SEQ ID | (SEQ ID NO: | DL (SEQ ID | (SEQ ID NO: | 134) | (SEQ ID NO: |
| | NO: 130) | 131) | NO: 138) | 133) | | 71) |
| MAB36 | GFTFSDY | ISRSGSTK | ARDETDYAY | QDISNY | DAS (SEQ ID NO: | HQYEDYPLT |
| | Y (SEQ ID | (SEQ ID NO: | DE (SEQ ID | (SEQ ID NO: | 134) | (SEQ ID NO: |
| | NO: 130) | 131) | NO: 139) | 133) | | 71) |
| MAB37 | GFTFSDY | ISRSGSTK | ARDETDYAY | QDISNY | DAS (SEQ ID NO: | QQYEDYPLV |
| | Y (SEQ ID | (SEQ ID NO: | DN (SEQ ID | (SEQ ID NO: | 134) | (SEQ ID NO: |
| | NO: 130) | 131) | NO: 140) | 133) | | 75) |
| MAB38 | GFTFSDY | ISRSGSTK | ARDESDYAY | QDISNY | DAS (SEQ ID NO: | QQHEDYPL |
| | Y (SEQ ID | (SEQ ID NO: | DY (SEQ ID | (SEQ ID NO: | 134) | (SEQ ID NO: |
| | NO: 130) | 131) | NO: 141) | 133) | | 77) |
| MAB39 | GFTFSDY | ISRSGSTK | ARDENYALD | QDISNY | DAS (SEQ ID NO: | QHYEDYPL |
| | Y (SEQ ID | (SEQ ID NO: | W (SEQ ID NO: | (SEQ ID NO: | 134) | (SEQ ID NO: |
| | NO: 130) | 131) | 142) | 133) | | 79) |
| MAB40 | GFTFSDY | ISRSGSTK | ARDESDYALD | QDISNY | DAS (SEQ ID NO: | QQYEDLIPT |
| | Y (SEQ ID | (SEQ ID NO: | F (SEQ ID NO: | (SEQ ID NO: | 134) | (SEQ ID NO: |
| | NO: 130) | 131) | 143) | 133) | | 81) |
| MAB41 | GDTLTEL | FDPEEGET | VTEGLAGVPF | QSISSW | KAS (SEQ ID NO: | QSYNSYVWT |
| | S (SEQ ID | (SEQ ID NO: | DL (SEQ ID | (SEQ ID NO: | 148) | (SEQ ID NO: |
| | NO: 144) | 145) | NO: 146) | 147) | | 87) |
| MAB42 | GDTLTEL | FDPEEGET | VTEGLGGRPF | QSISSW | KAS (SEQ ID NO: | QQVNSYDWT |
| | S (SEQ ID | (SEQ ID NO: | DH (SEQ ID | (SEQ ID NO: | 148) | (SEQ ID NO |
| | NO: 144) | 145) | NO: 149) | 147) | | 89) |
| MAB43 | GDTLTEL | FDPEEGET | VTEGLAGFPF | QSISSW | KAS (SEQ ID NO: | QQVNSYDWT |
| | S (SEQ ID | (SEQ ID NO: | DI (SEQ ID | (SEQ ID NO: | 148) | (SEQ ID NO |
| | NO: 144) | 145) | NO: 150) | 147) | | 89) |
| MAB44 | GDTLTEL | FDPEEGET | VTEGLAWRPT | QSISSW | KAS (SEQ ID NO: | QQYSSYAWT |
| | S (SEQ ID | (SEQ ID NO: | DS (SEQ ID | (SEQ ID NO: | 148) | (SEQ ID NO: |
| | NO: 144) | 145) | NO: 151) | 147) | | 92) |
| MAB45 | GDTLTEL | FDPEEGET | VTKGLAWLP | QSISSW | KAS (SEQ ID NO: | QQARSYSWT |
| | S (SEQ ID | (SEQ ID NO: | YYS (SEQ ID | (SEQ ID NO: | 148) | (SEQ ID NO: |
| | NO: 144) | 145) | NO: 152) | 147) | | 94) |
| MAB46 | GDTLTEL | FDPEEGET | VTEGLAGVPF | QSISSW | KAS (SEQ ID NO: | QQHNSYRWT |
| | S (SEQ ID | (SEQ ID NO: | DL (SEQ ID | (SEQ ID NO: | 148) | (SEQ ID NO: |
| | NO: 144) | 145) | NO: 146) | 147) | | 95) |
| MAB47 | GDTLTEL | FDPEEGET | VTEGEAGRPF | QSISSW | KAS (SEQ ID NO: | VQYPSYSWT |
| | S (SEQ ID | (SEQ ID NO: | DA (SEQ ID | (SEQ ID NO: | 148) | (SEQ ID NO: |
| | NO: 144) | 145) | NO: 153) | 147) | | 97) |
| MAB48 | GDTLTEL | FDPEEGET | VTEGLAGRPY | QSISSW | KAS (SEQ ID NO: | QQYNSYKLT |
| | S (SEQ ID | (SEQ ID NO: | DV (SEQ ID | (SEQ ID NO: | 148) | (SEQ ID NO: |
| | NO: 144) | 145) | NO: 154) | 147) | | 99) |
| MAB49 | GDTLTEL | FDPEEGET | VTEGLAGIPF | QSISSW | KAS (SEQ ID NO: | QQYNSPSWL |
| | S (SEQ ID | (SEQ ID NO: | DSW (SEQ ID | (SEQ ID NO: | 148) | (SEQ ID NO: |
| | NO: 144) | 145) | NO: 155) | 147) | | 101) |
| MAB50 | GDTLTEL | FDPEEGET | VTEGLA GIPF | QSISSW | KAS (SEQ ID NO: | QQVNSLSWT |
| | S (SEQ ID | (SEQ ID NO: | DSW (SEQ ID | (SEQ ID NO: | 148) | (SEQ ID NO: |
| | NO: 144) | 145) | NO: 155) | 147) | | 102) |
| MAB51 | GFTFDDY | INWEGGST | ARDEQLGGTT | QGIRND | TAS (SEQ ID NO: 160) | FQYNSYPLG |
| | G (SEQ ID | (SEQ ID NO: | YYYYYMDV | (SEQ ID NO: | | (SEQ ID NO: |
| | NO: 156) | 157) | (SEQ ID NO: | 159) | | 108) |
| | | | 158) | | | |
| MAB52 | GFTFDDY | INWEGGST | ARDEQLGGK | QGIRND | TAS (SEQ ID NO: 160) | LQYRSHPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 110) |
| | | | 161) | | | |
| MAB53 | GFTFDDY | INWEGGST | ARDEQLGGQ | QGIRND | TAS (SEQ ID NO: 160) | LQYNQVPLT |
| | G (SEQ ID | (SEQ ID NO | TYYYYYMDV | (SEQ ID NO: | | (SEQ ID NO: |
| | NO: 156) | 157) | (SEQ ID NO: | 159) | | 112) |
| | | | 162) | | | |
| MAB54 | GFTEDDY | INWEGGST | ARDEQLGGTT | QGIRND | TAS (SEQ ID NO: 160) | LQTNIYPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 113) |
| | | | 158) | | | |
| MAB55 | GFTFDDY | INWEGGST | ARDEQLGGIK | QGIRND | TAS (SEQ ID NO: 160) | LQYKQYPLT |
| | G (SEQ ID | (SEQ ID NO: | YYYYYMDV | (SEQ ID NO: | | (SEQ ID NO: |
| | NO: 156) | 157) | (SEQ ID NO: | 159) | | 115) |
| | | | 163) | | | |
| MAB56 | GFTFDDY | INWEGGST | ARDEQLGGL | QGIRND | TAS (SEQ ID NO: 160) | LQTNIYPLT |
| | G (SEQ ID | (SEQ ID NO: | KYYYYYMDV | (SEQ ID NO: | | (SEQ ID NO: |
| | NO: 156) | 157) | (SEQ ID NO: | 159) | | 113) |
| | | | 164) | | | |
| MAB57 | GFTFDDY | INWEGGST | ARDEQLGGK | QGIRND | TAS (SEQ ID NO: 160) | LQYNSYPLA |
| | G (SEQ ID | (SEQ ID NO: | VYYYYYMDV | (SEQ ID NO: | | (SEQ ID NO: |
| | NO: 156) | 157) | (SEQ ID NO: | 159) | | 118) |
| | | | 165) | | | |
| MAB58 | GFTFDDY | INWEGGST | ARDEQLGGL | QGIRND | TAS (SEQ ID NO: 160) | LQYHSYPLR |
| | G (SEQ ID | (SEQ ID NO: | KYYYYYMDV | (SEQ ID NO: | | (SEQ ID NO: |
| | NO: 156) | 157) | (SEQ ID NO: | 159) | | 119) |
| | | | 164) | | | |
| MAB59 | GFTFDDY | INWEGGST | ARDEQLGGN | QGIRND | TAS (SEQ ID NO: 160) | LQYASYPLK |
| | G (SEQ ID | (SEQ ID NO: | HYYYYYMDA | (SEQ ID NO: | | (SEQ ID NO: |
| | NO: 156) | 157) | (SEQ ID NO: | 159) | | 121) |
| | | | 166) | | | |
| MAB60 | GFTFDDY | INWEGGST | ARDEQLGGR | QGIRND | TAS (SEQ ID NO: 160) | LQYNAYPLI |
| | G (SEQ ID | (SEQ ID NO: | HYYYYYMDV | (SEQ ID NO: | | (SEQ ID NO: |
| | NO: 156) | 157) | (SEQ ID NO | 159) | | 123) |
| | | | 167) | | | |

In some embodiments, the antibody comprises a heavy chain variable region polypeptide having one of the sequences selected from the table below, or a variant thereof.

| SEQ ID NO: | AB ID NO. | VH Sequence |
|---|---|---|
| 1 | MAB1 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDQEDYALDYWGQGTLVTVSS |
| 3 | MAB2 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYGWDYWGQGTLVTVSS |
| 5 | MAB3 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYAFDEWGQGTLVTVSS |
| 7 | MAB4 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESQYALDYWGQGTLVTVSS |
| 9 | MAB5 | QVQLVESGGGLVKPGGSLRLSCAASGFTFSDYYMSWIRQAP |
| | | GKGLEWVSYISRSGSTKYYAHSVKGRFTISRDNAKNSLYLQ |
| | | MNSLRAEDTAVYYCARDNTDYALDLWGQGTLVTVSS |
| 11 | MAB6 | QVQLVESGGGLVKPGGSLRLSCAASGFTFSDYYMSWIRQAP |
| | | GKGLEWVSYISRSGSTKYYAHSVKGRFTISRDNAKNSLYLQ |
| | | MNSLRAEDTAVYYCARDETDYAYDEWGQGTLVTVSS |
| 13 | MAB7 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYAYDNWGQGTLVTVSS |
| 15 | MAB8 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESDYAYDYWGQGTLVTVSS |
| 17 | MAB9 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDENYALDWWGQGTLVTVSS |
| 19 | MAB10 | QVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESDYALDFWGQGTLVTVSS |
| 21 | MAB11 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAGVPFDLWGQGTLVTVSS |
| 23 | MAB12 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLGGRPFDHWGQGTLVTVSS |
| 25 | MAB13 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAGFPFDIWGQGTLVTVSS |
| 27 | MAB14 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAWRPTDSWGQGTLVTVSS |
| 29 | MAB15 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTKGLAWLPYYSWGQGTLVTVSS |
| 31 | MAB16 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAGVPFDLWGQGTLVTVSS |
| 33 | MAB17 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGEAGRPFDAWGQGTLVTVSS |
| 35 | MAB18 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSSLRSEDTAVYYCVTEGLAGRPYDVWGQGTLVTVSS |
| 37 | MAB19 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSYLRSEDTAVYYCVTEGLAGIPFDSWGQGTLVTVSS |
| 39 | MAB20 | QVQLVQSGAEVKKPGASVKVSCKVSGDTLTELSMHWVRQ |
| | | APGKGLEWMGTFDPEEGETIYAQKFQGRVTMTEDTSTDTA |
| | | YMELSYLRSEDTAVYYCVTEGLAGIPFDSWGQGTLVTVSS |
| 41 | MAB21 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGTTYYYYYMDVWGKGTT |
| | | VTVSS |
| 43 | MAB22 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGKYYYYYYMDVWGKGT |
| | | TVTVSS |
| 45 | MAB23 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGQTYYYYYMDVWGKGT |
| | | TVTVSS |
| 47 | MAB24 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGTTYYYYYMDVWGKGTT |
| | | VTVSS |
| 49 | MAB25 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGIKYYYYYMDVWGKGTT |
| | | VTVSS |
| 51 | MAB26 | EVQLVESGGGVVRPGGSLRLSCAASGFTFDDYGMSWVRQA |
| | | PGKGLEWVSGINWEGGSTGYADSVKGRFSISRDNAKNSLYL |
| | | QMNSLRAEDTALYYCARDEQLGGLKYYYYYMDVWGKGT |
| | | TVTVSS |
| 53 | MAB27 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGKVYYYYYMDVWGKGT |
| | | TVTVSS |
| 55 | MAB28 | EVQLVESGGGVVRPGGSLRLSCAASGFTFDDYGMSWVRQA |
| | | PGKGLEWVSGINWEGGSTGYADSVKGRFSISRDNAKNSLYL |
| | | QMNSLRAEDTALYYCARDEQLGGLKYYYYYMDVWGKGT |
| | | TVTVSS |
| 57 | MAB29 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGNHYYYYYMDAWGKGT |
| | | TVTVSS |
| 59 | MAB30 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGRHYYYYYMDVWGKGT |
| | | TVTVSS |
| 334 | MAB31 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDQEDYALDYWGQGTLVTVSS |
| 335 | MAB32 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYGWDYWGQGTLVTVSS |
| 336 | MAB33 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYAFDEWGQGTLVTVSS |
| 337 | MAB34 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESQYALDYWGQGTLVTVSS |
| 338 | MAB35 | EVQLVESGGGLVKPGGSLRLSCAASGFTFSDYYMSWIRQAP |
| | | GKGLEWVSYISRSGSTKYYAHSVKGRFTISRDNAKNSLYLQ |
| | | MNSLRAEDTAVYYCARDNTDYALDLWGQGTLVTVSS |
| 339 | MAB36 | EVQLVESGGGLVKPGGSLRLSCAASGFTFSDYYMSWIRQAP |
| | | GKGLEWVSYISRSGSTKYYAHSVKGRFTISRDNAKNSLYLQ |
| | | MNSLRAEDTAVYYCARDETDYAYDEWGQGTLVTVSS |
| 340 | MAB37 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDETDYAYDNWGQGTLVTVSS |
| 341 | MAB38 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESDYAYDYWGQGTLVTVSS |
| 342 | MAB39 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDENYALDWWGQGTLVTVSS |
| 343 | MAB40 | EVQLVESGGGLVKPGGSLRLSCAASGFTFSDYYMSWIRQAP |
| | | GKGLEWVSYISRSGSTKYYADSVKGRFTISRDNAKNSLYLQ |
| | | MNSLRAEDTAVYYCARDESDYALDFWGQGTLVTVSS |
| 344 | MAB41 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAGVPFDLWGQGTLVTVSS |
| 345 | MAB42 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLGGRPFDHWGQGTLVTVSS |
| 346 | MAB43 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAGFPFDIWGQGTLVTVSS |
| 347 | MAB44 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAWRPTDSWGQGTLVTVSS |
| 348 | MAB45 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTKGLAWLPYYSWGQGTLVTVSS |
| 349 | MAB46 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAGVPFDLWGQGTLVTVSS |
| 350 | MAB47 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGEAGRPFDAWGQGTLVTVSS |
| 351 | MAB48 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSSLRSEDTAVYYCVTEGLAGRPYDVWGQGTLVTVSS |
| 352 | MAB49 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSYLRSEDTAVYYCVTEGLAGIPFDSWGQGTLVTVSS |
| 353 | MAB50 | EVQLVQSGAEVKKPGASVKVSCKVSGDTLTELSMHWVRQA |
| | | PGKGLEWMGTFDPEEGETIYAQKFQGRVTMTEDTSTDTAY |
| | | MELSYLRSEDTAVYYCVTEGLAGIPFDSWGQGTLVTVSS |
| 354 | MAB51 | EVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGTTYYYYYMDVWGKGTT |
| | | VTVSS |
| 355 | MAB52 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGKYYYYYYMDVWGKGT |
| | | TVTVSS |
| 356 | MAB53 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGQTYYYYYMDVWGKGT |
| | | TVTVSS |
| 357 | MAB54 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGTTYYYYYMDVWGKGTT |
| | | VTVSS |
| 358 | MAB55 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGIKYYYYYMDVWGKGTT |
| | | VTVSS |
| 359 | MAB56 | QVQLVESGGGVVRPGGSLRLSCAASGFTFDDYGMSWVRQA |
| | | PGKGLEWVSGINWEGGSTGYADSVKGRFSISRDNAKNSLYL |
| | | QMNSLRAEDTALYYCARDEQLGGLKYYYYYMDVWGKGT |
| | | TVTVSS |
| 360 | MAB57 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGKVYYYYYMDVWGKGT |
| | | TVTVSS |
| 361 | MAB58 | QVQLVESGGGVVRPGGSLRLSCAASGFTFDDYGMSWVRQA |
| | | PGKGLEWVSGINWEGGSTGYADSVKGRFSISRDNAKNSLYL |
| | | QMNSLRAEDTALYYCARDEQLGGLKYYYYYMDVWGKGT |
| | | TVTVSS |
| 362 | MAB59 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGNHYYYYYMDAWGKGT |
| | | TVTVSS |
| 363 | MAB60 | QVQLVESGGGVVRPGGSLRLSCAASGFTFDDYGMSWVRQA |
| | | PGKGLEWVSGINWEGGSTGYADSVKGRFTISRDNAKNSLYL |
| | | QMNSLRAEDTALYYCARDEQLGGRHYYYYYMDVWGKGT |
| | | TVTVSS |

In some embodiments, the antibody comprises a light chain variable region polypeptide having one of the sequences selected from the table below, or a variant thereof.

| SEQ ID NO: | AB ID NO. | VLSequence |
|---|---|---|
| 2 | MAB1 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCEQYEDYPLTFGGGTKVEIK |
| 4 | MAB2 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCKQYEDYPLTFGGGTKVEIK |
| 6 | MAB3 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCKQYEDYPLTFGGGTKVEIK |
| 8 | MAB4 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCHQYEDYPLTFGGGTKVEIK |
| 10 | MAB5 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCHQYEDYPLTFGGGTKVEIK |
| 12 | MAB6 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCHQYEDYPLTFGGGTKVEIK |
| 14 | MAB7 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCQQYEDYPLVFGGGTKVEIK |
| 16 | MAB8 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCQQHEDYPLTFGGGTKVEIK |
| 18 | MAB9 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCQHYEDYPLTFGGGTKVEIK |
| 20 | MAB10 | DIQMTQSPSSLSASVGDRVTITCQASQDISNYLNWYQQKPG |
| | | KAPKLLIYDASNLETGVPSRFSGSGSGTDFTFTISSLQPEDIAT |
| | | YYCQQYEDLIPTFGGGTKVEIK |
| 22 | MAB11 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQSYNSYVWTFGQGTKVEIK |
| 24 | MAB12 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQVNSYDWTFGQGTKVEIK |
| 26 | MAB13 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQVNSYDWTFGQGTKVEIK |
| 28 | MAB14 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQYSSYAWTFGQGTKVEIK |
| 30 | MAB15 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQARSYSWTFGQGTKVEIK |
| 32 | MAB16 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQHNSYRWTFGQGTKVEIK |
| 34 | MAB17 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCVQYPSYSWTFGQGTKVEIK |
| 36 | MAB18 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQYNSYKLTFGQGTKVEIK |
| 38 | MAB19 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQYNSPSWLFGQGTKVEIK |
| 40 | MAB20 | DIQMTQSPSTLSASVGDRVTITCRASQSISSWLAWYQQKPGK |
| | | APKLLIYKASSLESGVPSRFSGSGSGTEFTLTISSLQPDDFATY |
| | | YCQQVNSLSWTFGQGTKVEIK |
| 42 | MAB21 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCFQYNSYPLGFGGGTKVEIK |
| 44 | MAB22 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYRSHPLTFGGGTKVEIK |
| 46 | MAB23 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYNQVPLTFGGGTKVEIK |
| 48 | MAB24 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQTNIYPLTFGGGTKVEIK |
| 50 | MAB25 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYKQYPLTFGGGTKVEIK |
| 52 | MAB26 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQTNIYPLTFGGGTKVEIK |
| 54 | MAB27 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYNSYPLAFGGGTKVEIK |
| 56 | MAB28 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYHSYPLRFGGGTKVEIK |
| 58 | MAB29 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYASYPLKFGGGTKVEIK |
| 60 | MAB30 | DIQMTQSPSSLSASVGDRVTITCRASQGIRNDLGWYQQKPG |
| | | KAPKRLIYTASNLQSGVPSRFSGSGSGTEFTLTISSLQPEDFAT |
| | | YYCLQYNAYPLIFGGGTKVEIK |

In some embodiments, the antibody comprises a heavy chain as set forth in the table below, wherein the heavy chain comprises a heavy chain variable region and a constant domain.

| SEQ ID | | |
|---|---|---|
| NO: | AB ID NO. | Heavy Chain Sequence |
| 168 | MAB1 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDQEDYALDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 169 | MAB1 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDQEDYALDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 170 | MAB2 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDETDYGWDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 171 | MAB2 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDETDYGWDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 172 | MAB3 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDETDYAFDEWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 173 | MAB3 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDETDYAFDEWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 174 | MAB4 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDESQYALDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 175 | MAB4 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDESQYALDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 176 | MAB5 with constant | QVQLVESGGGLVKPGGSLRLSCAASGFTFSDYYMSWIRQA |
| | domain PE and | PGKGLEWVSYISRSGSTKYYAHSVKGRFTISRDNAKNSLY |
| | LS mutations | LQMNSLRAEDTAVYYCARDNTDYALDLWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 177 | MAB5 with constant | QVQLVESGGGLVKPGGSLRLSCAASGFTFSDYYMSWIRQA |
| | domain PE and | PGKGLEWVSYISRSGSTKYYAHSVKGRFTISRDNAKNSLY |
| | YTE mutations | LQMNSLRAEDTAVYYCARDNTDYALDLWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 178 | MAB6 with constant | QVQLVESGGGLVKPGGSLRLSCAASGFTFSDYYMSWIRQA |
| | domain PE and | PGKGLEWVSYISRSGSTKYYAHSVKGRFTISRDNAKNSLY |
| | LS mutations | LQMNSLRAEDTAVYYCARDETDYAYDEWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 179 | MAB6 with constant | QVQLVESGGGLVKPGGSLRLSCAASGFTFSDYYMSWIRQA |
| | domain PE and | PGKGLEWVSYISRSGSTKYYAHSVKGRFTISRDNAKNSLY |
| | YTE mutations | LQMNSLRAEDTAVYYCARDETDYAYDEWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 180 | MAB7 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDETDYAYDNWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 181 | MAB7 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDETDYAYDNWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 182 | MAB8 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDESDYAYDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 183 | MAB8 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDESDYAYDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 184 | MAB9 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDENYALDWWGQGTLVTVSSAST |
| | | KGPSVFPLAPCSRSTSESTAALGCLVKDYFPEPVTVSWNS |
| | | GALTSGVHTFPAVLQSSGLYSLSSVVTVPSSSLGTKTYTC |
| | | NVDHKPSNTKVDKRVESKYGPPCPPCPAPEFEGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVLHEALHSHYTQKSLSL |
| | | SLG |
| 185 | MAB9 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDENYALDWWGQGTLVTVSSAST |
| | | KGPSVFPLAPCSRSTSESTAALGCLVKDYFPEPVTVSWNS |
| | | GALTSGVHTFPAVLQSSGLYSLSSVVTVPSSSLGTKTYTC |
| | | NVDHKPSNTKVDKRVESKYGPPCPPCPAPEFEGGPSVFLF |
| | | PPKPKDTLYITREPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| 186 | MAB10 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDESDYALDFWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 187 | MAB10 with constant | QVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDESDYALDFWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 188 | MAB11 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAGVPFDLWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 189 | MAB11 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAGVPFDLWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 190 | MAB12 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLGGRPFDHWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 191 | MAB12 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLGGRPFDHWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 192 | MAB13 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAGFPFDIWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 193 | MAB13 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAGFPFDIWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 194 | MAB14 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAWRPTDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 195 | MAB14 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAWRPTDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 196 | MAB15 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTKGLAWLPYYSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 197 | MAB15 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTKGLAWLPYYSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 198 | MAB16 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAGVPFDLWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 199 | MAB16 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAGVPFDLWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 200 | MAB17 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGEAGRPFDAWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 201 | MAB17 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGEAGRPFDAWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 202 | MAB18 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAGRPYDVWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 203 | MAB18 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAGRPYDVWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 204 | MAB19 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSYLRSEDTAVYYCVTEGLAGIPFDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 205 | MAB19 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSYLRSEDTAVYYCVTEGLAGIPFDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 206 | MAB20 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSYLRSEDTAVYYCVTEGLAGIPFDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 207 | MAB20 with constant | QVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSYLRSEDTAVYYCVTEGLAGIPFDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 208 | MAB21 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGTTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 209 | MAB21 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGTTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 210 | MAB22 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGKYYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 211 | MAB22 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGKYYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 212 | MAB23 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGQTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 213 | MAB23 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGQTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 214 | MAB24 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGTTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 215 | MAB24 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGTTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 216 | MAB25 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGIKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 217 | MAB25 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGIKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 218 | MAB26 with constant | EVQLVESGGGVVRPGGSLRLSCAASGFTFDDYGMSWVRQA |
| | domain PE and | PGKGLEWVSGINWEGGSTGYADSVKGRFSISRDNAKNSLY |
| | LS mutations | LQMNSLRAEDTALYYCARDEQLGGLKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 219 | MAB26 with constant | EVQLVESGGGVVRPGGSLRLSCAASGFTFDDYGMSWVRQA |
| | domain PE and | PGKGLEWVSGINWEGGSTGYADSVKGRFSISRDNAKNSLY |
| | YTE mutations | LQMNSLRAEDTALYYCARDEQLGGLKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 220 | MAB27 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGKVYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 221 | MAB27 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGKVYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 222 | MAB28 with constant | EVQLVESGGGVVRPGGSLRLSCAASGFTFDDYGMSWVRQA |
| | domain PE and | PGKGLEWVSGINWEGGSTGYADSVKGRFSISRDNAKNSLY |
| | LS mutations | LQMNSLRAEDTALYYCARDEQLGGLKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 223 | MAB28 with constant | EVQLVESGGGVVRPGGSLRLSCAASGFTFDDYGMSWVRQA |
| | domain PE and | PGKGLEWVSGINWEGGSTGYADSVKGRFSISRDNAKNSLY |
| | YTE mutations | LQMNSLRAEDTALYYCARDEQLGGLKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 224 | MAB29 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGNHYYYYYMDAWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 225 | MAB29 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGNHYYYYYMDAWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 226 | MAB30 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGRHYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 227 | MAB30 with constant | EVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGRHYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 364 | MAB31 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDQEDYALDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 365 | MAB31 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDQEDYALDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 366 | MAB32 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDETDYGWDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 367 | MAB32 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDETDYGWDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 368 | MAB33 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDETDYAFDEWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 369 | MAB33 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDETDYAFDEWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 370 | MAB34 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDESQYALDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 371 | MAB34 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDESQYALDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 372 | MAB35 with constant | EVQLVESGGGLVKPGGSLRLSCAASGFTFSDYYMSWIRQA |
| | domain PE and | PGKGLEWVSYISRSGSTKYYAHSVKGRFTISRDNAKNSLY |
| | LS mutations | LQMNSLRAEDTAVYYCARDNTDYALDLWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 373 | MAB35 with constant | EVQLVESGGGLVKPGGSLRLSCAASGFTFSDYYMSWIRQA |
| | domain PE and | PGKGLEWVSYISRSGSTKYYAHSVKGRFTISRDNAKNSLY |
| | YTE mutations | LQMNSLRAEDTAVYYCARDNTDYALDLWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 374 | MAB36 with constant | EVQLVESGGGLVKPGGSLRLSCAASGFTFSDYYMSWIRQA |
| | domain PE and | PGKGLEWVSYISRSGSTKYYAHSVKGRFTISRDNAKNSLY |
| | LS mutations | LQMNSLRAEDTAVYYCARDETDYAYDEWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 375 | MAB36 with constant | EVQLVESGGGLVKPGGSLRLSCAASGFTFSDYYMSWIRQA |
| | domain PE and | PGKGLEWVSYISRSGSTKYYAHSVKGRFTISRDNAKNSLY |
| | YTE mutations | LQMNSLRAEDTAVYYCARDETDYAYDEWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 376 | MAB37 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDETDYAYDNWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 377 | MAB37 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDETDYAYDNWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 378 | MAB38 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDESDYAYDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 379 | MAB38 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDESDYAYDYWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 380 | MAB39 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDENYALDWWGQGTLVTVSSAST |
| | | KGPSVFPLAPCSRSTSESTAALGCLVKDYFPEPVTVSWNS |
| | | GALTSGVHTFPAVLQSSGLYSLSSVVTVPSSSLGTKTYTC |
| | | NVDHKPSNTKVDKRVESKYGPPCPPCPAPEFEGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVLHEALHSHYTQKSLSL |
| | | SLG |
| 381 | MAB39 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDENYALDWWGQGTLVTVSSAST |
| | | KGPSVFPLAPCSRSTSESTAALGCLVKDYFPEPVTVSWNS |
| | | GALTSGVHTFPAVLQSSGLYSLSSVVTVPSSSLGTKTYTC |
| | | NVDHKPSNTKVDKRVESKYGPPCPPCPAPEFEGGPSVFLF |
| | | PPKPKDTLYITREPEVTCVVVDVSQEDPEVQFNWYVDGVE |
| | | VHNAKTKPREEQFNSTYRVVSVLTVLHQDWLNGKEYKCKV |
| | | SNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| 382 | MAB40 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | LS mutations | LQMNSLRAEDTAVYYCARDESDYALDFWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVLHEALHSHYTQKSLS |
| | | LSLG |
| 383 | MAB40 with constant | EVQLVESGGGLVKPGGSLRLSCAASGFTFSDYYMSWIRQA |
| | domain PE and | PGKGLEWVSYISRSGSTKYYADSVKGRFTISRDNAKNSLY |
| | YTE mutations | LQMNSLRAEDTAVYYCARDESDYALDFWGQGTLVTVSSAS |
| | | TKGPSVFPLAPCSRSTSESTAALGCLVKDYFPEPVTVSWN |
| | | SGALTSGVHTFPAVLQSSGLYSLSSVVTVPSSSLGTKTYT |
| | | CNVDHKPSNTKVDKRVESKYGPPCPPCPAPEFEGGPSVFL |
| | | FPPKPKDTLYITREPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSLS |
| | | LSLG |
| 384 | MAB41 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAGVPFDLWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 385 | MAB41 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAGVPFDLWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 386 | MAB42 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLGGRPFDHWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 387 | MAB42 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLGGRPFDHWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 388 | MAB43 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAGFPFDIWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 389 | MAB43 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAGFPFDIWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 390 | MAB44 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAWRPTDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 391 | MAB44 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAWRPTDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 392 | MAB45 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTKGLAWLPYYSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 393 | MAB45 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTKGLAWLPYYSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 394 | MAB46 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAGVPFDLWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 395 | MAB46 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAGVPFDLWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 396 | MAB47 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGEAGRPFDAWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 397 | MAB47 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGEAGRPFDAWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 398 | MAB48 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSSLRSEDTAVYYCVTEGLAGRPYDVWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 399 | MAB48 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSSLRSEDTAVYYCVTEGLAGRPYDVWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 400 | MAB49 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSYLRSEDTAVYYCVTEGLAGIPFDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 401 | MAB49 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSYLRSEDTAVYYCVTEGLAGIPFDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 402 | MAB50 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | LS mutations | MELSYLRSEDTAVYYCVTEGLAGIPFDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLMISRTPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVLHEALHSHYTQKSL |
| | | SLSLG |
| 403 | MAB50 with constant | EVQLVQSGAEVKKPGASVKVSCKVSGDTLTELSMHWVRQA |
| | domain PE and | PGKGLEWMGTFDPEEGETIYAQKFQGRVTMTEDTSTDTAY |
| | YTE mutations | MELSYLRSEDTAVYYCVTEGLAGIPFDSWGQGTLVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTY |
| | | TCNVDHKPSNTKVDKRVESKYGPPCPPCPAPEFEGGPSVF |
| | | LFPPKPKDTLYITREPEVTCVVVDVSQEDPEVQFNWYVDG |
| | | VEVHNAKTKPREEQFNSTYRVVSVLTVLHQDWLNGKEYKC |
| | | KVSNKGLPSSIEKTISKAKGQPREPQVYTLPPSQEEMTKN |
| | | QVSLTCLVKGFYPSDIAVEWESNGQPENNYKTTPPVLDSD |
| | | GSFFLYSRLTVDKSRWQEGNVFSCSVMHEALHNHYTQKSL |
| | | SLSLG |
| 404 | MAB51 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGTTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 405 | MAB51 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGTTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 406 | MAB52 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGKYYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 407 | MAB52 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGKYYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 408 | MAB53 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGQTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 409 | MAB53 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGQTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 410 | MAB54 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGTTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 411 | MAB54 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGTTYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 412 | MAB55 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGIKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 413 | MAB55 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGIKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 414 | MAB56 with constant | QVQLVESGGGVVRPGGSLRLSCAASGFTFDDYGMSWVRQA |
| | domain PE and | PGKGLEWVSGINWEGGSTGYADSVKGRFSISRDNAKNSLY |
| | LS mutations | LQMNSLRAEDTALYYCARDEQLGGLKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 415 | MAB56 with constant | QVQLVESGGGVVRPGGSLRLSCAASGFTFDDYGMSWVRQA |
| | domain PE and | PGKGLEWVSGINWEGGSTGYADSVKGRFSISRDNAKNSLY |
| | YTE mutations | LQMNSLRAEDTALYYCARDEQLGGLKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 416 | MAB57 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGKVYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 417 | MAB57 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGKVYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 418 | MAB58 with constant | QVQLVESGGGVVRPGGSLRLSCAASGFTFDDYGMSWVRQA |
| | domain PE and | PGKGLEWVSGINWEGGSTGYADSVKGRFSISRDNAKNSLY |
| | LS mutations | LQMNSLRAEDTALYYCARDEQLGGLKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 419 | MAB58 with constant | QVQLVESGGGVVRPGGSLRLSCAASGFTFDDYGMSWVRQA |
| | domain PE and | PGKGLEWVSGINWEGGSTGYADSVKGRFSISRDNAKNSLY |
| | YTE mutations | LQMNSLRAEDTALYYCARDEQLGGLKYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 420 | MAB59 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGNHYYYYYMDAWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 421 | MAB59 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGNHYYYYYMDAWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |
| 422 | MAB60 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | LS mutations | LQMNSLRAEDTALYYCARDEQLGGRHYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVLHEALHSH |
| | | YTQKSLSLSLG |
| 423 | MAB60 with constant | QVQLVESGGGVVRPGGSLRLSCAASGFTFDDYGMSWVRQA |
| | domain PE and | PGKGLEWVSGINWEGGSTGYADSVKGRFTISRDNAKNSLY |
| | YTE mutations | LQMNSLRAEDTALYYCARDEQLGGRHYYYYYMDVWGKGTT |
| | | VTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLYITREPEVTCVVVDVSQEDPEVQF |
| | | NWYVDGVEVHNAKTKPREEQFNSTYRVVSVLTVLHQDWLN |
| | | GKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHNH |
| | | YTQKSLSLSLG |

In some embodiments, the antibody comprises a light chain as set forth in the table below, wherein the light chain comprises a light chain variable region and a constant domain, such as the human kappa constant domain.

| SEQ ID | | |
|---|---|---|
| NO: | AB ID NO. | Light Chain Sequence |
| 228 | MAB1 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCEQYEDYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 229 | MAB2 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCKQYEDYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 230 | MAB3 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCKQYEDYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 231 | MAB4 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCHQYEDYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 232 | MAB5 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCHQYEDYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 233 | MAB6 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCHQYEDYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 234 | MAB7 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCQQYEDYPLVFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 235 | MAB8 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCQQHEDYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 236 | MAB9 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCQHYEDYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 237 | MAB10 plus kappa | DIQMTQSPSSLSASVGDRVTITCQASQDISNYLNWYQQKP |
| | constant domain | GKAPKLLIYDASNLETGVPSRFSGSGSGTDFTFTISSLQP |
| | | EDIATYYCQQYEDLIPTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 238 | MAB11 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQSYNSYVWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 239 | MAB12 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQQVNSYDWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 240 | MAB13 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQQVNSYDWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 241 | MAB14 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQQYSSYAWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 242 | MAB15 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQQARSYSWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 243 | MAB16 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQQHNSYRWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 244 | MAB17 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCVQYPSYSWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 245 | MAB18 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQQYNSYKLTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 246 | MAB19 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQQYNSPSWLFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 247 | MAB20 plus kappa | DIQMTQSPSTLSASVGDRVTITCRASQSISSWLAWYQQKP |
| | constant domain | GKAPKLLIYKASSLESGVPSRFSGSGSGTEFTLTISSLQP |
| | | DDFATYYCQQVNSLSWTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 248 | MAB21 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCFQYNSYPLGFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 249 | MAB22 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQYRSHPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 250 | MAB23 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQYNQVPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 251 | MAB24 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQTNIYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 252 | MAB25 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQYKQYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 253 | MAB26 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQTNIYPLTFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 254 | MAB27 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQYNSYPLAFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 255 | MAB28 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQYHSYPLRFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 256 | MAB29 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQYASYPLKFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 257 | MAB30 plus kappa | DIQMTQSPSSLSASVGDRVTITCRASQGIRNDLGWYQQKP |
| | constant domain | GKAPKRLIYTASNLQSGVPSRFSGSGSGTEFTLTISSLQP |
| | | EDFATYYCLQYNAYPLIFGGGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

In some embodiments, the antibody comprises an Fc domain. The Fc domain can be linked to the heavy or light chain of the antibody. In some embodiments, the Fc domain comprises a mutation to extend the half-life of the antibody. In some embodiments, the Fc domain comprises a mutation such as those described in U.S. Pat. No. 7,670,600, which is hereby incorporated by reference in its entirety. In some embodiment, the constant region comprises a mutation at position at amino acid residue 428 relative to a wild-type human IgG constant domain, numbered according to the EU numbering index of Kabat. Without being bound to any particular theory, an antibody comprising a mutation that corresponds to residue 428 can have an increased half-life compared to the half-life of an IgG having the wild-type human IgG constant domain. In some embodiments, the mutation is a substitution of the native residue with a threonine, leucine, phenylalanine or serine. In some embodiments, the antibody further comprises one or more amino acid substitutions relative to the corresponding wild-type human IgG constant domain at one or more of amino acid residues 251-256, 285-290, 308-314, 385-389, and 429-436, numbered according to the Kabat EU numbering index. The specific mutations or substitutions at these positions are described in U.S. Pat. No. 7,670,600, which is hereby incorporated by reference in its entirety.

Other mutations can be used in the Fc domain, such as those provided for in U.S. Pat. No. 8,394,925, which is hereby incorporated by reference in its entirety. In some embodiments, the Fc region is a variant Fc region comprising amino acid substitutions at positions 428 and 434, wherein the amino acid substitutions are a leucine that is not the wild-type amino acid at position 428 and a serine that is not the wild-type amino acid at position 434, wherein the polypeptide is an antibody and wherein numbering is according to the EU Index in Kabat et al. In some embodiments, the Fc region comprises a S228P, L235E, M428L, or N434S substitution. In some embodiments, the Fc region comprises a M428L substitution. In some embodiments, the Fc region comprises a N434S substitution. In some embodiments, the Fc region comprises a M428L and a N434S substitution. In some embodiments, the Fc region comprises a M252Y, S254T, and/or T256E substitution.

In some embodiments, the antibody comprises a constant region as set forth below with or without the mutations provided for in the list below:

(IgG4 S228P L235E LS; SEQ ID NO: 261)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVLHEALHSH
YTQKSLSLSLGK;
or
(IgG4 S228P L235E YTE; SEQ ID NO: 262)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLYITR
EPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK.

In some embodiments, the antibody comprises a constant region as provided herein, wherein the C-terminal lysine (K) amino acid has been deleted. In some embodiments, the antibody comprises a constant region as set follows:

(IgG4 S228P L235E LS-trunc; SEQ ID NO: 263)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVLHEALHSH
YTQKSLSLSLG;
or
(IgG4 S228P L235E YTE-trunc; SEQ ID NO: 264)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLYITR
EPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLG.

In some embodiments, the antibody, or the light chain of the antibody, comprises a kappa constant region, such as the human constant domain, which can comprise a sequence of:

(human kappa constant domain, SEQ ID NO: 265)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC.

The term “purified” with referenced to an antibody refers to an antibody that is substantially free of other material that associates with the molecule in its natural environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80%-90% (w/w) pure, 90-95% pure; and, at least 95%, 96%, 97%, 98%, 99%, or 100% (w/w) pure. In some embodiments, the antibody is purified.

### Pharmaceutical Compositions

In some embodiments, a pharmaceutical composition is provided. In some embodiments, the pharmaceutical composition comprises an antibody or antigen-binding fragment thereof as provided for herein.

In some embodiments, to prepare pharmaceutical or sterile compositions of the anti-C1s antibodies or other proteins provided herein, the antibody, or antigen binding fragment thereof, or other proteins provided herein are admixed with a pharmaceutically acceptable carrier or excipient. (see, e.g., Remington's Pharmaceutical Sciences and U.S. Pharmacopeia: National Formulary, Mack Publishing Company, Easton, PA (1984)).

Formulations of therapeutic or the antibodies provided herein may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY). In some embodiments, the antibodies are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In some embodiments, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In some embodiments, a composition is administered to a subject in accordance with the Physicians' Desk Reference 2003 (Thomson Healthcare; 57th edition (Nov. 1, 2002)).

In some embodiments, the mode of administration may vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In some embodiments, the composition is an injectable pharmaceutical composition. In some embodiments, the composition is formulated for intravenous or subcutaneous injection. In some embodiments, the composition is formulated for intravenous injection. In some embodiments, the composition is formulated for subcutaneous injection.

In some embodiments, the antibody, or antigen binding fragment thereof, can be administered by an invasive route such as by injection. In some embodiments, the antibodies or antigen binding fragment thereof, or pharmaceutical composition thereof, is administered intravenously, subcutaneously, intramuscularly, intraarterially, intra-articularly (e.g. in arthritis joints), intratumorally, or by inhalation, such as through an aerosol delivery. Administration by non-invasive routes (e.g., orally; for example, in a pill, capsule or tablet) is also within the scope of the present embodiments.

In some embodiments, the anti-CIs antibody, or antigen binding fragment thereof, is administered in combination with at least one additional therapeutic agent, such as, but not limited to any therapeutic used to treat the disorders provided for herein. In some embodiments, the antibody is administered in combination with another treatment for the disorders provided for herein.

Compositions can be administered with medical devices known in the art. For example, a pharmaceutical composition of the invention can be administered by injection with a hypodermic needle, including, e.g., a prefilled syringe or an autoinjector.

The pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Other devices that can also be used, including, but are not limited to those provided for in U.S. patent Ser. No. 11/779,704, U.S. patent Ser. No. 11/654,244, U.S. patent Ser. No. 11/612,690, U.S. patent Ser. No. 11/607,494, U.S. patent Ser. No. 11/602,596, U.S. patent Ser. No. 11/541,189, U.S. patent Ser. No. 11/534,556, U.S. patent Ser. No. 11/524,118, U.S. patent Ser. No. 11/458,257, U.S. patent Ser. No. 11/458,254, U.S. patent Ser. No. 11/426,529, U.S. patent Ser. No. 11/426,524, U.S. patent Ser. No. 11/406,768, U.S. patent Ser. No. 11/383,044, U.S. patent Ser. No. 11/331,437, U.S. patent Ser. No. 11/311,681, U.S. patent Ser. No. 11/291,771, U.S. patent Ser. No. 11/273,265, U.S. patent Ser. No. 11/253,653, U.S. patent Ser. No. 11/229,748, U.S. patent Ser. No. 11/224,697, U.S. patent Ser. No. 11/154,654, U.S. patent Ser. No. 11/141,536, U.S. patent Ser. No. 11/103,647, U.S. patent Ser. No. 11/103,646, U.S. patent Ser. No. 11/065,393, U.S. patent Ser. No. 11/058,822, U.S. patent Ser. No. 10/983,187, U.S. patent Ser. No. 10/980,947, U.S. patent Ser. No. 10/973,988, U.S. patent Ser. No. 10/960,143, U.S. patent Ser. No. 10/894,127, U.S. patent Ser. No. 10/881,799, U.S. patent Ser. No. 10/850,032, U.S. patent Ser. No. 10/814,066, U.S. patent Ser. No. 10/799,647, U.S. patent Ser. No. 10/709,840, U.S. patent Ser. No. 10/632,260, U.S. patent Ser. No. 10/610,646, U.S. patent Ser. No. 10/603,443, U.S. patent Ser. No. 10/596,327, U.S. patent Ser. No. 10/569,025, U.S. patent Ser. No. 10/569,019, U.S. patent Ser. No. 10/561,799, U.S. patent Ser. No. 10/537,685, U.S. patent Ser. No. 10/518,035, U.S. patent Ser. No. 10/493,213, U.S. patent Ser. No. 10/493,212, U.S. patent Ser. No. 10/485,933, U.S. patent Ser. No. 10/471,215, U.S. patent Ser. No. 10/441,729, U.S. patent Ser. No. 10/413,673, U.S. patent Ser. No. 10/376,641, U.S. patent Ser. No. 10/350,363, U.S. patent Ser. No. 10/350,359, U.S. patent Ser. No. 10/350,358, U.S. patent Ser. No. 10/350,356, U.S. patent Ser. No. 10/335,543, U.S. patent Ser. No. 10/331,996, U.S. patent Ser. No. 10/300,211, U.S. patent Ser. No. 10/201,659, U.S. patent Ser. No. 10/149,948, U.S. patent Ser. No. 10/149,945, U.S. patent Ser. No. 10/143,806, U.S. patent Ser. No. 10/137,253, U.S. patent Ser. No. 10/099,017, U.S. Pat. Nos. 9,962,496, 9,962,495, U.S. Pat. Nos. 9,867,948, 9,855,392, 9,821,121, 9,750,887, 9,744,311, 9,566,389, 9,539,385, 9,457,156, 9,320,853, 9,289,561, 9,278,178, 9,205,195, 9,138,543, 9,057,369, 8,998,876, 8,992,487, 8,956,330, 8,945,053, 8,939,944, 8,834,431, 8,827,963, 8,758,292, 8,734,403, 8,721,615, 8,690,837, 8,679,071, 8,672,901, 8,657,788, 8,647,307, 8,591,465, 8,562,570, 8,556,847, 8,491,538, 8,439,864, 8,409,149, 8,409,148, 8,409,145, 8,398,594, 8,398,593, 8,343,103, 8,298,175, 8,246,577, 8,216,180, 8,048,037, 8,043,262, 8,043,249, 8,016,797, 7,976,509, 7,955,303, 7,951,113, 7,815,598, 7,749,186, 7,511,480, 7,357,790, PCT Publication No. WO2023094119, PCT Publication No. WO2023072787, PCT Publication No. WO2023072728, PCT Publication No. WO2023062178, PCT Publication No. WO2023057301, PCT Publication No. WO2023057272, PCT Publication No. WO2023051963, PCT Publication No. WO2023046741, PCT Publication No. WO2023046348, PCT Publication No. WO2023017114, PCT Publication No. WO2023012030, PCT Publication No. WO2023001988, PCT Publication No. WO2023001430, PCT Publication No. WO2023001424, PCT Publication No. WO2023275273, PCT Publication No. WO2022268389, PCT Publication No. WO2022248119, PCT Publication No. WO2022248113, PCT Publication No. WO2022184539, PCT Publication No. WO2022184388, PCT Publication No. WO2022135921, PCT Publication No. WO2022128606, PCT Publication No. WO2022128385, PCT Publication No. WO2022106197, PCT Publication No. WO2022096352, PCT Publication No. WO2022090047, PCT Publication No. WO2022069617, PCT Publication No. WO2022069175, PCT Publication No. WO2022058221, PCT Publication No. WO2022053307, PCT Publication No. WO2022053248, PCT Publication No. WO2015127965, or PCT Publication No. WO2013170392, each of which is hereby incorporated by reference in its entirety.

Other devices that can be used to deliver injectables to a subject, including those that can handle larger volumes, include devices provided for in U.S. patent Ser. No. 11/833,326, U.S. patent Ser. No. 11/786,173, U.S. patent Ser. No. 11/744,777, U.S. patent Ser. No. 11/571,361, U.S. patent Ser. No. 11/571,360, U.S. patent Ser. No. 11/571,164, U.S. patent Ser. No. 11/478,583, U.S. patent Ser. No. 11/413,393, U.S. patent Ser. No. 11/109,800, U.S. patent Ser. No. 11/040,138, U.S. patent Ser. No. 11/033,680, U.S. patent Ser. No. 10/729,842, U.S. patent Ser. No. 10/507,285, U.S. patent Ser. No. 10/449,293, U.S. Pat. No. 9,925,333, U.S. Patent Application Publication No. 20230293108, U.S. Patent Application Publication No. 20230285243, U.S. Patent Application Publication No. 20230190582, U.S. Patent Application Publication No. 20230132382, U.S. Patent Application Publication No. 20230104373, U.S. Patent Application Publication No. 20220387705, U.S. Patent Application Publication No. 20220280385, U.S. Patent Application Publication No. 20220023533, U.S. Patent Application Publication No. 20210386621, U.S. Patent Application Publication No. 20210338928, U.S. Patent Application Publication No. 20200338257, U.S. Patent Application Publication No. 20200061292, U.S. Patent Application Publication No. 20180207369, PCT Publication No. WO2022031784, PCT Publication No. WO2022006063, PCT Publication No. WO2021016567, PCT Publication No. WO2020142544, PCT Publication No. WO2020118165, PCT Publication No. WO2020086581, PCT Publication No. WO2019079335, PCT Publication No. WO2019075337, PCT Publication No. WO2018232171, PCT Publication No. WO2018218082, PCT Publication No. WO2018165588, PCT Publication No. WO2017014847, PCT Publication No. WO2016154413, or PCT Publication No. WO2016153747, each of which is hereby incorporated by reference in its entirety.

In some embodiments, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. Such liposomes are then targeted to and taken up selectively by the afflicted tissue.

In some embodiments, the administration regimen may depend on several factors, including the serum or tissue turnover rate of the therapeutic antibody, the level of symptoms, the immunogenicity of the therapeutic antibody, and/or the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med 342:613-619; Ghosh et al. (2003) New Engl. J. Med. 348:24-32; Lipsky et al. (2000) New Engl. J. Med. 343:1594-1602).

Determination of the appropriate dose can be made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, chimeric, humanized and fully human antibodies may be desirable.

In some embodiments, antibodies, or antigen binding fragments thereof, can be provided by continuous infusion, or by doses administered, e.g., daily, 1-7 times per week, weekly, bi-weekly, monthly, bimonthly, or quarterly. In some embodiments, a total weekly dose is generally at least 0.05 g/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/mL, 10 mg/kg, 25 mg/kg, 50 mg/kg or more (see, e.g., Yang, et al. (2003) New Engl. J. Med. 349:427-434; Herold, et al. (2002)New Engl. J. Med. 346:1692-1698; Liu, et al. (1999) J. Neurol. Neurosurg. Psych. 67:451-456; Portielji, et al. (20003) Cancer Immunol. Immunother. 52:133-144). Doses may also be provided to achieve a pre-determined target concentration of the antibody in the subject's serum, such as 0.1, 0.3, 1, 3, 10, 30, 100, 300, 400, 500, 600 μg/ml or more. In other embodiments, a fully human antibody is administered subcutaneously or intravenously, on a weekly, biweekly, every 4 weeks, monthly, bimonthly, or quarterly basis at 10, 20, 50, 80, 100, 200, 300, 400, 500, 600, 1000 or 2500 mg/subject.

In some embodiments, a pharmaceutical composition is provided that comprises about 25 mg/mL to about 300 mg/mL of an antibody having a heavy chain and a light chain; one or more of a buffer, an antioxidant, a sugar, a viscosity modifying agent, or a surfactant; and wherein the pharmaceutical composition is at a pH of about 6.5 to about 8.0. In some embodiments, a pharmaceutical composition is provided that comprises about 25 mg/mL to about 300 mg/mL of an antibody having a heavy chain and a light chain; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0. In some embodiments, a pharmaceutical composition is provided that comprises about 25 mg/mL to about 300 mg/mL of an antibody having a heavy chain and a light chain; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 selected from any one HCDR1 provided herein, an HCDR2 selected from any one HCDR2 provided herein, and an HCDR3 selected from any one HCDR3 provided herein, and wherein the light chain comprises an LCDR1 selected from any one LCDR1 provided herein, an LCDR2 selected from any one LCDR2 provided herein, and an LCDR3 selected from any one LCDR3 provided herein; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 63, and wherein the light chain comprises an LCDR1 of SEQ ID NO: 64, an LCDR2 of SEQ ID NO: 65, and an LCDR3 of SEQ ID NO: 66; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 63, and wherein the light chain comprises an LCDR1 of SEQ ID NO: 64, an LCDR2 of SEQ ID NO: 65, and an LCDR3 of SEQ ID NO: 66; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 67, and wherein the light chain comprises an LCDR1 of SEQ ID NO: 64, an LCDR2 of SEQ ID NO: 65, and an LCDR3 of SEQ ID NO: 68; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 67, and wherein the light chain comprises an LCDR1 of SEQ ID NO: 64, an LCDR2 of SEQ ID NO: 65, and an LCDR3 of SEQ ID NO: 68; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 69, and wherein the light chain comprises an LCDR1 of SEQ ID NO: 64, an LCDR2 of SEQ ID NO: 65, and an LCDR3 of SEQ ID NO: 68; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 69, and wherein the light chain comprises an LCDR1 of SEQ ID NO: 64, an LCDR2 of SEQ ID NO: 65, and an LCDR3 of SEQ ID NO: 68; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 70, and wherein the light chain comprises an LCDR1 of SEQ ID NO: 64, an LCDR2 of SEQ ID NO: 65, and an LCDR3 of SEQ ID NO: 71; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 70, and wherein the light chain comprises an LCDR1 of SEQ ID NO: 64, an LCDR2 of SEQ ID NO: 65, and an LCDR3 of SEQ ID NO: 71; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 72, and wherein the light chain comprises an LCDR1 of SEQ ID NO: 64, an LCDR2 of SEQ ID NO: 65, and an LCDR3 of SEQ ID NO: 71; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 72, and wherein the light chain comprises an LCDR1 of SEQ ID NO: 64, an LCDR2 of SEQ ID NO: 65, and an LCDR3 of SEQ ID NO: 71; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 73, and wherein the light chain comprises an LCDR1 of SEQ ID NO: 64, an LCDR2 of SEQ ID NO: 65, and an LCDR3 of SEQ ID NO: 71; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 73, and wherein the light chain comprises an LCDR1 of SEQ ID NO: 64, an LCDR2 of SEQ ID NO: 65, and an LCDR3 of SEQ ID NO: 71; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 74, and wherein the light chain comprises an LCDR1 of SEQ ID NO: 64, an LCDR2 of SEQ ID NO: 65, and an LCDR3 of SEQ ID NO: 75; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 74, and wherein the light chain comprises an LCDR1 of SEQ ID NO: 64, an LCDR2 of SEQ ID NO: 65, and an LCDR3 of SEQ ID NO: 75; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 76, and wherein the light chain comprises an LCDR1 of SEQ ID NO: 64, an LCDR2 of SEQ ID NO: 65, and an LCDR3 of SEQ ID NO: 77; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 76, and wherein the light chain comprises an LCDR1 of SEQ ID NO: 64, an LCDR2 of SEQ ID NO: 65, and an LCDR3 of SEQ ID NO: 77; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 78, and wherein the light chain comprises an LCDR1 of SEQ ID NO: 64, an LCDR2 of SEQ ID NO: 65, and an LCDR3 of SEQ ID NO: 79; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 78, and wherein the light chain comprises an LCDR1 of SEQ ID NO: 64, an LCDR2 of SEQ ID NO: 65, and an LCDR3 of SEQ ID NO: 79; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 80, and wherein the light chain comprises an LCDR1 of SEQ ID NO: 64, an LCDR2 of SEQ ID NO: 65, and an LCDR3 of SEQ ID NO: 81; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 80, and wherein the light chain comprises an LCDR1 of SEQ ID NO: 64, an LCDR2 of SEQ ID NO: 65, and an LCDR3 of SEQ ID NO: 81; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 84, and wherein the light chain comprises an LCDR1 of SEQ ID NO: 85, an LCDR2 of SEQ ID NO: 86, and an LCDR3 of SEQ ID NO: 87; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 84, and wherein the light chain comprises an LCDR1 of SEQ ID NO: 85, an LCDR2 of SEQ ID NO: 86, and an LCDR3 of SEQ ID NO: 87; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 88, and wherein the light chain comprises an LCDR1 of SEQ ID NO: 85, an LCDR2 of SEQ ID NO: 86, and an LCDR3 of SEQ ID NO: 89; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 88, and wherein the light chain comprises an LCDR1 of SEQ ID NO: 85, an LCDR2 of SEQ ID NO: 86, and an LCDR3 of SEQ ID NO: 89; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 90, and wherein the light chain comprises an LCDR1 of SEQ ID NO: 85, an LCDR2 of SEQ ID NO: 86, and an LCDR3 of SEQ ID NO: 89; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 90, and wherein the light chain comprises an LCDR1 of SEQ ID NO: 85, an LCDR2 of SEQ ID NO: 86, and an LCDR3 of SEQ ID NO: 89; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 91, and wherein the light chain comprises an LCDR1 of SEQ ID NO: 85, an LCDR2 of SEQ ID NO: 86, and an LCDR3 of SEQ ID NO: 92; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 91, and wherein the light chain comprises an LCDR1 of SEQ ID NO: 85, an LCDR2 of SEQ ID NO: 86, and an LCDR3 of SEQ ID NO: 92; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 93, and wherein the light chain comprises an LCDR1 of SEQ ID NO: 85, an LCDR2 of SEQ ID NO: 86, and an LCDR3 of SEQ ID NO: 94; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 93, and wherein the light chain comprises an LCDR1 of SEQ ID NO: 85, an LCDR2 of SEQ ID NO: 86, and an LCDR3 of SEQ ID NO: 94; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 84, and wherein the light chain comprises an LCDR1 of SEQ ID NO: 85, an LCDR2 of SEQ ID NO: 86, and an LCDR3 of SEQ ID NO: 95; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 84, and wherein the light chain comprises an LCDR1 of SEQ ID NO: 85, an LCDR2 of SEQ ID NO: 86, and an LCDR3 of SEQ ID NO: 95; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 96, and wherein the light chain comprises an LCDR1 of SEQ ID NO: 85, an LCDR2 of SEQ ID NO: 86, and an LCDR3 of SEQ ID NO: 97; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 96, and wherein the light chain comprises an LCDR1 of SEQ ID NO: 85, an LCDR2 of SEQ ID NO: 86, and an LCDR3 of SEQ ID NO: 97; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 98, and wherein the light chain comprises an LCDR1 of SEQ ID NO: 85, an LCDR2 of SEQ ID NO: 86, and an LCDR3 of SEQ ID NO: 99; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 98, and wherein the light chain comprises an LCDR1 of SEQ ID NO: 85, an LCDR2 of SEQ ID NO: 86, and an LCDR3 of SEQ ID NO: 99; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 100, and wherein the light chain comprises an LCDR1 of SEQ ID NO: 85, an LCDR2 of SEQ ID NO: 86, and an LCDR3 of SEQ ID NO: 101; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 100, and wherein the light chain comprises an LCDR1 of SEQ ID NO: 85, an LCDR2 of SEQ ID NO: 86, and an LCDR3 of SEQ ID NO: 101; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 100, and wherein the light chain comprises an LCDR1 of SEQ ID NO: 85, an LCDR2 of SEQ ID NO: 86, and an LCDR3 of SEQ ID NO: 102; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 82, an HCDR2 of SEQ ID NO: 83, and an HCDR3 of SEQ ID NO: 100, and wherein the light chain comprises an LCDR1 of SEQ ID NO: 85, an LCDR2 of SEQ ID NO: 86, and an LCDR3 of SEQ ID NO: 102; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 105, and wherein the light chain comprises an LCDR1 of SEQ ID NO: 106, an LCDR2 of SEQ ID NO: 107, and an LCDR3 of SEQ ID NO: 108; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 105, and wherein the light chain comprises an LCDR1 of SEQ ID NO: 106, an LCDR2 of SEQ ID NO: 107, and an LCDR3 of SEQ ID NO: 108; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 109, and wherein the light chain comprises an LCDR1 of SEQ ID NO: 106, an LCDR2 of SEQ ID NO: 107, and an LCDR3 of SEQ ID NO: 110; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 109, and wherein the light chain comprises an LCDR1 of SEQ ID NO: 106, an LCDR2 of SEQ ID NO: 107, and an LCDR3 of SEQ ID NO: 110; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 111, and wherein the light chain comprises an LCDR1 of SEQ ID NO: 106, an LCDR2 of SEQ ID NO: 107, and an LCDR3 of SEQ ID NO: 112; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 111, and wherein the light chain comprises an LCDR1 of SEQ ID NO: 106, an LCDR2 of SEQ ID NO: 107, and an LCDR3 of SEQ ID NO: 112; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 105, and wherein the light chain comprises an LCDR1 of SEQ ID NO: 106, an LCDR2 of SEQ ID NO: 107, and an LCDR3 of SEQ ID NO: 113; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 105, and wherein the light chain comprises an LCDR1 of SEQ ID NO: 106, an LCDR2 of SEQ ID NO: 107, and an LCDR3 of SEQ ID NO: 113; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 114, and wherein the light chain comprises an LCDR1 of SEQ ID NO: 106, an LCDR2 of SEQ ID NO: 107, and an LCDR3 of SEQ ID NO: 115; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 114, and wherein the light chain comprises an LCDR1 of SEQ ID NO: 106, an LCDR2 of SEQ ID NO: 107 and an LCDR3 of SEQ ID NO: 115; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 116, and wherein the light chain comprises an LCDR1 of SEQ ID NO: 106, an LCDR2 of SEQ ID NO: 107, and an LCDR3 of SEQ ID NO: 113; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 116, and wherein the light chain comprises an LCDR1 of SEQ ID NO: 106, an LCDR2 of SEQ ID NO: 107, and an LCDR3 of SEQ ID NO: 113; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 117, and wherein the light chain comprises an LCDR1 of SEQ ID NO: 103, an LCDR2 of SEQ ID NO: 107, and an LCDR3 of SEQ ID NO: 118; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 117, and wherein the light chain comprises an LCDR1 of SEQ ID NO: 103, an LCDR2 of SEQ ID NO: 107, and an LCDR3 of SEQ ID NO: 118; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 116, and wherein the light chain comprises an LCDR1 of SEQ ID NO: 106, an LCDR2 of SEQ ID NO: 107, and an LCDR3 of SEQ ID NO: 119; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 116, and wherein the light chain comprises an LCDR1 of SEQ ID NO: 106, an LCDR2 of SEQ ID NO: 107, and an LCDR3 of SEQ ID NO: 119; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 120, and wherein the light chain comprises an LCDR1 of SEQ ID NO: 106, an LCDR2 of SEQ ID NO: 107, and an LCDR3 of SEQ ID NO: 121; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 120, and wherein the light chain comprises an LCDR1 of SEQ ID NO: 106, an LCDR2 of SEQ ID NO: 107, and an LCDR3 of SEQ ID NO: 121; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 122, and wherein the light chain comprises an LCDR1 of SEQ ID NO: 106, an LCDR2 of SEQ ID NO: 107, and an LCDR3 of SEQ ID NO: 123; about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant; and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain comprises an HCDR1 of SEQ ID NO: 103, an HCDR2 of SEQ ID NO: 104, and an HCDR3 of SEQ ID NO: 122, and wherein the light chain comprises an LCDR1 of SEQ ID NO: 106, an LCDR2 of SEQ ID NO: 107, and an LCDR3 of SEQ ID NO: 123; about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant; and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 1, and wherein the light chain variable region corresponds to SEQ ID NO: 2, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 1, and wherein the light chain variable region corresponds to SEQ ID NO: 2, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 3, and wherein the light chain variable region corresponds to SEQ ID NO: 4, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 3, and wherein the light chain variable region corresponds to SEQ ID NO: 4, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 5, and wherein the light chain variable region corresponds to SEQ ID NO: 6, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 5, and wherein the light chain variable region corresponds to SEQ ID NO: 6, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 7, and wherein the light chain variable region corresponds to SEQ ID NO: 8, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 7, and wherein the light chain variable region corresponds to SEQ ID NO: 8, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 9, and wherein the light chain variable region corresponds to SEQ ID NO: 10, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 9, and wherein the light chain variable region corresponds to SEQ ID NO: 10, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 11, and wherein the light chain variable region corresponds to SEQ ID NO: 12, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 11, and wherein the light chain variable region corresponds to SEQ ID NO: 12, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 13, and wherein the light chain variable region corresponds to SEQ ID NO: 14, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 13, and wherein the light chain variable region corresponds to SEQ ID NO: 14, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 15, and wherein the light chain variable region corresponds to SEQ ID NO: 16, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 15, and wherein the light chain variable region corresponds to SEQ ID NO: 16, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 17, and wherein the light chain variable region corresponds to SEQ ID NO: 18, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 17, and wherein the light chain variable region corresponds to SEQ ID NO: 18, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 19, and wherein the light chain variable region corresponds to SEQ ID NO: 20, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 19, and wherein the light chain variable region corresponds to SEQ ID NO: 20, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 21, and wherein the light chain variable region corresponds to SEQ ID NO: 22, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 21, and wherein the light chain variable region corresponds to SEQ ID NO: 22, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 23, and wherein the light chain variable region corresponds to SEQ ID NO: 24, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 23, and wherein the light chain variable region corresponds to SEQ ID NO: 24, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 25, and wherein the light chain variable region corresponds to SEQ ID NO: 26, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 25, and wherein the light chain variable region corresponds to SEQ ID NO: 26, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 27, and wherein the light chain variable region corresponds to SEQ ID NO: 28, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 27, and wherein the light chain variable region corresponds to SEQ ID NO: 28, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 29, and wherein the light chain variable region corresponds to SEQ ID NO: 30, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 29, and wherein the light chain variable region corresponds to SEQ ID NO: 30, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 31, and wherein the light chain variable region corresponds to SEQ ID NO: 32, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 31, and wherein the light chain variable region corresponds to SEQ ID NO: 32, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 33, and wherein the light chain variable region corresponds to SEQ ID NO: 34, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 33, and wherein the light chain variable region corresponds to SEQ ID NO: 34, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 35, and wherein the light chain variable region corresponds to SEQ ID NO: 36, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 35, and wherein the light chain variable region corresponds to SEQ ID NO: 36, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 37, and wherein the light chain variable region corresponds to SEQ ID NO: 38, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 37, and wherein the light chain variable region corresponds to SEQ ID NO: 38, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 39, and wherein the light chain variable region corresponds to SEQ ID NO: 40, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 39, and wherein the light chain variable region corresponds to SEQ ID NO: 40, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 41, and wherein the light chain variable region corresponds to SEQ ID NO: 42, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 41, and wherein the light chain variable region corresponds to SEQ ID NO: 42, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 43, and wherein the light chain variable region corresponds to SEQ ID NO: 44, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 43, and wherein the light chain variable region corresponds to SEQ ID NO: 44, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 45, and wherein the light chain variable region corresponds to SEQ ID NO: 46, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 45, and wherein the light chain variable region corresponds to SEQ ID NO: 46, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 47, and wherein the light chain variable region corresponds to SEQ ID NO: 48, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 47, and wherein the light chain variable region corresponds to SEQ ID NO: 48, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 49, and wherein the light chain variable region corresponds to SEQ ID NO: 50, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 49, and wherein the light chain variable region corresponds to SEQ ID NO: 50, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 51, and wherein the light chain variable region corresponds to SEQ ID NO: 52, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 51, and wherein the light chain variable region corresponds to SEQ ID NO: 52, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 53, and wherein the light chain variable region corresponds to SEQ ID NO: 54, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 53, and wherein the light chain variable region corresponds to SEQ ID NO: 54, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 55, and wherein the light chain variable region corresponds to SEQ ID NO: 56, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 55, and wherein the light chain variable region corresponds to SEQ ID NO: 56, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 57, and wherein the light chain variable region corresponds to SEQ ID NO: 58, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 57, and wherein the light chain variable region corresponds to SEQ ID NO: 58, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 59, and wherein the light chain variable region corresponds to SEQ ID NO: 60, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 59, and wherein the light chain variable region corresponds to SEQ ID NO: 60, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 334, and wherein the light chain variable region corresponds to SEQ ID NO: 2, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 334, and wherein the light chain variable region corresponds to SEQ ID NO: 2, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 335, and wherein the light chain variable region corresponds to SEQ ID NO: 4, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 335, and wherein the light chain variable region corresponds to SEQ ID NO: 4, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 336, and wherein the light chain variable region corresponds to SEQ ID NO: 6, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 336, and wherein the light chain variable region corresponds to SEQ ID NO: 6, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 337, and wherein the light chain variable region corresponds to SEQ ID NO: 8, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 337, and wherein the light chain variable region corresponds to SEQ ID NO: 8, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 338, and wherein the light chain variable region corresponds to SEQ ID NO: 10, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 338, and wherein the light chain variable region corresponds to SEQ ID NO: 10, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 339, and wherein the light chain variable region corresponds to SEQ ID NO: 12, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 339, and wherein the light chain variable region corresponds to SEQ ID NO: 12, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 340, and wherein the light chain variable region corresponds to SEQ ID NO: 14, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 340, and wherein the light chain variable region corresponds to SEQ ID NO: 14, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 341, and wherein the light chain variable region corresponds to SEQ ID NO: 16, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 341, and wherein the light chain variable region corresponds to SEQ ID NO: 16, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 342, and wherein the light chain variable region corresponds to SEQ ID NO: 18, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 342, and wherein the light chain variable region corresponds to SEQ ID NO: 18, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 343, and wherein the light chain variable region corresponds to SEQ ID NO: 20, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 343, and wherein the light chain variable region corresponds to SEQ ID NO: 20, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 344, and wherein the light chain variable region corresponds to SEQ ID NO: 22, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 344, and wherein the light chain variable region corresponds to SEQ ID NO: 22, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 345, and wherein the light chain variable region corresponds to SEQ ID NO: 24, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 345, and wherein the light chain variable region corresponds to SEQ ID NO: 24, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 346, and wherein the light chain variable region corresponds to SEQ ID NO: 26, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 346, and wherein the light chain variable region corresponds to SEQ ID NO: 26, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 347, and wherein the light chain variable region corresponds to SEQ ID NO: 28, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 347, and wherein the light chain variable region corresponds to SEQ ID NO: 28, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 348, and wherein the light chain variable region corresponds to SEQ ID NO: 30, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 348, and wherein the light chain variable region corresponds to SEQ ID NO: 30, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 349, and wherein the light chain variable region corresponds to SEQ ID NO: 32, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 349, and wherein the light chain variable region corresponds to SEQ ID NO: 32, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 350, and wherein the light chain variable region corresponds to SEQ ID NO: 34, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 350, and wherein the light chain variable region corresponds to SEQ ID NO: 34, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 351, and wherein the light chain variable region corresponds to SEQ ID NO: 36, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 351, and wherein the light chain variable region corresponds to SEQ ID NO: 36, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 352, and wherein the light chain variable region corresponds to SEQ ID NO: 38, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 352, and wherein the light chain variable region corresponds to SEQ ID NO: 38, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 353, and wherein the light chain variable region corresponds to SEQ ID NO: 40, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 353, and wherein the light chain variable region corresponds to SEQ ID NO: 40, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 354, and wherein the light chain variable region corresponds to SEQ ID NO: 42, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 354, and wherein the light chain variable region corresponds to SEQ ID NO: 42, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 355, and wherein the light chain variable region corresponds to SEQ ID NO: 44, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 355, and wherein the light chain variable region corresponds to SEQ ID NO: 44, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 356, and wherein the light chain variable region corresponds to SEQ ID NO: 46, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 356, and wherein the light chain variable region corresponds to SEQ ID NO: 46, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 357, and wherein the light chain variable region corresponds to SEQ ID NO: 48, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 357, and wherein the light chain variable region corresponds to SEQ ID NO: 48, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 358, and wherein the light chain variable region corresponds to SEQ ID NO: 50, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 358, and wherein the light chain variable region corresponds to SEQ ID NO: 50, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 359, and wherein the light chain variable region corresponds to SEQ ID NO: 52, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 359, and wherein the light chain variable region corresponds to SEQ ID NO: 52, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 360, and wherein the light chain variable region corresponds to SEQ ID NO: 54, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 360, and wherein the light chain variable region corresponds to SEQ ID NO: 54, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 361, and wherein the light chain variable region corresponds to SEQ ID NO: 56, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 361, and wherein the light chain variable region corresponds to SEQ ID NO: 56, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 362, and wherein the light chain variable region corresponds to SEQ ID NO: 58, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 362, and wherein the light chain variable region corresponds to SEQ ID NO: 58, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain variable region and a light chain variable region, wherein the heavy chain variable region corresponds to SEQ ID NO: 363, and wherein the light chain variable region corresponds to SEQ ID NO: 60, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain variable region and a light chain variable region, wherein the heavy chain variable region corresponds to SEQ ID NO: 363, and wherein the light chain variable region corresponds to SEQ ID NO: 60, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 168 or SEQ ID NO: 169, and the light chain corresponds to SEQ ID NO: 228, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 168 or SEQ ID NO: 169, and the light chain corresponds to SEQ ID NO: 228, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 170 or SEQ ID NO: 171, and the light chain corresponds to SEQ ID NO: 229, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 170 or SEQ ID NO: 171, and the light chain corresponds to SEQ ID NO: 229, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 172 or SEQ ID NO: 173, and the light chain corresponds to SEQ ID NO: 230, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 172 or SEQ ID NO: 173, and the light chain corresponds to SEQ ID NO: 230, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 174 or SEQ ID NO: 175, and the light chain corresponds to SEQ ID NO: 231, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 174 or SEQ ID NO: 175, and the light chain corresponds to SEQ ID NO: 231, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 176 or SEQ ID NO: 177, and the light chain corresponds to SEQ ID NO: 232, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 176 or SEQ ID NO: 177, and the light chain corresponds to SEQ ID NO: 232, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 178 or SEQ ID NO: 179, and the light chain corresponds to SEQ ID NO: 233, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 178 or SEQ ID NO: 179, and the light chain corresponds to SEQ ID NO: 233, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 180 or SEQ ID NO: 181, and the light chain corresponds to SEQ ID NO: 234, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 180 or SEQ ID NO: 181, and the light chain corresponds to SEQ ID NO: 234, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 182 or SEQ ID NO: 183, and the light chain corresponds to SEQ ID NO: 235, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 182 or SEQ ID NO: 183, and the light chain corresponds to SEQ ID NO: 235, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 184 or SEQ ID NO: 185, and the light chain corresponds to SEQ ID NO: 236, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 184 or SEQ ID NO: 185, and the light chain corresponds to SEQ ID NO: 236, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 186 or SEQ ID NO: 187, and the light chain corresponds to SEQ ID NO: 237, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 186 or SEQ ID NO: 187, and the light chain corresponds to SEQ ID NO: 237, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 188 or SEQ ID NO: 189, and the light chain corresponds to SEQ ID NO: 238, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 188 or SEQ ID NO: 189, and the light chain corresponds to SEQ ID NO: 238, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 190 or SEQ ID NO: 191, and the light chain corresponds to SEQ ID NO: 239, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 190 or SEQ ID NO: 191, and the light chain corresponds to SEQ ID NO: 239, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 192 or SEQ ID NO: 193, and the light chain corresponds to SEQ ID NO: 240, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 192 or SEQ ID NO: 193, and the light chain corresponds to SEQ ID NO: 240, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 194 or SEQ ID NO: 195, and the light chain corresponds to SEQ ID NO: 241, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 194 or SEQ ID NO: 195, and the light chain corresponds to SEQ ID NO: 241, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 196 or SEQ ID NO: 197, and the light chain corresponds to SEQ ID NO: 242, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 196 or SEQ ID NO: 197, and the light chain corresponds to SEQ ID NO: 242, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 198 or SEQ ID NO: 199, and the light chain corresponds to SEQ ID NO: 243, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 198 or SEQ ID NO: 199, and the light chain corresponds to SEQ ID NO: 243, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 200 or SEQ ID NO: 201, and the light chain corresponds to SEQ ID NO: 244, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 200 or SEQ ID NO: 201, and the light chain corresponds to SEQ ID NO: 244, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 202 or SEQ ID NO: 203, and the light chain corresponds to SEQ ID NO: 245, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 202 or SEQ ID NO: 203, and the light chain corresponds to SEQ ID NO: 245, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 204 or SEQ ID NO: 205, and the light chain corresponds to SEQ ID NO: 246, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 204 or SEQ ID NO: 205, and the light chain corresponds to SEQ ID NO: 246, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 206 or SEQ ID NO: 207, and the light chain corresponds to SEQ ID NO: 247, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 206 or SEQ ID NO: 207, and the light chain corresponds to SEQ ID NO: 247, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 208 or SEQ ID NO: 209, and the light chain corresponds to SEQ ID NO: 248, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 208 or SEQ ID NO: 209, and the light chain corresponds to SEQ ID NO: 248, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 210 or SEQ ID NO: 211, and the light chain corresponds to SEQ ID NO: 249, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 210 or SEQ ID NO: 211, and the light chain corresponds to SEQ ID NO: 249, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 212 or SEQ ID NO: 213, and the light chain corresponds to SEQ ID NO: 250, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 212 or SEQ ID NO: 213, and the light chain corresponds to SEQ ID NO: 250, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 214 or SEQ ID NO: 215, and the light chain corresponds to SEQ ID NO: 251, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 214 or SEQ ID NO: 215, and the light chain corresponds to SEQ ID NO: 251, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 216 or SEQ ID NO: 217, and the light chain corresponds to SEQ ID NO: 252, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 216 or SEQ ID NO: 217, and the light chain corresponds to SEQ ID NO: 252, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 218 or SEQ ID NO: 219, and the light chain corresponds to SEQ ID NO: 253, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 218 or SEQ ID NO: 219, and the light chain corresponds to SEQ ID NO: 253, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 220 or SEQ ID NO: 221, and the light chain corresponds to SEQ ID NO: 254, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 220 or SEQ ID NO: 221, and the light chain corresponds to SEQ ID NO: 254, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 222 or SEQ ID NO: 223, and the light chain corresponds to SEQ ID NO: 255, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 222 or SEQ ID NO: 223, and the light chain corresponds to SEQ ID NO: 255, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 224 or SEQ ID NO: 225, and the light chain corresponds to SEQ ID NO: 256, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 224 or SEQ ID NO: 225, and the light chain corresponds to SEQ ID NO: 256, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 226 or SEQ ID NO: 227, and the light chain corresponds to SEQ ID NO: 257, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 226 or SEQ ID NO: 227, and the light chain corresponds to SEQ ID NO: 257, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 364 or SEQ ID NO: 365, and the light chain corresponds to SEQ ID NO: 228, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 364 or SEQ ID NO: 365, and the light chain corresponds to SEQ ID NO: 228, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 366 or SEQ ID NO: 367, and the light chain corresponds to SEQ ID NO: 229, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 366 or SEQ ID NO: 367, and the light chain corresponds to SEQ ID NO: 229, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 368 or SEQ ID NO: 369, and the light chain corresponds to SEQ ID NO: 230, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 368 or SEQ ID NO: 369, and the light chain corresponds to SEQ ID NO: 230, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 370 or SEQ ID NO: 371, and the light chain corresponds to SEQ ID NO: 231, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 370 or SEQ ID NO: 371, and the light chain corresponds to SEQ ID NO: 231, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 372 or SEQ ID NO: 373, and the light chain corresponds to SEQ ID NO: 232, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 372 or SEQ ID NO: 373, and the light chain corresponds to SEQ ID NO: 232, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 374 or SEQ ID NO: 375, and the light chain corresponds to SEQ ID NO: 233, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 374 or SEQ ID NO: 375, and the light chain corresponds to SEQ ID NO: 233, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 376 or SEQ ID NO: 377, and the light chain corresponds to SEQ ID NO: 234, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 376 or SEQ ID NO: 377, and the light chain corresponds to SEQ ID NO: 234, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 378 or SEQ ID NO: 379, and the light chain corresponds to SEQ ID NO: 235, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 378 or SEQ ID NO: 379, and the light chain corresponds to SEQ ID NO: 235, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 380 or SEQ ID NO: 381, and the light chain corresponds to SEQ ID NO: 236, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 380 or SEQ ID NO: 381, and the light chain corresponds to SEQ ID NO: 236, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 382 or SEQ ID NO: 383, and the light chain corresponds to SEQ ID NO: 237, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 382 or SEQ ID NO: 383, and the light chain corresponds to SEQ ID NO: 237, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 384 or SEQ ID NO: 385, and the light chain corresponds to SEQ ID NO: 238, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 384 or SEQ ID NO: 385, and the light chain corresponds to SEQ ID NO: 238, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 386 or SEQ ID NO: 387, and the light chain corresponds to SEQ ID NO: 239, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 386 or SEQ ID NO: 387, and the light chain corresponds to SEQ ID NO: 239, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 388 or SEQ ID NO: 389, and the light chain corresponds to SEQ ID NO: 240, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 388 or SEQ ID NO: 389, and the light chain corresponds to SEQ ID NO: 240, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 390 or SEQ ID NO: 391, and the light chain corresponds to SEQ ID NO: 241, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 390 or SEQ ID NO: 391, and the light chain corresponds to SEQ ID NO: 241, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 392 or SEQ ID NO: 393, and the light chain corresponds to SEQ ID NO: 242, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 392 or SEQ ID NO: 393, and the light chain corresponds to SEQ ID NO: 242, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 394 or SEQ ID NO: 395, and the light chain corresponds to SEQ ID NO: 243, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 394 or SEQ ID NO: 395, and the light chain corresponds to SEQ ID NO: 243, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 396 or SEQ ID NO: 397, and the light chain corresponds to SEQ ID NO: 244, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 396 or SEQ ID NO: 397, and the light chain corresponds to SEQ ID NO: 244, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 398 or SEQ ID NO: 399, and the light chain corresponds to SEQ ID NO: 245, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 398 or SEQ ID NO: 399, and the light chain corresponds to SEQ ID NO: 245, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 400 or SEQ ID NO: 401, and the light chain corresponds to SEQ ID NO: 246, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 400 or SEQ ID NO: 401, and the light chain corresponds to SEQ ID NO: 246, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 402 or SEQ ID NO: 403, and the light chain corresponds to SEQ ID NO: 247, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 402 or SEQ ID NO: 403, and the light chain corresponds to SEQ ID NO: 247, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 404 or SEQ ID NO: 405, and the light chain corresponds to SEQ ID NO: 248, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 404 or SEQ ID NO: 405, and the light chain corresponds to SEQ ID NO: 248, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 406 or SEQ ID NO: 407, and the light chain corresponds to SEQ ID NO: 249, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 406 or SEQ ID NO: 407, and the light chain corresponds to SEQ ID NO: 249, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 408 or SEQ ID NO: 409, and the light chain corresponds to SEQ ID NO: 250, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 408 or SEQ ID NO: 409, and the light chain corresponds to SEQ ID NO: 250, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 410 or SEQ ID NO: 411, and the light chain corresponds to SEQ ID NO: 251, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 410 or SEQ ID NO: 411, and the light chain corresponds to SEQ ID NO: 251, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 412 or SEQ ID NO: 413, and the light chain corresponds to SEQ ID NO: 252, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 412 or SEQ ID NO: 413, and the light chain corresponds to SEQ ID NO: 252, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 414 or SEQ ID NO: 415, and the light chain corresponds to SEQ ID NO: 253, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 414 or SEQ ID NO: 415, and the light chain corresponds to SEQ ID NO: 253, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 416 or SEQ ID NO: 417, and the light chain corresponds to SEQ ID NO: 254, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 416 or SEQ ID NO: 417, and the light chain corresponds to SEQ ID NO: 254, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 418 or SEQ ID NO: 419, and the light chain corresponds to SEQ ID NO: 255, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 418 or SEQ ID NO: 419, and the light chain corresponds to SEQ ID NO: 255, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 420 or SEQ ID NO: 421, and the light chain corresponds to SEQ ID NO: 256, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 420 or SEQ ID NO: 421, and the light chain corresponds to SEQ ID NO: 256, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, a pharmaceutical composition is provided that comprises about 25 mg/mL to about 300 mg/mL of an antibody having a heavy chain and a light chain, wherein the heavy chain corresponds to SEQ ID NO: 422 or SEQ ID NO: 423, and the light chain corresponds to SEQ ID NO: 257, about 5 mM to about 50 mM of a buffer, about 5 mM to about 15 mM of an antioxidant, about 1% w/v to about 14% w/v of a sugar, about 20 mM to about 180 mM of a viscosity modifying agent, about 0.001% w/v to about 0.4% w/v of a surfactant, and wherein the pharmaceutical composition is at a pH of about 6.5 to about 8.0.

In some embodiments, a pharmaceutical composition is provided that comprises about 25 mg/mL to about 200 mg/mL of an antibody having a heavy chain and a light chain, wherein the heavy chain corresponds to SEQ ID NO: 422 or SEQ ID NO: 423, and the light chain corresponds to SEQ ID NO: 257, about 15 mM to about 25 mM of a buffer, about 5 mM to about 15 mM of an antioxidant, about 1% w/v to about 10% w/v of a sugar, about 60 mM to about 150 mM of a viscosity modifying agent, about 0.001% w/v to about 0.2% w/v of a surfactant, and wherein the pharmaceutical composition is at a pH of about 6.5 to about 8.0.

In some embodiments, the pharmaceutical composition comprises a buffer at a concentration of about 5 mM to about 15 mM. In some embodiments, the pharmaceutical composition comprises a buffer at a concentration of about 15 mM to about 20 mM. In some embodiments, the pharmaceutical composition comprises a buffer at a concentration of about 20 mM to about 25 mM.

In some embodiments, the pharmaceutical composition comprises a buffer at a concentration of about 5 mM. In some embodiments, the pharmaceutical composition comprises a buffer at a concentration of about 10 mM. In some embodiments, the pharmaceutical composition comprises a buffer at a concentration of about 15 mM. In some embodiments, the pharmaceutical composition comprises a buffer at a concentration of about 16 mM. In some embodiments, the pharmaceutical composition comprises a buffer at a concentration of about 17 mM. In some embodiments, the pharmaceutical composition comprises a buffer at a concentration of about 18 mM. In some embodiments, the pharmaceutical composition comprises a buffer at a concentration of about 19 mM. In some embodiments, the pharmaceutical composition comprises a buffer at a concentration of about 20 mM. In some embodiments, the pharmaceutical composition comprises a buffer at a concentration of about 21 mM. In some embodiments, the pharmaceutical composition comprises a buffer at a concentration of about 22 mM. In some embodiments, the pharmaceutical composition comprises a buffer at a concentration of about 23 mM. In some embodiments, the pharmaceutical composition comprises a buffer at a concentration of about 24 mM. In some embodiments, the pharmaceutical composition comprises a buffer at a concentration of about 25 mM. In some embodiments, the pharmaceutical composition comprises a buffer at a concentration of about 30 mM. In some embodiments, the pharmaceutical composition comprises a buffer at a concentration of about 35 mM. In some embodiments, the pharmaceutical composition comprises a buffer at a concentration of about 40 mM. In some embodiments, the pharmaceutical composition comprises a buffer at a concentration of about 45 mM. In some embodiments, the pharmaceutical composition comprises a buffer at a concentration of about 50 mM.

In some embodiments, the pharmaceutical compositions comprise a buffer (e.g., histidine, acetate, phosphate or citrate buffer) and/or a stabilizer agent (e.g. human albumin), etc., or a combination thereof. In some embodiments, the pharmaceutical compositions comprise one or more pharmaceutically acceptable carriers, including, e.g., ion exchangers, alumina, aluminum stearate, lecithin, serum proteins, such as human serum albumin, buffer substances such as phosphates, sucrose, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, polyethylene-polyoxypropylene-block polymers, and polyethylene glycol, or a combination thereof.

Various buffers can be present in the compositions of the present disclosure. In some embodiments, the pharmaceutical composition comprises a buffer, wherein the buffer is phosphoric acid buffer, citric acid buffer, acetic acid buffer, succinic acid buffer, citrate buffer, ascorbic acid buffer, glutamic acid buffer, lactic acid buffer, maleic acid buffer, trometamol buffer, and gluconic acid buffer, acetate buffer, succinate buffer, phosphate buffer, histidine buffer or any combination thereof. In some embodiments, the buffer is histidine buffer, histidine HCl buffer, glycine buffer, Tris/glycine buffer, acetate buffer, sodium acetate buffer, potassium acetate buffer, magnesium acetate buffer, phosphate buffer, citrate buffer, or succinate buffer. In some embodiments, the buffer is histidine buffer. In some embodiments, the buffer is histidine HCl buffer. In some embodiments, the buffer is glycine buffer. In some embodiments, the buffer is Tris/glycine buffer. In some embodiments, the buffer is acetate buffer. In some embodiments, the buffer is sodium acetate buffer. In some embodiments, the buffer is potassium acetate buffer. In some embodiments, the buffer is magnesium acetate buffer. In some embodiments, the buffer is phosphate buffer. In some embodiments, the buffer is citrate buffer. In some embodiments, the buffer is succinate buffer.

In some embodiments, the pharmaceutical composition comprises a buffer selected from tris buffer, histidine buffer, HEPES, phosphate buffer, acetate buffer, citrate buffer, succinate buffer, ascorbate buffer, glutamate buffer, lactate buffer, maleate buffer, trometamol buffer, gluconate buffer, or any combination thereof.

In some embodiments, the pharmaceutical composition comprises tris buffer. In some embodiments, the pharmaceutical composition comprises histidine buffer. In some embodiments, the pharmaceutical composition comprises HEPES. In some embodiments, the pharmaceutical composition comprises phosphate buffer. In some embodiments, the pharmaceutical composition comprises acetate buffer. In some embodiments, the pharmaceutical composition comprises citrate buffer. In some embodiments, the pharmaceutical composition comprises succinate buffer. In some embodiments, the pharmaceutical composition comprises ascorbate buffer. In some embodiments, the pharmaceutical composition comprises glutamate buffer. In some embodiments, the pharmaceutical composition comprises lactate buffer. In some embodiments, the pharmaceutical composition comprises maleate buffer. In some embodiments, the pharmaceutical composition comprises trometamol buffer. In some embodiments, the pharmaceutical composition comprises gluconate buffer.

In some embodiments, the pharmaceutical composition comprises a phosphate buffer. In some embodiments, the pharmaceutical composition comprises phosphate buffer at a concentration of about 5 mM to about 15 mM. In some embodiments, the pharmaceutical composition comprises phosphate buffer at a concentration of about 15 mM to about 20 mM. In some embodiments, the pharmaceutical composition comprises phosphate buffer at a concentration of about 20 mM to about 25 mM. In some embodiments, the pharmaceutical composition comprises phosphate buffer at a concentration of about 25 mM to about 50 mM.

In some embodiments, the pharmaceutical composition comprises phosphate buffer at a concentration of about 15 mM. In some embodiments, the pharmaceutical composition comprises phosphate buffer at a concentration of about 16 mM. In some embodiments, the pharmaceutical composition comprises phosphate buffer at a concentration of about 17 mM. In some embodiments, the pharmaceutical composition comprises phosphate buffer at a concentration of about 18 mM. In some embodiments, the pharmaceutical composition comprises phosphate buffer at a concentration of about 19 mM. In some embodiments, the pharmaceutical composition comprises phosphate buffer at a concentration of about 20 mM. In some embodiments, the pharmaceutical composition comprises phosphate buffer at a concentration of about 21 mM. In some embodiments, the pharmaceutical composition comprises phosphate buffer at a concentration of about 22 mM. In some embodiments, the pharmaceutical composition comprises phosphate buffer at a concentration of about 23 mM. In some embodiments, the pharmaceutical composition comprises phosphate buffer at a concentration of about 24 mM. In some embodiments, the pharmaceutical composition comprises phosphate buffer at a concentration of about 25 mM.

In some embodiments, the pharmaceutical composition comprises a tris buffer. In some embodiments, the pharmaceutical composition comprises tris buffer at a concentration of about 15 mM to about 20 mM. In some embodiments, the pharmaceutical composition comprises tris buffer at a concentration of about 20 mM to about 25 mM.

In some embodiments, the pharmaceutical composition comprises tris buffer at a concentration of about 15 mM. In some embodiments, the pharmaceutical composition comprises tris buffer at a concentration of about 16 mM. In some embodiments, the pharmaceutical composition comprises tris buffer at a concentration of about 17 mM. In some embodiments, the pharmaceutical composition comprises tris buffer at a concentration of about 18 mM. In some embodiments, the pharmaceutical composition comprises tris buffer at a concentration of about 19 mM. In some embodiments, the pharmaceutical composition comprises tris buffer at a concentration of about 20 mM. In some embodiments, the pharmaceutical composition comprises tris buffer at a concentration of about 21 mM. In some embodiments, the pharmaceutical composition comprises tris buffer at a concentration of about 22 mM. In some embodiments, the pharmaceutical composition comprises tris buffer at a concentration of about 23 mM. In some embodiments, the pharmaceutical composition comprises tris buffer at a concentration of about 24 mM. In some embodiments, the pharmaceutical composition comprises tris buffer at a concentration of about 25 mM.

In some embodiments, the pharmaceutical composition comprises a histidine buffer. In some embodiments, the pharmaceutical composition comprises histidine buffer at a concentration of about 15 mM to about 20 mM. In some embodiments, the pharmaceutical composition comprises histidine buffer at a concentration of about 20 mM to about 25 mM.

In some embodiments, the pharmaceutical composition comprises histidine buffer at a concentration of about 15 mM. In some embodiments, the pharmaceutical composition comprises histidine buffer at a concentration of about 16 mM. In some embodiments, the pharmaceutical composition comprises histidine buffer at a concentration of about 17 mM. In some embodiments, the pharmaceutical composition comprises histidine buffer at a concentration of about 18 mM. In some embodiments, the pharmaceutical composition comprises histidine buffer at a concentration of about 19 mM. In some embodiments, the pharmaceutical composition comprises histidine buffer at a concentration of about 20 mM. In some embodiments, the pharmaceutical composition comprises histidine buffer at a concentration of about 21 mM. In some embodiments, the pharmaceutical composition comprises histidine buffer at a concentration of about 22 mM. In some embodiments, the pharmaceutical composition comprises histidine buffer at a concentration of about 23 mM. In some embodiments, the pharmaceutical composition comprises histidine buffer at a concentration of about 24 mM. In some embodiments, the pharmaceutical composition comprises histidine buffer at a concentration of about 25 mM.

In some embodiments, the pharmaceutical composition comprises HEPES. In some embodiments, the pharmaceutical composition comprises HEPES at a concentration of about 15 mM to about 20 mM. In some embodiments, the pharmaceutical composition comprises HEPES at a concentration of about 20 mM to about 25 mM.

In some embodiments, the pharmaceutical composition comprises HEPES at a concentration of about 15 mM. In some embodiments, the pharmaceutical composition comprises HEPES at a concentration of about 16 mM. In some embodiments, the pharmaceutical composition comprises HEPES at a concentration of about 17 mM. In some embodiments, the pharmaceutical composition comprises HEPES at a concentration of about 18 mM. In some embodiments, the pharmaceutical composition comprises HEPES at a concentration of about 19 mM. In some embodiments, the pharmaceutical composition comprises HEPES at a concentration of about 20 mM. In some embodiments, the pharmaceutical composition comprises HEPES at a concentration of about 21 mM. In some embodiments, the pharmaceutical composition comprises HEPES at a concentration of about 22 mM. In some embodiments, the pharmaceutical composition comprises HEPES at a concentration of about 23 mM. In some embodiments, the pharmaceutical composition comprises HEPES at a concentration of about 24 mM. In some embodiments, the pharmaceutical composition comprises HEPES at a concentration of about 25 mM.

In some embodiments, the pharmaceutical composition comprises acetate buffer. In some embodiments, the pharmaceutical composition comprises acetate buffer at a concentration of about 15 mM to about 20 mM. In some embodiments, the pharmaceutical composition comprises acetate buffer at a concentration of about 20 mM to about 25 mM.

In some embodiments, the pharmaceutical composition comprises acetate buffer at a concentration of about 15 mM. In some embodiments, the pharmaceutical composition comprises acetate buffer at a concentration of about 16 mM. In some embodiments, the pharmaceutical composition comprises acetate buffer at a concentration of about 17 mM. In some embodiments, the pharmaceutical composition comprises acetate buffer at a concentration of about 18 mM. In some embodiments, the pharmaceutical composition comprises acetate buffer at a concentration of about 19 mM. In some embodiments, the pharmaceutical composition comprises acetate buffer at a concentration of about 20 mM. In some embodiments, the pharmaceutical composition comprises acetate buffer at a concentration of about 21 mM. In some embodiments, the pharmaceutical composition comprises acetate buffer at a concentration of about 22 mM. In some embodiments, the pharmaceutical composition comprises acetate buffer at a concentration of about 23 mM. In some embodiments, the pharmaceutical composition comprises acetate buffer at a concentration of about 24 mM. In some embodiments, the pharmaceutical composition comprises acetate buffer at a concentration of about 25 mM.

In some embodiments, the pharmaceutical composition comprises citrate buffer. In some embodiments, the pharmaceutical composition comprises citrate buffer at a concentration of about 15 mM to about 20 mM. In some embodiments, the pharmaceutical composition comprises citrate buffer at a concentration of about 20 mM to about 25 mM.

In some embodiments, the pharmaceutical composition comprises citrate buffer at a concentration of about 15 mM. In some embodiments, the pharmaceutical composition comprises citrate buffer at a concentration of about 16 mM. In some embodiments, the pharmaceutical composition comprises citrate buffer at a concentration of about 17 mM. In some embodiments, the pharmaceutical composition comprises citrate buffer at a concentration of about 18 mM. In some embodiments, the pharmaceutical composition comprises citrate buffer at a concentration of about 19 mM. In some embodiments, the pharmaceutical composition comprises citrate buffer at a concentration of about 20 mM. In some embodiments, the pharmaceutical composition comprises citrate buffer at a concentration of about 21 mM. In some embodiments, the pharmaceutical composition comprises citrate buffer at a concentration of about 22 mM. In some embodiments, the pharmaceutical composition comprises citrate buffer at a concentration of about 23 mM. In some embodiments, the pharmaceutical composition comprises citrate buffer at a concentration of about 24 mM. In some embodiments, the pharmaceutical composition comprises citrate buffer at a concentration of about 25 mM.

In some embodiments, the pharmaceutical composition comprises succinate buffer. In some embodiments, the pharmaceutical composition comprises succinate buffer at a concentration of about 15 mM to about 20 mM. In some embodiments, the pharmaceutical composition comprises succinate buffer at a concentration of about 20 mM to about 25 mM.

In some embodiments, the pharmaceutical composition comprises succinate buffer at a concentration of about 15 mM. In some embodiments, the pharmaceutical composition comprises succinate buffer at a concentration of about 16 mM. In some embodiments, the pharmaceutical composition comprises succinate buffer at a concentration of about 17 mM. In some embodiments, the pharmaceutical composition comprises succinate buffer at a concentration of about 18 mM. In some embodiments, the pharmaceutical composition comprises succinate buffer at a concentration of about 19 mM. In some embodiments, the pharmaceutical composition comprises succinate buffer at a concentration of about 20 mM. In some embodiments, the pharmaceutical composition comprises succinate buffer at a concentration of about 21 mM. In some embodiments, the pharmaceutical composition comprises succinate buffer at a concentration of about 22 mM. In some embodiments, the pharmaceutical composition comprises succinate buffer at a concentration of about 23 mM. In some embodiments, the pharmaceutical composition comprises succinate buffer at a concentration of about 24 mM. In some embodiments, the pharmaceutical composition comprises succinate buffer at a concentration of about 25 mM.

In some embodiments, the pharmaceutical composition comprises ascorbate buffer. In some embodiments, the pharmaceutical composition comprises ascorbate buffer at a concentration of about 15 mM to about 20 mM. In some embodiments, the pharmaceutical composition comprises ascorbate buffer at a concentration of about 20 mM to about 25 mM.

In some embodiments, the pharmaceutical composition comprises ascorbate buffer at a concentration of about 15 mM. In some embodiments, the pharmaceutical composition comprises ascorbate buffer at a concentration of about 16 mM. In some embodiments, the pharmaceutical composition comprises ascorbate buffer at a concentration of about 17 mM. In some embodiments, the pharmaceutical composition comprises ascorbate buffer at a concentration of about 18 mM. In some embodiments, the pharmaceutical composition comprises ascorbate buffer at a concentration of about 19 mM. In some embodiments, the pharmaceutical composition comprises ascorbate buffer at a concentration of about 20 mM. In some embodiments, the pharmaceutical composition comprises ascorbate buffer at a concentration of about 21 mM. In some embodiments, the pharmaceutical composition comprises ascorbate buffer at a concentration of about 22 mM. In some embodiments, the pharmaceutical composition comprises ascorbate buffer at a concentration of about 23 mM. In some embodiments, the pharmaceutical composition comprises ascorbate buffer at a concentration of about 24 mM. In some embodiments, the pharmaceutical composition comprises ascorbate buffer at a concentration of about 25 mM.

In some embodiments, the pharmaceutical composition comprises glutamate buffer. In some embodiments, the pharmaceutical composition comprises glutamate buffer at a concentration of about 15 mM to about 20 mM. In some embodiments, the pharmaceutical composition comprises glutamate buffer at a concentration of about 20 mM to about 25 mM.

In some embodiments, the pharmaceutical composition comprises glutamate buffer at a concentration of about 15 mM. In some embodiments, the pharmaceutical composition comprises glutamate buffer at a concentration of about 16 mM. In some embodiments, the pharmaceutical composition comprises glutamate buffer at a concentration of about 17 mM. In some embodiments, the pharmaceutical composition comprises glutamate buffer at a concentration of about 18 mM. In some embodiments, the pharmaceutical composition comprises glutamate buffer at a concentration of about 19 mM. In some embodiments, the pharmaceutical composition comprises glutamate buffer at a concentration of about 20 mM. In some embodiments, the pharmaceutical composition comprises glutamate buffer at a concentration of about 21 mM. In some embodiments, the pharmaceutical composition comprises glutamate buffer at a concentration of about 22 mM. In some embodiments, the pharmaceutical composition comprises glutamate buffer at a concentration of about 23 mM. In some embodiments, the pharmaceutical composition comprises glutamate buffer at a concentration of about 24 mM. In some embodiments, the pharmaceutical composition comprises glutamate buffer at a concentration of about 25 mM.

In some embodiments, the pharmaceutical composition comprises lactate buffer. In some embodiments, the pharmaceutical composition comprises lactate buffer at a concentration of about 15 mM to about 20 mM. In some embodiments, the pharmaceutical composition comprises lactate buffer at a concentration of about 20 mM to about 25 mM.

In some embodiments, the pharmaceutical composition comprises lactate buffer at a concentration of about 15 mM. In some embodiments, the pharmaceutical composition comprises lactate buffer at a concentration of about 16 mM. In some embodiments, the pharmaceutical composition comprises lactate buffer at a concentration of about 17 mM. In some embodiments, the pharmaceutical composition comprises lactate buffer at a concentration of about 18 mM. In some embodiments, the pharmaceutical composition comprises lactate buffer at a concentration of about 19 mM. In some embodiments, the pharmaceutical composition comprises lactate buffer at a concentration of about 20 mM. In some embodiments, the pharmaceutical composition comprises lactate buffer at a concentration of about 21 mM. In some embodiments, the pharmaceutical composition comprises lactate buffer at a concentration of about 22 mM. In some embodiments, the pharmaceutical composition comprises lactate buffer at a concentration of about 23 mM. In some embodiments, the pharmaceutical composition comprises lactate buffer at a concentration of about 24 mM. In some embodiments, the pharmaceutical composition comprises lactate buffer at a concentration of about 25 mM.

In some embodiments, the pharmaceutical composition comprises maleate buffer. In some embodiments, the pharmaceutical composition comprises maleate buffer at a concentration of about 15 mM to about 20 mM. In some embodiments, the pharmaceutical composition comprises maleate buffer at a concentration of about 20 mM to about 25 mM.

In some embodiments, the pharmaceutical composition comprises maleate buffer at a concentration of about 15 mM. In some embodiments, the pharmaceutical composition comprises maleate buffer at a concentration of about 16 mM. In some embodiments, the pharmaceutical composition comprises maleate buffer at a concentration of about 17 mM. In some embodiments, the pharmaceutical composition comprises maleate buffer at a concentration of about 18 mM. In some embodiments, the pharmaceutical composition comprises maleate buffer at a concentration of about 19 mM. In some embodiments, the pharmaceutical composition comprises maleate buffer at a concentration of about 20 mM. In some embodiments, the pharmaceutical composition comprises maleate buffer at a concentration of about 21 mM. In some embodiments, the pharmaceutical composition comprises maleate buffer at a concentration of about 22 mM. In some embodiments, the pharmaceutical composition comprises maleate buffer at a concentration of about 23 mM. In some embodiments, the pharmaceutical composition comprises maleate buffer at a concentration of about 24 mM. In some embodiments, the pharmaceutical composition comprises maleate buffer at a concentration of about 25 mM.

In some embodiments, the pharmaceutical composition comprises trometamol buffer. In some embodiments, the pharmaceutical composition comprises trometamol buffer at a concentration of about 15 mM to about 20 mM. In some embodiments, the pharmaceutical composition comprises trometamol buffer at a concentration of about 20 mM to about 25 mM.

In some embodiments, the pharmaceutical composition comprises trometamol buffer at a concentration of about 15 mM. In some embodiments, the pharmaceutical composition comprises trometamol buffer at a concentration of about 16 mM. In some embodiments, the pharmaceutical composition comprises trometamol buffer at a concentration of about 17 mM. In some embodiments, the pharmaceutical composition comprises trometamol buffer at a concentration of about 18 mM. In some embodiments, the pharmaceutical composition comprises trometamol buffer at a concentration of about 19 mM. In some embodiments, the pharmaceutical composition comprises trometamol buffer at a concentration of about 20 mM. In some embodiments, the pharmaceutical composition comprises trometamol buffer at a concentration of about 21 mM. In some embodiments, the pharmaceutical composition comprises trometamol buffer at a concentration of about 22 mM. In some embodiments, the pharmaceutical composition comprises trometamol buffer at a concentration of about 23 mM. In some embodiments, the pharmaceutical composition comprises trometamol buffer at a concentration of about 24 mM. In some embodiments, the pharmaceutical composition comprises trometamol buffer at a concentration of about 25 mM.

In some embodiments, the pharmaceutical composition comprises gluconate buffer. In some embodiments, the pharmaceutical composition comprises gluconate buffer at a concentration of about 15 mM to about 20 mM. In some embodiments, the pharmaceutical composition comprises gluconate buffer at a concentration of about 20 mM to about 25 mM.

In some embodiments, the pharmaceutical composition comprises gluconate buffer at a concentration of about 15 mM. In some embodiments, the pharmaceutical composition comprises gluconate buffer at a concentration of about 16 mM. In some embodiments, the pharmaceutical composition comprises gluconate buffer at a concentration of about 17 mM. In some embodiments, the pharmaceutical composition comprises gluconate buffer at a concentration of about 18 mM. In some embodiments, the pharmaceutical composition comprises gluconate buffer at a concentration of about 19 mM. In some embodiments, the pharmaceutical composition comprises gluconate buffer at a concentration of about 20 mM. In some embodiments, the pharmaceutical composition comprises gluconate buffer at a concentration of about 21 mM. In some embodiments, the pharmaceutical composition comprises gluconate buffer at a concentration of about 22 mM. In some embodiments, the pharmaceutical composition comprises gluconate buffer at a concentration of about 23 mM. In some embodiments, the pharmaceutical composition comprises gluconate buffer at a concentration of about 24 mM. In some embodiments, the pharmaceutical composition comprises gluconate buffer at a concentration of about 25 mM.

In some embodiments, the pharmaceutical composition has a pH of about 6.5-8.0. In some embodiments, the pharmaceutical composition has a pH of about 6.5 to about 7.7.

In some embodiments, the pharmaceutical composition has a pH of about 6.5. In some embodiments, the pharmaceutical composition has a pH of about 6.6. In some embodiments, the pharmaceutical composition has a pH of about 6.7. In some embodiments, the pharmaceutical composition has a pH of about 6.8. In some embodiments, the pharmaceutical composition has a pH of about 6.9. In some embodiments, the pharmaceutical composition has a pH of about 7.0. In some embodiments, the pharmaceutical composition has a pH of about 7.1. In some embodiments, the pharmaceutical composition has a pH of about 7.2. In some embodiments, the pharmaceutical composition has a pH of about 7.3. In some embodiments, the pharmaceutical composition has a pH of about 7.4. In some embodiments, the pharmaceutical composition has a pH of about 7.5. In some embodiments, the pharmaceutical composition has a pH of about 7.6. In some embodiments, the pharmaceutical composition has a pH of about 7.7. In some embodiments, the pharmaceutical composition has a pH of about 7.8. In some embodiments, the pharmaceutical composition has a pH of about 7.9. In some embodiments, the pharmaceutical composition has a pH of about 8.0. In some embodiments, the pharmaceutical composition has a pH of 6.5. In some embodiments, the pharmaceutical composition has a pH of 6.6. In some embodiments, the pharmaceutical composition has a pH of about 6.7. In some embodiments, the pharmaceutical composition has a pH of 6.8. In some embodiments, the pharmaceutical composition has a pH of 6.9. In some embodiments, the pharmaceutical composition has a pH of about 7.0. In some embodiments, the pharmaceutical composition has a pH of 7.1. In some embodiments, the pharmaceutical composition has a pH of 7.2. In some embodiments, the pharmaceutical composition has a pH of about 7.3. In some embodiments, the pharmaceutical composition has a pH of 7.4. In some embodiments, the pharmaceutical composition has a pH of about 7.5. In some embodiments, the pharmaceutical composition has a pH of 7.6. In some embodiments, the pharmaceutical composition has a pH of 7.7. In some embodiments, the pharmaceutical composition has a pH of about 7.8. In some embodiments, the pharmaceutical composition has a pH of 7.9. In some embodiments, the pharmaceutical composition has a pH of about 8.0.

In some embodiments, the pharmaceutical composition comprises an antioxidant present at a concentration of about 5 mM to about 15 mM. In some embodiments, the pharmaceutical composition comprises an antioxidant present at a concentration of about 5 mM to about 10 mM. In some embodiments, the pharmaceutical composition comprises an antioxidant present at a concentration of about 10 mM to about 15 mM.

In some embodiments, the pharmaceutical composition comprises an antioxidant present at a concentration of about 5 mM. In some embodiments, the pharmaceutical composition comprises an antioxidant present at a concentration of about 6 mM. In some embodiments, the pharmaceutical composition comprises an antioxidant present at a concentration of about 7 mM. In some embodiments, the pharmaceutical composition comprises an antioxidant present at a concentration of about 8 mM. In some embodiments, the pharmaceutical composition comprises an antioxidant present at a concentration of about 9 mM. In some embodiments, the pharmaceutical composition comprises an antioxidant present at a concentration of about 10 mM. In some embodiments, the pharmaceutical composition comprises an antioxidant present at a concentration of about 11 mM. In some embodiments, the pharmaceutical composition comprises an antioxidant present at a concentration of about 12 mM. In some embodiments, the pharmaceutical composition comprises an antioxidant present at a concentration of about 13 mM. In some embodiments, the pharmaceutical composition comprises an antioxidant present at a concentration of about 14 mM. In some embodiments, the pharmaceutical composition comprises an antioxidant present at a concentration of about 15 mM.

In some embodiments, the antibody compositions of the present disclosure comprise methionine. Methionine is an essential amino acid that can be represented by the structure of.

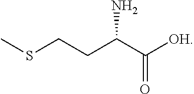

Methionine, as used herein, includes the free base form of methionine, as well as any and all salts thereof. In some embodiments, methionine includes a pharmaceutically acceptable salt thereof, e.g., methionine hydrochloride. Methionine, as used herein, also includes all enantiomers (e.g., L-methionine and S-methionine), and any combination of enantiomers (e.g., 50% L-methionine and 50% S-methionine; 90%-100% L-methionine and 10%-0% S-methionine, and the like). In some embodiments, the term “methionine” includes greater than 99% L-methionine and less than 1% S-methionine. In some embodiments, the term “methionine” includes an enantomerically pure L-methionine. In some embodiments, methionine is a pharmaceutical grade methionine.

In some embodiments, the antibody compositions of the present disclosure comprise arginine. Arginine is a conditionally non-essential amino acid that can be represented by the structure of.

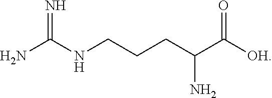 [SMILES: N=C(N)NCCCC(N)C(=O)O]

Arginine, as used herein, includes the free base form of arginine, as well as any and all salts thereof. In some embodiments, arginine includes a pharmaceutically acceptable salt thereof, e.g., arginine hydrochloride. Arginine, as used herein, also includes all enantiomers (e.g., L-arginine and S-arginine), and any combination of enantiomers (e.g., 50% L-arginine and 50% S-arginine; 90%-100% L-arginine and 10%-0% S-arginine, etc.). In some embodiments, the term “arginine” includes greater than 99% L-arginine and less than 1% S-arginine. In some embodiments, the term “arginine” includes an enantomerically pure L-arginine. In some embodiments, arginine is a pharmaceutical grade arginine.

Various concentrations of an antioxidant can be present in the compositions of the present disclosure. In some embodiments, the pharmaceutical composition comprises greater than 1 mM antioxidant, greater than 2 mM antioxidant, greater than 3 mM antioxidant, or greater than 4 mM antioxidant. In other aspects, the pharmaceutical composition comprises up to 5 mM antioxidant, up to 6 mM antioxidant, up to 7 mM antioxidant, up to 8 mM antioxidant, up to 9 mM antioxidant, up to 10 mM antioxidant, up to 11 mM antioxidant, up to 12 mM antioxidant, up to 13 mM antioxidant, up to 15 mM antioxidant, or up to 20 mM antioxidant. In other aspects, the pharmaceutical composition comprises about 1 mM to about 20 mM, about 1 mM to about 15 mM, about 1 mM to about 10 mM, about 1 mM to about 5 mM, about 5 mM to about 20 mM, about 5 mM to about 15 mM, about 5 mM to about 10 mM, about 7 mM to about 13 mM, or about 10 mM antioxidant. In some embodiments, the pharmaceutical composition comprises about 10 mM antioxidant. In some embodiments, the pharmaceutical composition comprises 10 mM antioxidant.

Various concentrations of methionine can be present in the compositions of the present disclosure. In some embodiments, the pharmaceutical composition comprises greater than 1 mM methionine, greater than 2 mM methionine, greater than 3 mM methionine, or greater than 4 mM methionine. In other aspects, the pharmaceutical composition comprises up to 5 mM methionine, up to 6 mM methionine, up to 7 mM methionine, up to 8 mM methionine, up to 9 mM methionine, up to 10 mM methionine, up to 11 mM methionine, up to 12 mM methionine, up to 13 mM methionine, up to 14 mM methionine, up to 15 mM methionine, or up to 20 mM methionine. In other aspects, the pharmaceutical composition comprises about 1 mM to about 20 mM, about 1 mM to about 15 mM, about 1 mM to about 10 mM, about 1 mM to about 5 mM, about 5 mM to about 20 mM, about 5 mM to about 15 mM, about 5 mM to about 10 mM, about 7 mM to about 13 mM, or about 10 mM methionine. In some embodiments, the pharmaceutical composition comprises about 10 mM methionine. In some embodiments, the pharmaceutical composition comprises 10 mM methionine. In some embodiments, methionine is added in an amount sufficient to maintain osmolality of the pharmaceutical composition. In some embodiments, methionine is added in an amount sufficient to achieve a hyper-tonic solution.

Various concentrations of L-methionine can be present in the compositions of the present disclosure. In some embodiments, the pharmaceutical composition comprises greater than 1 mM L-methionine, greater than 2 mM L-methionine, greater than 3 mM L-methionine, or greater than 4 mM L-methionine. In other aspects, the pharmaceutical composition comprises up to 5 mM L-methionine, up to 6 mM L-methionine, up to 7 mM L-methionine, up to 8 mM L-methionine, up to 9 mM L-methionine, up to 10 mM L-methionine, up to 11 mM L-methionine, up to 12 mM L-methionine, up to 13 mM L-methionine, up to 14 mM L-methionine, up to 15 mM L-methionine, or up to 20 mM L-methionine. In other aspects, the pharmaceutical composition comprises about 1 mM to about 20 mM, about 1 mM to about 15 mM, about 1 mM to about 10 mM, about 1 mM to about 5 mM, about 5 mM to about 20 mM, about 5 mM to about 15 mM, about 5 mM to about 10 mM, about 7 mM to about 13 mM, or about 10 mM L-methionine. In some embodiments, the pharmaceutical composition comprises about 10 mM L-methionine. In some embodiments, the pharmaceutical composition comprises 10 mM L-methionine.

In some embodiments, the antioxidant is L-methionine. In some embodiments the antioxidant is ascorbic acid. In some embodiments, the antioxidant is EDTA.

In some embodiments, the pharmaceutical composition comprises L-methionine present at about 5 mM to about 15 mM. In some embodiments, the pharmaceutical composition comprises L-methionine present at a concentration of about 5 mM to about 10 mM. In some embodiments, the pharmaceutical composition comprises L-methionine present at a concentration of about 10 mM to about 15 mM. In some embodiments, the pharmaceutical composition comprises L-methionine present at a concentration of about 5 mM. In some embodiments, the pharmaceutical composition comprises L-methionine present at a concentration of about 6 mM. In some embodiments, the pharmaceutical composition comprises L-methionine present at a concentration of about 7 mM. In some embodiments, the pharmaceutical composition comprises L-methionine present at a concentration of about 8 mM. In some embodiments, the pharmaceutical composition comprises L-methionine present at a concentration of about 9 mM. In some embodiments, the pharmaceutical composition comprises L-methionine present at a concentration of about 10 mM. In some embodiments, the pharmaceutical composition comprises L-methionine present at a concentration of about 11 mM. In some embodiments, the pharmaceutical composition comprises L-methionine present at a concentration of about 12 mM. In some embodiments, the pharmaceutical composition comprises L-methionine present at a concentration of about 13 mM. In some embodiments, the pharmaceutical composition comprises L-methionine present at a concentration of about 14 mM. In some embodiments, the pharmaceutical composition comprises L-methionine present at a concentration of about 15 mM.

In some embodiments, the pharmaceutical composition comprises a sugar present at a concentration of about 2% w/v to about 14% w/v. In some embodiments, the pharmaceutical composition comprises a sugar present at a concentration of about 2% w/v to about 12% w/v. In some embodiments, the pharmaceutical composition comprises a sugar present at a concentration of about 2% w/v to about 10% w/v. In some embodiments, the pharmaceutical composition comprises a sugar present at a concentration of about 2% w/v to about 8% w/v. In some embodiments, the pharmaceutical composition comprises a sugar present at a concentration of about 2% w/v to about 6% w/v. In some embodiments, the pharmaceutical composition comprises a sugar present at a concentration of about 2% w/v to about 4% w/v. In some embodiments, the pharmaceutical composition comprises a sugar present at a concentration of about 4% w/v to about 14% w/v. In some embodiments, the pharmaceutical composition comprises a sugar present at a concentration of about 4% w/v to about 12% w/v. In some embodiments, the pharmaceutical composition comprises a sugar present at a concentration of about 4% w/v to about 10% w/v. In some embodiments, the pharmaceutical composition comprises a sugar present at a concentration of about 4% w/v to about 8% w/v. In some embodiments, the pharmaceutical composition comprises a sugar present at a concentration of about 4% w/v to about 6% w/v. In some embodiments, the pharmaceutical composition comprises a sugar present at a concentration of about 6% w/v to about 14% w/v. In some embodiments, the pharmaceutical composition comprises a sugar present at a concentration of about 6% w/v to about 12% w/v. In some embodiments, the pharmaceutical composition comprises a sugar present at a concentration of about 6% w/v to about 10% w/v. In some embodiments, the pharmaceutical composition comprises a sugar present at a concentration of about 6% w/v to about 8% w/v. In some embodiments, the pharmaceutical composition comprises a sugar present at a concentration of about 8% w/v to about 14% w/v. In some embodiments, the pharmaceutical composition comprises a sugar present at a concentration of about 8% w/v to about 12% w/v. In some embodiments, the pharmaceutical composition comprises a sugar present at a concentration of about 8% w/v to about 10% w/v. In some embodiments, the pharmaceutical composition comprises a sugar present at a concentration of about 10% w/v to about 14% w/v. In some embodiments, the pharmaceutical composition comprises a sugar present at a concentration of about 10% w/v to about 12% w/v. In some embodiments, the pharmaceutical composition comprises a sugar present at a concentration of about 12% w/v to about 14% w/v.

In some embodiments, the pharmaceutical composition comprises a sugar present at a concentration of about 1% w/v. In some embodiments, the pharmaceutical composition comprises a sugar present at a concentration of about 2% w/v. In some embodiments, the pharmaceutical composition comprises a sugar present at a concentration of about 3% w/v. In some embodiments, the pharmaceutical composition comprises a sugar present at a concentration of about 4% w/v. In some embodiments, the pharmaceutical composition comprises a sugar present at a concentration of about 5% w/v. In some embodiments, the pharmaceutical composition comprises a sugar present at a concentration of about 6% w/v. In some embodiments, the pharmaceutical composition comprises a sugar present at a concentration of about 7% w/v. In some embodiments, the pharmaceutical composition comprises a sugar present at a concentration of about 8% w/v. In some embodiments, the pharmaceutical composition comprises a sugar present at a concentration of about 9% w/v. In some embodiments, the pharmaceutical composition comprises a sugar present at a concentration of about 10% w/v. In some embodiments, the pharmaceutical composition comprises a sugar present at a concentration of about 11% w/v. In some embodiments, the pharmaceutical composition comprises a sugar present at a concentration of about 12% w/v. In some embodiments, the pharmaceutical composition comprises a sugar present at a concentration of about 13% w/v. In some embodiments, the pharmaceutical composition comprises a sugar present at a concentration of about 14% w/v.

In some embodiments, the sugar is a polyol. In some embodiments, the sugar is a disaccharide. In some embodiments, the sugar is a polysaccharide. In some embodiments, the sugar is a sugar alcohol. In some embodiments, the pharmaceutical composition comprises a sugar selected from sucrose, trehalose, sorbitol, mannitol, or any combination thereof.

In some embodiments, the pharmaceutical composition comprises sucrose. In some embodiments, the pharmaceutical composition comprises trehalose. In some embodiments, the pharmaceutical composition comprises sorbitol. In some embodiments, the pharmaceutical composition comprises mannitol.

In some embodiments, the pharmaceutical composition comprises sucrose present at a concentration of about 2% w/v to about 14% w/v. In some embodiments, the pharmaceutical composition comprises sucrose present at a concentration of about 2% w/v to about 12% w/v. In some embodiments, the pharmaceutical composition comprises sucrose present at a concentration of about 2% w/v to about 10% w/v. In some embodiments, the pharmaceutical composition comprises sucrose present at a concentration of about 2% w/v to about 8% w/v. In some embodiments, the pharmaceutical composition comprises sucrose present at a concentration of about 2% w/v to about 6% w/v. In some embodiments, the pharmaceutical composition comprises sucrose present at a concentration of about 2% w/v to about 4% w/v. In some embodiments, the pharmaceutical composition comprises sucrose present at a concentration of about 4% w/v to about 14% w/v. In some embodiments, the pharmaceutical composition comprises sucrose present at a concentration of about 4% w/v to about 12% w/v. In some embodiments, the pharmaceutical composition comprises sucrose present at a concentration of about 4% w/v to about 10% w/v. In some embodiments, the pharmaceutical composition comprises sucrose present at a concentration of about 4% w/v to about 8% w/v. In some embodiments, the pharmaceutical composition comprises sucrose present at a concentration of about 4% w/v to about 6% w/v. In some embodiments, the pharmaceutical composition comprises sucrose present at a concentration of about 6% w/v to about 14% w/v. In some embodiments, the pharmaceutical composition comprises sucrose present at a concentration of about 6% w/v to about 12% w/v. In some embodiments, the pharmaceutical composition comprises sucrose present at a concentration of about 6% w/v to about 10% w/v. In some embodiments, the pharmaceutical composition comprises sucrose present at a concentration of about 6% w/v to about 8% w/v. In some embodiments, the pharmaceutical composition comprises sucrose present at a concentration of about 8% w/v to about 14% w/v. In some embodiments, the pharmaceutical composition comprises sucrose present at a concentration of about 8% w/v to about 12% w/v. In some embodiments, the pharmaceutical composition comprises sucrose present at a concentration of about 8% w/v to about 10% w/v. In some embodiments, the pharmaceutical composition comprises sucrose present at a concentration of about 10% w/v to about 14% w/v. In some embodiments, the pharmaceutical composition comprises sucrose present at a concentration of about 10% w/v to about 12% w/v. In some embodiments, the pharmaceutical composition comprises sucrose present at a concentration of about 12% w/v to about 14% w/v.

In some embodiments, the pharmaceutical composition comprises sucrose present at a concentration of about 1% w/v. In some embodiments, the pharmaceutical composition comprises sucrose present at a concentration of about 2% w/v. In some embodiments, the pharmaceutical composition comprises sucrose present at a concentration of about 3% w/v. In some embodiments, the pharmaceutical composition comprises sucrose that is present at a concentration of about 3.5% w/v. In some embodiments, the pharmaceutical composition comprises sucrose present at a concentration of about 4% w/v. In some embodiments, the pharmaceutical composition comprises sucrose present at a concentration of about 5% w/v. In some embodiments, the pharmaceutical composition comprises sucrose present at a concentration of about 6% w/v. In some embodiments, the pharmaceutical composition comprises sucrose present at a concentration of about 7% w/v. In some embodiments, the pharmaceutical composition comprises sucrose present at a concentration of about 8% w/v. In some embodiments, the pharmaceutical composition comprises sucrose present at a concentration of about 9% w/v. In some embodiments, the pharmaceutical composition comprises sucrose present at a concentration of about 10% w/v. In some embodiments, the pharmaceutical composition comprises sucrose present at a concentration of about 11% w/v. In some embodiments, the pharmaceutical composition comprises sucrose present at a concentration of about 12% w/v. In some embodiments, the pharmaceutical composition comprises sucrose present at a concentration of about 13% w/v. In some embodiments, the pharmaceutical composition comprises sucrose present at a concentration of about 14% w/v.

In some embodiments, the pharmaceutical composition comprises a viscosity modifying agent that is present at a concentration of about 20 mM to about 180 mM. In some embodiments, the pharmaceutical composition comprises a viscosity modifying agent that is present at a concentration of about 20 mM to about 170 mM. In some embodiments, the pharmaceutical composition comprises a viscosity modifying agent that is present at a concentration of about 20 mM to about 160 mM. In some embodiments, the pharmaceutical composition comprises a viscosity modifying agent that is present at a concentration of about 20 mM to about 150 mM. In some embodiments, the pharmaceutical composition comprises a viscosity modifying agent that is present at a concentration of about 20 mM to about 140 mM. In some embodiments, the pharmaceutical composition comprises a viscosity modifying agent that is present at a concentration of about 20 mM to about 130 mM. In some embodiments, the pharmaceutical composition comprises a viscosity modifying agent that is present at a concentration of about 20 mM to about 120 mM. In some embodiments, the pharmaceutical composition comprises a viscosity modifying agent that is present at a concentration of about 20 mM to about 110 mM. In some embodiments, the pharmaceutical composition comprises a viscosity modifying agent that is present at a concentration of about 20 mM to about 100 mM. In some embodiments, the pharmaceutical composition comprises a viscosity modifying agent that is present at a concentration of about 20 mM to about 90 mM. In some embodiments, the pharmaceutical composition comprises a viscosity modifying agent that is present at a concentration of about 20 mM to about 80 mM. In some embodiments, the pharmaceutical composition comprises a viscosity modifying agent that is present at a concentration of about 20 mM to about 70 mM. In some embodiments, the pharmaceutical composition comprises a viscosity modifying agent that is present at a concentration of about 20 mM to about 60 mM. In some embodiments, the pharmaceutical composition comprises a viscosity modifying agent that is present at a concentration of about 20 mM to about 50 mM. In some embodiments, the pharmaceutical composition comprises a viscosity modifying agent that is present at a concentration of about 20 mM to about 40 mM. In some embodiments, the pharmaceutical composition comprises a viscosity modifying agent that is present at a concentration of about 20 mM to about 30 mM. In some embodiments, the pharmaceutical composition comprises a viscosity modifying agent that is present at a concentration of about 30 mM to about 180 mM. In some embodiments, the pharmaceutical composition comprises a viscosity modifying agent that is present at a concentration of about 30 mM to about 170 mM. In some embodiments, the pharmaceutical composition comprises a viscosity modifying agent that is present at a concentration of about 30 mM to about 160 mM. In some embodiments, the pharmaceutical composition comprises a viscosity modifying agent that is present at a concentration of about 30 mM to about 150 mM. In some embodiments, the pharmaceutical composition comprises a viscosity modifying agent that is present at a concentration of about 30 mM to about 140 mM. In some embodiments, the pharmaceutical composition comprises a viscosity modifying agent that is present at a concentration of about 30 mM to about 130 mM. In some embodiments, the pharmaceutical composition comprises a viscosity modifying agent that is present at a concentration of about 30 mM to about 120 mM. In some embodiments, the pharmaceutical composition comprises a viscosity modifying agent that is present at a concentration of about 30 mM to about 110 mM. In some embodiments, the pharmaceutical composition comprises a viscosity modifying agent that is present at a concentration of about 30 mM to about 100 mM. In some embodiments, the pharmaceutical composition comprises a viscosity modifying agent that is present at a concentration of about 30 mM to about 90 mM. In some embodiments, the pharmaceutical composition comprises a viscosity modifying agent that is present at a concentration of about 30 mM to about 80 mM. In some embodiments, the pharmaceutical composition comprises a viscosity modifying agent that is present at a concentration of about 30 mM to about 70 mM. In some embodiments, the pharmaceutical composition comprises a viscosity modifying agent that is present at a concentration of about 30 mM to about 60 mM. In some embodiments, the pharmaceutical composition comprises a viscosity modifying agent that is present at a concentration of about 30 mM to about 50 mM. In some embodiments, the pharmaceutical composition comprises a viscosity modifying agent that is present at a concentration of about 30 mM to about 40 mM. In some embodiments, the pharmaceutical composition comprises a viscosity modifying agent that is present at a concentration of about 40 mM to about 180 mM. In some embodiments, the pharmaceutical composition comprises a viscosity modifying agent that is present at a concentration of about 40 mM to about 170 mM. In some embodiments, the pharmaceutical composition comprises a viscosity modifying agent that is present at a concentration of about 40 mM to about 160 mM. In some embodiments, the pharmaceutical composition comprises a viscosity modifying agent that is present at a concentration of about 40 mM to about 150 mM. In some embodiments, the pharmaceutical composition comprises a viscosity modifying agent that is present at a concentration of about 40 mM to about 140 mM. In some embodiments, the pharmaceutical composition comprises a viscosity modifying agent that is present at a concentration of about 40 mM to about 130 mM. In some embodiments, the pharmaceutical composition comprises a viscosity modifying agent that is present at a concentration of about 40 mM to about 120 mM. In some embodiments, the pharmaceutical composition comprises a viscosity modifying agent that is present at a concentration of about 40 mM to about 110 mM. In some embodiments, the pharmaceutical composition comprises a viscosity modifying agent that is present at a concentration of about 40 mM to about 100 mM. In some embodiments, the pharmaceutical composition comprises a viscosity modifying agent that is present at a concentration of about 40 mM to about 90 mM. In some embodiments, the pharmaceutical composition comprises a viscosity modifying agent that is present at a concentration of about 40 mM to about 80 mM. In some embodiments, the pharmaceutical composition comprises a viscosity modifying agent that is present at a concentration of about 40 mM to about 70 mM. In some embodiments, the pharmaceutical composition comprises a viscosity modifying agent that is present at a concentration of about 40 mM to about 60 mM. In some embodiments, the pharmaceutical composition comprises a viscosity modifying agent that is present at a concentration of about 40 mM to about 50 mM. In some embodiments, the pharmaceutical composition comprises a viscosity modifying agent that is present at a concentration of about 50 mM to about 180 mM. In some embodiments, the pharmaceutical composition comprises a viscosity modifying agent that is present at a concentration of about 50 mM to about 170 mM. In some embodiments, the pharmaceutical composition comprises a viscosity modifying agent that is present at a concentration of about 50 mM to about 160 mM. In some embodiments, the pharmaceutical composition comprises a viscosity modifying agent that is present at a concentration of about 50 mM to about 150 mM. In some embodiments, the pharmaceutical composition comprises a viscosity modifying agent that is present at a concentration of about 50 mM to about 140 mM. In some embodiments, the pharmaceutical composition comprises a viscosity modifying agent that is present at a concentration of about 50 mM to about 130 mM. In some embodiments, the pharmaceutical composition comprises a viscosity modifying agent that is present at a concentration of about 50 mM to about 120 mM. In some embodiments, the pharmaceutical composition comprises a viscosity modifying agent that is present at a concentration of about 50 mM to about 110 mM. In some embodiments, the pharmaceutical composition comprises a viscosity modifying agent that is present at a concentration of about 50 mM to about 100 mM. In some embodiments, the pharmaceutical composition comprises a viscosity modifying agent that is present at a concentration of about 50 mM to about 90 mM. In some embodiments, the pharmaceutical composition comprises a viscosity modifying agent that is present at a concentration of about 50 mM to about 80 mM. In some embodiments, the pharmaceutical composition comprises a viscosity modifying agent that is present at a concentration of about 50 mM to about 70 mM. In some embodiments, the pharmaceutical composition comprises a viscosity modifying agent that is present at a concentration of about 50 mM to about 60 mM. In some embodiments, the pharmaceutical composition comprises a viscosity modifying agent that is present at a concentration of about 60 mM to about 180 mM. In some embodiments, the pharmaceutical composition comprises a viscosity modifying agent that is present at a concentration of about 60 mM to about 170 mM. In some embodiments, the pharmaceutical composition comprises a viscosity modifying agent that is present at a concentration of about 60 mM to about 160 mM. In some embodiments, the pharmaceutical composition comprises a viscosity modifying agent that is present at a concentration of about 60 mM to about 150 mM. In some embodiments, the pharmaceutical composition comprises a viscosity modifying agent that is present at a concentration of about 60 mM to about 140 mM. In some embodiments, the pharmaceutical composition comprises a viscosity modifying agent that is present at a concentration of about 60 mM to about 130 mM. In some embodiments, the pharmaceutical composition comprises a viscosity modifying agent that is present at a concentration of about 60 mM to about 120 mM. In some embodiments, the pharmaceutical composition comprises a viscosity modifying agent that is present at a concentration of about 60 mM to about 110 mM. In some embodiments, the pharmaceutical composition comprises a viscosity modifying agent that is present at a concentration of about 60 mM to about 100 mM. In some embodiments, the pharmaceutical composition comprises a viscosity modifying agent that is present at a concentration of about 60 mM to about 90 mM. In some embodiments, the pharmaceutical composition comprises a viscosity modifying agent that is present at a concentration of about 60 mM to about 80 mM. In some embodiments, the pharmaceutical composition comprises a viscosity modifying agent that is present at a concentration of about 60 mM to about 70 mM. In some embodiments, the pharmaceutical composition comprises a viscosity modifying agent that is present at a concentration of about 70 mM to about 180 mM. In some embodiments, the pharmaceutical composition comprises a viscosity modifying agent that is present at a concentration of about 70 mM to about 170 mM. In some embodiments, the pharmaceutical composition comprises a viscosity modifying agent that is present at a concentration of about 70 mM to about 160 mM. In some embodiments, the pharmaceutical composition comprises a viscosity modifying agent that is present at a concentration of about 70 mM to about 150 mM. In some embodiments, the pharmaceutical composition comprises a viscosity modifying agent that is present at a concentration of about 70 mM to about 140 mM. In some embodiments, the pharmaceutical composition comprises a viscosity modifying agent that is present at a concentration of about 70 mM to about 130 mM. In some embodiments, the pharmaceutical composition comprises a viscosity modifying agent that is present at a concentration of about 70 mM to about 120 mM. In some embodiments, the pharmaceutical composition comprises a viscosity modifying agent that is present at a concentration of about 70 mM to about 110 mM. In some embodiments, the pharmaceutical composition comprises a viscosity modifying agent that is present at a concentration of about 70 mM to about 100 mM. In some embodiments, the pharmaceutical composition comprises a viscosity modifying agent that is present at a concentration of about 70 mM to about 90 mM. In some embodiments, the pharmaceutical composition comprises a viscosity modifying agent that is present at a concentration of about 70 mM to about 80 mM. In some embodiments, the pharmaceutical composition comprises a viscosity modifying agent that is present at a concentration of about 80 mM to about 180 mM. In some embodiments, the pharmaceutical composition comprises a viscosity modifying agent that is present at a concentration of about 80 mM to about 170 mM. In some embodiments, the pharmaceutical composition comprises a viscosity modifying agent that is present at a concentration of about 80 mM to about 160 mM. In some embodiments, the pharmaceutical composition comprises a viscosity modifying agent that is present at a concentration of about 80 mM to about 150 mM. In some embodiments, the pharmaceutical composition comprises a viscosity modifying agent that is present at a concentration of about 80 mM to about 140 mM. In some embodiments, the pharmaceutical composition comprises a viscosity modifying agent that is present at a concentration of about 80 mM to about 130 mM. In some embodiments, the pharmaceutical composition comprises a viscosity modifying agent that is present at a concentration of about 80 mM to about 120 mM. In some embodiments, the pharmaceutical composition comprises a viscosity modifying agent that is present at a concentration of about 80 mM to about 110 mM. In some embodiments, the pharmaceutical composition comprises a viscosity modifying agent that is present at a concentration of about 80 mM to about 100 mM. In some embodiments, the pharmaceutical composition comprises a viscosity modifying agent that is present at a concentration of about 80 mM to about 90 mM. In some embodiments, the pharmaceutical composition comprises a viscosity modifying agent that is present at a concentration of about 90 mM to about 180 mM. In some embodiments, the pharmaceutical composition comprises a viscosity modifying agent that is present at a concentration of about 90 mM to about 170 mM. In some embodiments, the pharmaceutical composition comprises a viscosity modifying agent that is present at a concentration of about 90 mM to about 160 mM. In some embodiments, the pharmaceutical composition comprises a viscosity modifying agent that is present at a concentration of about 90 mM to about 150 mM. In some embodiments, the pharmaceutical composition comprises a viscosity modifying agent that is present at a concentration of about 90 mM to about 140 mM. In some embodiments, the pharmaceutical composition comprises a viscosity modifying agent that is present at a concentration of about 90 mM to about 130 mM. In some embodiments, the pharmaceutical composition comprises a viscosity modifying agent that is present at a concentration of about 90 mM to about 120 mM. In some embodiments, the pharmaceutical composition comprises a viscosity modifying agent that is present at a concentration of about 90 mM to about 110 mM. In some embodiments, the pharmaceutical composition comprises a viscosity modifying agent that is present at a concentration of about 90 mM to about 100 mM. In some embodiments, the pharmaceutical composition comprises a viscosity modifying agent that is present at a concentration of about 100 mM to about 180 mM. In some embodiments, the pharmaceutical composition comprises a viscosity modifying agent that is present at a concentration of about 100 mM to about 170 mM. In some embodiments, the pharmaceutical composition comprises a viscosity modifying agent that is present at a concentration of about 100 mM to about 160 mM. In some embodiments, the pharmaceutical composition comprises a viscosity modifying agent that is present at a concentration of about 100 mM to about 150 mM. In some embodiments, the pharmaceutical composition comprises a viscosity modifying agent that is present at a concentration of about 100 mM to about 140 mM. In some embodiments, the pharmaceutical composition comprises a viscosity modifying agent that is present at a concentration of about 100 mM to about 130 mM. In some embodiments, the pharmaceutical composition comprises a viscosity modifying agent that is present at a concentration of about 100 mM to about 120 mM. In some embodiments, the pharmaceutical composition comprises a viscosity modifying agent that is present at a concentration of about 100 mM to about 110 mM. In some embodiments, the pharmaceutical composition comprises a viscosity modifying agent that is present at a concentration of about 110 mM to about 180 mM. In some embodiments, the pharmaceutical composition comprises a viscosity modifying agent that is present at a concentration of about 110 mM to about 170 mM. In some embodiments, the pharmaceutical composition comprises a viscosity modifying agent that is present at a concentration of about 110 mM to about 160 mM. In some embodiments, the pharmaceutical composition comprises a viscosity modifying agent that is present at a concentration of about 110 mM to about 150 mM. In some embodiments, the pharmaceutical composition comprises a viscosity modifying agent that is present at a concentration of about 110 mM to about 140 mM. In some embodiments, the pharmaceutical composition comprises a viscosity modifying agent that is present at a concentration of about 110 mM to about 130 mM. In some embodiments, the pharmaceutical composition comprises a viscosity modifying agent that is present at a concentration of about 110 mM to about 120 mM. In some embodiments, the pharmaceutical composition comprises a viscosity modifying agent that is present at a concentration of about 120 mM to about 180 mM. In some embodiments, the pharmaceutical composition comprises a viscosity modifying agent that is present at a concentration of about 120 mM to about 170 mM. In some embodiments, the pharmaceutical composition comprises a viscosity modifying agent that is present at a concentration of about 120 mM to about 160 mM. In some embodiments, the pharmaceutical composition comprises a viscosity modifying agent that is present at a concentration of about 120 mM to about 150 mM. In some embodiments, the pharmaceutical composition comprises a viscosity modifying agent that is present at a concentration of about 120 mM to about 140 mM. In some embodiments, the pharmaceutical composition comprises a viscosity modifying agent that is present at a concentration of about 120 mM to about 130 mM. In some embodiments, the pharmaceutical composition comprises a viscosity modifying agent that is present at a concentration of about 130 mM to about 180 mM. In some embodiments, the pharmaceutical composition comprises a viscosity modifying agent that is present at a concentration of about 130 mM to about 170 mM. In some embodiments, the pharmaceutical composition comprises a viscosity modifying agent that is present at a concentration of about 130 mM to about 160 mM. In some embodiments, the pharmaceutical composition comprises a viscosity modifying agent that is present at a concentration of about 130 mM to about 150 mM. In some embodiments, the pharmaceutical composition comprises a viscosity modifying agent that is present at a concentration of about 130 mM to about 140 mM. In some embodiments, the pharmaceutical composition comprises a viscosity modifying agent that is present at a concentration of about 140 mM to about 180 mM. In some embodiments, the pharmaceutical composition comprises a viscosity modifying agent that is present at a concentration of about 140 mM to about 170 mM. In some embodiments, the pharmaceutical composition comprises a viscosity modifying agent that is present at a concentration of about 140 mM to about 160 mM. In some embodiments, the pharmaceutical composition comprises a viscosity modifying agent that is present at a concentration of about 140 mM to about 150 mM. In some embodiments, the pharmaceutical composition comprises a viscosity modifying agent that is present at a concentration of about 150 mM to about 180 mM. In some embodiments, the pharmaceutical composition comprises a viscosity modifying agent that is present at a concentration of about 150 mM to about 170 mM. In some embodiments, the pharmaceutical composition comprises a viscosity modifying agent that is present at a concentration of about 150 mM to about 160 mM. In some embodiments, the pharmaceutical composition comprises a viscosity modifying agent that is present at a concentration of about 160 mM to about 180 mM. In some embodiments, the pharmaceutical composition comprises a viscosity modifying agent that is present at a concentration of about 160 mM to about 170 mM. In some embodiments, the pharmaceutical composition comprises a viscosity modifying agent that is present at a concentration of about 170 mM to about 180 mM.

In some embodiments, the pharmaceutical composition comprises a viscosity modifying agent that is present at a concentration of about 20 mM. In some embodiments, the pharmaceutical composition comprises a viscosity modifying agent that is present at a concentration of about 30 mM. In some embodiments, the pharmaceutical composition comprises a viscosity modifying agent that is present at a concentration of about 40 mM. In some embodiments, the pharmaceutical composition comprises a viscosity modifying agent that is present at a concentration of about 50 mM. In some embodiments, the pharmaceutical composition comprises a viscosity modifying agent that is present at a concentration of about 60 mM. In some embodiments, the pharmaceutical composition comprises a viscosity modifying agent that is present at a concentration of about 70 mM. In some embodiments, the pharmaceutical composition comprises a viscosity modifying agent that is present at a concentration of about 80 mM. In some embodiments, the pharmaceutical composition comprises a viscosity modifying agent that is present at a concentration of about 90 mM. In some embodiments, the pharmaceutical composition comprises a viscosity modifying agent that is present at a concentration of about 100 mM. In some embodiments, the pharmaceutical composition comprises a viscosity modifying agent that is present at a concentration of about 110 mM. In some embodiments, the pharmaceutical composition comprises a viscosity modifying agent that is present at a concentration of about 120 mM. In some embodiments, the pharmaceutical composition comprises a viscosity modifying agent that is present at a concentration of about 130 mM. In some embodiments, the pharmaceutical composition comprises a viscosity modifying agent that is present at a concentration of about 140 mM. In some embodiments, the pharmaceutical composition comprises a viscosity modifying agent that is present at a concentration of about 150 mM. In some embodiments, the pharmaceutical composition comprises a viscosity modifying agent that is present at a concentration of about 160 mM. In some embodiments, the pharmaceutical composition comprises a viscosity modifying agent that is present at a concentration of about 170 mM. In some embodiments, the pharmaceutical composition comprises a viscosity modifying agent that is present at a concentration of about 180 mM.

In some embodiments, the pharmaceutical composition comprises a viscosity modifying agent selected from L-Arginine Hydrochloric acid (L-Arg-HCL) or sodium chloride (NaCl), or any combination thereof. In some embodiments the pharmaceutical comprises L-Arg-HCL. In some embodiments, the pharmaceutical composition comprises sodium chloride.

In some embodiments, the pharmaceutical composition comprises L-Arg-HCL that is present at a concentration of about 20 mM to about 180 mM. In some embodiments, the pharmaceutical composition comprises L-Arg-HCL that is present at a concentration of about 20 mM to about 170 mM. In some embodiments, the pharmaceutical composition comprises L-Arg-HCL that is present at a concentration of about 20 mM to about 160 mM. In some embodiments, the pharmaceutical composition comprises L-Arg-HCL that is present at a concentration of about 20 mM to about 150 mM. In some embodiments, the pharmaceutical composition comprises L-Arg-HCL that is present at a concentration of about 20 mM to about 140 mM. In some embodiments, the pharmaceutical composition comprises L-Arg-HCL that is present at a concentration of about 20 mM to about 130 mM. In some embodiments, the pharmaceutical composition comprises L-Arg-HCL that is present at a concentration of about 20 mM to about 120 mM. In some embodiments, the pharmaceutical composition comprises L-Arg-HCL that is present at a concentration of about 20 mM to about 110 mM. In some embodiments, the pharmaceutical composition comprises L-Arg-HCL that is present at a concentration of about 20 mM to about 100 mM. In some embodiments, the pharmaceutical composition comprises L-Arg-HCL that is present at a concentration of about 20 mM to about 90 mM. In some embodiments, the pharmaceutical composition comprises L-Arg-HCL that is present at a concentration of about 20 mM to about 80 mM. In some embodiments, the pharmaceutical composition comprises L-Arg-HCL that is present at a concentration of about 20 mM to about 70 mM. In some embodiments, the pharmaceutical composition comprises L-Arg-HCL that is present at a concentration of about 20 mM to about 60 mM. In some embodiments, the pharmaceutical composition comprises L-Arg-HCL that is present at a concentration of about 20 mM to about 50 mM. In some embodiments, the pharmaceutical composition comprises L-Arg-HCL that is present at a concentration of about 20 mM to about 40 mM. In some embodiments, the pharmaceutical composition comprises L-Arg-HCL that is present at a concentration of about 20 mM to about 30 mM. In some embodiments, the pharmaceutical composition comprises L-Arg-HCL that is present at a concentration of about 30 mM to about 180 mM. In some embodiments, the pharmaceutical composition comprises L-Arg-HCL that is present at a concentration of about 30 mM to about 170 mM. In some embodiments, the pharmaceutical composition comprises L-Arg-HCL that is present at a concentration of about 30 mM to about 160 mM. In some embodiments, the pharmaceutical composition comprises L-Arg-HCL that is present at a concentration of about 30 mM to about 150 mM. In some embodiments, the pharmaceutical composition comprises L-Arg-HCL that is present at a concentration of about 30 mM to about 140 mM. In some embodiments, the pharmaceutical composition comprises L-Arg-HCL that is present at a concentration of about 30 mM to about 130 mM. In some embodiments, the pharmaceutical composition comprises L-Arg-HCL that is present at a concentration of about 30 mM to about 120 mM. In some embodiments, the pharmaceutical composition comprises L-Arg-HCL that is present at a concentration of about 30 mM to about 110 mM. In some embodiments, the pharmaceutical composition comprises L-Arg-HCL that is present at a concentration of about 30 mM to about 100 mM. In some embodiments, the pharmaceutical composition comprises L-Arg-HCL that is present at a concentration of about 30 mM to about 90 mM. In some embodiments, the pharmaceutical composition comprises L-Arg-HCL that is present at a concentration of about 30 mM to about 80 mM. In some embodiments, the pharmaceutical composition comprises L-Arg-HCL that is present at a concentration of about 30 mM to about 70 mM. In some embodiments, the pharmaceutical composition comprises L-Arg-HCL that is present at a concentration of about 30 mM to about 60 mM. In some embodiments, the pharmaceutical composition comprises L-Arg-HCL that is present at a concentration of about 30 mM to about 50 mM. In some embodiments, the pharmaceutical composition comprises L-Arg-HCL that is present at a concentration of about 30 mM to about 40 mM. In some embodiments, the pharmaceutical composition comprises L-Arg-HCL that is present at a concentration of about 40 mM to about 180 mM. In some embodiments, the pharmaceutical composition comprises L-Arg-HCL that is present at a concentration of about 40 mM to about 170 mM. In some embodiments, the pharmaceutical composition comprises L-Arg-HCL that is present at a concentration of about 40 mM to about 160 mM. In some embodiments, the pharmaceutical composition comprises L-Arg-HCL that is present at a concentration of about 40 mM to about 150 mM. In some embodiments, the pharmaceutical composition comprises L-Arg-HCL that is present at a concentration of about 40 mM to about 140 mM. In some embodiments, the pharmaceutical composition comprises L-Arg-HCL that is present at a concentration of about 40 mM to about 130 mM. In some embodiments, the pharmaceutical composition comprises L-Arg-HCL that is present at a concentration of about 40 mM to about 120 mM. In some embodiments, the pharmaceutical composition comprises L-Arg-HCL that is present at a concentration of about 40 mM to about 110 mM. In some embodiments, the pharmaceutical composition comprises L-Arg-HCL that is present at a concentration of about 40 mM to about 100 mM. In some embodiments, the pharmaceutical composition comprises L-Arg-HCL that is present at a concentration of about 40 mM to about 90 mM. In some embodiments, the pharmaceutical composition comprises L-Arg-HCL that is present at a concentration of about 40 mM to about 80 mM. In some embodiments, the pharmaceutical composition comprises L-Arg-HCL that is present at a concentration of about 40 mM to about 70 mM. In some embodiments, the pharmaceutical composition comprises L-Arg-HCL that is present at a concentration of about 40 mM to about 60 mM. In some embodiments, the pharmaceutical composition comprises L-Arg-HCL that is present at a concentration of about 40 mM to about 50 mM. In some embodiments, the pharmaceutical composition comprises L-Arg-HCL that is present at a concentration of about 50 mM to about 180 mM. In some embodiments, the pharmaceutical composition comprises L-Arg-HCL that is present at a concentration of about 50 mM to about 170 mM. In some embodiments, the pharmaceutical composition comprises L-Arg-HCL that is present at a concentration of about 50 mM to about 160 mM. In some embodiments, the pharmaceutical composition comprises L-Arg-HCL that is present at a concentration of about 50 mM to about 150 mM. In some embodiments, the pharmaceutical composition comprises L-Arg-HCL that is present at a concentration of about 50 mM to about 140 mM. In some embodiments, the pharmaceutical composition comprises L-Arg-HCL that is present at a concentration of about 50 mM to about 130 mM. In some embodiments, the pharmaceutical composition comprises L-Arg-HCL that is present at a concentration of about 50 mM to about 120 mM. In some embodiments, the pharmaceutical composition comprises L-Arg-HCL that is present at a concentration of about 50 mM to about 110 mM. In some embodiments, the pharmaceutical composition comprises L-Arg-HCL that is present at a concentration of about 50 mM to about 100 mM. In some embodiments, the pharmaceutical composition comprises L-Arg-HCL that is present at a concentration of about 50 mM to about 90 mM. In some embodiments, the pharmaceutical composition comprises L-Arg-HCL that is present at a concentration of about 50 mM to about 80 mM. In some embodiments, the pharmaceutical composition comprises L-Arg-HCL that is present at a concentration of about 50 mM to about 70 mM. In some embodiments, the pharmaceutical composition comprises L-Arg-HCL that is present at a concentration of about 50 mM to about 60 mM. In some embodiments, the pharmaceutical composition comprises L-Arg-HCL that is present at a concentration of about 60 mM to about 180 mM. In some embodiments, the pharmaceutical composition comprises L-Arg-HCL that is present at a concentration of about 60 mM to about 170 mM. In some embodiments, the pharmaceutical composition comprises L-Arg-HCL that is present at a concentration of about 60 mM to about 160 mM. In some embodiments, the pharmaceutical composition comprises L-Arg-HCL that is present at a concentration of about 60 mM to about 150 mM. In some embodiments, the pharmaceutical composition comprises L-Arg-HCL that is present at a concentration of about 60 mM to about 140 mM. In some embodiments, the pharmaceutical composition comprises L-Arg-HCL that is present at a concentration of about 60 mM to about 130 mM. In some embodiments, the pharmaceutical composition comprises L-Arg-HCL that is present at a concentration of about 60 mM to about 120 mM. In some embodiments, the pharmaceutical composition comprises L-Arg-HCL that is present at a concentration of about 60 mM to about 110 mM. In some embodiments, the pharmaceutical composition comprises L-Arg-HCL that is present at a concentration of about 60 mM to about 100 mM. In some embodiments, the pharmaceutical composition comprises L-Arg-HCL that is present at a concentration of about 60 mM to about 90 mM. In some embodiments, the pharmaceutical composition comprises L-Arg-HCL that is present at a concentration of about 60 mM to about 80 mM. In some embodiments, the pharmaceutical composition comprises L-Arg-HCL that is present at a concentration of about 60 mM to about 70 mM. In some embodiments, the pharmaceutical composition comprises L-Arg-HCL that is present at a concentration of about 70 mM to about 180 mM. In some embodiments, the pharmaceutical composition comprises L-Arg-HCL that is present at a concentration of about 70 mM to about 170 mM. In some embodiments, the pharmaceutical composition comprises L-Arg-HCL that is present at a concentration of about 70 mM to about 160 mM. In some embodiments, the pharmaceutical composition comprises L-Arg-HCL that is present at a concentration of about 70 mM to about 150 mM. In some embodiments, the pharmaceutical composition comprises L-Arg-HCL that is present at a concentration of about 70 mM to about 140 mM. In some embodiments, the pharmaceutical composition comprises L-Arg-HCL that is present at a concentration of about 70 mM to about 130 mM. In some embodiments, the pharmaceutical composition comprises L-Arg-HCL that is present at a concentration of about 70 mM to about 120 mM. In some embodiments, the pharmaceutical composition comprises L-Arg-HCL that is present at a concentration of about 70 mM to about 110 mM. In some embodiments, the pharmaceutical composition comprises L-Arg-HCL that is present at a concentration of about 70 mM to about 100 mM. In some embodiments, the pharmaceutical composition comprises L-Arg-HCL that is present at a concentration of about 70 mM to about 90 mM. In some embodiments, the pharmaceutical composition comprises L-Arg-HCL that is present at a concentration of about 70 mM to about 80 mM. In some embodiments, the pharmaceutical composition comprises L-Arg-HCL that is present at a concentration of about 80 mM to about 180 mM. In some embodiments, the pharmaceutical composition comprises L-Arg-HCL that is present at a concentration of about 80 mM to about 170 mM. In some embodiments, the pharmaceutical composition comprises L-Arg-HCL that is present at a concentration of about 80 mM to about 160 mM. In some embodiments, the pharmaceutical composition comprises L-Arg-HCL that is present at a concentration of about 80 mM to about 150 mM. In some embodiments, the pharmaceutical composition comprises L-Arg-HCL that is present at a concentration of about 80 mM to about 140 mM. In some embodiments, the pharmaceutical composition comprises L-Arg-HCL that is present at a concentration of about 80 mM to about 130 mM. In some embodiments, the pharmaceutical composition comprises L-Arg-HCL that is present at a concentration of about 80 mM to about 120 mM. In some embodiments, the pharmaceutical composition comprises L-Arg-HCL that is present at a concentration of about 80 mM to about 110 mM. In some embodiments, the pharmaceutical composition comprises L-Arg-HCL that is present at a concentration of about 80 mM to about 100 mM. In some embodiments, the pharmaceutical composition comprises L-Arg-HCL that is present at a concentration of about 80 mM to about 90 mM. In some embodiments, the pharmaceutical composition comprises L-Arg-HCL that is present at a concentration of about 90 mM to about 180 mM. In some embodiments, the pharmaceutical composition comprises L-Arg-HCL that is present at a concentration of about 90 mM to about 170 mM. In some embodiments, the pharmaceutical composition comprises L-Arg-HCL that is present at a concentration of about 90 mM to about 160 mM. In some embodiments, the pharmaceutical composition comprises L-Arg-HCL that is present at a concentration of about 90 mM to about 150 mM. In some embodiments, the pharmaceutical composition comprises L-Arg-HCL that is present at a concentration of about 90 mM to about 140 mM. In some embodiments, the pharmaceutical composition comprises L-Arg-HCL that is present at a concentration of about 90 mM to about 130 mM. In some embodiments, the pharmaceutical composition comprises L-Arg-HCL that is present at a concentration of about 90 mM to about 120 mM. In some embodiments, the pharmaceutical composition comprises L-Arg-HCL that is present at a concentration of about 90 mM to about 110 mM. In some embodiments, the pharmaceutical composition comprises L-Arg-HCL that is present at a concentration of about 90 mM to about 100 mM. In some embodiments, the pharmaceutical composition comprises L-Arg-HCL that is present at a concentration of about 100 mM to about 180 mM. In some embodiments, the pharmaceutical composition comprises L-Arg-HCL that is present at a concentration of about 100 mM to about 170 mM. In some embodiments, the pharmaceutical composition comprises L-Arg-HCL that is present at a concentration of about 100 mM to about 160 mM. In some embodiments, the pharmaceutical composition comprises L-Arg-HCL that is present at a concentration of about 100 mM to about 150 mM. In some embodiments, the pharmaceutical composition comprises L-Arg-HCL that is present at a concentration of about 100 mM to about 140 mM. In some embodiments, the pharmaceutical composition comprises L-Arg-HCL that is present at a concentration of about 100 mM to about 130 mM. In some embodiments, the pharmaceutical composition comprises L-Arg-HCL that is present at a concentration of about 100 mM to about 120 mM. In some embodiments, the pharmaceutical composition comprises L-Arg-HCL that is present at a concentration of about 100 mM to about 110 mM. In some embodiments, the pharmaceutical composition comprises L-Arg-HCL that is present at a concentration of about 110 mM to about 180 mM. In some embodiments, the pharmaceutical composition comprises L-Arg-HCL that is present at a concentration of about 110 mM to about 170 mM. In some embodiments, the pharmaceutical composition comprises L-Arg-HCL that is present at a concentration of about 110 mM to about 160 mM. In some embodiments, the pharmaceutical composition comprises L-Arg-HCL that is present at a concentration of about 110 mM to about 150 mM. In some embodiments, the pharmaceutical composition comprises L-Arg-HCL that is present at a concentration of about 110 mM to about 140 mM. In some embodiments, the pharmaceutical composition comprises L-Arg-HCL that is present at a concentration of about 110 mM to about 130 mM. In some embodiments, the pharmaceutical composition comprises L-Arg-HCL that is present at a concentration of about 110 mM to about 120 mM. In some embodiments, the pharmaceutical composition comprises L-Arg-HCL that is present at a concentration of about 120 mM to about 180 mM. In some embodiments, the pharmaceutical composition comprises L-Arg-HCL that is present at a concentration of about 120 mM to about 170 mM. In some embodiments, the pharmaceutical composition comprises L-Arg-HCL that is present at a concentration of about 120 mM to about 160 mM. In some embodiments, the pharmaceutical composition comprises L-Arg-HCL that is present at a concentration of about 120 mM to about 150 mM. In some embodiments, the pharmaceutical composition comprises L-Arg-HCL that is present at a concentration of about 120 mM to about 140 mM. In some embodiments, the pharmaceutical composition comprises L-Arg-HCL that is present at a concentration of about 120 mM to about 130 mM. In some embodiments, the pharmaceutical composition comprises L-Arg-HCL that is present at a concentration of about 130 mM to about 180 mM. In some embodiments, the pharmaceutical composition comprises L-Arg-HCL that is present at a concentration of about 130 mM to about 170 mM. In some embodiments, the pharmaceutical composition comprises L-Arg-HCL that is present at a concentration of about 130 mM to about 160 mM. In some embodiments, the pharmaceutical composition comprises L-Arg-HCL that is present at a concentration of about 130 mM to about 150 mM. In some embodiments, the pharmaceutical composition comprises L-Arg-HCL that is present at a concentration of about 130 mM to about 140 mM. In some embodiments, the pharmaceutical composition comprises L-Arg-HCL that is present at a concentration of about 140 mM to about 180 mM. In some embodiments, the pharmaceutical composition comprises L-Arg-HCL that is present at a concentration of about 140 mM to about 170 mM. In some embodiments, the pharmaceutical composition comprises L-Arg-HCL that is present at a concentration of about 140 mM to about 160 mM. In some embodiments, the pharmaceutical composition comprises L-Arg-HCL that is present at a concentration of about 140 mM to about 150 mM. In some embodiments, the pharmaceutical composition comprises L-Arg-HCL that is present at a concentration of about 150 mM to about 180 mM. In some embodiments, the pharmaceutical composition comprises L-Arg-HCL that is present at a concentration of about 150 mM to about 170 mM. In some embodiments, the pharmaceutical composition comprises L-Arg-HCL that is present at a concentration of about 150 mM to about 160 mM. In some embodiments, the pharmaceutical composition comprises L-Arg-HCL that is present at a concentration of about 160 mM to about 180 mM. In some embodiments, the pharmaceutical composition comprises L-Arg-HCL that is present at a concentration of about 160 mM to about 170 mM. In some embodiments, the pharmaceutical composition comprises L-Arg-HCL that is present at a concentration of about 170 mM to about 180 mM.

In some embodiments, the pharmaceutical composition comprises L-Arg-HCL that is present at a concentration of about 20 mM. In some embodiments, the pharmaceutical composition comprises L-Arg-HCL that is present at a concentration of about 30 mM. In some embodiments, the pharmaceutical composition comprises L-Arg-HCL that is present at a concentration of about 40 mM. In some embodiments, the pharmaceutical composition comprises L-Arg-HCL that is present at a concentration of about 50 mM. In some embodiments, the pharmaceutical composition comprises L-Arg-HCL that is present at a concentration of about 60 mM. In some embodiments, the pharmaceutical composition comprises L-Arg-HCL that is present at a concentration of about 70 mM. In some embodiments, the pharmaceutical composition comprises L-Arg-HCL that is present at a concentration of about 80 mM. In some embodiments, the pharmaceutical composition comprises L-Arg-HCL that is present at a concentration of about 90 mM. In some embodiments, the pharmaceutical composition comprises L-Arg-HCL that is present at a concentration of about 100 mM. In some embodiments, the pharmaceutical composition comprises L-Arg-HCL that is present at a concentration of about 110 mM. In some embodiments, the pharmaceutical composition comprises L-Arg-HCL that is present at a concentration of about 120 mM. In some embodiments, the pharmaceutical composition comprises L-Arg-HCL that is present at a concentration of about 130 mM. In some embodiments, the pharmaceutical composition comprises L-Arg-HCL that is present at a concentration of about 140 mM. In some embodiments, the pharmaceutical composition comprises L-Arg-HCL that is present at a concentration of about 150 mM. In some embodiments, the pharmaceutical composition comprises L-Arg-HCL that is present at a concentration of about 160 mM. In some embodiments, the pharmaceutical composition comprises L-Arg-HCL that is present at a concentration of about 170 mM. In some embodiments, the pharmaceutical composition comprises L-Arg-HCL that is present at a concentration of about 180 mM.

One of the major stresses that proteins (e.g., antibodies) may encounter is interfacial stress (e.g., from air/water interfaces in liquid compositions, or ice/water interfaces during freezing/thawing.) Surfactants are typically used to stabilize proteins in biopharmaceutical compositions while under stress or long-term storage to prevent or minimize aggregation and/or particle formation. Examples of a surfactant include, but are not limited to, anionic surfactants (e.g., ammonium lauryl sulfate, sodium lauryl sulfate, sodium laureth sulfate, sodium myreth sulfate, diocytl sodium sulfosuccinate, perfluorooctanesulfonate, perfluorobutanesulfonate, alkyl-aryl ether phosphates, alkyl ether phosphates, carboxylates, sodium lauroyl sarcosinate, perfluorononanoate, perfluorooctanoate); cationic surfactants (e.g., octenidine dihydrochloride, cetrimonium bromide, cetylpyridinium chloride, benzalkonium chloride, benzethonium chloride, dimethyldioctadecylammonium chloride, and dioctadecyldimethylammonium bromide); zwitterionic (amphoteric) surfactants (e.g., 3-[(3-cholamidopropyl)dimethylammonio]-1-propanesulfonate, cocamidopropyl hydroxysultaine, phosphatidylserine, phosphatidylethanolamine, phosphatidylcholine, sphingomyelins, lauryldimethylamine oxide and myristamine oxide); non-ionic surfactants (e.g., polysorbates, poloxmaers, or Brij series); ethoxylates (e.g., fatty alcohol ethoxylate (e.g., octaethylene glycol monododecyl ether and pentaethylene glycol monododecyl ether), alkylphenolethoxylates (e.g., nonoxynols and Triton X-100); fatty acid ethoxylates, ethoxylated amines and/or fatty acid amides (e.g., poly ethoxylated tallow amine, cocamide monoethanol amine, and cocamide diethanolamine); terminally blocked ethoxylates (e.g., pol oxamers); fatty acid esters of polyhydroxy compounds; fatty acid esters of glycerol (e.g., glycerol monostearate and glycerol monolaurate); fatty acid esters of sorbitol (e.g., spans such as sorbitan monolaurate, sorbitan monostearate, and sorbitan tristearate, and polysorbates (Tweens) such as Tween 20, Tween 40, Tween 60, and Tween 80); fatty acid esters of sucrose; alkyl poly glucosides (e.g., decyl glucoside, lauryl glucoside, and octyl glucoside); or a combination thereof.

In some embodiments, the pharmaceutical composition comprises a surfactant present at a concentration of about 0.001% w/v to about 0.4% w/v. In some embodiments, the pharmaceutical composition comprises a surfactant present at a concentration of about 0.001% w/v to about 0.2% w/v. In some embodiments, the pharmaceutical composition comprises a surfactant present at a concentration of about 0.001% w/v to about 0.1% w/v. In some embodiments, the pharmaceutical composition comprises a surfactant present at a concentration of about 0.001% w/v to about 0.01% w/v. In some embodiments, the pharmaceutical composition comprises a surfactant present at a concentration of about 0.01% w/v to about 0.2% w/v. In some embodiments, the pharmaceutical composition comprises a surfactant present at a concentration of about 0.05% w/v to about 0.15% w/v. In some embodiments, the pharmaceutical composition comprises a surfactant present at a concentration of about 0.1% w/v to about 0.2% w/v. In some embodiments, the pharmaceutical composition comprises a surfactant present at a concentration of about 0.2% w/v to about 0.4% w/v. In some embodiments, the pharmaceutical composition comprises a surfactant present at a concentration of about 0.001% w/v to about 0.2% w/v.

In some embodiments, the pharmaceutical composition comprises a surfactant present at a concentration of about 0.001% w/v. In some embodiments, the pharmaceutical composition comprises a surfactant present at a concentration of about 0.002% w/v. In some embodiments, the pharmaceutical composition comprises a surfactant present at a concentration of about 0.003% w/v. In some embodiments, the pharmaceutical composition comprises a surfactant present at a concentration of about 0.004% w/v. In some embodiments, the pharmaceutical composition comprises a surfactant present at a concentration of about 0.005% w/v. In some embodiments, the pharmaceutical composition comprises a surfactant present at a concentration of about 0.006% w/v. In some embodiments, the pharmaceutical composition comprises a surfactant present at a concentration of about 0.007% w/v. In some embodiments, the pharmaceutical composition comprises a surfactant present at a concentration of about 0.008% w/v. In some embodiments, the pharmaceutical composition comprises a surfactant present at a concentration of about 0.009% w/v. In some embodiments, the pharmaceutical composition comprises a surfactant present at a concentration of about 0.01% w/v. In some embodiments, the pharmaceutical composition comprises a surfactant present at a concentration of about 0.02% w/v. In some embodiments, the pharmaceutical composition comprises a surfactant present at a concentration of about 0.03% w/v. In some embodiments, the pharmaceutical composition comprises a surfactant present at a concentration of about 0.04% w/v. In some embodiments, the pharmaceutical composition comprises a surfactant present at a concentration of about 0.05% w/v. In some embodiments, the pharmaceutical composition comprises a surfactant present at a concentration of about 0.06% w/v. In some embodiments, the pharmaceutical composition comprises a surfactant present at a concentration of about 0.07% w/v. In some embodiments, the pharmaceutical composition comprises a surfactant present at a concentration of about 0.08% w/v. In some embodiments, the pharmaceutical composition comprises a surfactant present at a concentration of about 0.09% w/v. In some embodiments, the pharmaceutical composition comprises a surfactant present at a concentration of about 0.10% w/v. In some embodiments, the pharmaceutical composition comprises a surfactant present at a concentration of about 0.11% w/v. In some embodiments, the pharmaceutical composition comprises a surfactant present at a concentration of about 0.12% w/v. In some embodiments, the pharmaceutical composition comprises a surfactant present at a concentration of about 0.13% w/v. In some embodiments, the pharmaceutical composition comprises a surfactant present at a concentration of about 0.14% w/v. In some embodiments, the pharmaceutical composition comprises a surfactant present at a concentration of about 0.15% w/v. In some embodiments, the pharmaceutical composition comprises a surfactant present at a concentration of about 0.16% w/v. In some embodiments, the pharmaceutical composition comprises a surfactant present at a concentration of about 0.17% w/v. In some embodiments, the pharmaceutical composition comprises a surfactant present at a concentration of about 0.18% w/v. In some embodiments, the pharmaceutical composition comprises a surfactant present at a concentration of about 0.19% w/v. In some embodiments, the pharmaceutical composition comprises a surfactant present at a concentration of about 0.20% w/v. In some embodiments, the pharmaceutical composition comprises a surfactant present at a concentration of about 0.25% w/v. In some embodiments, the pharmaceutical composition comprises a surfactant present at a concentration of about 0.3% w/v. In some embodiments, the pharmaceutical composition comprises a surfactant present at a concentration of about 0.35% w/v. In some embodiments, the pharmaceutical composition comprises a surfactant present at a concentration of about 0.4% w/v.

In some embodiments, the pharmaceutical composition comprises a surfactant selected from polysorbate (PS)80, polysorbate (PS)20, polyethylene glycol (PG)3350, poloxamer (P)188, or any combination thereof. In some embodiments, the pharmaceutical composition comprises PS80. In some embodiments, the pharmaceutical composition comprises PS20. In some embodiments, the pharmaceutical composition comprises PG3350. In some embodiments, the pharmaceutical composition comprises P188.

In some embodiments, the surfactant is a poloxamer. Poloxamers are nonionic triblock copolymers composed of a central hydrophobic chain of polyoxypropylene (polypropylene oxide)) flanked by two hydrophilic chains of polyoxyethylene (poly(ethylene oxide)). Examples of a poloxamer include, but are not limited to, poloxamer 188, poloxamer 407, poloxamer 184, poloxamer 124, or a combination thereof.

In some embodiments, the pharmaceutical composition comprises P188 present at a concentration of about 0.001% w/v to about 0.1% w/v. In some embodiments, the pharmaceutical composition comprises P188 present at a concentration of about 0.001% w/v to about 0.01% w/v. In some embodiments, the pharmaceutical composition comprises P188 present at a concentration of about 0.01% w/v to about 0.2% w/v. In some embodiments, the pharmaceutical composition comprises P188 present at concentration of about 0.2% w/v to about 0.4% w/v. In some embodiments, the pharmaceutical composition comprises P188 present at a concentration of about 0.001% w/v to about 0.2% w/v. In some embodiments, the pharmaceutical composition comprises P188 present at a concentration of about 0.001% w/v to about 0.4% w/v.

In some embodiments, the pharmaceutical composition comprises P188 present at a concentration of about 0.001% w/v. In some embodiments, the pharmaceutical composition comprises P188 present at a concentration of about 0.002% w/v. In some embodiments, the pharmaceutical composition comprises P188 present at a concentration of about 0.003% w/v. In some embodiments, the pharmaceutical composition comprises P188 present at a concentration of about 0.004% w/v. In some embodiments, the pharmaceutical composition comprises P188 present at a concentration of about 0.005% w/v. In some embodiments, the pharmaceutical composition comprises P188 present at a concentration of about 0.006% w/v. In some embodiments, the pharmaceutical composition comprises P188 present at a concentration of about 0.007% w/v. In some embodiments, the pharmaceutical composition comprises P188 present at a concentration of about 0.008% w/v. In some embodiments, the pharmaceutical composition comprises P188 present at a concentration of about 0.009% w/v. In some embodiments, the pharmaceutical composition comprises P188 present at a concentration of about 0.01% w/v. In some embodiments, the pharmaceutical composition comprises P188 present at a concentration of about 0.02% w/v. In some embodiments, the pharmaceutical composition comprises P188 present at a concentration of about 0.03% w/v. In some embodiments, the pharmaceutical composition comprises P188 present at a concentration of about 0.04% w/v. In some embodiments, the pharmaceutical composition comprises P188 present at a concentration of about 0.05% w/v. In some embodiments, the pharmaceutical composition comprises P188 present at a concentration of about 0.06% w/v. In some embodiments, the pharmaceutical composition comprises P188 present at a concentration of about 0.07% w/v. In some embodiments, the pharmaceutical composition comprises P188 present at a concentration of about 0.08% w/v. In some embodiments, the pharmaceutical composition comprises P188 present at a concentration of about 0.09% w/v. In some embodiments, the pharmaceutical composition comprises P188 present at a concentration of about 0.10% w/v. In some embodiments, the pharmaceutical composition comprises P188 present at a concentration of about 0.11% w/v. In some embodiments, the pharmaceutical composition comprises P188 present at a concentration of about 0.12% w/v. In some embodiments, the pharmaceutical composition comprises P188 present at a concentration of about 0.13% w/v. In some embodiments, the pharmaceutical composition comprises P188 present at a concentration of about 0.14% w/v. In some embodiments, the pharmaceutical composition comprises P188 present at a concentration of about 0.15% w/v. In some embodiments, the pharmaceutical composition comprises P188 present at a concentration of about 0.16% w/v. In some embodiments, the pharmaceutical composition comprises P188 present at a concentration of about 0.17% w/v. In some embodiments, the pharmaceutical composition comprises P188 present at a concentration of about 0.18% w/v. In some embodiments, the pharmaceutical composition comprises P188 present at a concentration of about 0.19% w/v. In some embodiments, the pharmaceutical composition comprises P188 present at a concentration of about 0.20% w/v. In some embodiments, the pharmaceutical composition comprises P188 present at a concentration of about 0.25% w/v. In some embodiments, the pharmaceutical composition comprises P188 present at a concentration of about 0.3% w/v. In some embodiments, the pharmaceutical composition comprises P188 present at a concentration of about 0.35% w/v. In some embodiments, the pharmaceutical composition comprises P188 present at a concentration of about 0.4% w/v.

In some embodiments, the pharmaceutical composition comprises an antibody that is present at a concentration of about 25 mg/mL to about 300 mg/mL. In some embodiments, the pharmaceutical composition comprises an antibody that is present at a concentration of about 25 mg/mL to about 400 mg/mL. In some embodiments, the pharmaceutical composition comprises an antibody that is present at a concentration of about 25 mg/mL to about 500 mg/mL. In some embodiments, the pharmaceutical composition comprises an antibody that is present at a concentration of about 25 mg/mL to about 600 mg/mL. In some embodiments, the pharmaceutical composition comprises an antibody that is present at a concentration of about 25 mg/mL to about 200 mg/mL. In some embodiments, the pharmaceutical composition comprises an antibody present at a concentration of about 25 mg/mL to about 275 mg/mL In some embodiments, the pharmaceutical composition comprises an antibody present at a concentration of about 25 mg/mL to about 250 mg/mL In some embodiments, the pharmaceutical composition comprises an antibody present at a concentration of about 25 mg/mL to about 225 mg/mL In some embodiments, the pharmaceutical composition comprises an antibody present at a concentration of about 25 mg/mL to about 200 mg/mL. In some embodiments, the pharmaceutical composition comprises an antibody present at a concentration of about 25 mg/mL to about 175 mg/mL. In some embodiments, the pharmaceutical composition comprises an antibody present at a concentration of about 25 mg/mL to about 150 mg/mL. In some embodiments, the pharmaceutical composition comprises an antibody present at a concentration of about 25 mg/mL to about 125 mg/mL. In some embodiments, the pharmaceutical composition comprises an antibody present at a concentration of about 25 mg/mL to about 100 mg/mL. In some embodiments, the pharmaceutical composition comprises an antibody present at a concentration of about 25 mg/mL to about 75 mg/mL. In some embodiments, the pharmaceutical composition comprises an antibody present at a concentration of about 50 mg/mL to about 300 mg/mL. In some embodiments, the pharmaceutical composition comprises an antibody present at a concentration of about 50 mg/mL to about 275 mg/mL. In some embodiments, the pharmaceutical composition comprises an antibody present at a concentration of about 50 mg/mL to about 250 mg/mL. In some embodiments, the pharmaceutical composition comprises an antibody present at a concentration of about 50 mg/mL to about 225 mg/mL. In some embodiments, the pharmaceutical composition comprises an antibody present at a concentration of about 50 mg/mL to about 200 mg/mL. In some embodiments, the pharmaceutical composition comprises an antibody present at a concentration of about 50 mg/mL to about 175 mg/mL. In some embodiments, the pharmaceutical composition comprises an antibody present at a concentration of about 50 mg/mL to about 150 mg/mL. In some embodiments, the pharmaceutical composition comprises an antibody present at a concentration of about 50 mg/mL to about 125 mg/mL. In some embodiments, the pharmaceutical composition comprises an antibody present at a concentration of about 50 mg/mL to about 100 mg/mL. In some embodiments, the pharmaceutical composition comprises an antibody present at a concentration of about 50 mg/mL to about 75 mg/mL. In some embodiments, the pharmaceutical composition comprises an antibody present at a concentration of about 75 mg/mL to about 300 mg/mL. In some embodiments, the pharmaceutical composition comprises an antibody present at a concentration of about 75 mg/mL to about 275 mg/mL. In some embodiments, the pharmaceutical composition comprises an antibody present at a concentration of about 75 mg/mL to about 250 mg/mL. In some embodiments, the pharmaceutical composition comprises an antibody present at a concentration of about 75 mg/mL to about 225 mg/mL. In some embodiments, the pharmaceutical composition comprises an antibody present at a concentration of about 75 mg/mL to about 200 mg/mL. In some embodiments, the pharmaceutical composition comprises an antibody present at a concentration of about 75 mg/mL to about 175 mg/mL. In some embodiments, the pharmaceutical composition comprises an antibody present at a concentration of about 75 mg/mL to about 150 mg/mL. In some embodiments, the pharmaceutical composition comprises an antibody present at a concentration of about 75 mg/mL to about 125 mg/mL. In some embodiments, the pharmaceutical composition comprises an antibody present at a concentration of about 75 mg/mL to about 100 mg/mL. In some embodiments, the pharmaceutical composition comprises an antibody present at a concentration of about 100 mg/mL to about 300 mg/mL. In some embodiments, the pharmaceutical composition comprises an antibody present at a concentration of about 100 mg/mL to about 275 mg/mL. In some embodiments, the pharmaceutical composition comprises an antibody present at a concentration of about 100 mg/mL to about 250 mg/mL. In some embodiments, the pharmaceutical composition comprises an antibody present at a concentration of about 100 mg/mL to about 225 mg/mL. In some embodiments, the pharmaceutical composition comprises an antibody present at a concentration of about 100 mg/mL to about 200 mg/mL. In some embodiments, the pharmaceutical composition comprises an antibody present at a concentration of about 100 mg/mL to about 175 mg/mL. In some embodiments, the pharmaceutical composition comprises an antibody present at a concentration of about 100 mg/mL to about 150 mg/mL. In some embodiments, the pharmaceutical composition comprises an antibody present at a concentration of about 100 mg/mL to about 125 mg/mL. In some embodiments, the pharmaceutical composition comprises an antibody present at a concentration of about 125 mg/mL to about 300 mg/mL. In some embodiments, the pharmaceutical composition comprises an antibody present at a concentration of about 125 mg/mL to about 275 mg/mL. In some embodiments, the pharmaceutical composition comprises an antibody present at a concentration of about 125 mg/mL to about 250 mg/mL. In some embodiments, the pharmaceutical composition comprises an antibody present at a concentration of about 125 mg/mL to about 225 mg/mL. In some embodiments, the pharmaceutical composition comprises an antibody present at a concentration of about 125 mg/mL to about 200 mg/mL. In some embodiments, the pharmaceutical composition comprises an antibody present at a concentration of about 125 mg/mL to about 175 mg/mL. In some embodiments, the pharmaceutical composition comprises an antibody present at a concentration of about 125 mg/mL to about 150 mg/mL. In some embodiments, the pharmaceutical composition comprises an antibody present at a concentration of about 150 mg/mL to about 300 mg/mL. In some embodiments, the pharmaceutical composition comprises an antibody present at a concentration of about 150 mg/mL to about 275 mg/mL. In some embodiments, the pharmaceutical composition comprises an antibody present at a concentration of about 150 mg/mL to about 250 mg/mL. In some embodiments, the pharmaceutical composition comprises an antibody present at a concentration of about 150 mg/mL to about 225 mg/mL. In some embodiments, the pharmaceutical composition comprises an antibody present at a concentration of about 150 mg/mL to about 200 mg/mL. In some embodiments, the pharmaceutical composition comprises an antibody present at a concentration of about 150 mg/mL to about 175 mg/mL. In some embodiments, the pharmaceutical composition comprises an antibody present at a concentration of or about 175 mg/mL to about 300 mg/mL. In some embodiments, the pharmaceutical composition comprises an antibody present at a concentration of or about 175 mg/mL to about 275 mg/mL. In some embodiments, the pharmaceutical composition comprises an antibody present at a concentration of or about 175 mg/mL to about 250 mg/mL. In some embodiments, the pharmaceutical composition comprises an antibody present at a concentration of or about 175 mg/mL to about 225 mg/mL. In some embodiments, the pharmaceutical composition comprises an antibody present at a concentration of or about 175 mg/mL to about 200 mg/mL. In some embodiments, the pharmaceutical composition comprises an antibody present at a concentration of or about 200 mg/mL to about 300 mg/mL. In some embodiments, the pharmaceutical composition comprises an antibody present at a concentration of or about 200 mg/mL to about 275 mg/mL. In some embodiments, the pharmaceutical composition comprises an antibody present at a concentration of or about 200 mg/mL to about 250 mg/mL. In some embodiments, the pharmaceutical composition comprises an antibody present at a concentration of or about 200 mg/mL to about 225 mg/mL. In some embodiments, the pharmaceutical composition comprises an antibody present at a concentration of or about 225 mg/mL to about 300 mg/mL. In some embodiments, the pharmaceutical composition comprises an antibody present at a concentration of or about 225 mg/mL to about 275 mg/mL. In some embodiments, the pharmaceutical composition comprises an antibody present at a concentration of or about 225 mg/mL to about 250 mg/mL. In some embodiments, the pharmaceutical composition comprises an antibody present at a concentration of or about 250 mg/mL to about 300 mg/mL. In some embodiments, the pharmaceutical composition comprises an antibody present at a concentration of or about 250 mg/mL to about 275 mg/mL. In some embodiments, the pharmaceutical composition comprises an antibody present at a concentration of or about 275 mg/mL to about 300 mg/mL. In some embodiments, the pharmaceutical composition comprises an antibody present at a concentration of or about 100 mg/mL to about 200 mg/mL. In some embodiments, the pharmaceutical composition comprises an antibody present at a concentration of or about 200 mg/mL to about 300 mg/mL. In some embodiments, the pharmaceutical composition comprises an antibody present at a concentration of or about 300 mg/mL to about 400 mg/mL. In some embodiments, the pharmaceutical composition comprises an antibody present at a concentration of or about 400 mg/mL to about 500 mg/mL. In some embodiments, the pharmaceutical composition comprises an antibody present at a concentration of or about 500 mg/mL to about 600 mg/mL. In some embodiments, the pharmaceutical composition comprises an antibody present at a concentration of or about 100 mg/mL to about 300 mg/mL. In some embodiments, the pharmaceutical composition comprises an antibody present at a concentration of or about 200 mg/mL to about 400 mg/mL. In some embodiments, the pharmaceutical composition comprises an antibody present at a concentration of or about 300 mg/mL to about 500 mg/mL. In some embodiments, the pharmaceutical composition comprises an antibody present at a concentration of or about 400 mg/mL to about 600 mg/mL. In some embodiments, the pharmaceutical composition comprises an antibody present at a concentration of or about 100 mg/mL to about 400 mg/mL. In some embodiments, the pharmaceutical composition comprises an antibody present at a concentration of or about 200 mg/mL to about 500 mg/mL. In some embodiments, the pharmaceutical composition comprises an antibody present at a concentration of or about 300 mg/mL to about 600 mg/mL.

In some embodiments, the pharmaceutical composition comprises an antibody present at a concentration of about 25 mg/mL. In some embodiments, the pharmaceutical composition comprises an antibody present at a concentration of about 30 mg/mL. In some embodiments, the pharmaceutical composition comprises an antibody present at a concentration of about 40 mg/mL. In some embodiments, the pharmaceutical composition comprises an antibody present at a concentration of about 50 mg/mL. In some embodiments, the pharmaceutical composition comprises an antibody present at a concentration of about 60 mg/mL. In some embodiments, the pharmaceutical composition comprises an antibody present at a concentration of about 70 mg/mL. In some embodiments, the pharmaceutical composition comprises an antibody present at a concentration of about 80 mg/mL. In some embodiments, the pharmaceutical composition comprises an antibody present at a concentration of about 90 mg/mL. In some embodiments, the pharmaceutical composition comprises an antibody present at a concentration of about 100 mg/mL. In some embodiments, the pharmaceutical composition comprises an antibody present at a concentration of about 110 mg/mL. In some embodiments, the pharmaceutical composition comprises an antibody present at a concentration of about 120 mg/mL. In some embodiments, the pharmaceutical composition comprises an antibody present at a concentration of about 130 mg/mL. In some embodiments, the pharmaceutical composition comprises an antibody present at a concentration of about 140 mg/mL. In some embodiments, the pharmaceutical composition comprises an antibody present at a concentration of about 150 mg/mL. In some embodiments, the pharmaceutical composition comprises an antibody present at a concentration of about 160 mg/mL. In some embodiments, the pharmaceutical composition comprises an antibody present at a concentration of about 170 mg/mL. In some embodiments, the pharmaceutical composition comprises an antibody present at a concentration of about 175 mg/mL. In some embodiments, the pharmaceutical composition comprises an antibody present at a concentration of about 200 mg/mL. In some embodiments, the pharmaceutical composition comprises an antibody present at a concentration of about 225 mg/mL. In some embodiments, the pharmaceutical composition comprises an antibody present at a concentration of about 275 mg/mL. In some embodiments, the pharmaceutical composition comprises an antibody present at a concentration of about 300 mg/mL. In some embodiments, the pharmaceutical composition comprises an antibody present at a concentration of about 325 mg/mL. In some embodiments, the pharmaceutical composition comprises an antibody present at a concentration of about 350 mg/mL. In some embodiments, the pharmaceutical composition comprises an antibody present at a concentration of about 375 mg/mL. In some embodiments, the pharmaceutical composition comprises an antibody present at a concentration of about 400 mg/mL. In some embodiments, the pharmaceutical composition comprises an antibody present at a concentration of about 425 mg/mL. In some embodiments, the pharmaceutical composition comprises an antibody present at a concentration of about 450 mg/mL. In some embodiments, the pharmaceutical composition comprises an antibody present at a concentration of about 475 mg/mL. In some embodiments, the pharmaceutical composition comprises an antibody present at a concentration of about 500 mg/mL. In some embodiments, the pharmaceutical composition comprises an antibody present at a concentration of about 525 mg/mL. In some embodiments, the pharmaceutical composition comprises an antibody present at a concentration of about 550 mg/mL. In some embodiments, the pharmaceutical composition comprises an antibody present at a concentration of about 575 mg/mL. In some embodiments, the pharmaceutical composition comprises an antibody present at a concentration of about 600 mg/mL.

In some embodiments, the pharmaceutical composition comprises an antibody that is a monoclonal antibody. In some embodiments, the pharmaceutical composition comprises an antibody that is a polyclonal antibody. In some embodiments, the pharmaceutical composition comprises an antibody that is an immunoglobulin G₄ (IgG₄) type monoclonal antibody. In some embodiments, the pharmaceutical composition comprises an antibody selected from those provided herein. In some embodiments, the pharmaceutical composition comprises an antibody that comprises a heavy chain variable region selected from any heavy chain variable regions provided herein; and a light chain variable region selected from any light chain variable regions provided herein.

In some embodiments, the pharmaceutical composition comprises an antibody that comprises a heavy chain variable region with an amino acid sequence of SEQ ID NO: 17 and a light chain variable region with an amino acid sequence of SEQ ID NO: 18. In some embodiments, the pharmaceutical composition comprises an antibody that comprises a heavy chain variable region with an amino acid sequence of SEQ ID NO: 342 and a light chain variable region with an amino acid sequence of SEQ ID NO: 18. In some embodiments, the pharmaceutical composition comprises an antibody that comprises a heavy chain variable region with an amino acid sequence of SEQ ID NO: 17. In some embodiments, the pharmaceutical composition comprises an antibody that comprises a heavy chain variable region with an amino acid sequence of SEQ ID NO: 342. In some embodiments, the pharmaceutical composition comprises an antibody that comprises a light chain variable region with an amino acid sequence of SEQ ID NO: 18.

In some embodiments, the antibody or antigen binding fragment thereof, comprises a heavy chain variable region comprising heavy chain HCDR1, HCDR2, and HCDR3 sequences, wherein the HCDR1 sequence has the amino acid sequence of SEQ ID NO: 61, the heavy chain HCDR2 has the amino acid sequence of SEQ ID NO: 62, and the heavy chain HCDR3 sequence has the amino acid sequence of SEQ ID NO: 78, or variants or convention equivalents of any of the foregoing; and a light chain variable region comprising light chain LCDR1, LCDR2, and LCDR3 sequences, wherein the light chain LCDR1 sequence has the amino acid sequence SEQ ID NO: 64, the light chain LCDR2 sequence has the amino acid sequence of SEQ ID NO: 65, and the light chain LCDR3 sequence has the amino acid sequence of SEQ ID NO: 79, or variants or convention equivalents of any of the foregoing.

In some embodiments, the pharmaceutical composition is stable as defined herein. In some embodiments, the composition as described herein is stable under the conditions of manufacture and storage. In some embodiments, the composition remains stable and active even when subjected to stress and when stored in containers made of various materials, including glass. In some embodiments, the storage container is a vial with a stopper and a cover. In some embodiments, the container is a pre-filled glass syringe.

In some embodiments, the pharmaceutical composition is stable for 3 or more freeze/thaw cycles between about −70° C. and room temperature. In some embodiments, the pharmaceutical composition is stable for 4 or more freeze/thaw cycles between about −70° C. and room temperature. In some embodiments, the pharmaceutical composition is stable for 5 or more freeze/thaw cycles between about −70° C. and room temperature.

In some embodiments, the pharmaceutical composition is stable under agitation conditions from 80-120 rpm for 3 days. In some embodiments, the pharmaceutical composition is stable under agitation conditions from 80-120 rpm for 2 days. In some embodiments, the pharmaceutical composition is stable under agitation conditions from 80-120 rpm for 1 day. In some embodiments, the pharmaceutical composition is stable under agitation conditions from 80-120 rpm for up to 3 days. In some embodiments, the pharmaceutical composition is stable under agitation conditions at 100 rpm for up to 3 days. In some embodiments, the pharmaceutical composition is stable against agitation conditions. In some embodiments, the pharmaceutical composition is stable in agitation conditions at room temperature.

In some embodiments, the pharmaceutical composition is stable for up to 3 months when stored at about −80° C. In some embodiments, the pharmaceutical composition is stable for up to 3 months when stored at about −70° C. In some embodiments, the pharmaceutical composition is stable for up to 3 months when stored at about −60° C. In some embodiments, the pharmaceutical composition is stable for up to 3 months when stored at about −50° C. In some embodiments, the pharmaceutical composition is stable for up to 3 months when stored at about −40° C. In some embodiments, the pharmaceutical composition is stable for up to 3 months when stored at about −30° C. In some embodiments, the pharmaceutical composition is stable for up to 3 months when stored at about −20° C. In some embodiments, the pharmaceutical composition is stable for up to 3 months when stored at about −10° C. In some embodiments, the pharmaceutical composition is stable for up to 3 months when stored at about 0° C. In some embodiments, the pharmaceutical composition is stable for up to 3 months when stored at about 10° C. In some embodiments, the pharmaceutical composition is stable for up to 3 months when stored at about 20° C. In some embodiments, the pharmaceutical composition is stable for up to 3 months when stored at about 30° C. In some embodiments, the pharmaceutical composition is stable for up to 3 months when stored at about 40° C. In some embodiments, the pharmaceutical composition is stable for up to 3 months when stored at about 50° C.

In some embodiments, the liquid or dissolved lyophilized powder formulation of the pharmaceutical composition is colorless in appearance. In some embodiments, the liquid or dissolved lyophilized powder formulation of the pharmaceutical composition is slightly opalescent in appearance. In some embodiments, the liquid or dissolved lyophilized powder formulation of the pharmaceutical composition is free of visible particles. In some embodiments, the liquid or dissolved lyophilized powder formulation of the pharmaceutical composition is free of sub-visible particles. In some embodiments, the liquid or dissolved lyophilized powder formulation of the pharmaceutical composition is essentially free of sub-visible particles. In some embodiments, the liquid or dissolved lyophilized powder formulation of the pharmaceutical composition is almost free of sub-visible particles. In some embodiments, the liquid or dissolved lyophilized powder formulation of the pharmaceutical composition is free of visible particles comprising high molecular weight molecules. In some embodiments, the liquid or dissolved lyophilized powder formulation of the pharmaceutical composition is free of sub-visible particles comprising high molecular weight molecules. In some embodiments, the liquid or dissolved lyophilized powder formulation of the pharmaceutical composition is essentially free of sub-visible particles comprising high molecular weight molecules. In some embodiments, the liquid or dissolved lyophilized powder formulation of the pharmaceutical composition is almost free of sub-visible particles comprising high molecular weight molecules. In some embodiments, the liquid or dissolved lyophilized powder formulation of the pharmaceutical composition is free of low molecular weight molecule degradants of the antibody, or fragment thereof.

In some embodiments, the liquid or dissolved lyophilized powder formulation of the pharmaceutical composition is free of degradation products. In some embodiments, the liquid or dissolved lyophilized powder formulation of the pharmaceutical composition is consistently free of degradation products (e.g., HMWS, LMWS, or minor species) over time. In some embodiments, the liquid or dissolved lyophilized powder formulation of the pharmaceutical composition is free of degradation products at about 2° C. to about 8° C. In some embodiments, the liquid or dissolved lyophilized powder formulation of the pharmaceutical composition is free of product related impurities at about 2° C. to about 8° C. In some embodiments, the liquid or dissolved lyophilized powder formulation of the pharmaceutical composition is free of degradation products at about 0° C. or below. In some embodiments, the liquid or dissolved lyophilized powder formulation of the pharmaceutical composition is free of product related impurities at about 0° C. or below. In some embodiments, the liquid or dissolved lyophilized powder formulation of the pharmaceutical composition is consistently free of oxidation products. In some embodiments, the liquid or dissolved lyophilized powder formulation of the pharmaceutical composition is consistently free of oxidation degradation products. In some embodiments, the liquid or dissolved lyophilized powder formulation of the pharmaceutical composition is consistently free of tryptophan oxidation products. In some embodiments, the liquid or dissolved lyophilized powder formulation of the pharmaceutical composition is consistently free of advanced glycation end-product (AGE) products. In some embodiments, the liquid or dissolved lyophilized powder formulation of the pharmaceutical composition is consistently free of adduct degradation. In some embodiments, the color of the liquid or dissolved lyophilized powder formulation of the pharmaceutical composition is consistent over time. In some embodiments, the appearance (e.g., color and/or clarity) of the liquid or dissolved lyophilized powder formulation of the pharmaceutical composition is consistent over time. In some embodiments, the color and clarity of the liquid or dissolved lyophilized powder formulation of the pharmaceutical composition is consistent over time. In some embodiments, the color of the liquid or dissolved lyophilized powder formulation of the pharmaceutical composition does not significantly change over time. In some embodiments, the color of the liquid or dissolved lyophilized powder formulation of the pharmaceutical composition changes slightly over time.

A change in color of a formulation can indicate the presence of degradation products over time. It is important that degradation products are minimized in antibody formulations; therefore, it is important that the color does not substantially change. In some embodiments, degradation products, such as tryptophan oxidants, AGE products, and adduct degradation, may change the color of a formulation; therefore, it is critical that the color of a formulation remains consistent over time. In some embodiments, the color of a formulation is not critical to its safety and/or stability. In some embodiments, the liquid or dissolved lyophilized powder formulation of the pharmaceutical composition is free of degradation products and is clear in appearance after between about 1 day and about 12 weeks at about −20° C., at about 2° C. to 8° C., or at about 40° C. In some embodiments, the liquid or dissolved lyophilized powder formulation of the pharmaceutical composition is free of degradation products and is clear in appearance after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is free of degradation products and is clear in appearance after about 1, 2, 3, 4, 5, or more freeze/thaw cycles between about −70° C. and about 25° C. In some embodiments, the liquid or dissolved lyophilized powder formulation of the pharmaceutical composition is free of degradation products and is clear in appearance after about 3 or more freeze/thaw cycles between about −70° C. and about 25° C. In some embodiments, the liquid or dissolved lyophilized powder formulation of the pharmaceutical composition is free of degradation products and is clear in appearance after about 5 or more freeze/thaw cycles between about −70° C. and about 25° C. In some embodiments, the liquid or dissolved lyophilized powder formulation of the pharmaceutical composition is free of degradation products and is clear in appearance after agitation conditions at room temperature. In some embodiments, the liquid or dissolved lyophilized powder formulation of the pharmaceutical composition is free of degradation products and is free of degradation products and is clear in appearance after agitation conditions at room temperature after about 1, 2, 3, or more days. In some embodiments, the liquid or dissolved lyophilized powder formulation of the pharmaceutical composition is free of degradation products and is clear in appearance after agitation conditions at room temperature after about 1 or more days. In some embodiments, the liquid or dissolved lyophilized powder formulation of the pharmaceutical composition is free of degradation products and is clear in appearance after agitation conditions at room temperature after about 3 or more days.

In some embodiments, a substantial color change can be slight yellow, yellow, slight yellow brown, yellow brown, slight red, red, slight pink, pink, slight brown, or brown. In some embodiments, the liquid or dissolved lyophilized powder formulation of the pharmaceutical composition is mostly free of degradation products and is slightly yellow in appearance after between about 1 day and 12 weeks at about −20° C., about 2° C. to 8° C., or about 40° C. In some embodiments, the liquid or dissolved lyophilized powder formulation of the pharmaceutical composition is mostly free of degradation products and is slightly yellow in appearance after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is mostly free of degradation products and is slightly yellow in appearance after about 1, 2, 3, 4, 5, or more freeze/thaw cycles between about −70° C. and about 25° C. In some embodiments, the liquid or dissolved lyophilized powder formulation of the pharmaceutical composition is mostly free of degradation products and is slightly yellow in appearance after about 3 or more freeze/thaw cycles between about −70° C. and about 25° C. In some embodiments, the liquid or dissolved lyophilized powder formulation of the pharmaceutical composition is mostly free of degradation products and is slightly yellow in appearance after about 5 or more freeze/thaw cycles between about −70° C. and about 25° C. In some embodiments, the liquid or dissolved lyophilized powder formulation of the pharmaceutical composition is mostly free of degradation products and is slightly yellow in appearance after about 1, 2, 3, or more days under agitation conditions at room temperature. In some embodiments, the liquid or dissolved lyophilized powder formulation of the pharmaceutical composition is mostly free of degradation products and is slightly brown in appearance after about 1, 2, 3, or more days under agitation conditions at room temperature. In some embodiments, the liquid or dissolved lyophilized powder formulation of the pharmaceutical composition is mostly free of degradation products and is colorless in appearance after about 1, 2, 3, or more days under agitation conditions at room temperature. In some embodiments, the liquid or dissolved lyophilized powder formulation of the pharmaceutical composition is mostly free of degradation products and is slightly yellow brown in appearance after about 1, 2, 3, or more days under agitation conditions at room temperature.

Opalescence indicates physical instability of a formulation because of the presence of aggregates or liquid-liquid phase separation in solution and has been reported for monoclonal antibody (mAb) formulations. Increased solution opalescence can be attributed to attractive protein-protein interactions (PPIs). It is critical that aggregates or liquid-liquid phase separation in formulations is minimal and that the opalescence of the formulation does not exceed slight opalescence. In some embodiments, the liquid or dissolved lyophilized powder formulation of the pharmaceutical composition is slightly opalescent in appearance after between about 1 day and 12 weeks at about −20° C., about 2° C. to 8° C., or about 40° C. In some embodiments, the liquid or dissolved lyophilized powder formulation of the pharmaceutical composition is slightly opalescent in appearance after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is slightly opalescent in appearance after about 1, 2, 3, 4, 5, or more freeze/thaw cycles between about −70° C. and about 25° C. In some embodiments, the liquid or dissolved lyophilized powder formulation of the pharmaceutical composition is slightly opalescent in appearance after about 3 or more freeze/thaw cycles between about −70° C. and about 25° C. In some embodiments, the liquid or dissolved lyophilized powder formulation of the pharmaceutical composition is slightly opalescent in appearance after about 5 or more freeze/thaw cycles between about −70° C. and about 25° C. In some embodiments, the liquid or dissolved lyophilized powder formulation of the pharmaceutical composition is slightly opalescent in appearance after about 1, 2, 3, or more days under agitation conditions at room temperature.

In some embodiments, visible and sub-visible particulates in antibody products may pose safety and immunogenicity risks to patients and therefore it is important that formulations are free of visible and/or sub-visible particles. In some embodiments, the liquid or dissolved lyophilized powder formulation of the pharmaceutical composition is free of visible particles comprising high molecular weight molecules after between about 1 day and 12 weeks at about −20° C., about 2° C. to 8° C., or about 40° C. In some embodiments, the liquid or dissolved lyophilized powder formulation of the pharmaceutical composition is free of visible particles comprising high molecular weight molecules after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is free of visible particles comprising high molecular weight molecules after about 1, 2, 3, 4, 5, or more freeze/thaw cycles between about −70° C. and about 25° C. In some embodiments, the liquid or dissolved lyophilized powder formulation of the pharmaceutical composition is free of visible particles comprising high molecular weight molecules after about 3 or more freeze/thaw cycles between about −70° C. and about 25° C. In some embodiments, the liquid or dissolved lyophilized powder formulation of the pharmaceutical composition is free of visible particles comprising high molecular weight molecules after about 5 or more freeze/thaw cycles between about −70° C. and about 25° C. In some embodiments, the liquid or dissolved lyophilized powder formulation of the pharmaceutical composition is free of visible particles comprising high molecular weight molecules after about 1, 2, 3, or more days under agitation conditions at room temperature. In some embodiments, the liquid or dissolved lyophilized powder formulation of the pharmaceutical composition is free of sub-visible particles comprising high molecular weight molecules after between about 1 day and 12 weeks at about −20° C., about 2° C. to 8° C., or about 40° C. In some embodiments, the liquid or dissolved lyophilized powder formulation of the pharmaceutical composition is free of sub-visible particles comprising high molecular weight molecules after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is free of sub-visible particles comprising high molecular weight molecules after about 1, 2, 3, 4, 5, or more freeze/thaw cycles between about −70° C. and about 25° C. In some embodiments, the liquid or dissolved lyophilized powder formulation of the pharmaceutical composition is free of sub-visible particles comprising high molecular weight molecules after about 3 or more freeze/thaw cycles between about −70° C. and about 25° C. In some embodiments, the liquid or dissolved lyophilized powder formulation of the pharmaceutical composition is free of sub-visible particles comprising high molecular weight molecules after about 5 or more freeze/thaw cycles between about −70° C. and about 25° C. In some embodiments, the liquid or dissolved lyophilized powder formulation of the pharmaceutical composition is free of sub-visible particles comprising high molecular weight molecules after about 1, 2, 3, or more days under agitation conditions at room temperature. In some embodiments, the liquid or dissolved lyophilized powder formulation of the pharmaceutical composition is essentially free of visible particles comprising high molecular weight molecules after between about 1 day and 12 weeks at about −20° C., about 2° C. to 8° C., or about 40° C. In some embodiments, the liquid or dissolved lyophilized powder formulation of the pharmaceutical composition is essentially free of visible particles comprising high molecular weight molecules after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is essentially free of visible particles comprising high molecular weight molecules after about 1, 2, 3, 4, 5, or more freeze/thaw cycles between about −70° C. and about 25° C. In some embodiments, the liquid or dissolved lyophilized powder formulation of the pharmaceutical composition is essentially free of visible particles comprising high molecular weight molecules after about 3 or more freeze/thaw cycles between about −70° C. and about 25° C. In some embodiments, the liquid or dissolved lyophilized powder formulation of the pharmaceutical composition is essentially free of visible particles comprising high molecular weight molecules after about 5 or more freeze/thaw cycles between about −70° C. and about 25° C. In some embodiments, the liquid or dissolved lyophilized powder formulation of the pharmaceutical composition is essentially free of visible particles comprising high molecular weight molecules after about 1, 2, 3, or more days under agitation conditions at room temperature. In some embodiments, the liquid or dissolved lyophilized powder formulation of the pharmaceutical composition is essentially free of sub-visible particles comprising high molecular weight molecules after between about 1 day and 12 weeks at about −20° C., about 2° C. to 8° C., or about 40° C. In some embodiments, the liquid or dissolved lyophilized powder formulation of the pharmaceutical composition is essentially free of sub-visible particles comprising high molecular weight molecules after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is essentially free of sub-visible particles comprising high molecular weight molecules after about 1, 2, 3, 4, 5, or more freeze/thaw cycles between about −70° C. and about 25° C. In some embodiments, the liquid or dissolved lyophilized powder formulation of the pharmaceutical composition is essentially free of sub-visible particles comprising high molecular weight molecules after about 3 or more freeze/thaw cycles between about −70° C. and about 25° C. In some embodiments, the liquid or dissolved lyophilized powder formulation of the pharmaceutical composition is essentially free of sub-visible particles comprising high molecular weight molecules after about 5 or more freeze/thaw cycles between about −70° C. and about 25° C. In some embodiments, the liquid or dissolved lyophilized powder formulation of the pharmaceutical composition is essentially free of sub-visible particles comprising high molecular weight molecules after about 1, 2, 3, or more days under agitation conditions at room temperature. In some embodiments, the liquid or dissolved lyophilized powder formulation of the pharmaceutical composition is controlled for visible particles comprising high molecular weight molecules after between about 1 day and 12 weeks at about −20° C., about 2° C. to 8° C., or about 40° C. In some embodiments, the liquid or dissolved lyophilized powder formulation of the pharmaceutical composition is controlled for visible particles comprising high molecular weight molecules after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is controlled for visible particles comprising high molecular weight molecules after about 1, 2, 3, 4, 5, or more freeze/thaw cycles between about −70° C. and about 25° C. In some embodiments, the liquid or dissolved lyophilized powder formulation of the pharmaceutical composition is controlled for visible particles comprising high molecular weight molecules after about 3 or more freeze/thaw cycles between about −70° C. and about 25° C. In some embodiments, the liquid or dissolved lyophilized powder formulation of the pharmaceutical composition is controlled for visible particles comprising high molecular weight molecules after about 5 or more freeze/thaw cycles between about −70° C. and about 25° C. In some embodiments, the liquid or dissolved lyophilized powder formulation of the pharmaceutical composition is controlled for visible particles comprising high molecular weight molecules after about 1, 2, 3, or more days under agitation conditions at room temperature. In some embodiments, the liquid or dissolved lyophilized powder formulation of the pharmaceutical composition is controlled for sub-visible particles comprising high molecular weight molecules after between about 1 day and 12 weeks at about −20° C., about 2° C. to 8° C., or about 40° C. In some embodiments, the liquid or dissolved lyophilized powder formulation of the pharmaceutical composition is controlled for sub-visible particles comprising high molecular weight molecules after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is controlled for sub-visible particles comprising high molecular weight molecules after about 1, 2, 3, 4, 5, or more freeze/thaw cycles between about −70° C. and about 25° C. In some embodiments, the liquid or dissolved lyophilized powder formulation of the pharmaceutical composition is controlled for sub-visible particles comprising high molecular weight molecules after about 3 or more freeze/thaw cycles between about −70° C. and about 25° C. In some embodiments, the liquid or dissolved lyophilized powder formulation of the pharmaceutical composition is controlled for sub-visible particles comprising high molecular weight molecules after about 5 or more freeze/thaw cycles between about −70° C. and about 25° C. In some embodiments, the liquid or dissolved lyophilized powder formulation of the pharmaceutical composition is controlled for sub-visible particles comprising high molecular weight molecules after about 1, 2, 3, or more days under agitation conditions at room temperature.

In some embodiments, the liquid or dissolved lyophilized powder formulation of the pharmaceutical composition is free of low molecular weight molecule degradants after between about 1 day and 12 weeks at about −20° C., about 2° C. to 8° C., or about 40° C. In some embodiments, the liquid or dissolved lyophilized powder formulation of the pharmaceutical composition is free of low molecular weight molecule degradants after about 1 day, about 1 week, about 2 weeks, about 4 weeks, about 6 weeks, or about 12 weeks at about −20° C., about 2° C. to 8° C., or about 40° C. In some embodiments, the liquid or dissolved lyophilized powder formulation of the pharmaceutical composition is free of low molecular weight molecule degradants after about 1, 2, 3, 4, 5, or more freeze/thaw cycles between about −70° C. and about 25° C. In some embodiments, the liquid or dissolved lyophilized powder formulation of the pharmaceutical composition is free of low molecular weight molecule degradants after about 3 or more freeze/thaw cycles between about −70° C. and about 25° C. In some embodiments, the liquid or dissolved lyophilized powder formulation of the pharmaceutical composition is free of low molecular weight molecule degradants after about 5 or more freeze/thaw cycles between about −70° C. and about 25° C. In some embodiments, the liquid or dissolved lyophilized powder formulation of the pharmaceutical composition is free of low molecular weight molecule degradants after about 1, 2, 3, or more days under agitation conditions at room temperature.

In some embodiments, the purity is not significantly reduced after between about 1 day and 12 weeks at about −20° C., about 2° C. to 8° C., or about 40° C. In some embodiments, the purity is not significantly reduced after about 1 day, about 1 week, about 2 weeks, about 4 weeks, about 6 weeks, or about 12 weeks at about −20° C., about 2° C. to 8° C., or about 40° C. In some embodiments, the purity is not significantly reduced after about 1, 2, 3, 4, 5, or more freeze/thaw cycles between about −70° C. and about 25° C. In some embodiments, the purity is not significantly reduced after about 3 or more freeze/thaw cycles between about −70° C. and about 25° C. In some embodiments, the purity is not significantly reduced after about 5 or more freeze/thaw cycles between about −70° C. and about 25° C. In some embodiments, the purity is not significantly reduced after about 1, 2, 3, or more days under agitation conditions at room temperature.

In some embodiments, the pharmaceutical composition is stable at about −80° C. to about 40° C. In some embodiments, the pharmaceutical composition is stable at about −25° C. to about −15° C. In some embodiments, the pharmaceutical composition is stable at about 2-8° C.

In some embodiments, the pharmaceutical composition is stable at about −80° C. In some embodiments, the pharmaceutical composition is stable at about −70° C. In some embodiments, the pharmaceutical composition is stable at about −60° C. In some embodiments, the pharmaceutical composition is stable at about −50° C. In some embodiments, the pharmaceutical composition is stable at about −40° C. In some embodiments, the pharmaceutical composition is stable at about −30° C. In some embodiments, the pharmaceutical composition is stable at about −25° C. In some embodiments, the pharmaceutical composition is stable at about −20° C. In some embodiments, the pharmaceutical composition is stable at about −15° C. In some embodiments, the pharmaceutical composition is stable at about −10° C. In some embodiments, the pharmaceutical composition is stable at about −5° C. In some embodiments, the pharmaceutical composition is stable at about 0° C. In some embodiments, the pharmaceutical composition is stable at about 1° C. In some embodiments, the pharmaceutical composition is stable at about 2° C. In some embodiments, the pharmaceutical composition is stable at about 3° C. In some embodiments, the pharmaceutical composition is stable at 4 about ° C. In some embodiments, the pharmaceutical composition is stable at about 5° C. In some embodiments, the pharmaceutical composition in stable at about 6° C. In some embodiments, the pharmaceutical composition is stable at about 7° C. In some embodiments, the pharmaceutical composition is stable at about 8° C. In some embodiments, the pharmaceutical composition is stable at about 9° C. In some embodiments, the pharmaceutical composition is stable at about 10° C. In some embodiments, the pharmaceutical composition is stable at about 15° C. In some embodiments, the pharmaceutical composition is stable at about 20° C. In some embodiments, the pharmaceutical composition is stable at about 25° C. In some embodiments, the pharmaceutical composition is stable at about 30° C. In some embodiments, the pharmaceutical composition is stable at about 35° C. In some embodiments, the pharmaceutical composition is stable at about 40° C.

In some embodiments, the pharmaceutical composition is stable for about 1 week to about 36 months.

In some embodiments, the pharmaceutical composition is stable for about 1 week. In some embodiments, the pharmaceutical composition is stable for about 2 weeks. In some embodiments, the pharmaceutical composition is stable for about 3 weeks. In some embodiments, the pharmaceutical composition is stable for about 1 month. In some embodiments, the pharmaceutical composition is stable for about 2 months. In some embodiments, the pharmaceutical composition is stable for about 3 months. In some embodiments, the pharmaceutical composition is stable for about 4 months. In some embodiments, the pharmaceutical composition is stable for about 5 months. In some embodiments, the pharmaceutical composition is stable for about 6 months. In some embodiments, the pharmaceutical composition is stable for about 7 months. In some embodiments, the pharmaceutical composition is stable for about 8 months. In some embodiments, the pharmaceutical composition is stable for about 9 months. In some embodiments, the pharmaceutical composition is stable for about 10 months. In some embodiments, the pharmaceutical composition is stable for about 11 months. In some embodiments, the pharmaceutical composition is stable for about 12 months. In some embodiments, the pharmaceutical composition is stable for about 13 months. In some embodiments, the pharmaceutical composition is stable for about 14 months. In some embodiments, the pharmaceutical composition is stable for about 15 months. In some embodiments, the pharmaceutical composition is stable for about 16 months. In some embodiments, the pharmaceutical composition is stable for about 17 months. In some embodiments, the pharmaceutical composition is stable for about 18 months. In some embodiments, the pharmaceutical composition is stable for about 19 months. In some embodiments, the pharmaceutical composition is stable for about 20 months. In some embodiments, the pharmaceutical composition is stable for about 21 months. In some embodiments, the pharmaceutical composition is stable for about 22 months. In some embodiments, the pharmaceutical composition is stable for about 23 months. In some embodiments, the pharmaceutical composition is stable for about 24 months. In some embodiments, the pharmaceutical composition is stable for about 25 months. In some embodiments, the pharmaceutical composition is stable for about 26 months. In some embodiments, the pharmaceutical composition is stable for about 27 months. In some embodiments, the pharmaceutical composition is stable for about 28 months. In some embodiments, the pharmaceutical composition is stable for about 29 months. In some embodiments, the pharmaceutical composition is stable for about 30 months. In some embodiments, the pharmaceutical composition is stable for about 31 months. In some embodiments, the pharmaceutical composition is stable for about 32 months. In some embodiments, the pharmaceutical composition is stable for about 33 months. In some embodiments, the pharmaceutical composition is stable for about 34 months. In some embodiments, the pharmaceutical composition is stable for about 35 months. In some embodiments, the pharmaceutical composition is stable for about 36 months.

In some embodiments, the pharmaceutical composition is stable at about −20° C. for about 1 week. In some embodiments, the pharmaceutical composition is stable at about −20° C. for about 2 weeks. In some embodiments, the pharmaceutical composition is stable at about −20° C. for about 3 weeks. In some embodiments, the pharmaceutical composition is stable at about −20° C. for about 1 month. In some embodiments, the pharmaceutical composition is stable at about −20° C. for about 2 months. In some embodiments, the pharmaceutical composition is stable at about −20° C. for about 3 months. In some embodiments, the pharmaceutical composition is stable at about −20° C. for about 4 months. In some embodiments, the pharmaceutical composition is stable at about −20° C. for about 5 months. In some embodiments, the pharmaceutical composition is stable at about −20° C. for about 6 months. In some embodiments, the pharmaceutical composition is stable at about −20° C. for about 7 months. In some embodiments, the pharmaceutical composition is stable at about −20° C. for about 8 months. In some embodiments, the pharmaceutical composition is stable at about −20° C. for about 9 months. In some embodiments, the pharmaceutical composition is stable at about −20° C. for about 10 months. In some embodiments, the pharmaceutical composition is stable at about −20° C. for about 11 months. In some embodiments, the pharmaceutical composition is stable at about −20° C. for about 12 months. In some embodiments, the pharmaceutical composition is stable at about −20° C. for about 13 months. In some embodiments, the pharmaceutical composition is stable at about −20° C. for about 14 months. In some embodiments, the pharmaceutical composition is stable at about −20° C. for about 15 months. In some embodiments, the pharmaceutical composition is stable at about −20° C. for about 16 months. In some embodiments, the pharmaceutical composition is stable at about −20° C. for about 17 months. In some embodiments, the pharmaceutical composition is stable at about −20° C. for about 18 months. In some embodiments, the pharmaceutical composition is stable at about −20° C. for about 19 months. In some embodiments, the pharmaceutical composition is stable at about −20° C. for about 20 months. In some embodiments, the pharmaceutical composition is stable at about −20° C. for about 21 months. In some embodiments, the pharmaceutical composition is stable at about −20° C. for about 22 months. In some embodiments, the pharmaceutical composition is stable at about −20° C. for about 23 months. In some embodiments, the pharmaceutical composition is stable at about −20° C. for about 24 months. In some embodiments, the pharmaceutical composition is stable at about −20° C. for about 25 months. In some embodiments, the pharmaceutical composition is stable at about −20° C. for about 26 months. In some embodiments, the pharmaceutical composition is stable at about −20° C. for about 27 months. In some embodiments, the pharmaceutical composition is stable at about −20° C. for about 28 months. In some embodiments, the pharmaceutical composition is stable at about −20° C. for about 29 months. In some embodiments, the pharmaceutical composition is stable at about −20° C. for about 30 months. In some embodiments, the pharmaceutical composition is stable at about −20° C. for about 31 months. In some embodiments, the pharmaceutical composition is stable at about −20° C. for about 32 months. In some embodiments, the pharmaceutical composition is stable at about −20° C. for about 33 months. In some embodiments, the pharmaceutical composition is stable at about −20° C. for about 34 months. In some embodiments, the pharmaceutical composition is stable at about −20° C. for about 35 months. In some embodiments, the pharmaceutical composition is stable at about −20° C. for about 36 months.

In some embodiments, the pharmaceutical composition is stable at −20° C. for at least 3 months. In some embodiments, the pharmaceutical composition is stable at −20° C. for at least 6 months. In some embodiments, the pharmaceutical composition is stable at 2-8° C. for at least 3 months. In some embodiments, the pharmaceutical composition is stable at 2-8° C. for at least 6 months. In some embodiments, the pharmaceutical composition is stable at 2-8° C. for at least 9 months.

In some embodiments, the pharmaceutical composition comprises any of the preceding embodiments.

In some embodiments, the pharmaceutical composition is stored in aliquots of about 1 mL to about 6 mL.

In some embodiments, the pharmaceutical composition is stored in aliquots of about 1 mL. In some embodiments, the pharmaceutical composition is stored in aliquots of about 2 mL. In some embodiments, the pharmaceutical composition is stored in aliquots of about 3 mL. In some embodiments, the pharmaceutical composition is stored in aliquots of about 4 mL. In some embodiments, the pharmaceutical composition is stored in aliquots of about 5 mL. In some embodiments, the pharmaceutical composition is stored in aliquots of about 6 mL.

In some embodiments, the pharmaceutical composition is stored in 1 mL to about 10 mL vials. In some embodiments, the pharmaceutical composition is stored in 1 mL to about 10 mL syringes.

In some embodiments, the pharmaceutical composition is stored in 1 mL vials. In some embodiments, the pharmaceutical composition is stored in 2 mL vials. In some embodiments, the pharmaceutical composition is stored in 3 mL vials. In some embodiments, the pharmaceutical composition is stored in 4 mL vials. In some embodiments, the pharmaceutical composition is stored in 5 mL vials. In some embodiments, the pharmaceutical composition is stored in 6 mL vials. In some embodiments, the pharmaceutical composition is stored in 7 mL vials. In some embodiments, the pharmaceutical composition is stored in 8 mL vials. In some embodiments, the pharmaceutical composition is stored in 9 mL vials. In some embodiments, the pharmaceutical composition is stored in 10 mL vials. In some embodiments, the pharmaceutical composition is stored in 1 mL syringes. In some embodiments, the pharmaceutical composition is stored in 2 mL syringes. In some embodiments, the pharmaceutical composition is stored in 3 mL syringes. In some embodiments, the pharmaceutical composition is stored in 4 mL syringes. In some embodiments, the pharmaceutical composition is stored in 5 mL syringes. In some embodiments, the pharmaceutical composition is stored in 6 mL syringes. In some embodiments, the pharmaceutical composition is stored in 7 mL syringes. In some embodiments, the pharmaceutical composition is stored in 8 mL syringes. In some embodiments, the pharmaceutical composition is stored in 9 mL syringes. In some embodiments, the pharmaceutical composition is stored in 10 mL syringes.

In some embodiments, the pharmaceutical composition is stored in vials with a stopper and a cover. In some embodiments, the pharmaceutical composition is stored in vials with about a 10 mm to about a 30 mm stopper, and about a 10 mm to about a 30 mm cover.

In some embodiments, the pharmaceutical composition is stored in vials with about a 10 mm stopper. In some embodiments, the pharmaceutical composition is stored in vials with about a 15 mm stopper. In some embodiments, the pharmaceutical composition is stored in vials with about a 20 mm stopper. In some embodiments, the pharmaceutical composition is stored in vials with about a 25 mm stopper. In some embodiments, the pharmaceutical composition is stored in vials with about a 10 mm cover. In some embodiments, the pharmaceutical composition is stored in vials with about a 15 mm cover. In some embodiments, the pharmaceutical composition is stored in vials with about a 20 mm cover. In some embodiments, the pharmaceutical composition is stored in vials with about a 25 mm cover.

In some embodiments, the pharmaceutical composition is stored in vials with an aluminum plastic cover. In some embodiments, the pharmaceutical composition is stored in vials with a plastic cover.

In some embodiments, the pharmaceutical composition is stored in 2 mL aliquots at −20° C. in 6 mL vials with a 20 mm stopper and a 20 mm aluminum plastic cover.

In some embodiments, the pharmaceutical composition is filtered prior to storage. In some embodiments, the pharmaceutical composition is filtered with a 0.22 μM PVDF filter prior to storage.

In some embodiments, the pharmaceutical composition is for intravenous or subcutaneous injection. In some embodiments, the pharmaceutical composition is for intravenous injection. In some embodiments, the pharmaceutical composition is for subcutaneous injection. In some embodiments, the pharmaceutical composition is for intramuscular injection. In some embodiments, the pharmaceutical composition is for intracisternal injection.

In some embodiments, the pharmaceutical composition is a liquid pharmaceutical composition. In some embodiments, the pharmaceutical composition is a lyophilized pharmaceutical composition.

In some embodiments, a dosage form in a container is provided. In some embodiments, the dosage form comprises a pharmaceutical composition such as those provided herein. In some embodiments, the container is a plastic vial or a glass vial.

Compositions and formulations can be administered with medical devices known in the art. For example, a pharmaceutical composition of the invention can be administered by injection with a hypodermic needle, including, e.g., a prefilled syringe or autoinjector.

In some embodiments, a kit, comprising a pharmaceutical composition, such as those provided herein, is provided.

Alternately, one may administer the pharmaceutical composition in a local rather than systemic manner, for example, via injection of the antibody directly into an arthritic joint or pathogen-induced lesion characterized by immunopathology, often in a depot or sustained release pharmaceutical composition. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The pharmaceutical compositions provided herein can be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. In some embodiments, the pharmaceutical composition is administered every two weeks, every three weeks, every four weeks, every five weeks, every six weeks, every seven weeks, or every eight weeks. In some embodiments, the pharmaceutical composition is administered every two weeks. In some embodiments, the pharmaceutical composition is administered every three weeks. In some embodiments, the pharmaceutical composition is administered every four weeks. In some embodiments, the pharmaceutical composition is administered every five weeks. In some embodiments, the pharmaceutical composition is administered every six weeks. In some embodiments, the pharmaceutical composition is administered every seven weeks. In some embodiments, the pharmaceutical composition is administered every eight weeks. In some embodiments, the pharmaceutical composition is administered for at least 21-52 weeks or longer. In some embodiments, the pharmaceutical composition is administered on such a schedule for at least 21 weeks. In some embodiments, the pharmaceutical composition is administered on such a schedule for at least 24 weeks. In some embodiments, the pharmaceutical composition is administered on such a schedule for at least 32 weeks. In some embodiments, the pharmaceutical composition is administered on such a schedule for at least 36 weeks. In some embodiments, the pharmaceutical composition is administered on such a schedule for at least 40 weeks. In some embodiments, the pharmaceutical composition is administered on such a schedule for at least 42 weeks. In some embodiments, the pharmaceutical composition is administered (e.g. infusion or subcutaneous injection) once. In some embodiments, the pharmaceutical composition is administered (e.g. infusion or subcutaneous injection) twice. In some embodiments, the pharmaceutical composition is administered (e.g. infusion or subcutaneous injection) three times. In some embodiments, the pharmaceutical composition is administered (e.g. infusion or subcutaneous injection) four times. In some embodiments, the pharmaceutical composition is administered (e.g. infusion or subcutaneous injection) five times. In some embodiments, the pharmaceutical composition is administered (e.g. infusion or subcutaneous injection) six times. In some embodiments, the pharmaceutical composition is administered (e.g. infusion or subcutaneous injection) seven times. In some embodiments, the pharmaceutical composition is administered (e.g. infusion or subcutaneous injection) eight times. In some embodiments, the antibody is administered (e.g. infusion or subcutaneous injection) nine times. In some embodiments, the pharmaceutical composition is administered (e.g. infusion or subcutaneous injection) 10 times. In some embodiments, the pharmaceutical composition is administered (e.g. infusion or subcutaneous injection) 11 times. In some embodiments, the pharmaceutical composition is administered (e.g. infusion or subcutaneous injection) 12 times. In some embodiments, the pharmaceutical composition is administered (e.g. infusion or subcutaneous injection) 13 times. In some embodiments, the pharmaceutical composition is administered (e.g. infusion or subcutaneous injection) 14 times. In some embodiments, the pharmaceutical composition is administered (e.g. infusion or subcutaneous injection) 15 times. In some embodiments, the pharmaceutical composition is administered (e.g. infusion or subcutaneous injection) 16 times. In some embodiments, the pharmaceutical composition is administered (e.g. infusion or subcutaneous injection) 17 times. In some embodiments, the pharmaceutical composition is administered (e.g. infusion or subcutaneous injection) 18 times. In some embodiments, the pharmaceutical composition is administered (e.g. infusion or subcutaneous injection) 19 times. In some embodiments, the pharmaceutical composition is administered (e.g. infusion or subcutaneous injection) 20 times. When the pharmaceutical composition is administered more than once it can be administered according to a schedule, such as the schedules provided for herein.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose”, and “effective amount” refer to an amount of the antibody, or antigen binding fragment thereof, that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%; and most preferably by at least 50%. An effective amount can also result in an improvement in a subjective measure in cases where subjective measures are used to assess disease severity. In some embodiments, an amount is a therapeutically effective amount if it is an amount that can be used to treat or ameliorate a condition as provided for herein.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune cell activation compared to the immune cell activation detected in the absence of the composition. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

As used herein, the term “individual” or “subject” or “patient” used interchangeably, means any organism, including mammals, such as mice, rats, other rodents, rabbits, dogs, cats, swine, cattle, sheep, horses, or primates, such as humans. In one embodiment, the subject is a human. A subject can also be referred to as a patient. In some embodiments, the subject is a subject in need thereof. A subject that is “in need thereof” refers to a subject that has been identified as requiring treatment for the condition that is to be treated and is treated with the specific intent of treating such condition. The conditions can be, for example, any of the conditions described herein.

Accordingly, in some embodiments, methods of treating a subject with a C1s mediated disorder are provided. In some embodiments, the method comprises administering a pharmaceutical composition comprising an antibody, or antigen binding fragment thereof, as provided herein. In some embodiments, the disorder is a C1s mediated disorder. As provided for herein, the antibodies, or antigen binding fragments thereof, can be administered with other therapeutics. These can be administered simultaneously or sequentially.

In some embodiments, the subject is a subject who has previously been treated with a different antibody than those provided herein.

In some embodiments, the pharmaceutical composition comprises an antibody at about 100 mg/mL, phosphate buffer at about 20 mM, L-methionine at about 10 mM, sucrose at about 4% w/v, L-Arg-HCL at about 70 mM, and P188 at about 0.1% w/v, wherein the pharmaceutical composition is at a pH of about 7.2.

In some embodiments, the pharmaceutical composition comprises about 100 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 78, and wherein the light chain comprises an LCDR1 of SEQ ID NO: 64, an LCDR2 of SEQ ID NO: 65, and an LCDR3 of SEQ ID NO: 79; phosphate buffer at about 20 mM, L-methionine at about 10 mM, sucrose at about 4% w/v, L-Arg-HCL at about 70 mM, and P188 at about 0.1% w/v, wherein the pharmaceutical composition is at a pH of about 7.2.

In some embodiments, the pharmaceutical composition comprises an antibody at about 150 mg/mL, phosphate buffer at about 20 mM, L-methionine at about 10 mM, sucrose at about 3.5% w/v, L-Arg-HCL at about 70 mM, and P188 at about 0.1% w/v, wherein the pharmaceutical composition is at a pH of about 7.2.

In some embodiments, the pharmaceutical composition comprises about 150 mg/mL of an antibody having a heavy chain and a light chain, wherein the heavy chain comprises an HCDR1 of SEQ ID NO: 61, an HCDR2 of SEQ ID NO: 62, and an HCDR3 of SEQ ID NO: 78, and wherein the light chain comprises an LCDR1 of SEQ ID NO: 64, an LCDR2 of SEQ ID NO: 65, and an LCDR3 of SEQ ID NO: 79; phosphate buffer at about 20 mM, L-methionine at about 10 mM, sucrose at about 4% w/v, L-Arg-HCL at about 70 mM, and P188 at about 0.1% w/v, wherein the pharmaceutical composition is at a pH of about 7.2.

In some embodiments, the method provided herein comprise administering to a subject an antibody, or an antigen binding fragment thereof, that specifically binds to and inhibits C1s. In some embodiments, the antibody is as provided herein.

Kits are also provided which are useful for carrying out embodiments described herein. The present kits comprise a first container containing or packaged in association with the above-described antibodies. The kit may also comprise another container containing or packaged in association solutions necessary or convenient for carrying out the embodiments. The containers can be made of glass, polymer (e.g., plastic), metal (e.g., aluminum), and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

In some embodiments, antibodies that bind to a C1s protein are provided. In some embodiments, the antibody is isolated. In some embodiments, the antibody binds specifically. In some embodiments, the antibody binds to a C1s protein that is properly folded. In some embodiments, the antibody is specific for a specific C1s conformational state (open or closed). In some embodiments, the antibody binds to a C1s protein in a cell membrane. In some embodiments, the antibody binds to a C1s protein that is in a cell membrane in an intact cell. In some embodiments, the antibody inhibits or neutralizes the function of a Cls protein. As used herein, the term “neutralize” means that the activity or function of the protein is inhibited. The inhibition can be complete or partial. In some embodiments, the activity or function of the protein is inhibited at least 10, 20, 30, 40, 50, 60, 70, 80, 90, 95, or 99%. The percent inhibition can be based upon the function or activity of the protein in the absence of the antibody. In some embodiments, the antibody inhibits the glucose transport facilitated by C1s. In some embodiments, the antibody inhibits the internalization of the C1s protein.

In some embodiments, the antibody comprises a sequence as provided for herein or antigen binding fragment thereof. In some embodiments, the antibody comprises a heavy chain CDR or an antigen binding fragment thereof described herein. The heavy chain may be one or more of the heavy chains described herein. In some embodiments, the antibody comprises a light chain, or an antigen binding fragment thereof as described herein.

In some embodiments, methods of treating, inhibiting or ameliorating a C1s-associated pathology are provided. In some embodiments, the methods comprise administering a pharmaceutical composition described herein to a subject to treat, inhibit or ameliorate a C1s-associated pathology. In some embodiments, the pathology is as described herein.

In some embodiments, the pharmaceutical composition main peak, measured by SEC, decreases in area percent by less than 0.1%, less than 0.2%, less than 0.3%, less than 0.4%, less than 0.5%, less than 0.6%, less than 0.7%, less than 0.8%, less than 0.9%, or less than 1% after 24 months at a temperature of about −70° C., relative to the initial value. In some embodiments, the pharmaceutical composition main peak, measured by SEC, decreases in area percent by less than 0.2% after 24 months at a temperature of about −70° C., relative to the initial value.

In some embodiments, the pharmaceutical composition main peak, measured by SEC, decreases in area percent by less than 0.1%, less than 0.2%, less than 0.3%, less than 0.4%, less than 0.5%, less than 0.6%, less than 0.7%, less than 0.8%, less than 0.9%, or less than 1% after 24 months at a temperature of about −20° C., relative to the initial value. In some embodiments, the pharmaceutical composition main peak, measured by SEC, decreases in area percent by less than 0.4% after 24 months at a temperature of about −20° C., relative to the initial value.

In some embodiments, the pharmaceutical composition main peak, measured by SEC, decreases in area percent by less than 0.1%, less than 0.2%, less than 0.3%, less than 0.4%, less than 0.5%, less than 0.6%, less than 0.7%, less than 0.8%, less than 0.9%, or less than 1% after 6 months at a temperature of about 5° C., relative to the initial value. In some embodiments, the pharmaceutical composition main peak, measured by SEC, decreases in area percent by less than 0.5% after 6 months at a temperature of about 5° C., relative to the initial value.

In some embodiments, the pharmaceutical composition main peak, measured by SEC, decreases in area percent by less than 0.1%, less than 0.2%, less than 0.3%, less than 0.4%, less than 0.5%, less than 0.6%, less than 0.7%, less than 0.8%, less than 0.9%, less than 1%, less than 1.1%, less than 1.2%, less than 1.3%, less than 1.4%, less than 1.5%, less than 1.6%, less than 1.7%, less than 1.8%, less than 1.9%, less than 2%, less than 2.1%, less than 2.2%, less than 2.3%, less than 2.4%, less than 2.5%, less than 2.6%, less than 2.7%, less than 2.8%, less than 2.9%, less than 3%, less than 3.1%, less than 3.2%, less than 3.3%, less than 3.4%, less than 3.5%, less than 3.6%, less than 3.7%, less than 3.8%, less than 3.9%, less than 4%, less than 4.1%, less than 4.2%, less than 4.3%, less than 4.4%, less than 4.5%, less than 4.6%, less than 4.7%, less than 4.8%, less than 4.9%, or less than 5% after 6 months at a temperature of about 25° C., relative to the initial value. In some embodiments, the pharmaceutical composition main peak, measured by SEC, decreases in area percent by less than 3% after 6 months at a temperature of about 25° C., relative to the initial value.

In some embodiments, the pharmaceutical composition main peak, measured by SEC, decreases in area percent by less than 0.1%, less than 0.2%, less than 0.3%, less than 0.4%, less than 0.5%, less than 0.6%, less than 0.7%, less than 0.8%, less than 0.9%, less than 1%, less than 1.1%, less than 1.2%, less than 1.3%, less than 1.4%, less than 1.5%, less than 1.6%, less than 1.7%, less than 1.8%, less than 1.9%, less than 2%, less than 2.1%, less than 2.2%, less than 2.3%, less than 2.4%, less than 2.5%, less than 2.6%, less than 2.7%, less than 2.8%, less than 2.9%, less than 3%, less than 3.1%, less than 3.2%, less than 3.3%, less than 3.4%, less than 3.5%, less than 3.6%, less than 3.7%, less than 3.8%, less than 3.9%, less than 4%, less than 4.1%, less than 4.2%, less than 4.3%, less than 4.4%, less than 4.5%, less than 4.6%, less than 4.7%, less than 4.8%, less than 4.9%, less than 5%, less than 5.1%, less than 5.2%, less than 5.3%, less than 5.4%, less than 5.5%, less than 5.6%, less than 5.7%, less than 5.8%, less than 5.9%, less than 6%, less than 6.1%, less than 6.2%, less than 6.3%, less than 6.4%, less than 6.5%, less than 6.6%, less than 6.7%, less than 6.8%, less than 6.9%, less than 7%, less than 7.1%, less than 7.2%, less than 7.3%, less than 7.4%, less than 7.5%, less than 7.6%, less than 7.7%, less than 7.8%, less than 7.9%, less than 8%, less than 8.1%, less than 8.2%, less than 8.3%, less than 8.4%, less than 8.5%, less than 8.6%, less than 8.7%, less than 8.8%, less than 8.9%, less than 9%, less than 9.1%, less than 9.2%, less than 9.3%, less than 9.4%, less than 9.5%, less than 9.6%, less than 9.7%, less than 9.8%, less than 9.9%, or less than 10% after 1 month at a temperature of about 40° C., relative to the initial value. In some embodiments, the pharmaceutical composition main peak, measured by SEC, decreases in area percent by less than 7.8% after 1 month at a temperature of about 40° C., relative to the initial value.

In some embodiments, the main peak as measured by CE-SDS under non-reducing conditions is greater than 98% when stored at −20 C for up to 1, 3, 6, 9, 18, or 24 months.

In some embodiments, the percentage of low molecular weight species (LMWS) in the pharmaceutical composition as measured by CE-SDS under non-reducing conditions is less than, or about, 1.5, 1.1, or 1.0% when stored at −20 C for up to 1, 3, 6, 9, 18, or 24 months.

In some embodiments, the percentage of high molecular weight species (HMWS) in the pharmaceutical composition as measured by SEC is less than 2.0% when stored at −20 C for up to 1, 3, 6, 9, 18, or 24 months.

In some embodiments, the main peak as measured by CE-SDS under non-reducing conditions is greater than 97% when stored at 5 C for up to 1, 3, 6, 9, 18, or 24 months.

In some embodiments, the percentage of low molecular weight species (LMWS) in the pharmaceutical composition as measured by CE-SDS under non-reducing conditions is less than, or about, 2.5%, 2.0%, or 1.7% when stored at 5 C for up to 1, 3, 6, 9, 18, or 24 months.

In some embodiments, the percentage of high molecular weight species (HMWS) in the pharmaceutical composition as measured by SEC is less than 2.0% when stored at 5 C for up to 1, 3, 6, 9, 18, or 24 months.

In some embodiments, the main peak as measured by CE-SDS under non-reducing conditions is greater than 93% when stored at 25 C for up to 1, 3, 6, 9, 18, or 24 months.

In some embodiments, the percentage of low molecular weight species (LMWS) in the pharmaceutical composition as measured by CE-SDS under non-reducing conditions is less than, or about, 5%, 3.5%, or 1.5% when stored at 25 C for up to 1, 3, 6, 9, 18, or 24 months.

In some embodiments, the percentage of high molecular weight species (HMWS) as measured by SEC is less than 5%, 3.5%, or 1.5% when stored at 25 C for up to 1, 3, 6, 9, 18, or 24 months.

The pharmaceutical compositions provided herein provide for high concentration antibody formulations that are stable for periods of time, such as those provided for herein and can be used to treat the conditions, but not limited to, those provided for herein.

### Methods of Use

In some embodiments, the methods comprise administering a therapeutically or prophylactically effective amount of one or more antibodies or antigen binding fragments of the antibodies described herein to a susceptible subject or to one exhibiting a condition in which C1s is known to have caused the pathology observed. Any active form of the antibody can be administered, including, but not limited to scFv, Fab and F(ab′)2 fragments and other forms of antibodies provided for herein.

The present disclosure provides a method to treat an individual having a complement-mediated disease or disorder, the method comprising administering to the individual an anti-CIs antibody of any of the embodiments disclosed herein or a pharmaceutical composition thereof. In some embodiments, the individual is a mammal. In some embodiments, the individual is a human. In some embodiments, the administering is intravenous. In some embodiments, the administering is subcutaneous. In some embodiments, the administering is intrathecal. In some embodiments, the administering results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (1) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction in autoantibody mediated blister formation; (ad) a reduction in autoantibody induced pruritis; (ae) a reduction in autoantibody induced erythematosus; (af) a reduction in autoantibody mediated skin erosion; (ag) a reduction in red blood cell destruction due to transfusion reactions; (ah) a reduction in red blood cell lysis due to alloantibodies; (ai) a reduction in hemolysis due to transfusion reactions; (aj) a reduction in alloantibody mediated platelet lysis; (ak) a reduction in platelet lysis due to transfusion reactions; (al) a reduction in mast cell activation; (am) a reduction in mast cell histamine release; (an) a reduction in vascular permeability; (ao) a reduction in edema; (ap) a reduction in complement deposition on transplant graft endothelium; (aq) a reduction of anaphylatoxin generation in transplant graft endothelium; (ar) a reduction in the separation of the dermal-epidermal junction; (as) a reduction in the generation of anaphylatoxins in the dermal-epidermal junction; (at) a reduction in alloantibody mediated complement activation in transplant graft endothelium; (au) a reduction in antibody mediated loss of the neuromuscular junction; (av) a reduction in complement activation at the neuromuscular junction; (aw) a reduction in anaphylatoxin generation at the neuromuscular junction; (ax) a reduction in complement deposition at the neuromuscular junction; (ay) a reduction in paralysis; (az) a reduction in numbness; (ba) increased bladder control; (bb) increased bowel control; (bc) a reduction in mortality associated with autoantibodies; and (bd) a reduction in morbidity associated with autoantibodies. In some embodiments, the reduction in glial cell activation comprises reduction in astrocyte activation or reduction in microglia activation.

In some embodiments, a method of treating a subject with a C1s mediated disorder is provided. In some embodiments, the method comprises administering to the subject an antibody or antigen-binding fragment thereof as provided for herein or a pharmaceutical composition comprising an antibody or antigen-binding fragment thereof as provided for herein, thereby treating the C1s mediated disorder. The antibody or antigen-binding fragment thereof of any of the embodiments provided for herein or the pharmaceutical compositions of any of the embodiments provided for herein inhibit complement Cis activity in an individual having a complement-mediated disease or disorder. In some embodiments, the individual is a mammal. In some embodiments, the individual is a human. In some embodiments, the administering is intravenous. In some embodiments, the administering is subcutaneous. In some embodiments, the administering is intrathecal. In some embodiments, the administering results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (1) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction in autoantibody mediated blister formation; (ad) a reduction in autoantibody induced pruritis; (ae) a reduction in autoantibody induced erythematosus; (af) a reduction in autoantibody mediated skin erosion; (ag) a reduction in red blood cell destruction due to transfusion reactions; (ah) a reduction in red blood cell lysis due to alloantibodies; (ai) a reduction in hemolysis due to transfusion reactions; (aj) a reduction in alloantibody mediated platelet lysis; (ak) a reduction in platelet lysis due to transfusion reactions; (al) a reduction in mast cell activation; (am) a reduction in mast cell histamine release; (an) a reduction in vascular permeability; (ao) a reduction in edema; (ap) a reduction in complement deposition on transplant graft endothelium; (aq) a reduction of anaphylatoxin generation in transplant graft endothelium; (ar) a reduction in the separation of the dermal-epidermal junction; (as) a reduction in the generation of anaphylatoxins in the dermal-epidermal junction; (at) a reduction in alloantibody mediated complement activation in transplant graft endothelium; (au) a reduction in antibody mediated loss of the neuromuscular junction; (av) a reduction in complement activation at the neuromuscular junction; (aw) a reduction in anaphylatoxin generation at the neuromuscular junction; (ax) a reduction in complement deposition at the neuromuscular junction; (ay) a reduction in paralysis; (az) a reduction in numbness; (ba) increased bladder control; (bb) increased bowel control; (bc) a reduction in mortality associated with autoantibodies; and (bd) a reduction in morbidity associated with autoantibodies. In some embodiments, the reduction in glial cell activation comprises reduction in astrocyte activation or reduction in microglia activation.

In some embodiments, a method is provided comprising treating a subject with a C1s mediated disorder, the method comprising administering to the subject the pharmaceutical composition described in any one of the embodiments described herein. In some embodiments, the C1s mediated disorder is selected from the group including, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, these conditions can be caused by stroke or due to spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomysositis. In some embodiments, the C1s mediated disorder is Anti MAG neuropathy. In some embodiments, the C1s mediated disorder is due to stroke. In some embodiments, the C1s mediated disorder is due to spinal cord injury.

In some embodiments, the antibody or antigen-binding fragment thereof of any of the embodiments as provided for herein, or a pharmaceutical composition comprising the antibody or antigen-binding fragment thereof of any of the embodiments as provided for herein is for the use in the treatment of a complement-mediated disease or disorder. In some embodiments, the complement-mediated disorder is selected from the group including, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, these conditions can be due to stroke or due to spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the C1s mediated disorder is due to spinal cord injury. In some embodiments, the C1s mediated disorder is selected from the group including, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the Cls mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the Cls mediated disorder is due to Spinal Cord Injury.

The present disclosure provides use of an anti-C1s antibody of any of the embodiments in the manufacture of a medicament for the treatment of an individual having a complement-mediated disease or disorder. Accordingly, in some embodiments, an antibody, or antigen binding fragment thereof, or a pharmaceutical composition is provided for use as a medicament. In some embodiments, an antibody, or antigen binding fragment thereof, is provided for use as a medicament. In some embodiments, a pharmaceutical composition is provided for use as a medicament. In some embodiments, the antibody, or antigen binding fragment thereof, is an antibody or antigen binding fragment as provided for herein. In some embodiments, the pharmaceutical composition comprises an antibody or antigen binding fragment as provided for herein. In some embodiments, the pharmaceutical composition is as provided for herein. In some embodiments, the medicament is for use in treatment of a C1s mediated disease or disorder. In some embodiments, the complement-mediated disorder is, but not limited to, hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, Chronic inflammatory demyelinating polyneuropathy (CIDP), Multifocal motor neuropathy (MMN), Dermatomyositis, or Anti MAG neuropathy. In some embodiments, the conditions can be due to stroke or due to spinal cord injury. In some embodiments, the C1s mediated disorder is hemolysis. In some embodiments, the C1s mediated disorder is Cold Agglutinin Disease. In some embodiments, the C1s mediated disorder is Immune Thrombocytopenia (ITP). In some embodiments, the C1s mediated disorder is Myasthenia Gravis. In some embodiments, the C1s mediated disorder is Glomerulopathies. In some embodiments, the C1s mediated disorder is Atypical Hemolytic uremic syndrome. In some embodiments, the C1s mediated disorder is antiphospholipid antibody syndrome. In some embodiments, the C1s mediated disorder is transplant rejection. In some embodiments, the C1s mediated disorder is Chronic inflammatory demyelinating polyneuropathy (CIDP). In some embodiments, the C1s mediated disorder is Multifocal motor neuropathy (MMN). In some embodiments, the C1s mediated disorder is Dermatomyositis. In some embodiments, the C1s mediated disorder is Anti MAG neuropathy. In some embodiments, the C1s mediated disorder is due to stroke. In some embodiments, the C1s mediated disorder is due to spinal cord injury.

In some embodiments, a use of an antibody or antigen binding fragment as provided for herein or a pharmaceutical composition as provided for herein is provided. In some embodiments, the use is for the treatment of a C1s mediated disorder. In some embodiments, a use of an antibody or antigen binding fragment as provided for herein is provided, the use for the treatment of a C1s mediated disorder. In some embodiments, a use of a pharmaceutical composition comprising an antibody or antigen binging fragment as provided for herein is provided, the use for the treatment of a C1s mediated disorder. In some embodiments, the pharmaceutical composition is as provided for herein. The antibody of any of the embodiments or pharmaceutical compositions thereof inhibit complement C1s activity in an individual having a complement-mediated disease or disorder.

The present disclosure provides use of an anti-C1s antibody of any of the embodiments or a pharmaceutical composition thereof in the manufacture of a medicament for inhibiting complement C1s activity. In some embodiments, the present disclosure provides use of an anti-C1s antibody of any of the embodiments or a pharmaceutical composition thereof in the manufacture of a medicament for inhibiting complement C1s activity in an individual having a complement-mediated disease or disorder.

The present disclosure provides an anti-C1s antibody of any of the embodiments or a pharmaceutical composition thereof for use in medical therapy.

The present disclosure provides an anti-C1s antibody of any of the embodiments or a pharmaceutical composition thereof for treating an individual having a complement-mediated disease or disorder.

The present disclosure provides an anti-C1s antibody of any of the embodiments or a pharmaceutical composition thereof for inhibiting complement C1s protein activity. The present disclosure provides an anti-C1s antibody of any of the embodiments or a pharmaceutical composition thereof for inhibiting complement C1s protein activity in an individual having a complement-mediated disease or disorder.

The present disclosure provides a method to diagnose a complement-mediated disease or disorder in an individual, the method comprising: (a) determining the amount of a complement C1s protein in a biological sample obtained from the individual, wherein the step of determining comprises: (i) contacting the biological sample with an anti-C1s antibody of any of the embodiments; and (ii) quantitating binding of the antibody to complement C1s protein present in the sample; and (b) comparing the amount of the complement C1s protein to a normal control value that indicates the amount of complement C1s protein in a normal control individual, wherein a significant difference between the amount of C1s protein in the biological sample and the normal control value indicates that the individual has a complement-mediated disease or disorder. In some embodiments, the biological sample is selected from the group consisting of blood, serum, plasma, urine, saliva, cerebrospinal fluid, interstitial fluid, ocular fluid, synovial fluid, solid tissue sample, tissue culture sample, and cellular sample.

The present disclosure provides a method to monitor progression of a complement-mediated disease or disorder in an individual, the method comprising: (a) determining a first amount of complement a C1s protein in a biological sample obtained from the individual at a first time point; (b) determining a second amount of complement a C1s protein in a biological sample obtained from the individual at a second time point; and (c) comparing the second amount of complement C1s protein with the first amount of complement C1s protein. The steps of determining comprise: (i) contacting the biological sample with an anti-C1s antibody of any of the embodiments; and (ii) quantitating binding of the antibody to complement C1s protein present in the sample. In some embodiments, the first time point is a time point before initiation of a treatment regimen, and the second time point is a time point after initiation of a treatment regimen. In some embodiments, the biological sample is selected from the group consisting of blood, serum, plasma, urine, saliva, cerebrospinal fluid, interstitial fluid, ocular fluid, synovial fluid, solid tissue sample, tissue culture sample, and cellular sample.

The present disclosure provides an in vitro method to detect complement C1s protein in a biological sample obtained from an individual, the method comprising: (a) contacting the biological sample with an anti-C1s antibody of any of the embodiments; and (b) detecting binding of the antibody to complement C1s protein present in the sample. In some embodiments, the biological sample is selected from the group consisting of blood, serum, plasma, urine, saliva, cerebrospinal fluid, interstitial fluid, ocular fluid, synovial fluid, solid tissue sample, tissue culture sample, and cellular sample. In some embodiments, the method is quantitative.

The present disclosure provides a method to detect complement C1s protein in a living individual in vivo, the method comprising: (a) administering to the individual an anti-C1s antibody of any of the embodiments; and (b) detecting binding of the antibody to complement C1s protein in the individual using an imaging method. In some embodiments, the binding is detected in the individual at a site altered by a complement-mediated disease or disorder. In some embodiments, the binding is detected in the brain of the individual. In some of the embodiments, the antibody comprises a contrast agent suitable for use in the imaging method. In some embodiments, the imaging method is selected from the group consisting of magnetic resonance imaging, positron emission tomography, and IVIS instrumentation. In some embodiments, the method is quantitative.

In some embodiments, the biological sample is selected from the group consisting of blood, serum, plasma, urine, saliva, cerebrospinal fluid, interstitial fluid, ocular fluid, synovial fluid, solid tissue sample, tissue culture sample, and a cellular sample.

In some embodiments, the methods of the present disclosure provide that the individual is suspected of having a complement-mediated disease or disorder, has been diagnosed as having a complement-mediated disease or disorder, or has a genetic predisposition to developing a complement-mediated disease or disorder.

The present disclosure provides a composition comprising: (a) an anti-C1s antibody of any of the embodiments; and (b) a solution comprising one or more agents that maintain an organ or a tissue intended for transplantation into a recipient individual. In some embodiments, the solution is an organ preservation solution or a tissue preservation solution. In some embodiments, the solution is an organ perfusion solution or a tissue perfusion solution. In some embodiments, the solution comprises: i) a salt; ii) an agent that reduces edema; iii) an oxygen free radical scavenger; and iii) an energy supply system component. In some embodiments, the composition comprises potassium lactobionate, KH₂PO₄, MgSO₄, raffinose, adenosine, glutathione, allopurinol, and/or hydroxyethyl starch.

The present disclosure provides an organ or tissue preservation solution comprising an anti-C1s antibody of any of the embodiments or a pharmaceutical composition thereof.

The present disclosure provides an organ or tissue perfusion solution comprising an anti-C1s antibody of any of the embodiments or a pharmaceutical composition thereof.

The present disclosure provides a method for maintaining an organ or tissue for transplant, the method comprising contacting the organ or the tissue with a composition comprising: (a) an anti-C1s antibody of any of the embodiments; and (b) an organ or tissue preservation solution of any of the embodiments or an organ or tissue perfusion solution of any of the embodiments.

The present disclosure provides an isolated organ or tissue maintained in a composition comprising: (a) an anti-C1s antibody of any of the embodiments; and (b) an organ or tissue preservation solution of any of the embodiments or an organ or tissue perfusion solution of any of the embodiments. In some embodiments, the organ is selected from the group consisting of an eye, a heart, an intestine, a kidney, a liver, a lung, a pancreas, a stomach, and a thymus. In some embodiments, the tissue is selected from the group consisting of bone, bone marrow, cornea, heart valve, islet of Langerhans, tendon, skin, and vein.

The present disclosure provides an in vitro method for inhibiting complement C1s activity in an organ or a tissue, the method comprising contacting the organ or the tissue with an anti-C1s antibody of any of the embodiments or a pharmaceutical composition thereof.

Treatment of individuals may comprise the administration of a therapeutically effective amount of the antibodies described herein. The antibodies can be provided in a kit, such as those provided herein. The antibodies can be used or administered alone or in admixture with another therapeutic, analgesic, or diagnostic agent, such as provided for herein. In providing a patient with an antibody, or fragment thereof, capable of binding to C1s, or an antibody capable of protecting against C1s in a recipient patient, the dosage of administered agent will vary depending upon such factors as the patient's age, weight, height, sex, general medical condition, previous medical history, etc.

An antibody, capable treating a condition associated with C1s activity or use to treat a C1s related pathology, is intended to be provided to subjects in an amount sufficient to affect a reduction, resolution, or amelioration in the C1s related symptom or pathology. Examples of such pathologies are provided for herein.

Accordingly, in some embodiments, methods of treating a subject with a C1s mediated disorder are provided. In some embodiments, the method comprises administering a pharmaceutical composition comprising an antibody, or antigen binding fragment thereof, as provided herein. In some embodiments, the disorder is as provided for herein.

As provided for herein, the antibodies, or antigen binding fragments thereof, can be administered with other therapeutics. These can be administered simultaneously or sequentially.

Kits are also provided which are useful for carrying out embodiments described herein. The present kits can comprise a first container containing or packaged in association with the above-described antibodies. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

In some embodiments, antibodies that bind to a C1s protein are provided. In some embodiments, the antibodies are antibodies or antigen binding fragments as provided for herein. In some embodiments, the antibodies or antigen binding fragments comprise an amino acid sequence as provided for herein, or a variant thereof as provided for herein. In some embodiments, the antibody is isolated. In some embodiments, the antibody binds specifically to the active form of C1s.

In some embodiments, the antibody inhibits or neutralizes the function of an active form of C1s protein. As used herein, the term “neutralize” means that the activity or function of the protein is inhibited. The inhibition can be complete or partial. In some embodiments, the activity or function of the protein is inhibited at least 10, 20, 30, 40, 50, 60, 70, 80, 90, 95, or 99%. The percent inhibition can be based upon the function or activity of the protein in the absence of the antibody. In some embodiments, the antibody inhibits the function facilitated by C1s.

Throughout the present disclosure, all expressions of percentage, ratio, and the like are “by weight” unless otherwise indicated. As used herein, “by weight” is synonymous with the term “by mass,” and indicates that a ratio or percentage defined herein is done according to weight rather than volume, thickness, or some other measure.

### ENUMERATED EMBODIMENTS

In some embodiments, the following embodiments are provided.

1. A pharmaceutical composition comprising:
about 25 mg/mL to about 300 mg/mL of an antibody having a heavy chain and a light chain, wherein the heavy chain comprises a HCDR1 of SEQ ID NO: 61, a HCDR2 of SEQ ID NO: 62, and a HCDR3 of SEQ ID NO: 78, and wherein the light chain comprises a LCDR1 of SEQ ID NO: 64, a LCDR2 of SEQ ID NO: 65, and a LCDR3 of SEQ ID NO: 79;
one or more of a buffer, an antioxidant, a sugar, a viscosity modifying agent, or a surfactant; and
wherein the pharmaceutical composition is at a pH of about 6.5 to about 8.0.
   2. The pharmaceutical composition of embodiment 1, wherein the pharmaceutical composition comprises:
a buffer at about 5 mM to about 50 mM,
an antioxidant at about 5 mM to about 15 mM,
a sugar at about 1% w/v to about 14% w/v,
a viscosity modifying agent at about 20 mM to about 180 mM, and
a surfactant at about 0.001% w/v to about 0.4% w/v.
   3. The pharmaceutical composition of embodiment 1, wherein the pharmaceutical composition comprises:
a buffer at about 15 mM to about 25 mM,
an antioxidant at about 5 mM to about 15 mM,
a sugar at about 1% w/v to about 10% w/v,
a viscosity modifying agent at about 60 mM to about 150 mM, and
a surfactant at about 0.001% w/v to about 0.2% w/v.
   4. The pharmaceutical composition of embodiment 1, wherein the buffer is at a concentration of about 5 mM to about 15 mM, about 15 mM to about 20 mM, about 20 mM to about 25 mM, about 25 mM to about 30 mM, about 30 mM to about 35 mM, about 35 mM to about 40 mM, about 40 mM to about 45 mM, about 45 mM to about 50 mM, about 5 mM, about 10 mM, about 15 mM, about 20 mM, about 25 mM, about 30 mM, about 35 mM, about 40 mM, about 45 mM, or about 50 mM.
   5. The pharmaceutical composition of embodiment 1, wherein the buffer is selected from tris buffer, histidine buffer, HEPES, phosphate buffer, acetate buffer, citrate buffer, succinate buffer, ascorbate buffer, glutamate buffer, lactate buffer, maleate buffer, trometamol buffer, gluconate buffer, or any combination thereof.
   6. The pharmaceutical composition of any one of embodiments 1-5, wherein the buffer is a phosphate buffer.
   7. The pharmaceutical composition of embodiment 6, wherein the phosphate buffer is present at about 5 mM to about 15 mM, about 25 mM to about 50 mM, or about 20 mM.
   8. The pharmaceutical composition of embodiment 1, wherein the pharmaceutical composition has a pH of about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.
   9. The pharmaceutical composition of embodiment 1-8, wherein the pharmaceutical composition has a pH of about 6.5 to about 7.7.
   10. The pharmaceutical composition of any one of embodiments 1-9, wherein the pharmaceutical composition has a pH of about 7.2.
   11. The pharmaceutical composition of any one of embodiments 1-10, wherein the buffer is a phosphate buffer and the pharmaceutical composition has a pH of about 7.2.
   12. The pharmaceutical composition of embodiment 1, wherein the antioxidant is present at about 10 mM to about 15 mM, or about 5 mM to about 10 mM.
   13. The pharmaceutical composition of embodiment 1, wherein the antioxidant is present at a concentration of about 10 mM.
   14. The pharmaceutical composition of embodiment 12 or 13, wherein the antioxidant is selected from L-methionine, ascorbic acid, EDTA, or any combination thereof.
   15. The pharmaceutical composition of embodiment 14, wherein the antioxidant is L-methionine.
   16. The pharmaceutical composition of embodiment 15, wherein the L-methionine is present at about 5 mM to about 15 mM, about 10 mM to about 15 mM, or about 5 mM to about 10 mM.
   17. The pharmaceutical composition of embodiment 15 or 16, wherein the L-methionine is present at about 10 mM.
   18. The pharmaceutical composition of embodiment 1, wherein the sugar is present at a concentration of about 1% w/v to about 14% w/v, about 2% w/v to about 14% w/v, about 2% w/v to about 12% w/v, 2% w/v to about 10% w/v, about 2% w/v to about 8% w/v, about 2% w/v to about 6% w/v, about 2% w/v to about 4% w/v, about 4% w/v to about 14% w/v, about 4% w/v to about 12% w/v, about 4% w/v to about 10% w/v, about 4% w/v to about 8% w/v, about 4% w/v to about 6% w/v, about 6% w/v to about 14% w/v, about 6% w/v to about 12% w/v, about 6% w/v to about 10% w/v, about 6% w/v to about 8% w/v, about 8% w/v to about 14% w/v, about 8% w/v to about 12% w/v, about 8% w/v to about 10% w/v, about 10% w/v to about 14% w/v, about 10% w/v to about 12% w/v, or about 12% w/v to about 14% w/v.
   19. The pharmaceutical composition of embodiment 1, wherein the sugar is sucrose, trehalose, sorbitol, mannitol, or any combination thereof.
   20. The pharmaceutical composition of embodiment 19, wherein the sugar is sucrose.
   21. The pharmaceutical composition of embodiment 20, wherein the sucrose is present at a concentration of about 1% to about 14% w/v.
   22. The pharmaceutical composition of embodiment 21, wherein the sucrose is present at a concentration of about 2% w/v to about 10% w/v.
   23. The pharmaceutical composition of any one of embodiments 20-22, wherein the sucrose is present at a concentration of about 4% w/v.
   24. The pharmaceutical composition of any one of embodiments 20-23, wherein the sucrose is present at a concentration of about 3.5% w/v.
   25. The pharmaceutical composition of embodiment 1, wherein the viscosity modifying agent is present at a concentration of about 20 mM to about 180 mM, about 20 mM to about 170 mM, about 20 mM to about 160 mM, about 20 mM to about 150 mM, about 20 mM to about 140 mM, about 20 mM to about 130 mM, about 20 mM to about 120 mM, about 20 mM to about 110 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 40 mM to about 180 mM, about 40 mM to about 170 mM, about 40 mM to about 160 mM, about 40 mM to about 150 mM, about 40 mM to about 140 mM, about 40 mM to about 130 mM, about 40 mM to about 120 mM, about 40 mM to about 110 mM, about 40 mM to about 100 mM, about 40 mM to about 90 mM, about 40 mM to about 80 mM, about 40 mM to about 70 mM, about 40 mM to about 60 mM, about 40 mM to about 50 mM, about 60 mM to about 180 mM, about 60 mM to about 170 mM, about 60 mM to about 160 mM, about 60 mM to about 150 mM, about 60 mM to about 140 mM, about 60 mM to about 130 mM, about 60 mM to about 120 mM, about 60 mM to about 110 mM, about 60 mM to about 100 mM, about 60 mM to about 90 mM, about 60 mM to about 80 mM, about 60 mM to about 70 mM, about 70 mM to about 180 mM, about 70 mM to about 170 mM, about 70 mM to about 160 mM, about 70 mM to about 150 mM, about 70 mM to about 140 mM, about 70 mM to about 130 mM, about 70 mM to about 120 mM, about 70 mM to about 110 mM, about 70 mM to about 100 mM, about 70 mM to about 90 mM, about 70 mM to about 80 mM, about 80 mM to about 180 mM, about 80 mM to about 170 mM, about 80 mM to about 160 mM, about 80 mM to about 150 mM, about 80 mM to about 140 mM, about 80 mM to about 130 mM, about 80 mM to about 120 mM, about 80 mM to about 110 mM, about 80 mM to about 100 mM, about 80 mM to about 90 mM, about 90 mM to about 180 mM, about 90 mM to about 170 mM, about 90 mM to about 160 mM, about 90 mM to about 150 mM, about 90 mM to about 140 mM, about 90 mM to about 130 mM, about 90 mM to about 120 mM, about 90 mM to about 110 mM, about 90 mM to about 100 mM, about 100 mM to about 180 mM, about 100 mM to about 170 mM, about 100 mM to about 160 mM, about 100 mM to about 150 mM, about 100 mM to about 140 mM, about 100 mM to about 130 mM, about 100 mM to about 120 mM, about 100 mM to about 110 mM, about 110 mM to about 180 mM, about 110 mM to about 170 mM, about 110 mM to about 160 mM, about 110 mM to about 150 mM, about 110 mM to about 140 mM, about 110 mM to about 130 mM, about 110 mM to about 120 mM, about 120 mM to about 180 mM, about 120 mM to about 170 mM, about 120 mM to about 160 mM, about 120 mM to about 150 mM, about 120 mM to about 140 mM, about 120 mM to about 130 mM, about 130 mM to about 180 mM, about 130 mM to about 170 mM, about 130 mM to about 160 mM, about 130 mM to about 150 mM, about 130 mM to about 140 mM, about 140 mM to about 180 mM, about 140 mM to about 170 mM, about 140 mM to about 160 mM, about 140 mM to about 150 mM, about 150 mM to about 180 mM, about 150 mM to about 170 mM, about 150 mM to about 160 mM, about 160 mM to about 180 mM, about 160 mM to about 170 mM, or about 170 mM to about 180 mM.
   26. The pharmaceutical composition of embodiment 25, wherein the viscosity modifying agent is present at a concentration of about 70 mM.
   27. The pharmaceutical composition of embodiment 25 or 26, wherein the viscosity modifying agent is L-arginine hydrochloride (L-Arg-HCL) or sodium chloride (NaCl).
   28. The pharmaceutical composition of embodiment 25-27, wherein the viscosity modifying agent is L-Arg-HCL.
   29. The pharmaceutical composition of embodiment 1, wherein the surfactant is present at a concentration of about 0.001% w/v to about 0.10% w/v, about 0.001% w/v to about 0.2% w/v, about 0.01% w/v to about 0.2% w/v, about 0.10% w/v to about 0.2% w/v, about 0.2% w/v to about 0.4% w/v, about 0.050% w/v to about 0.150% w/v, or about 0.001% w/v to about 0.01% w/v.
   30. The pharmaceutical composition of embodiment 1, wherein the surfactant is Polysorbate 80, Polysorbate 20, polyethylene glycol 3350 (PEG3350), or Poloxamer (P)188.
   31. The pharmaceutical composition of embodiment 30, wherein the surfactant is P188.
   32. The pharmaceutical composition of any one of embodiments 30 or 31, wherein the P188 is present at a concentration of about 0.1% w/v.
   33. The pharmaceutical composition of embodiment 1, wherein the antibody is present at a concentration of about 25 mg/mL to about 300 mg/mL, about 25 mg/mL to about 275 mg/mL, about 25 mg/mL to about 250 mg/mL, about 25 mg/mL to about 225 mg/mL, about 25 mg/mL to about 200 mg/mL, about 25 mg/mL to about 175 mg/mL, about 25 mg/mL to about 150 mg/mL, about 25 mg/mL to about 125 mg/mL, about 25 mg/mL to about 100 mg/mL, about 25 mg/mL to about 75 mg/mL, about 50 mg/mL to about 300 mg/mL, about 50 mg/mL to about 275 mg/mL, about 50 mg/mL to about 250 mg/mL, about 50 mg/mL to about 225 mg/mL, about 50 mg/mL to about 200 mg/mL, about 50 mg/mL to about 175 mg/mL, about 50 mg/mL to about 150 mg/mL, about 50 mg/mL to about 125 mg/mL, about 50 mg/mL to about 100 mg/mL, about 50 mg/mL to about 75 mg/mL, about 75 mg/mL to about 300 mg/mL, about 75 mg/mL to about 275 mg/mL, about 75 mg/mL to about 250 mg/mL, about 75 mg/mL to about 225 mg/mL, about 75 mg/mL to about 200 mg/mL, about 75 mg/mL to about 175 mg/mL, about 75 mg/mL to about 150 mg/mL, about 75 mg/mL to about 125 mg/mL, about 75 mg/mL to about 100 mg/mL, about 100 mg/mL to about 300 mg/mL, about 100 mg/mL to about 275 mg/mL, about 100 mg/mL to about 250 mg/mL, about 100 mg/mL to about 225 mg/mL, about 100 mg/mL to about 200 mg/mL, about 100 mg/mL to about 175 mg/mL, about 100 mg/mL to about 150 mg/mL, about 100 mg/mL to about 125 mg/mL, about 125 mg/mL to about 300 mg/mL, about 125 mg/mL to about 275 mg/mL, about 125 mg/mL to about 250 mg/mL, about 125 mg/mL to about 225 mg/mL, about 125 mg/mL to about 200 mg/mL, about 125 mg/mL to about 175 mg/mL, about 125 mg/mL to about 150 mg/mL, about 150 mg/mL to about 300 mg/mL, about 150 mg/mL to about 275 mg/mL, about 150 mg/mL to about 250 mg/mL, about 150 mg/mL to about 225 mg/mL, about 150 mg/mL to about 200 mg/mL, about 150 mg/mL to about 175 mg/mL, about 175 mg/mL to about 300 mg/mL, about 175 mg/mL to about 275 mg/mL, about 175 mg/mL to about 250 mg/mL, about 175 mg/mL to about 225 mg/mL, about 175 mg/mL to about 200 mg/mL, about 200 mg/mL to about 300 mg/mL, about 200 mg/mL to about 275 mg/mL, about 200 mg/mL to about 250 mg/mL, about 200 mg/mL to about 225 mg/mL, about 225 mg/mL to about 300 mg/mL, about 225 mg/mL to about 275 mg/mL, about 225 mg/mL to about 250 mg/mL, about 250 mg/mL to about 300 mg/mL, about 250 mg/mL to about 275 mg/mL, about 275 mg/mL to about 300 mg/mL, about 100 mg/mL to about 200 mg/mL, or about 200 mg/mL to about 300 mg/mL, about 300 mg/mL to about 400 mg/mL, about 400 mg/mL to about 500 mg/mL, about 500 mg/mL to about 600 mg/mL, about 100 mg/mL to about 300 mg/mL, about 200 mg/mL to about 400 mg/mL, about 300 mg/mL to about 500 mg/mL, about 400 mg/mL to about 600 mg/mL, about 100 mg/mL to about 400 mg/mL, about 200 mg/mL to about 500 mg/mL, or about 300 mg/mL to about 600 mg/mL.
   34. The pharmaceutical composition of embodiment 33, wherein the antibody is a monoclonal antibody.
   35. The pharmaceutical composition of embodiment 34, wherein the monoclonal antibody is an immunoglobulin G₄ (IgG₄) type monoclonal antibody.
   36. The pharmaceutical composition of embodiment 1, wherein the antibody comprises a heavy chain variable region with an amino acid sequence of SEQ ID NO: 17 and a light chain variable region with an amino acid sequence of SEQ ID NO: 18.
   37. The pharmaceutical composition of embodiment 1, wherein the antibody comprises a heavy chain variable region with an amino acid sequence of SEQ ID NO: 342 and a light chain variable region with an amino acid sequence of SEQ ID NO: 18.
   38. The antibody of embodiment 1-37, or an antigen binding fragment thereof, wherein the antibody or antibody fragment comprises a heavy chain variable region comprising heavy chain HCDR1, HCDR2, and HCDR3 sequences, wherein the heavy chain HCDR1 sequence has the amino acid sequence of SEQ ID NO: 61; the heavy chain HCDR2 has the amino acid sequence of SEQ ID NO: 62; and the heavy chain HCDR3 sequence has the amino acid sequence of SEQ ID NO: 78, or variants or convention equivalents of any of the foregoing; and (ii) a light chain variable region comprising light chain LCDR1, LCDR2, and LCDR3 sequences, wherein the light chain LCDR1 sequence has the amino acid sequence SEQ ID NO: 64; the light chain LCDR2 sequence has the amino acid sequence of SEQ ID NO: 65; and the light chain LCDR3 sequence has the amino acid sequence of SEQ ID NO: 79; or variants or convention equivalents of any of the foregoing.
   39. The pharmaceutical composition of any one of preceding embodiments, wherein the antibody is present at a concentration of about 100 mg/mL.
   40. The pharmaceutical composition of any one of preceding embodiments, wherein the antibody is present at a concentration of about 150 mg/mL.
   41. The pharmaceutical composition of embodiment 1-40, wherein the pharmaceutical composition is stable at about −25° C., about −20° C., about −15° C., about −5° C., about 0° C., about 5° C., or about 10° C.
   42. The pharmaceutical composition of embodiment 39, wherein the pharmaceutical composition is stable for 1 week, 2 weeks, 3 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months.
   43. The pharmaceutical composition of embodiment 1-42, wherein the pharmaceutical composition is stable at −20° C. for at least 3 months.
   44. The pharmaceutical composition of embodiment 1-43, wherein the pharmaceutical composition is stable at −20° C. for at least 6 months.
   45. The pharmaceutical composition of embodiment 1-44, wherein the pharmaceutical composition is stable at 2-8° C. for at least 3 months.
   46. The pharmaceutical composition of embodiment 1-45, wherein the pharmaceutical composition is stable at 2-8° C. for at least 6 months.
   47. The pharmaceutical composition of embodiment 1-46, wherein the pharmaceutical composition is stable at 2-8° C. for at least 9 months.
   48. The pharmaceutical composition of embodiment 1-47, wherein the pharmaceutical composition is stored in pre-filled syringes or 2 mL aliquots in 6 mL vials.
   49. The pharmaceutical composition of embodiment 1-48, wherein the pharmaceutical composition is stored in 2 mL aliquots in 6 mL vials with a 20 mm stopper.
   50. The pharmaceutical composition of embodiment 1-49, wherein the pharmaceutical composition is stored in 2 mL aliquots at −20° C. in 6 mL vials with a 20 mm stopper and a 20 mm aluminum plastic cover.
   51. The pharmaceutical composition of embodiment 1-50, wherein the pharmaceutical composition is for intravenous or subcutaneous injection.
   52. A method of treating a subject with C1s mediated disorder, the method comprising administering to the subject the pharmaceutical composition of any one of embodiments 1-51.
   53. The method of embodiment 52, wherein the C1s mediated disorder is hemolysis, Cold Agglutinin Disease, Immune Thrombocytopenia (ITP), Myasthenia Gravis, Glomerulopathies, Atypical Hemolytic uremic syndrome, antiphospholipid antibody syndrome, transplant rejection, chronic inflammatory demyelinating polyneuropathy (CIDP), multifocal motor neuropathy (MMN), dermatomyositis, anti MAG neuropathy, due to stroke, or due to spinal cord injury.
   54. The pharmaceutical composition of embodiment 1, wherein:
the antibody is at about 100 mg/mL,
the buffer is phosphate buffer at about 20 mM,
the antioxidant is L-methionine at about 10 mM,
the sugar is sucrose at about 4% w/v,
the viscosity modifying agent is L-arginine hydrochloride at about 70 mM, and
the surfactant is P188 at about 0.1% w/v;
wherein the pharmaceutical composition is at a pH of about 7.2.
   55. The pharmaceutical composition of embodiment 1, wherein:
the antibody is at about 150 mg/mL,
the buffer is phosphate buffer at about 20 mM,
the antioxidant is L-methionine at about 10 mM,
the sugar is sucrose at about 3.5% w/v,
the viscosity modifying agent is L-arginine hydrochloride at about 70 mM, and
the surfactant is P188 at about 0.1% w/v;
wherein the pharmaceutical composition is at a pH of about 7.2.

### EXAMPLES

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

Example 1: High concentration formulation development. The formulation development utilizing 150 mg/mL protein was divided into two parts: 1) excipient and surfactant strength screening study to evaluate and select appropriate excipients and surfactants according to their stabilizing effect on the protein under stress conditions and 2) selected formulation evaluation and confirmation study, undertaken through a 6 month period under various conditions where a plethora of biophysical and analytical methods were employed to determine the potential chemical and physical changes that might occur to the formulated protein.

The analytical methods performed throughout both studies include examination of appearance, pH, protein concentration, osmolality, viscosity, SEC-HPLC, SEC-UPLC, Micro Flow Imaging, Sub-Visible Particles (HIAC), iCIEF, capillary-SDS-NR & R, CD-SDS-NR & R, ELISA, binding ELISA, DSC, peptide mapping, CDC potency assay, and mDSC and are described below.

The appearance of samples, including color, clarity and visible particles, was examined against black and white background using a YB-2 lightbox. The light intensity of lightbox was set as 2000˜3750 lux.

The pH value was measured using a pH meter with a glass electrode. The pH meter was calibrated with three different standard solutions (pH 4.01, 7.00 and 9.21) prior to usage. The calibration pass criteria were the slope of calibration landed between 95.0%-105.0%. Each sample was measured twice, an average result was reported.

Protein concentration was determined by a Big Lunatic UV Spectrophotometer. According to the Lambert-Beer law, the concentration of a protein solution can be calculated based on its absorbance at a given wavelength, the cuvette cell path length, and extinction coefficient value. The absorbance in 280 nm relies on the absorption properties of the aromatic amino acid residues in protein. The EC used in all evaluation studies was 1.53 AU*mL*mg⁻¹*cm⁻¹. All samples were performed without any dilution. After loading a 2.0 μL sample volume, Lunatic measured the absorbance in two cuvettes in parallel and calculated the concentration value. All sample measurements were conducted after a blank control using water.

Osmolality was measured using an Advanced 2020 Multi-Sample Osmometer. Before and after the sample tests, the accuracy of osmometer was confirmed with a Clinitrol 290 mOsm/kg reference solution. The sample volume for each testing was 20 μL and only one test was performed for each sample.

Viscosity was measured by a DHR-2 Rotational Rheometer. The testing temperature was controlled at 21° C. The accuracy of the Rheometer was confirmed with a certified viscosity reference N35. The testing shear rate was raised from 10 to 1500 s⁻¹ and data acquisition frequency was set as 10 points per decade. Prior to recording a point, the sample was allowed to equilibrate for 30.0 s. 650 L sample was loaded for each testing.

Size exclusion-high-performance liquid chromatography (SEC-HPLC) was used in pH buffer screening, excipients, and surfactant screening. SEC-HPLC is a purity analysis method that separates proteins based on their sizes. Measurements were performed on an Agilent HPLC system with an SEC column (300×7.8 mm, 5 μm). The sampler temperature was set to 5±3° C., and the column oven temperature was set to 25±3° C. The mobile phase was 50 mM PB, 300 mM sodium chloride (NaCl), pH 6.8±0.1, and the flow rate was set as 1.0 mL/min. Samples were diluted to 10 mg/mL with mobile phase, and 100 μg samples were injected. UV detection wavelength was set at 280 nm and the run time was 20 minutes (min). Data were analyzed by Agilent CDS Software.

Size exclusion-ultra-performance liquid chromatography (SEC-UPLC) was used in the formulation confirmation study. In this study, 10 μg protein samples were injected into a Waters H-Class, separated on a Waters ACQUITY UPLC Protein BEH SEC Column (200 Å, 1.7 μm, 4.6 mm×150 mm). UV absorbance at 280 nm wavelength was monitored and logged to generate chromatograms. 50 mM PB, 300 mM NaCl, pH 6.8±0.1 was used as the mobile phase and an isocratic gradient was applied for 8 min at a flow rate of 0.4 mL/min. All the results were rounded to one decimal place.

Sub-visible particles were monitored by an MFI system (MFI 5200). 1.3 mL of each sample was transferred into an MFI 96-well plate in a bio-safety hood for analysis. The results were analyzed by the vendor's MVAS 1.3 software. The sub-visible particle amount in the equivalent circular diameter between 2˜5 μm, 5˜10 μm, 10˜25 μm and over 25 μm was reported.

A Liquid Particle Counting Systems (HIAC 9703+) was utilized to measure the size and counts of sub-visible particles under a laminar flow cabinet. To avoid introducing air bubbles and interference during examination, all samples were held in the cabinet before testing for approximately 15-20 minutes. Each sample was tested for four consecutive runs and 1 mL for each run. The first run was discarded and results were presented as average number of particles ≥2 μm, ≥5 μm, ≥10 μm, and ≥25 μm per mL from the last 3 runs.

Imaged capillary isoelectric focusing (iCIEF) is a purity method used to monitor the charge variant species by determining the isoelectric point (pI) and distribution of each variant. Charge variants of the protein are separated based on their unique pI. pI is an intrinsic property of each charge variant of the protein and is equal to the pH at which the protein molecule does not carry any net charge. Under an external electric field, the charge variants move along a continuous pH gradient formed by the ampholytes and stop at the position where the pH equals to its pI. In this study, protein samples were diluted to 1.0 mg/mL with ultrapure water. Then, a 20 μL diluted sample was mixed with 80 μL master mixture (composed of pI markers 7.65/10.10, carrier ampholytes (3-10 and 8-10.5), methyl cellulose, arginine, and urea), before being loaded into a capillary for electric focusing by ICE3. Data were processed with the Empower 3. The percentage of the main peak, acidic peaks and basic peaks was reported in the final results, along with the pI of the main peak. All the results were rounded to one decimal place.

Capillary-Sodium Dodecyl Sulfate (capillary-SDS) is a purity method utilized to determine the truncation or fragmentation level of the biomolecules during the production and storage processes. Capillary-SDS employs a microchip to separate proteins based on their electrophoretic mobility, with proteins of smaller size migrating further than proteins of larger size in a capillary. In this study, the samples were diluted to 1.0 mg/mL with ultrapure water, and then mixed with the denaturing solution (composed of sample buffer, SDS and N-ethylmaleimide (NEM, for the NR method) or dithiothreitol (DTT, for the R method)). The mixtures were incubated at 70° C. for 10 min, and then transferred into a 96-well plate. After the plate was loaded onto the plate holder of the instrument, samples were injected, stained, separated and detected in the microchip. Data were acquired with LabChip GX Reviewer and analyzed with the Empower 3. The percentage of the main peak was reported as the purity of the sample in the non-reduced capillary-SDS. While for the reduced method, the percentage of light chain (LC), heavy chain (HC), and the sum of LC+HC (purity) was reported. All results were rounded to one decimal place.

Capillary Electrophoresis Sodium Dodecyl Sulfate (CE-SDS) is a popular technology employed to determine the truncation or fragmentation level of the biomolecules during the production and storage processes. CE-SDS separates proteins based on their electrophoretic mobility, with proteins of smaller sizes move faster and of larger sizes move slower in a capillary. In this study, both NR and R methods were applied. In the non-reduced CE-SDS analysis, samples were diluted to 4.0 mg/mL with the diluent (PB-CA buffer). 25 μL of the diluted sample was transferred to a 1.5 mL centrifuge tube, followed by the addition of 5 μL alkylation reagent (100 mM NEM). 75 μL of 1.0% (w/v) SDS sample buffer was then added into the vial to make a final volume of 105 μL. The samples were incubated at 60° C. for 10 minutes, followed by the analysis with PA800 Plus. In contrast, reduced CE-SDS shares the same principle but differs in the sample preparation. Samples were diluted to 4.0 mg/mL with the diluent (PB-CA buffer). 25 μL of the diluted sample was transferred to a 1.5 mL centrifuge tube, followed by the addition of 75 μL 1.0% (w/v) SDS sample buffer. Then, 5 μL of β-mercaptoethanol (BME) was added to make a total volume of 105 μL. The samples were incubated at 70° C. for 10 minutes, followed by the analysis with PA800 Plus. The percentage of the total protein in the main peak was reported as the purity of the sample in the non-reduced CE-SDS. In addition, total proportion of pre-peaks and HMWS were also reported. The SOP method (PD-AS-LAB-173) is followed. While for the reduced method, the percentage of LC, HC, and the sum of LC+HC (purity) was reported. Meanwhile, the total percentage of minor species was reported as well. All the results were rounded to one decimal place.

Enzyme-linked immunosorbent assay (ELISA) is a binding activity assay where the antibody molecule binds to the antigen, Cis, which had been immobilized on the plate and blocked by 2% non-fat milk. Serially diluted antibody was bound to the plate through CIs upon addition and incubation. After washing the plate, Goat anti-Human IgG (Fc specific)-Peroxidase, was added to the wells allowing interaction with antibody captured during previous step. After a final wash step, the 3,3″,5,5″-Tetramethylbenzidine (TMB) substrate solution was added to wells. TMB reacted with the peroxide for 3˜25 min followed by loading 1 M sulfuric acid. The coated plate was placed in the plate reader, and absorbance read at 450 nm. The dose response curves were fitted using a 4-parameter logistic model and the results reported as Relative Potency using the EC50 values of the Reference Standard (RS) vs. the Sample.

Differential scanning calorimetry (DSC) is a thermo-analytical technique used to characterize the thermal stability of protein samples and assess conformational differences between them. Measurements were performed on MicroCal™ VP-Capillary DSC (Malvern) for thermal transition midpoint (Tm) and onset of unfolding (TmOnset) testing. Samples were diluted to 1 mg/mL with the reference buffer. 400 μL of respective reference buffers were added into the odd-numbered wells of a 96-well plate and 400 μL of samples were added into the even-numbered wells of the same plate. Experimental parameters were set such that the scan temperature ramped from 10 to 95° C. at a scan rate of 200° C./h. Data analysis was performed in MicroCal™ VP-Capillary DSC (software: MicroCal VP-Capillary DSC Software 2.0) automated data analysis software.

Tg′ is the glass transition temperature (glass transition temperature is the temperature below which the phase change happens for amorphous (not crystalline) solids) of the maximally frozen concentrated solute, and is a critical parameter in determining the state of frozen DS. Below the Tg′, the formulated protein would be in glass state where potential physical or chemical change would be substantially limited. The Tg′ of the formulations were measured by a TA Q2000 DSC. 20 μL of the samples were sealed in aluminum pans and transferred into the equipment. A sealed empty pan was used as the reference. The samples were cooled to −60° C. by a cooling rate of 10° C./minute, and kept at this temperature for 5 minutes, then were heated to 20° C. with heating rate of 10° C./minute. The results were recorded and analyzed by Universal Analysis Software.

Part 1: Excipient and Surfactant Strength Screening Study. The objective of this study was to screen the optimal excipient and surfactant that help to stabilize the selected concentration of protein the most. Briefly, For 150 mg/mL antibody, various concentrations of sucrose (2%, 3.5%, 5%), L-Arg-HCl (40 mM, 70 mM, 100 mM and 140 mM) and P188 (0.05%, 0.1%, 0.2%, w/v) were evaluated under stress conditions (40° C. for up to 3 M, agitation at 300 rpm for 3 days (D), and freeze/thaw (FT) for up to 5 cycles (CYS). Appearance, pH, protein conc., osmolality, SEC-HPLC, capillary-SDS-NR & R, iCIEF, MFI, DSC and potency testing were performed to test the quality of DS in this study. Table 2 below summarizes the results described in further detail below.

TABLE 2
Formulation candidates list of excipients and surfactant strength
screening study. All % in the table means % (w/v).

| Formulation | Buffer System, pH, Protein | |
|---|---|---|
| Number | Concentration | Excipients/Surfactant |
| F1-1 | 20 mM Phosphate Buffer, 10 mM | 5% sucrose, 40 mM L-Arg-HCl, 0.1% P188 |
| F1-2 | L-Met, pH 7.2, 150 mg/mL Protein | 3.5% sucrose, 40 mM L-Arg-HCl, 0.1% P188 |
| F1-3 | | 2% sucrose, 100 mM L-Arg-HCl, 0.1% P188 |
| F1-4 | | 140 mM L-Arg-HCl, 0.1% P188 |
| F1-5 | | 3.5% sucrose, 70 mM L-Arg-HCl, 0.05% P188 |
| F1-6 | | 3.5% sucrose, 70 mM L-Arg-HCl, 0.2% P188 |

Excipients and surfactant strength screening study was conducted with 150 mg/mL protein. In this study, all samples remained unchanged in appearance, protein conc. and pH. And the viscosity was in the range of 3.9-5.3 cP. F1-4 (140 mM L-Arginine-HCl, 0.1% (w/v) P188) which only formulated with L-Arginine-HCl had lower viscosity (3.9 cP) than other formulations. The excipient L-Methionine is added to this formula to counteract oxidation that may occur in the sample. The SEC-HPLC results demonstrated that the monomer of all tested formulations declined in the range of 5.4%-6.6% after storage at 40° C. for 4 weeks. Among 6 candidates, F1-3 and F1-4 displayed slightly lower monomer than other formulations. The iCIEF data displayed that all samples remained stable under FT and agitation stress, but the main peak decreased by up to 39.8% when stored at 40° C. for 4 weeks. There was no obvious difference among all candidate formulations. Caliper-SDS-NR & R results showed that the purity for all formulations slightly decreased in the NR & R testing when stored at 40° C. for 4 weeks. For DSC, F1-5 showed a slightly higher TmOnset of 52.0° C. than other formulation candidates. Since F1-2 and F1-5 showed less sub-visible particles after 8-week incubation at 25° C., and then were chosen for longer stability evaluation at 5° C. and 25° C. for 3 months. After 3 months storage at 5° C., both samples were stable in the quality attributes of pH, concentration, purities of SEC-HPLC, iCIEF, caliper-SDS-NR & R. For samples stored at 25° C. for 3 months, the pH, concentration, purity of Caliper-SDS-R and ELISA binding affinity remained stable. And the purities of iCIEF, SEC-HPLC and Caliper-SDS-NR reduced by similar levels in both formulations. The difference between the two formulations is the appearance results. F1-2 showed visible particles in 5° C. and 25° C. In summary, F1-2 and F1-5 showed comparable stability during excipients and surfactant screening study. But the P188 content of F1-5 (0.05%) is lower and might be further decreased due to degradation in long-term storage. Thus the protection effect of P188 for the protein was limited. Together, F1-2 (20 mM Phosphate Buffer, 10 mM L-Methionine, 70 mM L-Arginine-HCl, 3.5% (w/v) sucrose, 0.1% (w/v) P188, at pH 7.2) was chosen as the formulation system for 150 mg/mL protein and evaluated in the following study.

Part 2: Formulation Evaluation and Confirmation Study. The objective of this study was to confirm the final selected formulation in the selected container closure system using the drug substance materials. Briefly, the selected formulation was confirmed through a 6 month stability study (−20° C., 2-8° C., 25° C., and 40° C. incubation), agitation at 100 rpm for up to 3 days (D), and freeze/thaw for up to 5 cycles (CYS). Appearance, pH, protein conc., osmolality, Tg′, viscosity, SEC-UPLC, CE-SDS-NR & R, iCIEF, HIAC and potency (ELISA) testing were conducted in this study.

TABLE 3
Sampling and testing plan of formulation confirmation
study (X = Appearance, pH, Protein concentration,
SEC-UPLC, CE-SDS-NR & R, iCIEF; Y = Sub-visible
particles (HIAC); Z = potency (ELISA); W =
Tg′ (mDSC), osmolality, viscosity; ( ) =
Samples reserved, testing optional).

| | | | Sampling Points and Assay | | | |
|---|---|---|---|---|---|---|
| Condition | | T0 | 2 W | 4 W | 12 W | 6 M |
| −20° | C. | X, Y, Z, W | / | X, Y, (Z) | X, Y, (Z) | X, Y, Z |
| 5° | C. | | / | X, Y, (Z) | X, Y, (Z) | X, Y, Z |
| 25° | C. | | / | X, Y, (Z) | X, Y, Z | / |
| 40° | C. | | X, (Z) | X, Y, Z | / | / |

| | | |
|---|---|---|
| Agitation-100 | 1 D | 3 D |
| rpm, 25° C. | X | X, Y, Z |
| Freeze/Thaw - | 3 CYS | 5 CYS |
| 20° C./RT | X | X, Y, Z |

The selected formulation for the protein with the determined filling volume (2 mL) and container closure system (6 R vials and 20 mm nested stopper) was utilized in this formulation confirmation study. All samples remained colorless, slightly opalescent, and free of visible particles in the selected formulation (150 mg/mL protein (target), 20 mM Phosphate Buffer, 10 mM L-Methionine, 3.5% (w/v) sucrose, 70 mM L-Arginine-HCl, 0.1% (w/v) P188, at pH 7.2) for the protein under all testing conditions in this study with the exception of purity measured by CE-SDS-NR & R, and the measured main peak from the SEC-HPLC and iCIEF analysis that declined following incubation at 40° C. for 4 weeks. Furthermore, the testing results also indicated no obvious effect on the stability of the protein under agitation and FT conditions. Overall, the ongoing confirmation study supported the selection of 20 mM phosphate buffer, 10 mM L-Methionine, 3.5% (w/v) sucrose, 70 mM L-Arginine-HCl, 0.1% (w/v) P188, at pH 7.2 as the final formulation for 150 mg/mL antibody protein in the final container closure system.

In conclusion, the excipients and surfactant screening study for 150 mg/mL protein, 6 designed formulations were evaluated in terms of their ability to maintain the protein stability under treatments of thermal stress condition at 25° C. and 40° C., freeze/thaw cycles, and agitation. Finally, 20 mM Phosphate Buffer, 10 mM L-Methionine, 3.5% (w/v) sucrose, 70 mM L-Arginine-HCl, 0.1% (w/v) P188, at pH 7.2, was recommended as the final formulation for 150 mg/mL protein. And the potency remained stable under thermal stress in this selected formulation. In the confirmation study, no obvious differences in appearance, protein conc., pH and sub-visible particles were observed under −20° C., 2-8° C., 25° C., agitation and freeze/thaw stress conditions compared to T0. While the purity data from SEC-UPLC, iCIEF and CE-SDS-NR & R declined following incubation at 40° C. for 4 weeks.

Example 2: Low concentration formulation development. The design of the formulation development study consisted of four parts: 1) pH/buffer screening study under stress conditions to select the optimal pH/buffer system which provided the most stabilization for the protein, 2) excipient and surfactant strength screening study for 50 mg/mL protein was performed to evaluate and select appropriate excipients and surfactants according to their stabilizing effect on the protein under stress conditions, 3) excipient and surfactant strength screening study for 100 mg/mL protein was conducted to evaluate its stability under stress conditions and to select an optimal formulation according to the stability results, and 4) selected formulation of the protein was evaluated and confirmed through a 3-month stability study under various conditions. Biophysical and analytical methods including appearance, pH, protein concentration, SE-UPLC, iCIEF, CE-SDS-NR &R, mDSC, sub-visible particles, and potency testing were used to determine the potential chemical and physical changes that might occur to formulated protein under these conditions. These methods were conducted as described previously in example 1 with the exception of the below parameters:

Protein concentration was determined at the absorbance at 280 nm using a Thermo UV (280 nm) (model: Nanodrop 2000) spectrophotometer. The extinction coefficient (E1%) used in all evaluation studies was 1.53 AU*mL*mg-1*cm-1. All samples were performed without any dilution and measurements were repeated twice with 2.5 μL of sample for each sample and an average result was reported.

For reduced peptide mapping experiment, the protein was denatured with guanidine hydrochloride (Gdn-HCl) in Tris-HCl buffer, reduced by dithiothreitol (DTT), and followed by cysteine-alkylation with Iodoacetamide (IAM). After sample clean-up, the protein was digested with endoproteinase Lys-C/trypsin sequential digestion to obtain peptides suitable for subsequent analysis. The online LC-MS analysis was performed on an Agilent/UHPLC 1290 system coupled to an Orbitrap mass spectrometer. The proteolytic peptides were separated by reversed phase liquid chromatography with an Agilent/Poroshell SB-C18 column, UV chromatograms were acquired with detector set at wavelength of 214 nm. The peptides were further detected by the mass spectrometer operating with full MS scan followed by tandem mass spectrometry (MS/MS) scans. Extracted ion chromatograms (EIC) ratio was reported to semi-quantitatively indicate the ratio of PTMs.

Part 1: pH/buffer screening study. The objective of this screening study was to select the optimal pH/buffer system to stabilize the protein most under stress conditions. Thus, eight pH/buffer systems under stress conditions (40° C. for up to 4 weeks (W)) were investigated in the pH/buffer screening study, and appearance, pH, protein conc., SEC-HPLC, iCIEF, DSC and caliper-SDS-NR & R were tested. Candidates and conditions tested are shown in table 4 below.

TABLE 4
Formulation candidates list of pH/buffer screening study.

| Formulation No. | Buffer System | Target pH |
|---|---|---|
| F2-1 | 20 mM acetate | 5.0 |
| F2-2 | | 5.5 |
| F2-3 | 20 mM histidine | 5.5 |
| F2-4 | | 7.0 |
| F2-5 | 20 mM phosphate | 7.0 |
| F2-6 | | 7.5 |
| F2-7 | 20 mM Tris | 7.0 |
| F2-8 | | 7.5 |

In summary, the pH/buffer screening study investigated 8 pH/buffer systems under stress conditions (40° C. for up to 4 weeks), and the results showed that the stability of the antibody was closely related with both pH and buffer type. From the appearance results, F2-1 (acetate, pH 5.0), F2-5 (PB3, pH 7.0) and F2-6 (PB, pH 7.5) performed better than the other formulations. No obvious pH or protein concentration changes were observed in all tested buffer systems. The SEC-HPLC results indicated F2-5 (PB, pH 7.0) and F2-6 (PB, pH 7.5) supported higher purity when the protein was stored at 40° C. for 4 weeks. The iCIEF results indicated that, the main peak of F12-3 (histidine buffer, pH 5.5), F2-2 (acetate buffer, pH 5.5), and F2-1 (acetate buffer, pH 5.0) was higher than other candidates. The caliper-SDS-NR & R results showed no significant difference in purity among all formulations. Taking all results into consideration, samples in F2-2, F2-5 and F2-6 were selected to test by ELISA. The results showed that EC50 was comparable to the value at TO after incubation at 40° C. for 4 weeks, indicating the stable potency of the antibody. In summary, the antibody protein showed better appearance and higher purity tested by SEC-HPLC, indicating higher physical stability in 20 mM PB buffer. Though the iCIEF data displayed slightly lower chemical stability in PB buffer, the potency remained stable under thermal stress. Subsequently, 20 mM PB buffer was recommended for the antibody. For the sake of having buffer capacity of pH range, the buffer systems of 20 mM PB at pH 7.2 was selected for the excipients and surfactants screening study.

Part 2: Excipients/Surfactant strength screening study I (50 mg/mL). The objective of this study was to identify the optimal excipient and surfactant that would help to maximize the stability of the 50 mg/mL protein, and was tested under stress conditions. Thus, for 50 mg/mL antibody, different types of excipients (sucrose, trehalose dihydrate, sorbitol, etc.), various conc. of L-Arg-HCl (70 mM and 140 mM) and various conc. of PS80 (0.02%. (w/v) and 0.04%7 (w/v)), PS20 (0.02, w/v) and P88 (0, w/v) were evaluated under stress conditions (40° C. for up to 4 weeks (W), agitation at 300 rpm for 3 days (D), and freeze/thaw for up to 5 cycles (CYS). Appearance, pH, protein conc., osmolality, SE-HPLC, caliper-SDS-NR & R, iCIEF, MFI, DSC and potency (ELISA) testing were performed in this study. Candidates and conditions tested are shown in table 5 below.

TABLE 5
Formulation Candidates list of excipients and surfactant strength
screening study I (all % in table means % (w/v)).

| Formulation | Buffer System, pH, | |
|---|---|---|
| No. | Protein Conc. | Excipients/Surfactant |
| F3-1 | 20 mM PB, 10 mM | 8% sucrose, 0.02% PS80 |
| F3-2 | L-Methionine, pH | 8.8% trehalose 2H₂O, |
| | 7.2, 50 mg/mL Protein | 0.02% PS80 |
| F3-3 | | 4.5% sorbitol, 0.02% PS80 |
| F3-4 | | 70 mM L-Arg-HCl, 4% sucrose, |
| | | 0.02% PS80 |
| F3-5 | | 70 mM NaCl, 4% sucrose, |
| | | 0.02% PS80 |
| F3-6 | | 140 mM L-Arg-HCl, 0.02% PS80 |
| F3-7 | | 8% sucrose, 0.04% PS80 |
| F3-8 | | 8% sucrose, 0.02% PS20 |
| F3-9 | | 8% sucrose, 0.1% P188 |
| F3-10 | | 8% sucrose, 0.02% PS80, |
| | | 0.1% P188 |

In summary, Excipients and surfactants strength screening study I was conducted with 50 mg/mL protein. In this study, F3-9 (8% (w/v) sucrose and 0.1% (w/v) P188) showed a better appearance and less sub-visible particles than other formulations after 4-week storage at 40° C. P188 might be a better surfactant than PS20 and PS80 to inhibit particle formation. The SEC-HPLC results demonstrated that the monomer of all tested formulations declined by less than 5%. The iCIEF data displayed that all samples remained stable under FT and agitation stress, but the main peak decreased by up to 38.2% when stored at 40° C. for 4 weeks. There was no obvious difference among all candidate formulations. Caliper-SDS-NR & R results showed that the purity for all formulations slightly decreased in the NR testing and remained stable in the R testing when stored at 40° C. for 4 weeks. For DSC, F3-3 showed a slightly higher TmOnset of 56.9° C. than other formulation candidates. In summary, F3-9 (20 mM PB, 10 mM Met, 8% (w/v) sucrose, 0.1% (w/v) P188, at pH 7.2), as well as F3-4 and F3-6 the formulations with L-Arg-HCl, were chosen as the formulation systems for 100 mg/mL protein formulation development and the formulation containing P188 and L-Arg-HCl with sucrose was also chosen to be evaluated in the following study. Though F3-4 and F3-6 performed poor in appearance and sub-visible particle tests under thermal stress, L-Arg-HCl may reduce the viscosity at high concentration. And whether PS80 was suitable for the antibody was further evaluated.

Part 3: Excipients/Surfactant strength screening study II (100 mg/mL). The objective of this study was to identify the optimal excipients that stabilize the antibody at a higher concentration (100 mg/mL) under stress conditions. Thus, for 100 mg/mL antibody, different conc. of sucrose (7% (w/v) and

4% (w/v)), various conc. of L-Arg-HCl (70 mM and 140 mM), 0.02% PS80 and 0.1% P188 were evaluated under stress conditions (40° C. for up to 4 weeks, agitation at 300 rpm for 3 days (D), and freeze/thaw for up to 5 cycles (CYS). Appearance, pH, protein conc., osmolality, SEC-HPLC, caliper-SDS-NR & R, iCIEF, MFI, DSC and potency (ELISA) testing were performed in this study. Candidates and conditions tested are shown in table 6 below.

TABLE 6
Formulation candidates list of excipients and surfactant strength screening study
(all % in the table means % (w/v); * to maintain the osmolality close to 300 mOsm/kg,
the sucrose conc was reduced to 7%, compared to F3-9 with 8% sucrose).

| Formulation | Buffer System, pH, Protein | |
|---|---|---|
| No. | Conc. | Excipients/Surfactant |
| F3-11 | 20 mM PB, 10 mM L-Methionine, | 7% sucrose*, 0.1% P188 |
| F3-12 | pH 7.2, 100 mg/mL protein | 70 mM L-Arg-HCl, 4% sucrose, |
| | | 0.02% PS80 |
| F3-13 | | 140 mM L-Arg-HCl, 0.02% PS80 |
| F3-14 | | 70 mM L-Arg-HCl, 4% sucrose, |
| | | 0.1% P188 |

In summary, In the Excipients and Surfactant Strength Screening Study II, the pH value and protein conc. of all formulations remained stable. Appearance and MFI analysis showed that F11 (7% (w/v) sucrose, 0.1% (w/v) P188) and F3-14 (4% (w/v) sucrose, 70 mM L-Arg-HCl, 0.1% (w/v) P188 exhibited less particles than other formulations after a 4-week storage at 40° C. In this study, formulations containing P188 showed better appearance and fewer sub-visible particles than ones containing PS80 but it is uncertain that the protection of P188 or the degradation of PS80 can be attributed to this result. Based on the current result, P188 was more suitable for the antibody molecule. Although F3-11 possessed a higher TmOnset (57.0° C.), in the 4 tested formulations, it also presented a higher viscosity of 8.7 cP, which might bring difficulties for the production of high concentration DS. The 7% (w/v) sucrose might contribute to the elevated viscosity. The SEC-HPLC results demonstrated the monomer of all tested formulations had a slightly higher decline than 5% and there were no significant differences of monomer among 4 formulations. The iCIEF showed all samples remained stable under FT and agitation stress but the main peak decreased when stored at 40° C. for 4 weeks. The decline ratio of main peak among all tested formulations was consistent. Caliper-SDS-NR & R results displayed that the purity for all formulations slightly decreased in the NR & R testing when stored at 40° C. for 4 weeks. In conclusion, P188 was better to reduce particle numbers, while lower sucrose concentration and L-Arg-HCl was necessary to reduce viscosity. Therefore, F3-14 (20 mM PB, 10 mM L-Methionine, 4% (w/v) sucrose, 70 mM L-Arg-HCl, 0.1% (w/v) P188, at pH 7.2), was chosen as the final buffer and excipient system for the antibody formulation. The protein showed an 8% reduction in potency following 4 weeks at 40° C. in this final formulation.

Part 4: Formulation confirmation study. The objective of this study was to confirm the final selected formulation in the selected container closure system using the processed locked materials. Thus, the selected formulation was confirmed through a 3-month stability study (−70° C., 2-8° C., 25° C., and 40° C. incubation), agitation at 100 rpm for up to 3 days (D), and freeze/thaw for up to 5 cycles (CYS). Appearance, pH, protein conc., osmolality, viscosity, SEC-UPLC, CE-SDS-NR & R, iCIEF, HIAC and potency (ELISA) testing were conducted in this study. Table 7 below shows the sampling and testing plan for the formulation confirmation study.

TABLE 7
Sampling and testing plan of formulation confirmation
study (X = Appearance, pH, protein concentration,
SEC-UPLC, CE-SDS-NR & R, iCIEF; Y = Sub-visible
particles (HIAC); Z = potency (ELISA); W =
Tg' (mDSC), osmolality, viscosity; ( ) =
Samples reserved, testing optional).

| | | Sampling Points and Assay | | |
|---|---|---|---|---|
| Condition | T0 | 2 W | 4 W | 12 W |
| −70° C. | X, Y, Z, W | / | X, Y, (Z) | X, Y, (Z) |
| 5° C. | | / | X, Y, (Z) | X, Y, (Z) |
| 25° C. | | / | X, Y, (Z) | X, Y, Z |
| 40° C. | | X, (Z) | X, Y, Z | / |
| Agitation- 100 | | 1 D | | 3 D |
| rpm, 25° C. | | X | | X, Y |
| Freeze/Thaw- | | 3 CYS | | 5 CYS |
| 70° C./RT | | x | | X, Y, Z |

In Summary, the selected formulation for the antibody protein with the determined filling volume (2 mL) and container closure system (6 mL vials, 20 mm stopper and 20 mm aluminum-plastic cover) was utilized in this formulation confirmation study. All samples remained colorless, slightly opalescent, and free of visible particles in the selected formulation (100 mg/mL protein (target), 20 mM PB, 10 mM L-Methionine, 400 (w/v) sucrose, 70 mM L-Arginine-HCl, 0.100 (w/v) P188, at pH 7.2) for protein under all testing conditions in this study, except for samples stored at 25° C. for 9 weeks. The purity measured by CE-SDS-NR & R, and the measured the proportion of main peak from the SEC-HPLC and iCIEF analysis declined following incubation at 40° C. for 4 weeks. Furthermore, the testing results indicated no obvious effects on the stability of the protein under agitation and FT conditions. Overall, the confirmation study supported the selection of 20 mM PB, 10 mM L-Methionine, 4% (w/v) sucrose, 70 mM L-Arg-HCl, 0.1% (w/v) P188, at pH 7.2 as the final formulation for 100 mg/mL protein in the final container closure system. As for the long-term storage temperature, the temperature of −20° C. is implemented into the stability study of DP from engineering batch, since visible particles were observed at 25° C.

In conclusion, In the pH/buffer screening study, 8 formulations designed from 4 buffer systems were evaluated under the accelerated thermal condition of 40° C. Based on the results obtained from appearance, pH, protein conc. SEC-HPLC, caliper-SDS-NR & R, iCIEF, and DSC, 20 mM PB with 10 mM L-Methionine buffer systems at pH 7.2 were selected for the excipients and surfactant screening study. Excipients and surfactant screening study for 50 mg/mL protein was performed first. In this study, 10 designed formulations (F3-1-F3-10) were evaluated in terms of their ability to maintain the protein stability under treatments of thermal stress condition at 25° C. and 40° C., freeze/thaw cycles, and agitation. Based on the results obtained from appearance, pH and protein conc. measurements, SEC-HPLC, caliper-SDS-NR & R, iCIEF, MFI and DSC, 20 mM PB, 10 mM L-Methionine, 8% (w/v) sucrose, 0.1% (w/v) P188, at pH 7.2, was chosen as the base formulation for high concentration antibody formulation development. In the excipients and surfactant screening study for 100 mg/mL protein, 4 designed formulations (F3-11-F3-14) were evaluated in terms of their ability to maintain the protein stability under treatments of thermal stress condition at 25° C. and 40° C., freeze/thaw cycles, and agitation. Based on the results obtained from appearance, pH and protein conc., SEC-HPLC, caliper-SDS-NdR & R, iCIEF, MFI and DSC, 20 mM PB, 10 mM L-Methionine, 4% (w/v) sucrose, 70 mM L-Arg-HCl, 0.1% (w/v) P188, at pH 7.2, was recommended as the final formulation for 100 mg/mL protein and the potency remained stable under thermal stress in this selected formulation. In the confirmation study, no obvious differences in appearance, protein conc., pH and sub-visible particles were observed under 2-8° C., −70° C., agitation and freeze/thaw stress conditions compared to T0. It was observed that purity data from SE-UPLC, iCIEF and CE-SDS-NR & R declined following incubation at 40° C. for 4 weeks and the visible particles were observed in samples stored at 25° C. for 9 weeks. The particulate trend will be further monitored in other DP stability studies. And −20° C. is recommended as the long-term storage temperature of DP in the early stage.

Overall, 20 mM PB, 10 mM L-Methionine, 4% (w/v) sucrose, 70 mM L-Arg-HCl, 0.1% (w/v) P188, at pH 7.2 was recommended as the final formulation for 100 mg/mL antibody development.

Example 3: Formulation stability determination. The antibody was stored as a liquid, filled in glass vials to allow 2.0 mL to be extracted. Formulation testing lot 1 (process 1) comprised 100 mg/mL of the antibody, 20 mM phosphate buffer, 10 mM L-methionine, 70 mM L-arginine monohydrochloride, 4% (w/v) sucrose, 0.1% (w/v) poloxamer 188, and a pH 7.2. Formulation testing lot 2 (process 2) comprised 150 mg/mL of the antibody, 20 mM phosphate buffer, 10 mM L-methionine, 70 mM L-arginine monohydrochloride, 3.5% (w/v) sucrose, 0.1% (w/v) poloxamer 188, at a pH 7.2. Process 1 was stored using a 6 mL Type I glass vials, 20 mm chlorobutyl rubber stopper, and 20 mm Aluminum-plastic cap. Process 2 was stored using 6 mL Type I glass vials, 20 mm chlorobutyl rubber stopper nested in 20 mm polypropylene cap; or 6 mL Type I glass injection vials, made of glass tubing 20 mm chlorobutyl rubber stopper, and 20 mm Aluminum-plastic flip cap.

The presence of visible particles, sub-visible particulate matter, color, clarity, pH, protein concentration, iCIEF, SEC, CE-SDS (Reduced), CE-SDS (Non-reduced), ELISA binding, sterility, and container closure system integrity testing (CCIT), were measured. Sterility was performed at TO and at the end point of the stability study. CCIT was performed annually and at the end point of the stability study at the long-term stability condition. Starting with the DP lot 20231002, poloxamer 188 concentration was also monitored for the antibody lots on stability every 6 months for up to 24 months, and then at the annual time points. Analytical methods were as according to methods described in Example 1 and Example 2 above.

Container and Closure System Integrity Testing in Lieu of Sterility Testing as a Component of the Stability Protocol for Sterile Products (CCIT) was used as a substitute for sterility testing in drug product stability research to verify the package integrity based on USP. A vacuum decay leak testing, which is a nondestructive method, was used for stability studies. The container closure system was enclosed in a chamber that had a defined vacuum setting applied within a given time interval. The vacuum extracted headspace gas or liquid vapor content from the interior of the sample. This caused an increase in pressure of the enclosed test chamber. Vacuum decay measured this pressure increase (or vacuum decay), which needed to be above a threshold value to represent a leak. The result was reported as “pass” or “fail”.

Stability conditions and testing frequency are listed in Tables 64-68 below.

TABLE 64
Stability Conditions and Testing Time Points

| | | | |
|---|---|---|---|
| | Stability | | |
| Lot No. | Type | Stability Condition | Time Points (Month) ^{a} |

DP Lots (100 mg/mL & 150 mg/mL) monitored under Legacy Stability
Panel and the Stability Conditions

| | | | |
|---|---|---|---|
| 20220502 ^{b} | Long Term | −20 ± 5° C., Upright | 0, 1, 3, 6, 9, 12, 18, 24 |
| | Accelerated | 5 ± 3° C., Inverted | 0, 1, 3, 6, 9, 12, 18, 24 |
| | Stress | 25 ± 2° C. / 60 ± 5% RH, Inverted | 0, 1, 3, 6 |
| | Stress | 40 ± 2° C. / 75 ± 5% RH, Inverted | 0, 1 W, 2 W, 1 |
| 20220902 ^{b} | Long Term | −20 ± 5° C., Upright | 0, 1, 3, 6, 9, 12, 18, 24 |
| | Long Term | 5 ± 3° C., Inverted | 0, 1, 3, 6, 9, 12, 18 |
| | Accelerated | 25 ± 2° C. / 60% ± 5% RH, Inverted | 0, 1, 3, 6 |
| 20230201 ^{b} | Long Term | −20 ± 5° C., Upright | 0, 1, 3, 6, 9, 12, 18, 24 |
| | Long Term | 5 ± 3° C., Inverted | 0, 1, 3, 6, 9, 12, 18, 24 |
| | Long Term | 5 ± 3° C., Upright | 0, 1, 3, 6, 9, 12 |
| | Accelerated | 25 ± 2° C. / 60 ± 5% RH, Inverted | 0, 1, 3, 6, 9 |
| | Stress | 40 ± 2° C. / 75 ± 5% RH, Inverted | 0, 1 W, 2 W, 1 |

DP Lots (150 mg/mL ONLY) monitored under Current Stability
Testing Panel, and the Stability Conditions

| | | | |
|---|---|---|---|
| 20230603 ^{c} | Long Term | −20 ± 5° C., Upright | 0, 1, 3 |
| | Long Term | 5 ± 3° C., Inverted | 0, 1, 3, 6, 9, 12, 18, 24, 36 |
| | Long Term | 5 ± 3° C., Upright | 0, 1, 3, 6, 9, 12, 18, 24 |
| | Accelerated | 25 ± 2° C. / 60 ± 5% RH, Inverted | 0, 1, 3, 6 |
| | Stress | 40 ± 2° C. / 75 ± 5% RH, Inverted | 0, 1 W, 2 W, 1 |
| 20240603 | Accelerated | 25 ± 2° C. / 60 ± 5% RH, Inverted | 0, 1 |
| | Stress | 40 ± 2° C. / 75 ± 5% RH, Inverted | 0, 1 W, 2 W, 1 |
| | Long Term | 5 ± 3° C., Inverted | 0, 1 |
| | Long Term | 5 ± 3° C., Upright | 0, 1 |
| 20231002 | Long-Term | 5° C. ± 3° C., Inverted | 0, 1, 3, 6, 9, 12, 18, 24, 36, 39 d |
| | Long-Term | 5° C. ± 3° C., Upright | 0, 1, 3, 6, 9, 12, 18, 24 |
| | Accelerated | 25° C. ± 2° C. / 60 ± 5% RH, Inverted | 0, 1, 3, 6 |
| | Stress | 40° C. ± 2° C. / 75 ± 5% RH, Inverted | 0, 1 W, 2 W, 1 |
| 20240302 | Long-Term | 5° C. ± 3° C., Inverted | 0, 1, 3, 6, 9, 12, 18, 24, 36, 39 d |
| | Long-Term | 5° C. ± 3° C., Upright | 0, 1, 3, 6, 9, 12, 18, 24 |
| | Accelerated | 25° C. ± 2° C. / 60 ± 5% RH, Inverted | 0, 1, 3, 6 |
| | Stress | 40° C. ± 2° C. / 75 ± 5% RH, Inverted | 0, 1 W, 2 W, 1 |

Abbreviations:
RH = relative humidity;
W = week;
^{a} The timepoints are in months unless otherwise noted;
^{b} Long-term storage condition remained at −20 ± 5° C. condition for this batch, irrespective of the manufacturing process (Process 1 or Process 2);
^{c} Long-term storage condition for the DP lot 20230603 was revised to 5 ± 3° C. from the legacy storage condition −20 ± 5° C.
This change in long-term storage temperature was supported by real-time stability data on the DP lot at 5 ± 3° C.;
^{d} A 39-month timepoint was added to demonstrate the stability of the product beyond 36 months.

TABLE 65
Legacy Stability Testing Panel for MAB39 Drug Product for 24 Months Long-Term Stability

| Stability Condition: 5° C. ± 3° C. (Inverted)^{a} | | | | | | | | | |
|---|---|---|---|---|---|---|---|---|---|
| and −20° C. ± 5° C. (Upright) | | Testing Timepoint (Month) | | | | | | | |
| Test Item | Test Method | 0 | 1 | 3 | 6 | 9 | 12 | 18 | 24 |
| Visible particles | USP | X | X | X | X | X | X | X | X |
| Appearance (Color) | USP | X | X | X | X | X | X | X | X |
| Appearance (Clarity) | USP | X | X | X | X | X | X | X | X |
| pH | USP | X | X | X | X | X | X | X | X |
| Subvisible Particulate Matter | USP; Light obscuration | X | X | X | X | X | X | X | X |
| Protein Concentration | UV | X | X | X | X | X | X | X | X |
| Purity | iCIEF (Charge Variants) | X | X | X | X | X | X | X | X |
| Purity | CE-SDS (Reduced) | X | X | X | X | X | X | X | X |
| Purity | CE-SDS (Non-reduced) | X | X | X | X | X | X | X | X |
| Purity | SEC | X | X | X | X | X | X | X | X |
| Potency | ELISA Binding (Bioasssay) | X | X | X | X | X | X | X | X |
| Sterility | USP; Membrane filtration | X | NT | NT | NT | NT | NT | NT | X |
| Container Closure Integrity | USP; Vacuum Decay Leak Test | NT | NT | NT | NT | NT | X | NT | X |
| (CCIT) | | | | | | | | | |

Abbreviations: CE-SDS = capillary electrophoresis sodium dodecyl sulfate; iCIEF = imaged capillary isoelectric focusing; NT = not tested; SEC = size exclusion chromatography; X = tested;
^{a}Upright condition at 5° C. ± 3° C. is being evaluated for up to 12 months. Applicable for Lots 20220502 (100 m/mL), and 150 mg/mL DP lots 20220902, 20220903, and 20230201.

TABLE 66
Current Stability Testing Panel for MAB39 Drug Product for 36 Months Long-Term Stability

| Stability Stability Condition: | | | | | | | | | | | |
|---|---|---|---|---|---|---|---|---|---|---|---|
| 5° C. ± 3° C. (Inverted or Upright^{a}) | | Testing Timepoint (Month) | | | | | | | | | |
| Test Item | Test Method | 0 | 1 | 3 | 6 | 9 | 12 | 18 | 24 | 36 | 39^{b} |
| Visible particles | USP | X | X | X | X | X | X | X | X | X | X |
| Appearance (Color) | USP | X | X | X | X | X | X | X | X | X | X |
| Appearance (Clarity) | USP | X | X | X | X | X | X | X | X | X | X |
| pH | USP | X | X | X | X | X | X | X | X | X | X |
| Subvisible Particulate Matter ^{c} | USP; Light obscuration | X | X | X | X | X | X | X | X | X | X |
| Protein Concentration | UV | X | X | X | X | X | X | X | X | X | X |
| Poloxamer 188 Content ^{d} | UPLC-ELSD | X | NT | NT | X | NT | X | X | X | X | X |
| Purity | iCIEF (Charge Variants) | X | X | X | X | X | X | X | X | X | X |
| Purity | CE-SDS (Reduced) | X | X | X | X | X | X | X | X | X | X |
| Purity | CE-SDS (Non-reduced) | X | X | X | X | X | X | X | X | X | X |
| Purity | SEC | X | X | X | X | X | X | X | X | X | X |
| Potency | ELISA Binding | X | X | X | X | X | X | X | X | X | X |
| | (Bioasssay) | | | | | | | | | | |
| Sterility | USP; Membrane filtration | X | NT | NT | NT | NT | NT | NT | NT | X | X |
| Container Closure Integrity | USP; Vacuum Decay Leak | NT | NT | NT | NT | NT | X | NT | X | X | X |
| (CCIT) | Test | | | | | | | | | | |

Abbreviations: CE-SDS = capillary electrophoresis sodium dodecyl sulfate; iCIEF = imaged capillary isoelectric focusing; NT = not tested; SEC = size exclusion chromatography; X = tested;
^{a}Upright condition was evaluated for up to 24 months;
^{b}Tested for DP lot 20231002 only at 5° C. ± 3° C. (inverted) condition to evaluate stability of the DP beyond target shelf-life of 36 months;
^{c} For the upright condition (5 ± 3° C.), sub-visible particulate was performed at the yearly timepoints. For the inverted, testing was performed at every timepoint for all lots, except for DP Lot 20230201 where all timepoints at 5° C. were tested for sub-visible particulates (both upright and inverted);
^{d} Added to all lots with long-term stability (5° C. ± 3° C. (inverted)) and accelerated (25° C. ± 2° C.) conditions. P188 was tested for all future DP lots on stability at 6 mo time points until 24 mo, then at annual timepoints only.

TABLE 67
Stability Testing Panel for MAB39 Drug Product for Accelerated Condition

| | | Testing | | | | |
|---|---|---|---|---|---|---|
| Stability Condition: 25° C. ± 2° C. / 60 ± 5% RH (Inverted) | | Timepoint (Month) | | | | |
| Test Item | Test Method | 0 | 1 | 3 | 6 | 9 |
| Visible particles | USP | X | X | X | X | X |
| Appearance (Color) | USP | X | X | X | X | X |
| Appearance (Clarity) | USP | X | X | X | X | X |
| pH | USP | X | X | X | X | X |
| Subvisible Particulate Matter | USP; Light obscuration | X | X | X | X | X |
| Protein Concentration | UV | X | X | X | X | X |
| Poloxamer 188 Content | UPLC-ELSD | X | NT | X | X | NT |
| Purity | iCIEF (Charge Variants) | X | X | X | X | X |
| Purity | CE-SDS (Reduced) | X | X | X | X | X |
| Purity | CE-SDS (Non-reduced) | X | X | X | X | X |
| Purity | SEC | X | X | X | X | X |
| Potency | ELISA Binding (Bioasssay) | X | X | X | X | X |

Abbreviations: CE-SDS-capillary electrophoresis sodium dodecyl sulfate; iCIEF = imaged capillary isoelectric focusing; NT = not tested; SEC = size exclusion chromatography; X = tested.

TABLE 68
Stability Testing Pannel for MAB39 Drug Product for Stress Condition

| Stability Condition: | | | | | |
|---|---|---|---|---|---|
| 40° C. ± 2° C./75 ± 5% RH (Inverted) | | Testing Timepoint | | | |
| Test Item | Test Method | 0 | 1 W | 2 W | 1 M |
| Visible particles | USP | X | X | X | X |
| Appearance (Color) | USP | X | X | X | X |
| Appearance (Clarity) | USP | X | X | X | X |
| pH | USP | X | X | X | X |
| Subvisible Particulate Matter | USP; Light obscuration | X | X | X | X |
| Protein Concentration | UV | X | X | X | X |
| Purity | iCIEF (Charge Variants) | X | X | X | X |
| Purity | CE-SDS (Reduced) | X | X | X | X |
| Purity | CE-SDS (Non-reduced) | X | X | X | X |
| Purity | SEC | X | X | X | X |
| Potency | ELISA Binding | X | X | X | X |
| | (Bioasssay) | | | | |

Abbreviations: CE-SDS = capillary electrophoresis sodium dodecyl sulfate; iCIEF = imaged capillary isoelectric focusing; SEC = size exclusion chromatography; M = month; W = week, X = tested.

Stability study results support long-term storage conditions of 5° C.±3° C. and −20° C.±5° C. for the antibody formulation. Data for Process 1 (100 mg/mL), stored at −20° C. as a long-term stability condition, demonstrates no significant change over time and no significant variability. Stability results met all acceptance criteria through the 18-month time point. The Process 2 drug product (150 mg/mL) was initially evaluated at −20° C. as a risk mitigation storage condition pending availability of additional stability data at 5 C. As the stability data became available, it was concluded that Process 2 drug product stored at either −20° C. or 5° C. and demonstrates no significant change over time and no significant variability. Stability results meet all acceptance criteria through the 18-month time point.

In a separate experiment, the containers used for the stability studies were made of polycarbonate (PC), which was the same material used for the drug substance container closure system. Formulation testing lot 1 (process 1) comprised 100 mg/mL of the antibody, 20 mM phosphate buffer, 10 mM L-methionine, 70 mM L-arginine monohydrochloride, 4% (w/v) sucrose, 0.1% (w/v) poloxamer 188, and a pH 7.2. Formulation testing lot 2 (process 2) comprised 150 mg/mL of the antibody, 20 mM phosphate buffer, 10 mM L-methionine, 70 mM L-arginine monohydrochloride, 3.5% (w/v) sucrose, 0.1% (w/v) poloxamer 188, at a pH 7.2. All formulations were stored in 5 mL PC bottles.

The quality attributes monitored include color, clarity, pH, protein concentration, iCIEF, CE-SDS (Reduced), CE-SDS (Non-reduced), SEC, and ELISA Binding. Analytical methods were as according to methods described in Example 1 and Example 2 above.

Stability conditions and testing frequency are listed in Tables 8-12 below.

TABLE 8
Stability Conditions and Testing Time Points for MAB39 DS

| | | | | |
|---|---|---|---|---|
| | | | | Available |
| | | | | Stability |
| | Stability | Stability | | Data |
| Batch No. | Type | Condition | Time Points (Month) | (Months) |

100 mg/mL DS

| | | | | |
|---|---|---|---|---|
| 2441S211215Y | Long Term | −70° C. ± 10° C. | 0, 1, 3, 6, 9, 12, 18, 24, 36 | 24 |
| | Accelerated | 5° C. ± 3° C. | 0, 1, 3, 6 | 6 (completed) |
| | Stress | 25° C. ± 2° C./60 ± 5% RH | 0, 1, 2, 3 | 3 (completed) |
| 24412201 | Long Term | −70° C. ± 10° C. | 0, 1, 3, 6, 9, 12, 18, 24, 36 | 18 |
| | Accelerated | 5° C. ± 3° C. | 0, 1, 3, 6 | 6 (completed) |
| | Stress | 25° C. ± 2° C./60 ± 5% RH | 0, 1, 2, 3 | 3 (completed) |

150 mg/mL DS

| | | | | |
|---|---|---|---|---|
| 2441S220510Y | Long Term | −70° C. ± 10° C. | 0, 1, 3, 6, 9, 12, 18, 24 | 18 |
| | Accelerated | 5° C. ± 3° C. | 0, 1, 3, 6 | 6 (completed) |
| | Stress | 25° C. ± 2° C./60 ± 5% RH | 0, 1, 2, 3 | 3 (completed) |
| 24412202 | Long Term | −70° C. ± 10° C. | 0, 1, 3, 6, 9, 12, 18, 24, 36, 39a | 18 |
| | Accelerated | 5° C. ± 3° C. | 0, 1, 3, 6 | 6 (completed) |
| | Stress | 25° C. ± 2° C./60 ± 5% RH | 0, 1, 2, 3 | 3 (completed) |
| 0000097275 | Long Term | −70° C. ± 10° C. | 0, 1, 3, 6, 9, 12, 18, 24, 36 | 9 |
| | Accelerated | 5° C. ± 3° C. | 0, 1, 3, 6, 9, 12 | 6 |
| | Stress | 25° C. ± 2° C./60 ± 5% RH | 0, 1, 2, 3, 6 | 6 (completed) |
| 0000114279 | Long Term | −70° C. ± 10° C. | 0, 1, 3, 6, 9, 12, 18, 24, 36, 39a | 3 |
| | Accelerated | 5° C. ± 3° C. | 0, 1, 3, 6, 9, 12 | 3 |
| | Stress | 25° C. ± 2° C./60 ± 5% RH | 0, 1, 2, 3, 6 | 3 |
| 0000124360 | Long Term | −70° C. ± 10° C. | 0, 1, 3, 6, 9, 12, 18, 24, 36, 39a | 1 |
| | Accelerated | 5° C. ± 3° C. | 0, 1, 3, 6, 9, 12 | 1 |
| | Stress | 25° C. ± 2° C./60 ± 5% RH | 0, 1, 2, 3, 6 | 1 |

^{a}A 39- month timepoint was added for 3 batches only, to demonstrate the stability of the DS beyond the maximum shelf-life (36 months).

TABLE 9
Long-term Stability Testing Plan for MAB39 100 mg/mL Drug Substance

| Stability Condition: −70° C. ± 10° C. | | Testing Timepoint (Month) | | | | | | | | |
|---|---|---|---|---|---|---|---|---|---|---|
| Attribute | Test Method | 0 | 1 | 3 | 6 | 9 | 12 | 18 | 24 | 36^{a} |
| Appearance (Color) | USP | X | X | X | X | X | X | X | X | X |
| Appearance (Clarity) | USP | X | X | X | X | X | X | X | X | X |
| pH | USP | X | X | X | X | X | X | X | X | X |
| Protein | UV | X | X | X | X | X | X | X | X | X |
| Concentration | | | | | | | | | | |
| Main Peak | Charge Variants | X | X | X | X | X | X | X | X | X |
| Acidic Peaks | by iCIEF | X | X | X | X | X | X | X | X | X |
| Basic Peaks | | X | X | X | X | X | X | X | X | X |
| LC + HC | Purity by | X | X | X | X | X | X | X | X | X |
| Total Minor Species | CE-SDS (Reduced) | X | X | X | X | X | X | X | X | X |
| Main Peak | Purity by CE-SDS | X | X | X | X | X | X | X | X | X |
| Total LMWS | (Non-reduced) | X | X | X | X | X | X | X | X | X |
| Monomer (Main Peak) | Purity by SEC | X | X | X | X | X | X | X | X | X |
| HMWS | | X | X | X | X | X | X | X | X | X |
| Binding Bioassay | ELISA | X | X | X | X | X | X | X | X | X |

Abbreviations: CE-SDS = capillary electrophoresis; HMWS = high molecular weight species; iCIEF = imaged capillary isoelectric focusing; LC + HC = Light Chain + Heavy Chain; LMWS = high molecular weight species; SEC = size exclusion chromatography.
^{a}The long-term stability plan for batch 2441S220510Y ends at 24 months.

TABLE 10
Long-term Stability Testing Plan for MAB39 150 mg/mL Drug Substance

| Stability Condition: −70° C. ± 10° C. | | Testing Timepoint (Month) | | | | | | | | | |
|---|---|---|---|---|---|---|---|---|---|---|---|
| Attribute | Test Method | 0 | 1 | 3 | 6 | 9 | 12 | 18 | 24 | 36 | 39^{a} |
| Appearance (Color) | USP | X | X | X | X | X | X | X | X | X | X |
| Appearance (Clarity) | USP | X | X | X | X | X | X | X | X | X | X |
| pH | USP | X | X | X | X | X | X | X | X | X | X |
| Protein | UV | X | X | X | X | X | X | X | X | X | X |
| Concentration | | | | | | | | | | | |
| Main Peak | Charge Variants | X | X | X | X | X | X | X | X | X | X |
| Acidic Peaks | by iCIEF | X | X | X | X | X | X | X | X | X | X |
| Basic Peaks | | X | X | X | X | X | X | X | X | X | X |
| LC + HC | Purity by | X | X | X | X | X | X | X | X | X | X |
| Total Minor Species | CE-SDS (Reduced) | X | X | X | X | X | X | X | X | X | X |
| Main Peak | Purity by CE-SDS | X | X | X | X | X | X | X | X | X | X |
| Total LMWS | (Non-reduced) | X | X | X | X | X | X | X | X | X | X |
| Monomer (Main Peak) | Purity by SEC | X | X | X | X | X | X | X | X | X | X |
| HMWS | | X | X | X | X | X | X | X | X | X | X |
| Binding Bioassay | ELISA | X | X | X | X | X | X | X | X | X | X |

Abbreviations: CE-SDS = capillary electrophoresis; HMWS = high molecular weight species; iCIEF = imaged capillary isoelectric focusing; LC + HC = Light Chain + Heavy Chain; LMWS = high molecular weight species; SEC = size exclusion chromatography.
^{a}A 39- month timepoint was added for 3 batches only.

TABLE 11
Accelerated Stability Testing Plan for MAB39
Drug Substance (100 and 150 mg/mL)

| Stability Condition: 5° C. ± 3° C. | | Testing Timepoint (months) | | | | | |
|---|---|---|---|---|---|---|---|
| Attribute | Test Method | 0 | 1 | 3 | 6 | 9^{a} | 12^{a} |
| Appearance (Color) | USP | X | X | X | X | X | X |
| Appearance (Clarity) | USP | X | X | X | X | X | X |
| pH | USP | X | X | X | X | X | X |
| Protein | UV | X | X | X | X | X | X |
| Concentration | | | | | | | |
| Main Peak | Charge Variants | X | X | X | X | X | X |
| Acidic Peaks | Purity by iCIEF | X | X | X | X | X | X |
| Basic Peaks | | X | X | X | X | X | X |
| LC + HC | Purity by | X | X | X | X | X | X |
| Total Minor Species | CE-SDS (Reduced) | X | X | X | X | X | X |
| Main Peak | Purity by CE-SDS | X | X | X | X | X | X |
| Total LMWS | (Non-reduced) | X | X | X | X | X | X |
| Monomer (Main Peak) | Purity by SEC | X | X | X | X | X | X |
| HMWS | | X | X | X | X | X | X |
| Binding Bioassay | ELISA | X | X | X | X | X | X |

Abbreviations: CE-SDS = capillary electrophoresis; HMWS = high molecular weight species; iCIEF = imaged capillary isoelectric focusing; LC + HC = Light Chain + Heavy Chain; LMWS = high molecular weight species; SEC = size exclusion chromatography.
^{a}9- and 12-month timepoints were included only for some GMP batches for information only.

TABLE 12
Stress Stability Testing Plan for MAB39 Drug Substance (100 and 150 mg/mL)

| Stability Condition: 25° C. ± | | | | | |
|---|---|---|---|---|---|
| 2° C., 60% ± 5% RH | | Testing Timepoint (Month) | | | |
| Attribute | Test Method | 0 | 1 | 3 | 6^{a} |
| Appearance (Color) | USP | X | X | X | X |
| Appearance (Clarity) | USP | X | X | X | X |
| pH | USP | X | X | X | X |
| Protein Concentration | UV | X | | X | X |
| Main Peak | Charge Variants Purity by iCIEF | X | | | X |
| Acidic Peaks | | X | | | X |
| Basic Peaks | | X | X | | |
| LC + HC | Purity by CE-SDS (Reduced) | X | | | |
| Total Minor Species | | X | X | | |
| Main Peak | Purity by CE-SDS (Non-reduced) | | | | |
| Total LMWS | | X | X | X | X |
| Monomer (Main Peak) | Purity by SEC | X | | | X |
| HMWS | | X | X | X | X |
| Binding Bioassay | ELISA | | | X | X |

Abbreviations: CE-SDS = capillary electrophoresis; HMWS = high molecular weight species; iCIEF = imaged capillary isoelectric focusing; LC + HC = Light Chain + Heavy Chain; LMWS = high molecular weight species; NT = not tested; SEC = size exclusion chromatography.
^{a}6-month timepoint was included only for some GMP batches.

Long-term stability of pharmaceutical formulations according to Table 13 was determined according to methods described herein and in Example 1 and Example 2.

TABLE 13
Stability Study Batch and Test Stability Condition Summary

| | | | Individual |
|---|---|---|---|
| | | | Data Table |
| Process (Strength) | Lot # | Stability Condition | Number |
| #1 (100 mg/mL) | 20220502 | −20° C. ± 5° C. (Upright) | Table 14 |
| | | 5° C. ± 3° C. (Inverted) | Table 18 |
| | | 25° C. ± 2° C. / 60% ± 5% RH | Table 28 |
| | | (Inverted) | |
| | | 40° C. ± 2° C. / 75% ± 5% RH | Table 24 |
| | | (Inverted) | |
| #2 (150 mg/mL) | 20220902 | −20° C. ± 5° C. (Upright) | Table 15 |
| #2 (150 mg/mL) | | 5° C. ± 3° C. (Inverted) | Table 19 |
| | | 25° C. ± 2° C. / 60% ± 5% RH | Table 29 |
| | | (Inverted) | |
| | 20230201 | −20° C. ± 5° C. (Upright) | Table 16 |
| | | 5° C. ± 3° C. (Inverted) | Table 20 |
| | | 5° C. ± 3° C. (Upright) | Table 24 |
| | | 25° C. ± 2° C. / 60% ± 5% RH | Table 30 |
| | | (Inverted) | |
| | | 40° C. ± 2° C. / 75% ± 5% RH | Table 35 |
| | | (Inverted) | |
| | 20230603 | −20° C. ± 5° C. (Upright) | Table 17 |
| | | 5° C. ± 3° C. (Inverted) | Table 21 |
| | | 5° C. ± 3° C. (Upright) | Table 25 |
| | | 25° C. ± 2° C. / 60% ± 5% RH | Table 31 |
| | | (Inverted) | |
| | | 40° C. ± 2° C. / 75% ± 5% RH | Table 36 |
| | | (Inverted) | |
| | 20240603 | 25 ± 2° C. / 60 ± 5% RH, Inverted | Table 60 |
| | | 40 ± 2° C. / 75 ± 5% RH, Inverted | Table 61 |
| | | 5 ± 3° C., Inverted | Table 62 |
| | | 5 ± 3° C., Upright | Table 63 |
| | 20231002 | 5° C. ± 3° C. (Inverted) | Table 22 |
| | | 5° C. ± 3° C. (Upright) | Table 26 |
| | | 25° C. ± 2° C. / 60% ± 5% RH | Table 22 |
| | | (Inverted) | |
| | | 40° C. ± 2° C. / 75% ± 5% RH | Table 37 |
| | | (Inverted) | |
| | 20240302 | 5° C. ± 3° C. (Inverted) | Table 23 |
| | | 5° C. ± 3° C. (Upright) | Table 27 |

Data for lots stored in the upright position at −20° C. is shown in Table 14 through Table 17. Data for the inverted composition at 5° C. (accelerated condition for lot 20220502) is shown in Table 18 through Table 23. Data for lots stored in the upright position at 5° C. (accelerated condition for lot 20220502) are in Table 24 through Table 27. Data at the current accelerated condition at 25° C., 6000 RH (stress condition for lot 20220502) is shown in Table 28 through Table 33. Data at the current stress condition at 40° C., 75% RH is shown in Table 34 through Table 38. Abbreviations: CCIT=container closure integrity testing; CE-SDS=capillary electrophoresis; EFOVP=essentially free of visible particles; ELISA=enzyme-linked immunosorbent assay; HMN/WS=high molecular weight species; iCIEF=imaged capillary isoelectric focusing; L=liquid; LC+HC=light chain+heavy chain; LMWS=low molecular weight species; ND=not detected; NG=no growth; NT=not tested; NTU=nephelometric turbidity units; p/c=particles per container; RH=relative humidity; SEC=size exclusion chromatography; w/v=weight per volume; and =data not yet available.

TABLE 14
Long-term Stability Data for Clinical Lot 20220502 at −20° C. ± 5° C., Upright

| | | | Acceptance | Timepoints (months) | | | | | | | |
|---|---|---|---|---|---|---|---|---|---|---|---|
| Test/Attribute | | | Criteria | 0 | 1 | 3 | 6 | 9 | 12 | 18 | 24 |
| Visible particles | | | Liquid | L | L | L | L | L | L | L | L |
| | | | EFOVP | EFOVP | EFOVP | EFOVP | EFOVP | EFOVP | EFOVP | EFOVP | NEFOVP |
| Color, compared to | | | Not more | B6 | B6 | B6 | B6 | B6 | B6 | B6 | B6 |
| Standard Solution | | | colored | | | | | | | | |
| | | | than No. 3 | | | | | | | | |
| Clarity, NTU | | | ≤60.0 | 20.4 | 20.3 | 20.9 | 19.6 | 20.3 | 24.5 | 21.4 | 20.7 |
| pH | | | 6.7-7.7 | 7.2 | 7.2 | 7.2 | 7.2 | 7.2 | 7.3 | 7.2 | 7.2 |
| Subvisible | ≥25 | μm | ≤600 | 0 | 0 | 0 | 0 | 0 | 0 | 0 | 0 |
| Particulate | ≥10 | μm | ≤6000 | 7 | 12 | 6 | 2 | 3 | 0 | 4 | 6 |
| Matter, p/c | ≥5 | μm | Report Result | 31 | 86 | 90 | 34 | 40 | 20 | 46 | 46 |
| | ≥2 | μm | Report Result | 232 | 533 | 1010 | 350 | 529 | 388 | 327 | 394 |

| Protein | | 90.0-110.0 | 97.7 | 96.5 | 96.0 | 97.1 | 95.9 | 98.6 | 96.0 | 96.2 |
|---|---|---|---|---|---|---|---|---|---|---|
| Concentration, mg/mL | | mg/mL | | | | | | | | |
| Charge | Main Peak | ≥30.0 | 73.3 | 74.1 | 74.4 | 73.6 | 73.5 | 74.9 | 75.1 | 74.7 |
| Variants | Acidic Peaks | ≤60.0 | 20.2 | 20.8 | 20.6 | 21.1 | 21.0 | 20.6 | 20.4 | 20.8 |
| Purity by | Basic Peaks | ≤20.0 | 6.5 | 5.1 | 5.1 | 5.3 | 5.5 | 4.4 | 4.6 | 4.4 |
| iCIEF, % | | | | | | | | | | |
| Purity by | LC + HC | ≥90.0 | 98.9 | 99.0 | 98.6 | 98.6 | 98.9 | 98.3 | 98.3 | 98.3 |
| CE-SDS | Total Minor | ≤10.0 | 1.1 | 1.0 | 1.4 | 1.4 | 1.1 | 1.7 | 1.7 | 1.7 |
| (Reduced), % | Species | | | | | | | | | |
| Purity by | Main Peak | ≥90.0 | 99.0 | 98.9 | 98.8 | 98.8 | 98.8 | 98.9 | 98.9 | 98.7 |
| CE-SDS | Total LMWS | ≤10.0 | 0.9 | 1.0 | 1.1 | 1.1 | 1.0 | 1.0 | 1.0 | 1.1 |
| (Non- | | | | | | | | | | |
| reduced), % | | | | | | | | | | |
| Purity by | Monomer | ≥95.0 | 98.7 | 98.8% | 98.5 | 98.5 | 98.6 | 98.4 | 98.5 | 98.5 |
| SEC, % | (Main Peak) | | | | | | | | | |
| | HMWS | ≤5.0 | 1.3 | 1.2 | 1.5 | 1.5 | 1.4 | 1.6 | 1.5 | 1.5 |

| | | | | | | | | | |
|---|---|---|---|---|---|---|---|---|---|
| (ELISA Binding) Bioassay, | 60-140 | 100 | 98 | 87 | 107 | 102 | 88 | 94 | 93 |
| % Relative Potency | | | | | | | | | |
| Sterility | No Growth | NG | NT | NT | NT | NT | NT | NT | NG |
| CCIT | Pass | NT | NT | NT | NT | NT | Pass | NT | Pass |

TABLE 15
Long-term Stability Data for Clinical Lot 20220902 at −20° C. ± 5° C., Upright

| | | | Acceptance | Timepoints (months) | | | | | | | |
|---|---|---|---|---|---|---|---|---|---|---|---|
| Test/Attribute | | | Criteria | 0 | 1 | 3 | 6 | 9 | 12 | 18 | 24 |
| Visible particles | | | Liquid | L | L | L | L | L | L | L | L |
| (Refer to USP/Ph. Eur.) | | | EFOVP | EFOVP | EFOVP | EFOVP | EFOVP | EFOVP | EFOVP | EFOVP | EFOVP |
| Color, compared to | | | Not more | B6 | B6 | B6 | B6 | B6 | B6 | B6 | <B5 |
| Standard Solution | | | colored | | | | | | | | |
| (Refer to Ph. Eur.) | | | than No. 2 | | | | | | | | |
| Clarity, NTU | | | ≤60.0 | 19.7 | 19.7 | 19.0 | 21.7 | 22.9 | 20.7 | 19.1 | 20.1 |
| pH | | | 6.7-7.7 | 7.2 | 7.2 | 7.3 | 7.2 | 7.2 | 7.2 | 7.2 | 7.2 |
| Subvisible | ≥25 | μm | ≤600 | 1 | 0 | 0 | 0 | 0 | 0 | 0 | 0 |
| Particulate | ≥10 | μm | ≤6000 | 16 | 21 | 4 | 2 | 0 | 6 | 0 | 24 |
| Matter, p/c | ≥5 | μm | Report Result | 168 | 164 | 24 | 16 | 44 | 50 | 6 | 189 |
| | ≥2 | μm | Report Result | 1502 | 829 | 303 | 690 | 917 | 679 | 205 | 1317 |

| Protein | | 135.0-165.0 | 153.8 | 151.0 | 146.4 | 151.0 | 155.4 | 154.1 | 155.9 | 158.2 |
|---|---|---|---|---|---|---|---|---|---|---|
| Concentration, mg/mL | | | | | | | | | | |
| Charge | Main Peak | ≥30.0 | 73.1 | 72.2 | 72.7 | 72.5 | 70.9 | 72.9 | 73.0 | 72.7 |
| Variants | Acidic Peaks | ≤60.0 | 22.2 | 22.8 | 22.4 | 22.5 | 22.7 | 21.9 | 22.3 | 22.1 |
| Purity by | Basic Peaks | ≤20.0 | 4.7 | 5.0 | 4.8 | 5.0 | 5.1 | 5.1 | 4.7 | 5.3 |
| iCIEF, % | | | | | | | | | | |
| Purity by | LC + HC | ≥90.0 | 98.6 | 98.4 | 98.5 | 98.5 | 98.5 | 98.4 | 98.6 | 98.6 |
| CE-SDS | Total Minor | Report Result | 1.4 | 1.6 | 1.5 | 1.5 | 1.5 | 1.6 | 1.4 | 1.4 |
| (Reduced), % | Species | | | | | | | | | |
| Purity by | Main Peak | 85.0 | 98.7 | 98.6 | 98.7 | 98.7 | 98.6 | 98.6 | 98.7 | 98.6 |
| CE-SDS | Total LMWS | Report Result | 1.1 | 1.1 | 1.1 | 1.1 | 1.2 | 1.1 | 1.0 | 1.1 |
| (Non- | | | | | | | | | | |
| reduced), % | | | | | | | | | | |
| Purity by | Monomer | ≥90.0 | 98.3 | 98.3 | 98.4 | 98.2 | 98.3 | 98.3 | 98.2 | 98.1 |
| SEC, % | (Main Peak) | | | | | | | | | |
| | HMWS | ≤5.0 | 1.7 | 1.7 | 1.6 | 1.7 | 1.7 | 1.7 | 1.8 | 1.9 |
| | LMWS | Report Result | ND | ND | ND | 0.1% | ND | ND | ND | ND |

| | | | | | | | | | |
|---|---|---|---|---|---|---|---|---|---|
| (ELISA Binding) Bioassay, | 60-140 | 107 | 110 | 107 | 102 | 104 | 103 | 94 | 100 |
| % Relative Potency | | | | | | | | | |
| Sterility | No Growth | NG | NT | NT | NT | NT | NT | NT | NG |
| CCIT | Pass | NT | NT | NT | NT | NT | Pass | NT | Pass |

TABLE 16
Long-term Stability Data for Clinical Lot 20230201 at −20° C. ± 5° C., Upright

| | | | Acceptance | Timepoints (months) | | | | | | |
|---|---|---|---|---|---|---|---|---|---|---|
| Test/Attribute | | | Criteria | 0 | 1 | 3 | 6 | 9 | 12 | 18 |
| Visible particles | | | Liquid | L | L | L | L | L | L | L |
| (Refer to USP/Ph. Eur.) | | | EFOVP | EFOVP | EFOVP | EFOVP | EFOVP | EFOVP | EFOVP | EFOVP |
| Color, compared to | | | Not more | B6 | B6 | B6 | B6 | B6 | B6 | B5 |
| Standard Solution | | | colored | | | | | | | |
| (Refer to Ph. Eur.) | | | than No. 2 | | | | | | | |
| Clarity, NTU | | | ≤60.0 | 19.1 | 21.7 | 23.3 | 20.0 | 20.8 | 19.6 | 20.0 |
| pH | | | 6.7-7.7 | 7.2 | 7.2 | 7.2 | 7.2 | 7.2 | 7.2 | 7.2 |
| Subvisible | ≥25 | μm | ≤600 | 0 | 0 | 0 | 0 | 2 | 0 | 1 |
| Particulate | ≥10 | μm | ≤6000 | 2 | 3 | 1 | 10 | 4 | 3 | 24 |
| Matter, p/c | ≥5 | μm | Report Result | 26 | 12 | 3 | 29 | 46 | 29 | 180 |
| | ≥2 | μm | Report Result | 252 | 152 | 116 | 509 | 506 | 514 | 1338 |

| Protein | | 135.0-165.0 | 152.0 | 159.2 | 152.2 | 153.4 | 152.8 | 154.5 | 150.6 |
|---|---|---|---|---|---|---|---|---|---|
| Concentration, mg/mL | | | | | | | | | |
| Charge | Main Peak | ≥30.0 | 72.8 | 72.5 | 73.1 | 74.0 | 73.7 | 72.1 | 73.0 |
| Variants | Acidic Peaks | ≤60.0 | 22.0 | 22.4 | 22.5 | 21.3 | 21.7 | 23.3 | 22.1 |
| Purity by | Basic Peaks | ≤20.0 | 5.2 | 5.0 | 4.3 | 4.7 | 4.5 | 4.7 | 5.0 |
| iCIEF, % | | | | | | | | | |
| Purity by | LC + HC | ≥90.0% | 98.7 | 98.5 | 98.4 | 98.5 | 98.3 | 98.4 | 98.5 |
| CE-SDS | Total Minor | Report Result | 1.3 | 1.5 | 1.6 | 1.5 | 1.7 | 1.6 | 1.5 |
| (Reduced), % | Species | | | | | | | | |
| Purity by | Main Peak | ≥85.0% | 98.7 | 98.8 | 98.8 | 98.6 | 98.9 | 98.7 | 98.7 |
| CE-SDS | Total LMWS | Report Result | 1.1 | 1.0 | 1.1 | 1.1 | 1.0 | 1.1 | 1.2 |
| (Non- | | | | | | | | | |
| reduced), % | | | | | | | | | |
| Purity by | Monomer | ≥90.0% | 98.3 | 98.3 | 98.3 | 98.3 | 98.4 | 98.3 | 98.2 |
| SEC, % | (Main Peak) | | | | | | | | |
| | HMWS | ≤5.0% | 1.7 | 1.7 | 1.7 | 1.7 | 1.6 | 1.7 | 1.8 |
| | LMWS | Report Result | ND | 0.1% | ND | ND | ND | ND | ND |

| | | | | | | | | |
|---|---|---|---|---|---|---|---|---|
| (ELISA Binding) Bioassay, | 60-140 | 103 | 100 | 108 | 102 | 87 | 104 | 105 |
| % Relative Potency | | | | | | | | |
| Sterility | No Growth | NG | NT | NT | NT | NT | NT | NT |
| CCIT | Pass | NT | NT | NT | NT | NT | Pass | NT |

TABLE 17
Long Term Stability Data for Clinical Lot 20230603 at −20° C. ± 5° C., Upright

| | | Acceptance | Timepoints (months) | | |
|---|---|---|---|---|---|
| Test/ Attribute | | Criteria | 0 | 1 | 3 |
| Visible particles | | Liquid | L | L | L |
| (Refer to USP/Ph. | | EFOVP | EFOVP | EFOVP | EFOVP |
| Eur.) | | | | | |
| Color, compared to Standard | | Not more | B6 | B6 | B6 |
| Solution | | colored than | | | |
| (Refer to Ph. Eur.) | | No. 2 | | | |
| Clarity, NTU | | ≤60.0 | 24.1 | 23.8 | 20.7 |
| pH | | 6.7-7.7 | 7.2 | 7.2 | 7.2 |
| Subvisible | ≥25 μm | ≤600 | 0 | 0 | 0 |
| Particulate | ≥10 μm | ≤6000 | 0 | 0 | 1 |
| Matter, p/c | ≥5 μm | Report Result | 2 | 0 | 19 |
| | ≥2 μm | Report Result | 11 | 21 | 236 |
| Protein Concentration, | | 135.0-165.0 | 152.2 | 149.7 | 151.3 |
| mg/mL | | | | | |
| Charge | Main Peak | ≥30.0 | 76.3% | 75.6% | 76.1% |
| Variants | Acidic Peaks | ≤60.0 | 19.7% | 19.7% | 19.9% |
| Purity | Basic Peaks | ≤20.0 | 4.0% | 4.7% | 4.1% |
| by iCIEF, % | | | | | |
| Purity | LC + HC | ≥90.0 | 98.3% | 98.6% | 98.7% |
| by CE- | Total Minor | Report Result | 1.7% | 1.4% | 1.3% |
| SDS (Reduced), % | Species | | | | |
| Purity | Main Peak | ≥85.0 | 98.7% | 98.7% | 98.6% |
| by CE- | | | | | |
| SDS | Total LMWS | Report Result | 1.1% | 1.1% | 1.2% |
| (Non- | | | | | |
| reduced), % | | | | | |
| Purity by | Monomer | ≥90.0 | 98.5% | 98.5% | 98.4% |
| | (Main Peak) | | | | |
| SEC, % | HMWS | ≤5.0 | 1.5% | 1.5% | 1.6% |
| | LMWS | Report Result | ND | ND | ND |

| | | | | |
|---|---|---|---|---|
| (ELISA Binding) Bioassay, | 60-140 | 98 | 101 | 98 |
| % Relative | | | | |
| Potency | | | | |
| Sterility | No Growth | NG | NT | NT |
| CCIT | Pass | NT | NT | NT |

TABLE 18
Accelerated Stability Data for Clinical Lot 20220502 at 5° C. ± 3° C., Inverted

| | | | Acceptance | Timepoints (months) | | | | | | | |
|---|---|---|---|---|---|---|---|---|---|---|---|
| Test/Attribute | | | Criteria | 0 | 1 | 3 | 6 | 9 | 12 | 18 | 24 |
| Visible particles | | | Liquid | L | L | L | L | L | L | L | L |
| (Refer to USP/Ph. Eur.) | | | EFOVP | EFOVP | EFOVP | EFOVP | EFOVP | EFOVP | EFOVP | EFOVP | EFOVP |
| Color, compared to | | | Not more | B6 | B6 | B6 | B6 | B6 | B6 | B5 | B5 |
| Standard Solution | | | colored | | | | | | | | |
| (Refer to Ph. Eur.) | | | than No. 3 | | | | | | | | |
| Clarity, NTU | | | ≤60.0 | 20.4 | 20.2 | 20.2 | 19.7 | 20.3 | 23.4 | 21.2 | 20.3 |
| pH | | | 6.7-7.7 | 7.2 | 7.2 | 7.2 | 7.2 | 7.2 | 7.3 | 7.2 | 7.2 |
| Subvisible | ≥25 | μm | ≤600 | 0 | 0 | 0 | 0 | 0 | 0 | 1 | 2 |
| Particulate | ≥10 | μm | ≤6000 | 7 | 12 | 12 | 5 | 6 | 5 | 22 | 30 |
| Matter, p/c | ≥5 | μm | Report Result | 31 | 110 | 128 | 44 | 34 | 27 | 146 | 165 |
| | ≥2 | μm | Report Result | 232 | 638 | 778 | 297 | 288 | 239 | 738 | 940 |

| Protein | | 90.0-110.0 | 97.7 | 95.4 | 95.2 | 98.2 | 95.7 | 99.1 | 96.0 | 96.8 |
|---|---|---|---|---|---|---|---|---|---|---|
| Concentration, mg/mL | | | | | | | | | | |
| Charge | Main Peak | ≥30.0 | 73.3% | 73.4 | 72.0 | 70.7 | 69.3 | 68.9 | 66.6 | 63.2 |
| Variants | Acidic Peaks | ≤60.0 | 20.2% | 21.8 | 22.4 | 24.2 | 25.5 | 27.0 | 29.4 | 32.7 |
| Purity by | Basic Peaks | ≤20.0 | 6.5% | 4.9 | 5.6 | 5.1 | 5.2 | 4.1 | 4.0 | 4.1 |
| iCIEF, % | | | | | | | | | | |
| Purity by | LC + HC | ≥90.0 | 98.9 | 98.8 | 98.6 | 98.4 | 98.6 | 98.0 | 97.6 | 97.4 |
| CE-SDS | Total Minor | ≤10.0 | 1.1 | 1.2 | 1.4 | 1.6 | 1.4 | 2.0 | 2.4 | 2.6 |
| (Reduced), % | Species | | | | | | | | | |
| Purity by | Main Peak | ≥90.0 | 99.0 | 98.9 | 98.7 | 98.6 | 98.4 | 98.5 | 98.2 | 97.6 |
| CE-SDS | Total LMWS | ≤10.0 | 0.% | 1.0 | 1.2 | 1.2 | 1.3 | 1.3 | 1.4 | 1.8 |
| (Non- | | | | | | | | | | |
| reduced), % | | | | | | | | | | |
| Purity by | Monomer | ≥95.0 | 98.7 | 98.7 | 98.4 | 98.3 | 98.3 | 97.9 | 97.9 | 97.6 |
| SEC, % | (Main Peak) | | | | | | | | | |
| | HMWS | ≤5.0 | 1.3 | 1.3 | 1.6 | 1.7 | 1.7 | 2.0 | 2.0 | 2.3 |

| | | | | | | | | | |
|---|---|---|---|---|---|---|---|---|---|
| (ELISA Binding) Bioassay, | 60-140 | 100 | 99 | 106 | 107 | 104 | 85 | 86 | 95 |
| % Relative Potency | | | | | | | | | |
| Sterility | No growth | NG | NT | NT | NT | NT | NT | NT | NT |
| CCIT | Pass | NT | NT | NT | NT | NT | NT | NT | NT |

TABLE 19
Long-term Stability Data for Clinical Lot 20220902 at 5° C. ± 3° C., Inverted

| | | | Acceptance | Timepoints (months) | | | | | | |
|---|---|---|---|---|---|---|---|---|---|---|
| Test/Attribute | | | Criteria | 0 | 1 | 3 | 6 | 9 | 12 | 18 |
| Visible particles | | | Liquid | L | L | L | L | L | L | L |
| (Refer to USP/Ph. Eur.) | | | EFOVP | EFOVP | EFOVP | EFOVP | EFOVP | EFOVP | EFOVP | EFOVP |
| Color, compared to | | | Not more | B6 | B6 | B5 | B5 | B5 | B5 | B5 |
| Standard Solution | | | colored | | | | | | | |
| (Refer to Ph. Eur.) | | | than No. 2 | | | | | | | |
| Clarity, NTU | | | ≤60.0 | 19.7 | 19.8 | 18.8 | 21.4 | 23.9 | 19.9 | 19.6 |
| pH | | | 6.7-7.7 | 7.2 | 7.2 | 7.3 | 7.2 | 7.3 | 7.2 | 7.3 |
| Subvisible | ≥25 | μm | ≤600 | 1 | 0 | 0 | 1 | 0 | 3 | 0 |
| Particulate | ≥10 | μm | ≤6000 | 16 | 24 | 3 | 6 | 20 | 18 | 2 |
| Matter, p/c | ≥5 | μm | Report Result | 168 | 192 | 20 | 45 | 120 | 107 | 48 |
| | ≥2 | μm | Report Result | 1502 | 960 | 300 | 305 | 525 | 794 | 434 |

| Protein | | 135.0-165.0 | 153.8 | 150.0 | 147.3 | 152.1 | 155.0 | 153.9 | 156.5 |
|---|---|---|---|---|---|---|---|---|---|
| Concentration, mg/mL | | | | | | | | | |
| Charge | Main Peak | ≥30.0 | 73.1 | 71.1 | 71.7 | 69.9 | 67.2 | 67.9 | 66.5 |
| Variants | Acidic Peaks | ≤60.0 | 22.2 | 24.2 | 23.6 | 25.3 | 26.5 | 27.5 | 28.8 |
| Purity by | Basic Peaks | ≤20.0 | 4.7 | 4.7 | 14.8 | 4.8 | 6.2 | 4.5 | 4.7 |
| iCIEF, % | | | | | | | | | |
| Purity by | LC + HC | ≥90.0% | 98.6 | 98.5 | 98.3 | 98.3 | 98.2 | 98.1 | 97.9 |
| CE-SDS | Total Minor | Report Result | 1.4 | 1.5 | 1.7 | 1.7 | 1.8 | 1.9 | 2.1 |
| (Reduced), % | Species | | | | | | | | |
| Purity by | Main Peak | ≥85.0 | 98.7 | 98.6 | 98.6 | 98.4 | 98.1 | 98.1 | 97.8 |
| CE-SDS | Total LMWS | Report Result | 1.1 | 1.2 | 1.2 | 1.2 | 1.4 | 1.4 | 1.5 |
| (Non- | | | | | | | | | |
| reduced), % | | | | | | | | | |
| Purity by | Monomer | ≥90.0 | 98.3 | 98.2 | 98.2 | 97.8 | 97.7 | 97.6 | 97.4 |
| SEC, % | (Main Peak) | | | | | | | | |
| | HMWS | ≤5.0 | 1.7 | 1.8 | 1.8 | 2.1 | 2.3 | 2.3 | 2.5 |
| | LMWS | Report Result | ND | ND | ND | 0.1% | ND | 0.0 | 0.1 |

| | | | | | | | | |
|---|---|---|---|---|---|---|---|---|
| (ELISA Binding) Bioassay, | 60-140 | 107 | 113 | 111 | 105 | 103 | 102 | 88 |
| % Relative Potency | | | | | | | | |
| Sterility | No Growth | NG | NT | NT | NT | NT | NT | NG |
| CCIT | Pass | NT | NT | NT | NT | NT | Pass | Pass |

TABLE 20
Long-term Stability Data for Clinical Lot 20230201 at 5° C. ± 3° C., Inverted

| | | | Acceptance | Timepoints (months) | | | | | | |
|---|---|---|---|---|---|---|---|---|---|---|
| Test/Attribute | | | Criteria | 0 | 1 | 3 | 6 | 9 | 12 | 18 |
| Visible particles | | | Liquid | L | L | L | L | L | L | L |
| (Refer to USP/Ph. Eur.) | | | EFOVP | EFOVP | EFOVP | EFOVP | EFOVP | EFOVP | EFOVP | EFOVP |
| Color, compared to | | | Not more | B6 | B6 | B6 | B5 | B5 | B5 | B5 |
| Standard Solution | | | colored | | | | | | | |
| (Refer to Ph. Eur.) | | | than No. 2 | | | | | | | |
| Clarity, NTU | | | ≤60.0 | 19.1 | 22.0 | 23.8 | 20.0 | 20.2 | 19.4 | 20.3 |
| pH | | | 6.7-7.7 | 7.2 | 7.2 | 7.2 | 7.2 | 7.2 | 7.3 | 7.2 |
| Subvisible | ≥25 | μm | ≤600 | 0 | 1 | 0 | 0 | 0 | 1 | 3 |
| Particulate | ≥10 | μm | ≤6000 | 2 | 2 | 3 | 6 | 9 | 7 | 70 |
| Matter, p/c | ≥5 | μm | Report Result | 26 | 20 | 25 | 43 | 96 | 50 | 390 |
| | ≥2 | μm | Report Result | 252 | 154 | 151 | 232 | 690 | 359 | 1789 |

| Protein | | 135.0-165.0 | 152.0 | 157.1 | 153.1 | 153.1 | 153.0 | 153.8 | 150.3 |
|---|---|---|---|---|---|---|---|---|---|
| Concentration, mg/mL | | | | | | | | | |
| Charge | Main Peak | ≥30.0 | 72.8 | 71.9 | 71.9 | 71.6 | 70.6 | 67.2 | 66.4 |
| Variants | Acidic Peaks | ≤60.0 | 22.0 | 23.1 | 23.8 | 23.8 | 25.1 | 28.1 | 29.4 |
| Purity by | Basic Peaks | ≤20.0 | 5.2 | 5.0 | 4.4 | 4.7 | 4.3 | 4.7 | 4.2 |
| iCIEF, % | | | | | | | | | |
| Purity by | LC + HC | ≥90.0 | 98.7 | 98.6 | 98.4 | 98.3 | 98.0 | 98.1 | 97.9 |
| CE-SDS | Total Minor | Report Result | 1.3 | 1.4 | 1.6 | 1.7 | 2.0 | 1.9 | 2.1 |
| (Reduced), % | Species | | | | | | | | |
| Purity by | Main Peak | ≥85.0 | 98.7 | 98.7 | 98.6 | 98.4 | 98.5 | 98.2 | 98.0 |
| CE-SDS | Total LMWS | Report Result | 1.1 | 1.1 | 1.2 | 1.2 | 1.2 | 1.4 | 1.6 |
| (Non- | | | | | | | | | |
| reduced), % | | | | | | | | | |
| Purity by | Monomer | ≥90.0 | 98.3 | 98.1 | 98.0 | 97.9 | 97.9 | 97.6 | 97.4 |
| SEC, % | (Main Peak) | | | | | | | | |
| | HMWS | ≤5.0 | 1.7 | 1.8 | 2.0 | 2.1 | 2.1 | 2.3 | 2.4 |
| | LMWS | Report Result | ND | 0.1% | ND | ND | ND | 0.1% | 0.2 |

| | | | | | | | | |
|---|---|---|---|---|---|---|---|---|
| (ELISA Binding) Bioassay, | 60-140 | 103 | 99 | 105 | 103 | 102 | 103 | 103 |
| % Relative Potency | | | | | | | | |
| Sterility | No Growth | NG | NT | NT | NT | NT | NT | NT |
| CCIT | Pass | NT | NT | NT | NT | NT | Pass | NT |

TABLE 21
Long Term Stability Data for Clinical Lot 20230603 at 5° C. ± 3° C., Inverted

| | | | Acceptance | Timepoints (months) | | | | | |
|---|---|---|---|---|---|---|---|---|---|
| Test/Attribute | | | Criteria | 0 | 1 | 3 | 6 | 9 | 12 |
| Visible particles | | | Liquid | L | L | L | L | L | L |
| (Refer to USP/Ph. Eur.) | | | EFOVP | EFOVP | EFOVP | EFOVP | EFOVP | EFOVP | EFOVP |
| Color, compared to | | | Not more | B6 | B6 | B6 | B6 | B6 | B5 |
| Standard Solution | | | colored | | | | | | |
| (Refer to Ph. Eur.) | | | than No. 2 | | | | | | |
| Clarity, NTU | | | ≤60.0 | 24.1 | 23.9 | 20.4 | 21.1 | 19.6 | 19.5 |
| pH | | | 6.7-7.7 | 7.2 | 7.2 | 7.2 | 7.2 | 7.2 | 7.2 |
| Subvisible | ≥25 | μm | ≤600 | 0 | 0 | 0 | 0 | 0 | 0 |
| Particulate | ≥10 | μm | ≤6000 | 0 | 0 | 0 | 1 | 4 | 14 |
| Matter, p/c | ≥5 | μm | Report Result | 2 | 0 | 11 | 14 | 38 | 112 |
| | ≥2 | μm | Report Result | 11 | 32 | 92 | 108 | 275 | 543 |

| Protein | | 135.0-165.0 | 152.2 | 151.3 | 153.7 | 150.6 | 151.0 | \|152.9 |
|---|---|---|---|---|---|---|---|---|
| Concentration, mg/mL | | | | | | | | |
| Charge | Main Peak | ≥30.0 | 76.3 | 75.4 | 74.2 | 72.5 | 71.6 | 71.5 |
| Variants | Acidic Peaks | ≤60.0 | 19.7 | 20.1 | 21.5 | 22.1 | 23.5 | 24.5 |
| Purity by | Basic Peaks | ≤20.0 | 4.0 | 4.5 | 4.3 | 5.4 | 4.9 | 4.0 |
| iCIEF, % | | | | | | | | |
| Purity by | LC + HC | ≥90.0 | 98.3 | 98.5 | 98.5 | 98.5 | 98.3 | 98.1 |
| CE-SDS | Total Minor | Report Result | 1.7 | 1.5 | 1.5 | 1.5 | 1.7 | 1.9 |
| (Reduced), % | Species | | | | | | | |
| Purityby | Main Peak | ≥85.0 | 98.7 | 98.7 | 98.6 | 98.5 | 98.5 | 98.3 |
| CE-SDS | Total LMWS | Report Result | 1.1 | 1.2 | 1.2 | 1.2 | 1.3 | 1.3 |
| (Non- | | | | | | | | |
| reduced), % | | | | | | | | |
| Purity by | Monomer | ≥90.0 | 98.5 | 98.4 | 98.3 | 98.2 | 98.1 | 98.0 |
| SEC, % | (Main Peak) | | | | | | | |
| | HMWS | 5.0 | 1.5 | 1.6 | 1.7 | 1.8 | 1.9 | 2.0 |
| | LMWS | Report Result | ND | ND | ND | ND | ND | ND |

| | | | | | | | |
|---|---|---|---|---|---|---|---|
| Poloxamer 188 Content, %(w/v) | Report Result | 0.09 | NT | NT | 0.11 | NT | 0.10 |
| (ELISA Binding) Bioassay, | 60-140 | 98 | 107 | 100 | 116 | 101 | 99 |
| % Relative Potency | | | | | | | |
| Sterility | No Growth | NG | NT | NT | NT | NT | NT |
| CCIT | Pass | NT | NT | NT | NT | NT | Pass |

TABLE 22
Long Term Stability Data for Clinical Lot 20231002 at 5° C. ± 3° C., Inverted

| | | | Acceptance | Timepoints (months) | | | |
|---|---|---|---|---|---|---|---|
| Test/Attribute | | | Criteria | 0 | 1 | 3 | 6 |
| Visible particles | | | Liquid | L | L | L | L |
| (Refer to USP/Ph. Eur.) | | | EFOVP | EFOVP | EFOVP | EFOVP | EFOVP |
| Color, compared to | | | Not more | B6 | B6 | B6 | B6 |
| Standard Solution | | | colored | | | | |
| (Refer to Ph. Eur.) | | | than No. 2 | | | | |
| Clarity, NTU | | | ≤60.0 | 21.5 | 20.5 | 20.1 | 20.0 |
| pH | | | 6.7-7.7 | 7.2 | 7.2 | 7.2 | 7.2 |
| Subvisible | ≥25 | μm | ≤600 | 4 | 0 | 0 | 1 |
| Particulate | ≥10 | μm | ≤6000 | 6 | 9 | 4 | 3 |
| Matter, p/c | ≥5 | μm | Report Result | 14 | 74 | 27 | 16 |
| | ≥2 | μm | Report Result | 178 | 568 | 220 | 190 |

| Protein | | 135.0-165.0 | 152.1 | 151.7 | 149.8 | 151.0 |
|---|---|---|---|---|---|---|
| Concentration, mg/mL | | | | | | |
| Charge | Main Peak | ≥30.0 | 74.6 | 75.1 | 74.8 | 73.1 |
| Variants | Acidic Peaks | ≤60.0 | 20.5 | 20.7 | 20.7 | 23.0 |
| Purity by | Basic Peaks | ≤20.0 | 4.9 | 4.2 | 4.5 | 3.8 |
| iCIEF, % | | | | | | |
| Purity by | LC + HC | ≥90.0 | 98.6 | 98.3 | 98.5 | 98.4 |
| CE-SDS | Total Minor | Report Result | 1.4 | 1.7 | 1.5 | 1.6 |
| (Reduced), % | Species | | | | | |
| Purity by | Main Peak | ≥85.0 | 98.6 | 98.8 | 98.6 | 98.4 |
| CE-SDS | Total LMWS | Report Result | 1.1 | 1.1 | 1.2 | 1.3 |
| (Non- | | | | | | |
| reduced), % | | | | | | |
| Purity by | Monomer | ≥90.0 | 98.5 | 98.4 | 98.4 | 98.2 |
| SEC, % | (Main Peak) | | | | | |
| | HMWS | ≤5.0 | 1.5 | 1.6 | 1.6 | 1.8 |
| | LMWS | Report Result | ND | ND | ND | ND |

| | | | | | |
|---|---|---|---|---|---|
| Poloxamer 188 Content, %(w/v) | Report Result | 0.09 | NT | NT | 0.11 |
| (ELISA Binding Bioassay, | 60-140 | 97 | 99 | 105 | 102 |
| % Relative Potency | | | | | |
| Sterility | No Growth | NG | NT | NT | NT |
| CCIT | Pass | NT | NT | NT | NT |

TABLE 23
Long-term Stability Data for Clinical Lot 20240302 at 5° C. ± 3° C., Inverted

| | | | Acceptance | Timepoints (months) | | | |
|---|---|---|---|---|---|---|---|
| Test/Attribute | | | Criteria | 0 | 1 | 3 | 6 |
| Visible particles | | | Liquid | L | L | L | L |
| (Refer to USP/Ph. Eur.) | | | EFOVP | EFOVP | EFOVP | EFOVP | EFOVP |
| Color, compared to | | | Not more | B6 | BY5 | B5 | B5 |
| Standard Solution | | | colored | | | | |
| (Refer to Ph. Eur.) | | | than No. 2 | | | | |
| Clarity, NTU | | | ≤60.0 | 19.6 | 19.4 | 20.3 | 19.9 |
| pH | | | 6.7-7.7 | 7.2 | | 7.2 | 7.2 |
| Subvisible | ≥25 | μm | ≤600 | 0 | 0 | 0 | 0 |
| Particulate | ≥10 | μm | ≤6000 | 0 | 0 | 4 | 25 |
| Matter, p/c | ≥5 | μm | Report Result | 16 | 12 | 21 | 130 |
| | ≥2 | μm | Report Result | 469 | 166 | 304 | 676 |

| Protein | | 135.0-165.0 | 151.9 | 153.3 | 151.8 | 159.9 |
|---|---|---|---|---|---|---|
| Concentration, mg/mL | | | | | | |
| Charge | Main Peak | ≥30.0 | 75.3 | 73.7 | 73.1 | 71.8 |
| Variants | Acidic Peaks | ≤60.0 | 20.5 | 22.4 | 22.9 | 23.8 |
| Purity by | Basic Peaks | ≤20.0 | 4.2 | 3.9 | 4.0 | 4.4 |
| iCIEF, % | | | | | | |
| Purity by | LC + HC | ≥90.0 | 98.5 | 98.5 | 98.4 | 98.5 |
| CE-SDS | Total Minor | Report Result | 1.5 | 1.5 | 1.6 | 1.5 |
| (Reduced), % | Species | | | | | |
| Purity by | Main Peak | ≥85.0 | 98.7 | 98.5 | 98.6 | 98.4 |
| CE-SDS | Total LMWS | Report Result | 1.1 | 1.3 | 1.1 | 1.2 |
| (Non- | | | | | | |
| reduced), % | | | | | | |
| Purity by | Monomer | ≥90.0 | 98.4 | 98.3 | 98.2 | 98.0 |
| SEC, % | (Main Peak) | | | | | |
| | HMWS | ≤5.0 | 1.6 | 1.7 | 1.8 | 2.0 |
| | LMWS | Report Result | ND | ND | ND | ND |

| | | | | | |
|---|---|---|---|---|---|
| Poloxamer 188 Content, %(w/v) | Report Result | 0.08 | NT | NT | 0.09 |
| (ELISA Binding) Bioassay, | 60-140 | 101 | 88 | 96 | 99 |
| % Relative Potency | | | | | |
| Sterility | No Growth | NG | NT | NT | NT |
| CCIT | Pass | NT | NT | NT | NT |

TABLE 24
Long-term Stability Data for Clinical Lot 20230201 at 5° C. ± 3° C., Upright

| | | | Acceptance | Timepoints (months) | | | | | |
|---|---|---|---|---|---|---|---|---|---|
| Test/Attribute | | | Criteria | 0 | 1 | 3 | 6 | 9 | 12 |
| Visible particles | | | Liquid | L | L | L | L | L | L |
| (Refer to USP/Ph. Eur.) | | | EFOVP | EFOVP | EFOVP | EFOVP | EFOVP | EFOVP | EFOVP |
| Color, compared to | | | Not more | B6 | B6 | B6 | B5 | B5 | B5 |
| Standard Solution | | | colored | | | | | | |
| (Refer to Ph. Eur.) | | | than No. 2 | | | | | | |
| Clarity, NTU | | | ≤60.0 | 19.1 | 21.6 | 23.1 | 20.3 | 20.3 | 19.7 |
| pH | | | 6.7-7.7 | 7.2 | 7.2 | 7.2 | 7.2 | 7.2 | 7.3 |
| Subvisible | ≥25 | μm | ≤600 | 0 | 0 | 0 | 0 | 0 | 2 |
| Particulate | ≥10 | μm | ≤6000 | 2 | 4 | 2 | 3 | 10 | 10 |
| Matter, p/c | ≥5 | μm | Report Result | 26 | 22 | 22 | 40 | 56 | 44 |
| | ≥2 | μm | Report Result | 252 | 212 | 193 | 316 | 428 | 380 |

| Protein | | 135.0-165.0 | 152.0 | 158.6 | 153.4 | 153.2 | 153.5 | 154.7 |
|---|---|---|---|---|---|---|---|---|
| Concentration, mg/mL | | | | | | | | |
| Charge | Main Peak | ≥30.0 | 72.8 | 72.5 | 72.4 | 71.9 | 71.2 | 66.0 |
| Variants | Acidic Peaks | ≤60.0 | 22.0 | 22.5 | 23.6 | 23.5 | 25.0 | 28.3 |
| Purity by | Basic Peaks | ≤20.0 | 5.2 | 4.9 | 4.0 | 4.6 | 3.9 | 5.7 |
| iCIEF, % | | | | | | | | |
| Purity by | LC + HC | ≥90.0 | 98.7 | 98.3 | 98.2 | 98.1 | 98.1 | 98.1 |
| CE-SDS | Total Minor | Report Result | 1.3 | 1.7 | 1.8 | 1.9 | 1.9 | 1.9 |
| (Reduced), % | Species | | | | | | | |
| Purity by | Main Peak | ≥85.0 | 98.7 | 98.6 | 98.7 | 98.3 | 98.6 | 98.2 |
| CE-SDS | Total LMWS | Report Result | 1.1 | 1.2 | 1.1 | 1.2 | 1.1 | 1.4 |
| (Non- | | | | | | | | |
| reduced), % | | | | | | | | |
| Purity by | Monomer | ≥90.0 | 98.3 | 98.1 | 98.0 | 98.0 | 97.9 | 97.6 |
| SEC, % | (Main Peak) | | | | | | | |
| | HMWS | ≤5.0 | 1.7 | 1.8 | 2.0 | 2.0 | 2.1 | 2.3 |
| | LMWS | Report | ND | 0.1 | 0.0 | ND | ND | 0.1 |
| | | Result | | | | | | |

| | | | | | | | |
|---|---|---|---|---|---|---|---|
| (ELISA Binding) Bioassay, | 60-140 | 103 | 99 | 104 | 104 | 87 | 106 |
| % Relative Potency | | | | | | | |
| Sterility | No Growth | NG | NT | NT | NT | NT | NT |
| CCIT | Pass | NT | NT | NT | NT | NT | NT |

TABLE 25
Long Term Stability Data for Clinical Lot 20230603 at 5° C. ± 3° C., Upright

| | | | Acceptance | Timepoints (months) | | | | | |
|---|---|---|---|---|---|---|---|---|---|
| Test/Attribute | | | Criteria | 0 | 1 | 3 | 6 | 9 | 12 |
| Visible particles | | | Liquid | L | L | L | L | L | L |
| (Refer to USP/Ph. Eur.) | | | EFOVP | EFOVP | EFOVP | EFOVP | EFOVP | EFOVP | EFOVP |
| Color, compared to | | | Not more | B6 | B6 | B6 | B6 | B6 | B5 |
| Standard Solution | | | colored | | | | | | |
| (Refer to Ph. Eur.) | | | than No. 2 | | | | | | |
| Clarity, NTU | | | ≤60.0 | 24.1 | 23.4 | 20.5 | 20.7 | 19.6 | 19.7 |
| pH | | | 6.7-7.7 | 7.2 | 7.2 | 7.2 | 7.2 | 7.2 | 7.2 |
| Subvisible | ≥25 | μm | ≤600 | 0 | NT | NT | NT | NT | 1 |
| Particulate | ≥10 | μm | ≤6000 | 0 | NT | NT | NT | NT | 13 |
| Matter, p/c | ≥5 | μm | Report Result | 2 | NT | NT | NT | NT | 120 |
| | ≥2 | μm | Report Result | 11 | NT | NT | NT | NT | 717 |

| Protein | | 135.0-165.0 | 152.2 | 153.0 | 151.4 | 150.6 | 152.7 | 152.0 |
|---|---|---|---|---|---|---|---|---|
| Concentration, mg/mL | | | | | | | | |
| Charge | Main Peak | ≥30.0 | 76.3 | 75.5 | 74.8 | 72.7 | 71.1 | 71.8 |
| Variants | Acidic Peaks | ≤60.0 | 19.7 | 19.8 | 21.3 | 22.6 | 23.9 | 24.2 |
| Purity by | Basic Peaks | ≤20.0 | 4.0 | 4.7 | 3.9 | 4.6 | 4.9 | 4.0 |
| iCIEF, % | | | | | | | | |
| Purity by | LC + HC | ≥90.0 | 98.3 | 98.5 | 98.7 | 98.3 | 98.3 | 98.3 |
| CE-SDS | Total Minor | Report Result | 1.7 | 1.5 | 1.3 | 1.7 | 1.7 | 1.7 |
| (Reduced), % | Species | | | | | | | |
| Purity by | Main Peak | ≥85.0 | 98.7 | 98.7 | 98.6 | 98.4 | 98.4 | 98.2 |
| CE-SDS | Total LMWS | Report Result | 1.1 | 1.2 | 1.1 | 1.3 | 1.2 | 1.4 |
| (Non- | | | | | | | | |
| reduced), % | | | | | | | | |
| Purity by | Monomer | ≥90.0 | 98.5 | 98.4 | 98.3 | 98.2 | 98.1 | 98.0 |
| SEC, % | (Main Peak) | | | | | | | |
| | HMWS | ≤5.0 | 1.5 | 1.6 | 1.7 | 1.8 | 1.9 | 2.0 |
| | LMWS | Report Result | ND | ND | ND | ND | ND | ND |

| | | | | | | | |
|---|---|---|---|---|---|---|---|
| Poloxamer 188 Content, %(w/v) | Report Result | 0.09 | NT | NT | 0.11 | NT | 0.09 |
| (ELISA Binding) Bioassay, | 60-140 | 98 | 105 | 99 | 106 | 98 | 96 |
| % Relative Potency | | | | | | | |
| Sterility | No Growth | NG | NT | NT | NT | NT | NT |
| CCIT | Pass | NT | NT | NT | NT | NT | Pass |

TABLE 26
Long Term Stability Data for Clinical Lot 20231002 at 5° C. ± 3° C., Upright

| | | | Acceptance | Timepoints (months) | | | |
|---|---|---|---|---|---|---|---|
| Test/Attribute | | | Criteria | 0 | 1 | 3 | 6 |
| Visible particles | | | Liquid | L | L | L | |
| Refer to USP/Ph. Eur.) | | | EFOVP | EFOVP | EFOVP | EFOVP | EFOVP |
| Color, compared to | | | Not more | B6 | B6 | B6 | B6 |
| Standard Solution | | | colored | | | | |
| (Refer to Ph. Eur.) | | | than No. 2 | | | | |
| Clarity, NTU | | | ≤60.0 | 21.5 | 20.3 | 20.3 | 19.8 |
| pH | | | 6.7-7.7 | 7.2 | 7.2 | 7.2 | 7.2 |
| Subvisible | ≥25 | μm | ≤600 | 4 | NT | NT | −NT |
| Particulate | ≥10 | μm | ≤6000 | 6 | NT | NT | −NT |
| Matter, p/c | ≥5 | μm | Report Result | 14 | NT | NT | NT |
| | ≥2 | μm | Report Result | 178 | NT | NT | NT |

| Protein | | 135.0-165.0 | 152.1 | 151.7 | 150.1 | 149.8 |
|---|---|---|---|---|---|---|
| Concentration, mg/mL | | | | | | |
| Charge | Main Peak | ≥30.0 | 74.6 | 74.6 | 74.6 | 72.8− |
| Variants | Acidic Peaks | ≤60.0 | 20.5 | 21.1 | 20.9 | 22.9 |
| Purity by | Basic Peaks | ≤20.0 | 4.9 | 4.3 | 4.4 | 4.3 |
| iCIEF, % | | | | | | |
| Purity by | LC + HC | ≥90.0 | 98.6 | 98.5 | 98.8 | 98.4 |
| CE-SDS | Total Minor | Report Result | 1.4 | 1.5 | 1.2 | 1.6 |
| (Reduced), % | Species | | | | | |
| Purity by | Main Peak | ≥85.0 | 98.6 | 98.8 | 98.7 | 98.4 |
| CE-SDS | Total LMWS | Report Result | 1.1 | 1.1 | 1.1 | 1.2 |
| (Non- | | | | | | |
| reduced), % | | | | | | |
| Purity by | Monomer | ≥90.0 | 98.5 | 98.4 | 98.4 | 98.2 |
| SEC, % | (Main Peak) | | | | | |
| | HMWS | ≤5.0 | 1.5 | 1.6 | 1.6 | 1.8 |
| | LMWS | Report Result | ND | ND | ND | ND |

| | | | | | |
|---|---|---|---|---|---|
| Poloxamer 188 Content, %(w/v) | Report Result | 0.09 | NT | NT | 0.11 |
| (ELISA Binding) Bioassay, | 60-140 | 97 | 105 | 104 | 110 |
| % Relative Potency | | | | | |
| Sterility | No Growth | NG | NT | NT | INT |
| CCIT | Pass | NT | NT | NT | NT |

TABLE 27
Long-term Stability Data for Clinical
Lot 20240302 at 5° C. ± 3° C., Upright

| | | Acceptance | Timepoints (months) | | | |
|---|---|---|---|---|---|---|
| Test/Attribute | | Criteria | 0 | 1 | 3 | 6 |
| Visible particles (Refer to | | Liquid | L | L | L | L |
| USP/Ph. Eur.) | | EFOVP | EFOVP | EFOVP | EFOVP | EFOVP |
| Color, compared to Standard | | Not more | B6 | BY5 | B5 | B5 |
| Solution (Refer to Ph. Eur.) | | colored | | | | |
| | | than No. 2 | | | | |
| Clarity, NTU | | ≤60.0 | 19.6 | 19.3 | 19.8 | 19.8 |
| pH | | 6.7-7.7 | 7.2 | 7.2 | 7.2 | 7.2 |
| Subvisible | ≥25 μm | ≤600 | 0 | NT | NT | NT |
| Particulate | ≥10 μm | ≤6000 | 0 | NT | NT | NT |
| Matter, | ≥5 μm | Report | 16 | NT | NT | NT |
| p/c | | Result | | | | |
| | ≥2 μm | Report | 469 | NT | NT | NT |
| | | Result | | | | |
| Protein Concentration, | | 135.0-165.0 | 151.9 | 153.3 | 151.2 | 160.6 |
| mg/mL | | | | | | |
| Charge | Main Peak | ≥30.0 | 75.3 | 73.5 | 73.7 | 71.6 |
| Variants | Acidic Peaks | ≤60.0 | 20.5 | 22.3 | 22.4 | 24.1 |
| Purity | Basic Peaks | ≤20.0 | 4.2 | 4.2 | 3.9 | 4.4 |
| by iCIEF, | | | | | | |
| % | | | | | | |
| Purity | LC + HC | ≥90.0 | 98.5 | 98.4 | 98.3 | 98.4 |
| by | Total | Report | 1.5 | 1.6 | 1.7 | 1.6 |
| CE-SDS | Minor | Result | | | | |
| (Reduced), % | Species | | | | | |
| Purity | Main Peak | ≥85.0 | 98.7 | 98.6 | 98.5 | 98.4 |
| by CE-SDS | Total | Report | 1.1 | 1.2 | 1.1 | 1.3 |
| (Non-reduced), % | LMWS | Result | | | | |
| Purity by | Monomer | ≥90.0 | 98.4 | 98.3 | 98.2 | 98.0 |
| SEC, % | (Main Peak) | | | | | |
| | HMWS | ≤5.0 | 1.6 | 1.7 | 1.8 | 2.0 |
| | LMWS | Report | ND | ND | ND | ND |
| | | Result | | | | |

| | | | | | |
|---|---|---|---|---|---|
| Poloxamer 188 Content, | Report | 0.08 | NT | NT | 0.10 |
| % (w/v) | Result | | | | |
| (ELISA Binding) Bioassay, | 60-140 | 101 | 91 | 98 | 98 |
| % Relative Potency | | | | | |
| Sterility^{a} | No Growth | NG | NT | NT | NT |
| CCIT^{b} | Pass | NT | NT | NT | NT |

TABLE 28
Stress Stability Data for Clinical Lot 20220502 at
25° C. ± 2° C./60% ± 5%RH, Inverted

| | | Acceptance | Timepoints (months) | | | |
|---|---|---|---|---|---|---|
| Test/Attribute | | Criteria | 0 | 1 | 3 | 6 |
| Visible particles (Refer to | | Liquid | L | L | L | L |
| USP/Ph. Eur.) | | EFOVP | EFOVP | EFOVP | EFOVP | EFOVP |
| Color, compared to Standard | | Not more | B6 | B6 | B5 | B5 |
| Solution (Refer to Ph. Eur.) | | colored than | | | | |
| | | No. 3 | | | | |
| Clarity, NTU | | ≤60.0 | 20.4 | 20.4 | 20.0 | 19.3 |
| pH | | 6.7-7.7 | 7.2 | 7.2 | 7.2 | 7.2 |
| Subvisible | ≥25 μm | ≤600 | 0 | 2 | 2 | 1 |
| Particulate | ≥10 μm | ≤6000 | 7 | 52 | 38 | 19 |
| Matter, | ≥5 μm | Report Result | 31 | 279 | 154 | 87 |
| p/c | ≥2 μm | Report Result | 232 | 1370 | 716 | 512 |
| Protein Concentration, mg/ml | | 90.0-110.0 | 97.7 | 95.8 | 96.4 | 97.9 |
| Charge | Main Peak | ≥30.0 | 73.3 | 65.2 | 50.6 | 34.0 |
| Variants | Acidic Peaks | ≤60.0 | 20.2 | 29.4 | 44.9 | |
| Purity by | Basic Peaks | ≤20.0 | 6.5 | 5.5 | 4.4 | 5.4 |
| iCIEF, % | | | | | | |
| Purity | LC + HC | ≥90.0 | 98.9 | 98.3 | 96.6 | 93.5 |
| by CE-SDS | Total | ≤10.0 | 1.1 | 1.7 | 3.4 | 6.5 |
| (Reduced), % | Minor | | | | | |
| | Species | | | | | |
| Purity | Main Peak | ≥90.0 | 99.0 | 98.2 | 96.1 | 93.8 |
| by CE-SDS | Total LMWS | ≤10.0 | 0.9 | 1.5 | 3.2 | 4.7 |
| (Non-reduced), % | | | | | | |
| Purity by | Monomer | ≥95.0 | 98.7 | 98.2 | 96.8 | 95.3 |
| SEC, % | (Main Peak) | | | | | |
| | HMWS | ≤5.0 | 1.3 | 1.8 | 3.1 | 4.6 |

| | | | | | |
|---|---|---|---|---|---|
| (ELISA Binding) Bioassay, | 60-140 | 100 | 93 | 102 | 96 |
| % Relative Potency | | | | | |

^{a} Acceptance criterion was exceeded, indicating a significant change in the charge profile at the accelerated condition.

TABLE 29
Accelerated Stability Data for Clinical Lot 20220902
at 25° C. ± 2° C./60% ± 5% RH, Inverted

| | | Acceptance | Timepoints (months) | | | |
|---|---|---|---|---|---|---|
| Test/Attribute | | Criteria | 0 | 1 | 3 | 6 |
| Visible particles (Refer to | | Liquid | L | L | L | L |
| USP/Ph. Eur.) | | EFOVP | EFOVP | EFOVP | EFOVP | EFOVP |
| Color, compared to Standard | | Not more | B6 | B5 | B5 | B5 |
| Solution (Refer to Ph. Eur.) | | colored than | | | | |
| | | No. 2 | | | | |
| Clarity, NTU | | ≤60.0 | 19.7 | 19.5 | 18.6 | 20.5 |
| pH | | 6.7-7.7 | 7.2 | 7.2 | 7.3 | 7.2 |
| Subvisible | ≥25 μm | ≤600 | 1 | 2 | 1 | 7 |
| Particulate | ≥10 μm | ≤6000 | 16 | 50 | 28 | 135 |
| Matter, | ≥5 μm | Report Result | 168 | 262 | 183 | 578 |
| p/c | ≥2 μm | Report Result | 1502 | 1453 | 984 | 2378 |
| Protein Concentration, mg/mL | | 135.0-165.0 | 153.8 | 154.3 | 148.3 | 152.9 |
| Charge | Main Peak | ≥30.0 | 73.1 | 64.1 | 49.5 | 34.2 |
| Variants | Acidic Peaks | ≤60.0 | 22.2 | 31.9 | 45.2 | 60.6 |
| Purity | Basic Peaks | ≤20.0 | 4.7 | 4.0 | 5.3 | 60.6 ^{a} |
| by iCIEF, | | | | | | |
| % | | | | | | |
| Purity | LC + HC | ≥90.0 | 98.6 | 97.9 | 96.5 | 5.2 |
| by CE- | Total | Report Result | 1.4 | 2.1 | 3.5 | 94.3 |
| SDS | Minor | | | | | |
| (Reduced), % | Species | | | | | |
| Purity | Main Peak | ≥85.0 | 98.7 | 97.7 | 96.1 | 5.7 |
| by CE-SDS | Total LMWS | Report Result | 1.1 | 1.7 | 2.9 | 93.9 |
| (Non- | | | | | | |
| reduced), % | | | | | | |
| Purity by | Monomer | ≥90.0 | 98.3 | 97.5 | 96.7 | 4.4 |
| SEC, % | (Main Peak) | | | | | |
| | HMWS | ≤ 5.0 | 1.7 | 2.5 | 3.3 | 94.8 |
| | LMWS | Report Result | IND | 0.1 | ND | 5.0 |

| | | | | | |
|---|---|---|---|---|---|
| ELISA Binding) Bioassay, | 60-140 | 107 | 106 | 103 | 100 |
| % Relative Potency | | | | | |

TABLE 30
Accelerated Stability Data for Clinical Lot 20230201
at 25° C. ± 2° C./60% ± 5% RH, Inverted

| | | Acceptance | Timepoints (months) | | | | |
|---|---|---|---|---|---|---|---|
| Test/Attribute | | Criteria | 0 | 1 | 3 | 6 | 9 |
| Visible particles (Refer to | | Liquid | L | L | L | L | L |
| USP/Ph. Eur.) | | EFOVP | EFOVP | EFOVP | EFOVP | EFOVP | EFOVP |
| Color, compared to Standard | | Not more | B6 | B5 | B5 | B5 | B4 |
| Solution (Refer to Ph. Eur.) | | colored than | | | | | |
| | | No. 2 | | | | | |
| Clarity, NTU | | ≤60.0 | 19.1 | 21.3 | 23.0 | 19.3 | 19.9 |
| pH | | 6.7-7.7 | 7.2 | 7.2 | 7.2 | 7.3 | 7.3 |
| Subvisible | ≥25 μm | ≤600 | 0 | 4 | 2 | 2 | 2 |
| Particulate | ≥10 μm | ≤6000 | 2 | 36 | 21 | 36 | 42 |
| Matter, | ≥5 μm | Report Result | 26 | 166 | 108 | 175 | 222 |
| p/c | ≥2 μm | Report Result | 252 | 822 | 41 | 791 | 1293 |
| Protein Concentration, mg/mL | | 135.0-165.0 | 152.0 | 156.0 | 153.1 | 154.7 | 153.4 |
| Charge | Main Peak | ≥30.0 | 72.8 | 64.7 | 51.1 | 35.2 | 25.2 ^{a} |
| Variants | Acidic Peaks | ≤60.0 | 22.0 | 30.2 | 45.2 | 59.7 | 70.0 ^{a} |
| Purity by | Basic Peaks | ≤20.0 | 5.2 | 5.0 | 3.7 | 5.1 | 4.8 |
| iCIEF, % | | | | | | | |
| Purity | LC + HC | ≥90.0 | 98.7 | 98.0 | 96.5 | 94.6 | 92.6 |
| by CE-SDS | Total | Report Result | 1.3 | 2.0 | 3.5 | 5.4 | 5.9 |
| (Reduced), % | Minor | | | | | | |
| | Species | | | | | | |
| Purity | Main Peak | ≥85.0 | 98.7 | 98.0 | 96.5 | 94.2 | 92.1 |
| by CE-SDS | Total LMWS | Report Result | 1.1 | 1.6 | 2.5 | 4.2 | 7.4 |
| (Non-reduced), % | | | | | | | |
| Purity by | Monomer | ≥90.0 | 98.3 | 97.6 | 96.7 | 95.4 | 93.9 |
| SEC, % | (Main Peak) | | | | | | |
| | HMWS | 5.0 | 1.7 | 2.3 | 3.3 | 4.5 | 5.9 |
| | LMWS | Report Result | ND | 0.1 | 0.1 | 0.1 | 0.2 |

| | | | | | | |
|---|---|---|---|---|---|---|
| (ELISA Binding) Bioassay, | 60-140 | 103 | 104 | 105 | 96 | 94 |
| %Relative Potency | | | | | | |

^{a} Acceptance criterion was exceeded, indicating a significant change in the charge profile at the accelerated condition.

TABLE 31
Accelerated Stability Data for Clinical Lot 20230603 at
25° C. ± 2° C./ 60% RH ± 5% RH, Inverted

| | | | Timepoints (months) | | | |
|---|---|---|---|---|---|---|
| | | Acceptance | | 1 | 3 | 6 |
| Test/Attribute | | Criteria | T0 | Month | Months | Months |
| Visible particles (Refer to USP/Ph. Eur.) | | Liquid | L | L | L | L |
| | | EFOVP | EFOVP | EFOVP | EFOVP | EFOVP |
| Color, compared to Standard Solution | | Not more colored | B6 | B5 | B5 | B5 |
| (Refer to Ph. Eur.) | | than No. 2 | | | | |
| Clarity, NTU | | ≤60.0 | 24.1 | 23.7 | 19.7 | 20.1 |
| pH | | 6.7-7.7 | 7.2 | 7.2 | 7.2 | 7.2 |
| Subvisible | ≥25 μm | ≤600 | 0 | 0 | 1 | 0 |
| Particulate | ≥10 μm | ≤6000 | 0 | 6 | 32 | 40 |
| Matter, | ≥5 μm | Report Result | 2 | 27 | 194 | 255 |
| p/c | ≥2 μm | Report Result | 11 | 159 | 998 | 1406 |
| Protein Concentration, mg/mL | | 135.0-165.0 | 152.2 | 153.0 | 153.7 | 150.7 |
| Charge Variants | Main Peak | ≥30.0 | 76.3 | 68.4 | 50.9 | 36.4 |
| Purity by iCIEF, | Acidic Peaks | ≤60.0 | 19.7 | 27.0 | 43.8 | 57.8 |
| % | Basic Peaks | ≤20.0 | 4.0 | 4.5 | 5.3 | 5.8 |
| Purity | LC + HC | ≥90.0 | 98.3 | 97.9 | 96.6 | 94.5 |
| by CE-SDS | Total Minor | Report Result | 1.7 | 2.1 | 3.4 | 5.5 |
| (Reduced), % | Species | | | | | |
| Purity by CE-SDS | Main Peak | ≥90.0 | 98.7 | 98.2 | 96.5 | 94.4 |
| (Non-reduced), % | Total LMWS | Report Result | 1.1 | 1.4 | 2.6 | 4.0 |
| Purity by SEC, | Monomer | ≥95.0 | 98.5 | 97.9 | 96.8 | 95.5 |
| % | (Main Peak) | | | | | |
| | HMWS | ≤5.0 | 1.5 | 2.1 | 3.1 | 4.4 |
| | LMWS | Report Result | ND | 0.1 | 0.1 | 0.1 |

| | | | | | |
|---|---|---|---|---|---|
| Poloxamer 188 Content % (w/v) | Report Result | 0.09 | NT | NT | 0.11 |
| (ELISA Binding) Bioassay, % Relative | 60-140 | 98 | 99 | 94 | 102 |
| Potency | | | | | |

TABLE 32
Accelerated Stability Data for Clinical Lot 20231002
at 25° C. ± 2° C./60% RH ± 5% RH, Inverted

| | | Acceptance | Timepoints (months) | | | |
|---|---|---|---|---|---|---|
| Test/Attribute | | Criteria | 0 | 1 | 3 | 6 |
| Visible particles (Refer to USP/Ph. Eur.) | | Liquid | L | L | L | L |
| | | EFOVP | EFOVP | EFOVP | EFOVP | EFOVP |
| Color, compared to Standard Solution | | Not more colored | B6 | B5 | B5 | B5 |
| (Refer to Ph. Eur.) | | than No. 2 | | | | |
| Clarity, NTU | | ≤60.0 | 21.5 | 20.7 | 19.5 | 19.2 |
| pH | | 6.7-7.7 | 7.2 | 7.2 | 7.2 | 7.3 |
| Subvisible | ≥25 μm | ≤600 | 4 | 0 | 0 | 0 |
| Particulate | ≥10 μm | ≤6000 | 6 | 12 | 16 | 10 |
| Matter, p/c | ≥5 μm | Report Result | 14 | 77 | 98 | 63 |
| | ≥2 μm | Report Result | 178 | 399 | 608 | 410 |
| Protein Concentration, mg/ml | | 135.0-165.0 | 152.1 | 151.6 | 150.1 | 150.9 |
| Charge | Main Peak | ≥30.0 | 74.6 | 67.7 | 53.9 | 38.2 |
| Variants | Acidic Peaks | ≤60.0 | 20.5 | 28.2 | 41.8 | 59.1 |
| Purity by | Basic Peaks | ≤20.0 | 4.9 | 4.1 | 4.2 | 2.8 |
| iCIEF, % | | | | | | |
| Purity by CE-SDS | LC + HC | ≥90.0 | 98.6 | 98.0 | 96.6 | 94.3 |
| (Reduced), % | Total Minor | Report Result | 1.4 | 2.0 | 3.4 | 5.7 |
| | Species | | | | | |
| Purity by | Main Peak | ≥90.0 | 98.6 | 98.1 | 96.6 | 94.2 |
| CE-SDS (Non- | Total LMWS | Report Result | 1.1 | 1.5 | 2.5 | 4.2 |
| reduced), % | | | | | | |
| Purity by SEC, | Monomer (Main Peak) | ≥95.0 | 98.5 | 97.9 | 97.1 | 95.4 |
| % | HMWS | ≤5.0 | 1.5 | 2.1 | 2.7 | 4.4 |
| | LMWS | Report Result | ND | ND | 0.2 | 0.2 |

| | | | | | |
|---|---|---|---|---|---|
| Poloxamer 188 Content % (w/v)^{a} | Report Result | 0.09 | NT | 0.10 | 0.11 |
| (ELISA Binding) Bioassay, | 60-140 | 97 | 100 | 99 | 102 |
| % Relative Potency | | | | | |

TABLE 33
Accelerated Stability Data for Clinical Lot 20240302
at 25° C. ± 2° C., 60% ± 5% RH, Inverted

| | | Acceptance | Timepoints (months) | | | |
|---|---|---|---|---|---|---|
| Test/Attribute | | Criteria | 0 | 1 | 3 | 6 |
| Visible particles | | Liquid | L | L | L | L |
| (Refer to USP/Ph. Eur.) | | EFOVP | EFOVP | EFOVP | EFOVP | EFOVP |
| Color, compared to | | Not more colored | B6 | B5 | B5 | B5 |
| Standard Solution | | than No. 2 | | | | |
| (Refer to Ph. Eur.) | | | | | | |
| Clarity, NTU | | ≤60.0 | 19.6 | 19.1 | 19.4 | 20.1 |
| pH | | 6.7-7.7 | 7.2 | 7.2 | 7.2 | 7.2 |
| Subvisible | ≥25 μm | ≤600 | 0 | 0 | 0 | 9 |
| Particulate | ≥10 μm | ≤6000 | 0 | 12 | 26 | 106 |
| Matter, | ≥5 μm | Report Result | 16 | 56 | 126 | 537 |
| p/c | ≥2 μm | Report Result | 469 | 442 | 691 | 2628 |
| Protein Concentration, mg/mL | | 135.0-165.0 | 151.9 | 154.2 | 152.5 | 160.8 |
| Charge | Main Peak | ≥30.0 | 75.3 | 66.6 | 52.0 | 35.9 |
| Variants | Acidic Peaks | ≤60.0 | 20.5 | 29.4 | 44.3 | 59.6 |
| Purity | Basic Peaks | ≤20.0 | 4.2 | 4.0 | 3.8 | 4.5 |
| by iCIEF, % | | | | | | |
| Purity | LC + HC | ≥90.0 | 98.5 | 97.7 | 96.2 | 94.5 |
| by CE-SDS | Total Minor Species | Report Result | 1.5 | 2.3 | 3.8 | 5.5 |
| (Reduced), % | | | | | | |
| Purity | Main Peak | ≥85.0 | 98.7 | 97.9 | 96.7 | 94.2 |
| by CE-SDS | Total LMWS | Report Result | 1.1 | 1.6 | 2.5 | 4.3 |
| (Non-reduced), % | | | | | | |
| Purity by | Monomer (Main Peak) | ≥90.0 | 98.4 | 97.7 | 96.8 | 95.1 |
| SEC, % | HMWS | ≤5.0 | 1.6 | 2.2 | 3.0 | 4.7 |
| | LMWS | Report Result | ND | 0.1 | 0.2 | 0.3 |

| | | | | | |
|---|---|---|---|---|---|
| Poloxamer 188 Content % (w/v) | Report Result | 0.08 | NT | 0.09 | 0.10 |
| (ELISA Binding) Bioassay, | 60-140 | 101 | 97 | 96 | 96 |
| % Relative Potency | | | | | |

TABLE 34
Stress Stability Data for Clinical Lot 20220502
at 40° C. ± 2° C./75% ± 5% RH, Inverted

| | | | Timepoints | | | |
|---|---|---|---|---|---|---|
| | | Acceptance | | 1 | 2 | 1 |
| Test/Attribute | | Criteria | T0 | Week | Weeks | Month |
| Visible particles (Refer to USP/Ph. Eur.) | | Liquid | L | L | L | L |
| | | EFOVP | EFOVP | EFOVP | EFOVP | EFOVP |
| Color, compared to Standard Solution | | Not more colored | B6 | B5 | B5 | B5 |
| (Refer to Ph. Eur.) | | than No. 3 | | | | |
| Clarity, NTU | | ≤60.0 | 20.4 | 20.3 | 20.8 | 21.4 |
| pH | | 6.7-7.7 | 7.2 | 7.2 | 7.1 | 7.2 |
| Subvisible | ≥25 μm | ≤600 | 0 | 0 | 0 | 1 |
| Particulate | ≥10 μm | ≤6000 | 7 | 5 | 15 | 19 |
| Matter, p/c | ≥5 μm | Report Result | 31 | 30 | 77 | 118 |
| | ≥2 μm | Report Result | 232 | 316 | 607 | 810 |
| Protein Concentration, mg/mL | | 90.0-110.0 | 97.7 | 97.5 | 96.7 | 96.3 |
| Charge Variants | Main Peak | ≥30.0 | 73.3 | 60.4 | 48.0 | 28.7 ^{a} |
| Purity by iCIEF, | Acidic Peaks | ≤60.0 | 20.2 | 33.9 | 46.7 | 67.0 ^{a} |
| % | Basic Peaks | ≤20.0 | 6.5 | 5.7 | 5.3 | 4.3 |
| Purity by CE-SDS | LC + HC | ≥90.0 | 98.9 | 98.2 | 96.2 | 93.9 |
| (Reduced), % | Total Minor Species | ≤10.0 | 1.1 | 1.8 | 3.8 | 6.1 |
| | Main Peak | ≥90.0 | 99.0 | 98.0 | 96.2 | 92.6 |
| Purity by CE- | Total LMWS | ≤10.0 | 0.9 | 1.7 | 3.1 | 5.9 |
| SDS (Non- | | | | | | |
| reduced), % | | | | | | |
| Purity by SEC, % | Monomer | ≥95.0 | 98.7 | 97.0 | 95.6 | 92.3 |
| | (Main Peak) | | | | | |
| | HMWS | ≤5.0 | 1.3 | 2.7 | 4.2 | 7.1 |

| | | | | | |
|---|---|---|---|---|---|
| (ELISA Binding) Bioassay, % | 60-140 | 100 | 98 | 97 | 89 |
| Relative Potency | | | | | |

^{a} Acceptance criterion was exceeded, indicating a significant change in the charge profile at the stress condition.

TABLE 35
Stress Stability Data for Clinical Lot 20230201
at 40° C. ± 2° C./75% ± 5% RH, Inverted

| | | | Timepoints | | | |
|---|---|---|---|---|---|---|
| | | Acceptance | | 1 | 2 | 1 |
| Test/Attribute | | Criteria | T0 | Week | Weeks | Month |
| Visible particles (Refer to USP/Ph. Eur.) | | Liquid | L | L | L | L |
| | | EFOVP | EFOVP | EFOVP | EFOVP | EFOVP |
| Color, compared to Standard Solution | | Not more colored | B6 | B5 | B5 | B5 |
| (Refer to Ph. Eur.) | | than No. 2 | | | | |
| Clarity, NTU | | ≤60.0 | 19.1 | 19.8 | 20.0 | 22.6 |
| pH | | 6.7-7.7 | 7.2 | 7.2 | 7.2 | 7.2 |
| Subvisible | ≥25 μm | ≤600 | 0 | 0 | 0 | 0 |
| Particulate | ≥10 μm | ≤6000 | 2 | 7 | 3 | 11 |
| Matter, p/c | ≥5 μm | Report Result | 26 | 62 | 31 | 68 |
| | ≥2 μm | Report Result | 252 | 213 | 244 | 482 |
| Protein Concentration, mg/mL | | 135.0-165.0 | 152.0 | 154.2 | 155.8 | 156.0 |
| Charge Variants | Main Peak | ≥30.0 | 72.8 | 59.4 | 48.2 | 29.2 ^{a} |
| Purity by iCIEF, % | Acidic Peaks | ≤60.0 | 22.0 | 35.2 | 46.7 | 67.0 ^{a} |
| | Basic Peaks | ≤20.0 | 5.2 | 5.4 | 5.1 | 3.9 |
| Purity by CE-SDS | LC + HC | ≥90.0 | 98.7 | 97.8 | 96.4 | 93.7 |
| (Reduced) | Total Minor Species | Report Result | 1.3 | 2.2 | 3.6 | 6.3 |
| Purity by CE- | Main Peak | ≥85.0 | 98.7 | 97.3 | 96.6 | 92.9 |
| SDS (Non- | Total LMWS | Report Result | 1.1 | 2.1 | 2.8 | 5.7 |
| reduced), % | | | | | | |
| Purity by SEC, % | Monomer | ≥90.0 | 98.3 | 96.9 | 95.2 | 91.4 |
| | (Main Peak) | | | | | |
| | HMWS | ≤5.0 | 1.7 | 3.1 | 4.6 | 8.2 |
| | LMWS | Report Result | ND | ND | 0.3 | 0.4 |

| | | | | | |
|---|---|---|---|---|---|
| (ELISA Binding) Bioassay, | 60-140 | 103 | 102 | 99 | 95 |
| % Relative Potency | | | | | |

^{a} Acceptance criterion was exceeded, indicating a significant change in the charge profile at the stress condition.

TABLE 36
Stress Stability Data for Clinical Lot 20230603
at 40° C. ± 2° C./75% RH ± 5% RH, Inverted

| | | | Timepoints | | | |
|---|---|---|---|---|---|---|
| | | Acceptance | | 1 | 2 | 1 |
| Test/Attribute | | Criteria | T0 | Week | Weeks | Month |
| Visible particles (Refer to USP/Ph. Eur.) | | Liquid | L | L | L | L |
| | | EFOVP | EFOVP | EFOVP | EFOVP | EFOVP |
| Color, compared to Standard Solution | | Not more colored | B6 | B5 | B5 | B5 |
| (Refer to Ph. Eur.) | | than No. 2 | | | | |
| Clarity, NTU | | ≤60.0 | 24.1 | 23.3 | 23.4 | 23.6 |
| pH | | 6.7-7.7 | 7.2 | 7.2 | 7.2 | 7.2 |
| Subvisible | ≥25 μm | ≤600 | 0 | 0 | 0 | 0 |
| Particulate | ≥10 μm | ≤6000 | 0 | 0 | 8 | 0 |
| Matter, p/c | ≥5 μm | Report Result | 2 | 8 | 50 | 9 |
| | ≥2 μm | Report Result | 11 | 65 | 455 | 146 |
| Protein Concentration, mg/mL | | 135.0-165.0 | 152.2 | 149.3 | 152.3 | 153.5 |
| Charge Variants | Main Peak | ≥30.0 | 76.3 | 63.3 | 50.1 | 32.2 |
| Purity by iCIEF, | Acidic Peaks | ≤60.0 | 19.7 | 32.6 | 44.4 | 63.6 ^{a} |
| % | Basic Peaks | ≤20.0 | 4.0 | 4.1 | 5.6 | 14.1 |
| Purity by CE-SDS | LC + HC | ≥90.0 | 98.3 | 97.2 | 96.2 | 93.8 |
| (Reduced), % | Total Minor Species | Report Result | 1.7 | 2.8 | 3.8 | 6.2 |
| Purity by CE- | Main Peak | ≥90.0 | 98.7 | 97.6 | 96.2 | 93.5 |
| SDS (Non- | Total LMWS | Report Result | 1.1 | 2.0 | 3.1 | 5.2 |
| reduced), % | | | | | | |
| Purity by SEC, % | Monomer | ≥95.0 | 98.5 | 97.0 | 95.5 | 92.3 |
| | (Main Peak) | | | | | |
| | HMWS | ≤5.0 | 1.5 | 3.0 | 4.4 | 7.5 |
| | LMWS | Report Result | ND | 0.0 | 0.1 | 0.2 |

| | | | | | |
|---|---|---|---|---|---|
| (ELISA Binding) Bioassay, | 60-140 | 98 | 106 | 98 | 100 |

% Relative Potency

^{a} Acceptance criterion was exceeded, indicating a significant change in the charge profile at the stress condition.

TABLE 37
Stress Stability Data for Clinical Lot 20231002
at 40° C. ± 2° C./75% RH ± 5% RH, Inverted

| | | | Timepoints | | | |
|---|---|---|---|---|---|---|
| | | Acceptance | | 1 | 2 | 1 |
| Test/Attribute | | Criteria | T0 | Week | Weeks | Month |
| Visible particles (Refer to USP/Ph. Eur.) | | Liquid | L | L | L | L |
| | | EFOVP | EFOVP | EFOVP | EFOVP | EFOVP |
| Color, compared to Standard Solution | | Not more colored | B6 | B5 | B5 | B5 |
| (Refer to Ph. Eur.) | | than No. 2 | | | | |
| Clarity, NTU | | ≤60.0 | 21.5 | 21.8 | 22.2 | 20.6 |
| pH | | 6.7-7.7 | 7.2 | 7.2 | 7.2 | 7.2 |
| Subvisible | ≥25 μm | ≤600 | 4 | 2 | 0 | 0 |
| Particulate | ≥10 μm | ≤6000 | 6 | 4 | 2 | 10 |
| Matter, | ≥5 μm | Report Result | 14 | 18 | 16 | 48 |
| p/c | ≥2 μm | Report Result | 178 | 183 | 202 | 281 |
| Protein Concentration, mg/mL | | 135.0-165.0 | 152.1 | 152.7 | 157.1 | 147.4 |
| Charge Variants | Main Peak | ≥30.0 | 74.6 | 61.9 | 50.2 | 30.8 |
| Purity by iCIEF, | Acidic Peaks | ≤60.0 | 20.5 | 33.3 | 45.1 | 64.8 ^{a} |
| % | Basic Peaks | ≤20.0 | 4.9 | 4.8 | 4.7 | 4.4 |
| Purity by CE- | LC + HC | ≥90.0 | 98.6 | 97.2 | 96.6 | 93.4 |
| SDS | Total Minor Species | Report Result | 1.4 | 2.8 | 3.4 | 6.6 |
| (Reduced), % | | | | | | |
| Purity by CE- | Main Peak | ≥90.0 | 98.6 | 97.5 | 96.4 | 93.3 |
| SDS (Non- | Total LMWS | Report Result | 1.1 | 2.0 | 2.8 | 5.5 |
| reduced), % | | | | | | |
| Purity by SEC, % | Monomer (Main Peak) | ≥95.0 | 98.5 | 96.9 | 95.4 | 92.1 |
| | HMWS | ≤5.0 | 1.5 | 3.0 | 4.4 | 7.7 |
| | LMWS | Report Result | ND | 0.1 | 0.1 | 0.3 |

| | | | | | |
|---|---|---|---|---|---|
| (ELISA Binding) | 60-140 | 97 | 105 | 102 | 98 |
| Bioassay, % Relative Potency | | | | | |

^{a} Acceptance criterion was exceeded, indicating a significant change in the charge profile at the stress condition.

TABLE 38
Stress Stability Data for Clinical Lot 20240302
at 40° C. ± 2° C./75% RH ± 5% RH, Inverted

| | | | Timepoints | | | |
|---|---|---|---|---|---|---|
| | | Acceptance | | 1 | 2 | 1 |
| Test/Attribute | | Criteria | T0 | Week | Weeks | Month |
| Visible particles (Refer to USP/Ph. Eur.) | | Liquid | L | L | L | L |
| | | EFOVP | EFOVP | EFOVP | EFOVP | EFOVP |
| Color, compared to Standard Solution | | Not more colored | B6 | B5 | B5 | B5 |
| (Refer to Ph. Eur.) | | than No. 2 | | | | |
| Clarity, NTU | | ≤60.0 | 19.6 | 19.1 | 20.0 | 20.0 |
| pH | | 6.7-7.7 | 7.2 | 7.2 | 7.2 | 7.3 |
| Subvisible | ≥25 μm | ≤600 | 0 | 5 | 0 | 0 |
| Particulate | ≥10 μm | ≤6000 | 0 | 13 | 2 | 1 |
| Matter, | ≥5 μm | Report Result | 16 | 61 | 28 | 14 |
| p/c | ≥2 μm | Report Result | 469 | 342 | 301 | 226 |
| Protein Concentration, mg/mL | | 135.0-165.0 | 151.9 | 152.4 | 154.0 | 153.2 |
| Charge Variants | Main Peak | ≥30.0 | 75.3 | 61.5 | 19.6 | 31.3 |
| Purity by iCIEF, | Acidic Peaks | ≤60.0 | 20.5 | 33.9 | 45.9 | 65.9 ^{a} |
| % | Basic Peaks | ≤20.0 | 4.2 | 4.6 | 4.5 | 2.8 |
| Purity by CE- | LC + HC | ≥90.0 | 98.5 | 98.0 | 96.4 | 93.4 |
| SDS | Total Minor Species | Report Result | 1.5 | 2.0 | 3.6 | 6.6 |
| (Reduced), % | | | | | | |
| Purity by CE- | Main Peak | ≥90.0 | 98.7 | 97.4 | 96.0 | 92.5 |
| SDS (Non- | Total LMWS | Report Result | 1.1 | 2.1 | 3.1 | 6.0 |
| reduced), % | | | | | | |
| Purity by SEC, % | Monomer (Main Peak) | ≥95.0 | 98.4 | 96.8 | 95.4 | 91.6 |
| | HMWS | ≤5.0 | 1.6 | 3.1 | 4.5 | 8.2 ^{a} |
| | LMWS | Report Result | ND | 0.1 | 0.1 | 0.2 |

| | | | | | |
|---|---|---|---|---|---|
| (ELISA Binding) Bioassay, % Relative | 60-140 | 101 | 96 | 97 | 89 |
| Potency | | | | | |

^{a} Acceptance criterion was exceeded, indicating a significant change in the charge profile at the stress condition.

The stability data for development batches are summarized in Table 39 through Table 44 and data for clinical batches are summarized in Table 45 through Table 59.

TABLE 39
Long-term Stability Data for Development and
Non-clinical Batch 2441S211215Y at −70° C. ± 10° C.

| | | Timepoints (months) | | | | | | | |
|---|---|---|---|---|---|---|---|---|---|
| Test/Attribute | | 0 | 1 | 3 | 6 | 9 | 12 | 18 | 24 |
| Color, compared to Standard Solution | | B6 | B6 | B6 | B6 | B6 | B6 | B6 | B6 |
| (Refer to Ph. Eur.) | | | | | | | | | |
| Clarity, NTU | | 21.5 | 21.5 | 22.4 | 21.6 | 22.6 | 21.8 | 21.4 | 22.2 |
| pH | | 7.2 | 7.2 | 7.2 | 7.1 | 7.1 | 7.1 | 7.1 | 7.0 |
| Protein Concentration, mg/mL | | 101.2 | 101.2 | 103.1 | 101.2 | 102.5 | 102.5 | 100.5 | 102.1 |
| Charge Variants | Main Peak | 74.8 | 74.1 | 73.9 | 74.5 | 74.3 | 75.5 | 75.4 | 76.7 |
| Purity by iCIEF, % | Acidic Peaks | 20.3 | 20.4 | 20.6 | 20.9 | 20.8 | 20.0 | 19.9 | 19.0 |
| | Basic Peaks | 4.8 | 5.5 | 5.5 | 4.5 | 4.9 | 4.5 | 4.7 | 4.3 |
| Purity by CE-SDS | LC + HC | 98.4 | 98.8 | 98.1 | 98.6 | 98.4 | 98.7 | 98.4 | 98.7 |
| (Reduced), % | Total Minor Species | 1.6 | 1.2 | 1.9 | 1.4 | 1.6 | 1.3 | 1.6 | 1.3 |
| Purity by CE- | Main Peak | 99.0 | 98.8 | 99.1 | 99.0 | 98.8 | 99.0 | 198.9 | 98.9 |
| SDS (Non- | Total LMWS | 0.8 | 1.1 | 0.9 | 1.0 | 1.0 | 0.9 | 1.1 | 1.0 |
| reduced), % | | | | | | | | | |
| Purity by SEC, % | Monomer | 98.8 | 98.8 | 98.8 | 98.9 | 198.8 | 98.8 | 98.7 | 98.7 |
| | (Main Peak) | | | | | | | | |
| | HMWS | 1.2 | 1.2 | 1.2 | 1.1 | 1.2 | 1.2 | 1.3 | 1.3 |

| | | | | | | | | |
|---|---|---|---|---|---|---|---|---|
| Binding Bioassay (ELISA), | 102 | 105 | 98 | 94 | 104 | 104 | 102 | 100 |
| % Relative potency | | | | | | | | |

TABLE 40
Long-term Stability Data for Development
Batch 2441S220510Y at −70° C. ± 10° C.

| | | Timepoints (months) | | | | | | | |
|---|---|---|---|---|---|---|---|---|---|
| Test/Attribute | | 0 | 1 | 3 | 6 | 9 | 12 | 18 | 24 |
| Color, compared to Standard Solution | | B6 | B6 | B6 | B6 | B6 | B6 | B6 | B6 |
| (Refer to Ph. Eur.) | | | | | | | | | |
| Clarity, NTU | | 21.0 | 21.2 | 20.7 | 21.2 | 21.3 | 20.7 | 21.6 | 21.3 |
| pH | | 7.2 | 7.2 | 7.1 | 7.1 | 7.1 | 7.2 | 7.1 | 7.2 |
| Protein Concentration, mg/mL | | 154.5 | 152.1 | 151.2 | 151.8 | 150.7 | 148.7 | 149.9 | 152.9 |
| Charge | Main Peak | 72.9 | 73.7 | 73.6 | 74.0 | 75.1 | 74.5 | 76.2 | 74.3 |
| Variants | Acidic Peaks | 21.3 | 21.1 | 21.3 | 20.6 | 20.0 | 19.9 | 19.6 | 20.1 |
| Purity by | Basic Peaks | 5.8 | 5.2 | 5.0 | 5.4 | 4.9 | 5.5 | 4.3 | 5.5 |
| iCIEF | | | | | | | | | |
| Purity by CE-SDS | LC + HC | 98.7 | 98.8 | 98.7 | 198.5 | 99.0 | 98.5 | 198.8 | 98.5 |
| (Reduced), % | Total Minor Species | 1.3 | 1.2 | 1.3 | 1.5 | 1.0 | 1.5 | 1.2 | 1.5 |
| Purity by CE- | Main Peak | 98.9 | 98.9 | 98.9 | 98.8 | 98.9 | 98.9 | 98.8 | 99.0 |
| SDS (Non- | Total LMWS | 1.0 | 1.1 | 1.0 | 1.1 | 1.0 | 1.0 | 1.1 | 0.9 |
| reduced), % | | | | | | | | | |
| Purity by SEC, % | Monomer | 98.8 | 98.8 | 98.7 | 98.7 | 198.8 | 98.7 | 98.6 | 98.8 |
| | (Main Peak) | | | | | | | | |
| | HMWS | 1.2 | 1.2 | 1.3 | 1.3 | 1.2 | 1.3 | 1.4 | 1.2 |

| | | | | | | | | |
|---|---|---|---|---|---|---|---|---|
| Binding Bioassay (ELISA), | 88 | 95 | 99 | 99 | 98 | 100 | 102 | 102 |
| % Relative potency | | | | | | | | |

TABLE 41
Accelerated Stability Data for Development and Non-clinical Batch
2441S211215Y at 5° C. ± 3° C.

| | | Timepoints (months) | | | |
|---|---|---|---|---|---|
| Test/ Attribute | | 0 | 1 | 3 | 6 |
| Color, compared to Standard Solution | | B6 | B6 | B6 | B6 |
| (Refer to Ph. Eur.) | | | | | |
| Clarity, NTU | | 21.5 | 20.9 | 22.5 | 21.2 |
| pH | | 7.2 | 7.2 | 7.2 | 7.2 |
| Protein Concentration, mg/mL | | 101.2 | 102.3 | 102.8 | 103.4 |
| Charge Variants | Main Peak | 74.8 | 73.3 | 73.2 | 72.6 |
| Purity by iCIEF, % | Acidic Peaks | 20.3 | 21.3 | 21.7 | 22.7 |
| | Basic Peaks | 4.8 | 5.4 | 5.1 | 4.7 |
| Purity by CE-SDS | LC + HC | 98.4 | 99.0 | 97.7 | 98.5 |
| (Reduced), % | Total Minor Species | 1.6 | 1.0 | 2.3 | 1.5 |
| Purity by CE- | Main Peak | 99.0 | 98.8 | 98.9 | 98.8 |
| SDS (Non- | Total LMWS | 0.8 | 1.1 | 0.9 | 1.0 |
| reduced), % | | | | | |
| Purity by SEC, % | Monomer | 98.8 | 98.7 | 98.6 | 98.7 |
| | (Main | | | | |
| | Peak) | | | | |
| | HMWS | 1.2 | 1.3 | 1.4 | 1.3 |

| | | | | |
|---|---|---|---|---|
| Binding Bioassay (ELISA), | 102 | 101 | 94 | 89 |
| % Relative Potency | | | | |

TABLE 42
Accelerated Stability Data for Development Batch 2441S220510Y
at 5° C. ± 3° C.

| | | Timepoints (months) | | | |
|---|---|---|---|---|---|
| Test/ Attribute | | 0 | 1 | 3 | 6 |
| Color, compared to Standard Solution | | B6 | B6 | B5 | B5 |
| (Refer | | | | | |
| to Ph. Eur.) | | | | | |
| Clarity, NTU | | 21.0 | 20.8 | 19.4 | 19.6 |
| pH | | 7.2 | 7.2 | 7.2 | 7.2 |
| Protein Concentration, mg/mL | | 154.5 | 151.4 | 153.2 | 158.3 |
| Charge Variants | Main Peak | 72.9 | 73.1 | 73.0 | 72.2 |
| Purity by iCIEF, % | Acidic Peaks | 21.3 | 22.1 | 22.3 | 22.6 |
| | Basic Peaks | 5.8 | 4.8 | 4.7 | 5.2 |
| Purity by CE-SDS | LC + HC | 98.7 | 98.6 | 98.2 | 98.3 |
| (Reduced), % | Total Minor Species | 1.3 | 1.4 | 1.8 | 1.7 |
| Purity by CE- | Main Peak | 98.9 | 98.8 | 98.6 | 98.9 |
| SDS (Non- | Total LMWS | 1.0 | 1.1 | 1.2 | 1.0 |
| reduced), % | | | | | |
| Purity by SEC, % | Monomer | 98.8 | 98.7 | 98.4 | 98.3 |
| | (Main | | | | |
| | Peak) | | | | |
| | HMWS | 1.2 | 1.3 | 1.6 | 1.7 |

| | | | | |
|---|---|---|---|---|
| Binding Bioassay (ELISA), | 88 | 106 | 94 | 92 |
| % Relative potency | | | | |

TABLE 43
Stress Stability Data for Development and
Non-clinical Batch 2441S211215Y at 25°
C. ± 2° C. / 60 ± 5% RH

| | | Timepoints (months) | | | |
|---|---|---|---|---|---|
| Test/ Attribute | | 0 | 1 | 2 | 3 |
| Color, compared to Standard Solution | | B6 | B6 | B5 | B5 |
| (Refer to Ph. Eur.) | | | | | |
| Clarity, NTU | | 21.5 | 20.4 | 20.5 | 22.1 |
| pH | | 7.2 | 7.3 | 7.2 | 7.2 |
| Protein Concentration, mg/mL | | 101.2 | 105.5 | 103.4 | 108.9 |
| Charge Variants | Main Peak | 74.8 | 67.1 | 61.4 | 53.8 |
| Purity by iCIEF, % | Acidic Peaks | 20.3 | 27.5 | 34.0 | 41.6 |
| | Basic Peaks | 4.8 | 5.4 | 4.5 | 4.7 |
| Purity by CE-SDS | LC + HC | 98.4 | 98.5 | 96.7 | 96.1 |
| (Reduced), % | Total Minor Species | 1.6 | 1.5 | 3.3 | 3.9 |
| Purity by CE- | Main Peak | 99.0 | 98.4 | 98.0 | 97.1 |
| SDS (Non- | Total LMWS | 0.8 | 1.5 | 1.8 | 2.3 |
| reduced), % | | | | | |
| Purity by SEC, % | Monomer (Main Peak) | 98.8 | 98.3 | 97.9 | 97.6 |
| | HMWS | 1.2 | 1.7 | 2.0 | 2.4 |

| | | | | |
|---|---|---|---|---|
| Binding Bioassay (ELISA), | 102 | 101 | 103 | 100 |
| % Relative potency | | | | |

TABLE 44
Stress Stability Data for Development Batch 2441S220510Y at 25°
C. ± 2° C. / 60 ± 5% RH

| | | Timepoints (months) | | | |
|---|---|---|---|---|---|
| Test/ Attribute | | 0 | 1 | 2 | 3 |
| Color, compared to Standard Solution (Refer | | B6 | B5 | B5 | B5 |
| to Ph. Eur.) | | | | | |
| Clarity, NTU | | 21.0 | 20.2 | 19.5 | 18.8 |
| pH | | 7.2 | 7.2 | 7.2 | 7.2 |
| Protein Concentration, mg/mL | | 154.5 | 154.8 | 157.9 | 161.3 |
| Charge | Main Peak | 72.9 | 66.1 | 60.1 | 53.7 |
| Variants | Acidic Peaks | 21.3 | 28.7 | 34.9 | 41.6 |
| Purity by | Basic Peaks | 5.8 | 5.3 | 5.1 | 4.7 |
| iCIEF | | | | | |
| Purity by CE-SDS | LC + HC | 98.7 | 98.3 | 97.2 | 97.0 |
| (Reduced) | Total Minor Species | 1.3 | 1.7 | 2.8 | 3.0 |
| Purity by CE- | Main Peak | 98.9 | 98.1 | 97.5 | 97.2 |
| SDS (Non- | | | | | |
| reduced) | Total LMWS | 1.0 | 1.6 | 1.8 | 2.3 |
| Purity by SEC | Monomer (Main Peak) | 98.8 | 98.2 | 97.6 | 97.1 |
| | HMWS | 1.2 | 1.8 | 2.3 | 2.8 |

| | | | | |
|---|---|---|---|---|
| Binding Bioassay (ELISA), | 88 | 99 | 100 | 88 |
| % Relative potency | | | | |

TABLE 45
Long-term Stability Data for 100 mg/mL Clinical
Batch 24412201 at −70° C. ± 10° C.

| | | Acceptance | Timepoints (months) | | | | | | | |
|---|---|---|---|---|---|---|---|---|---|---|
| Test/Attribute | | Criteria | 0 | 1 | 3 | 6 | 9 | 12 | 18 | 24 |
| Color, compared to Standard | | Not more | B6 | B6 | B6 | B6 | B6 | B6 | B6 | B6 |
| Solution | | colored | | | | | | | | |
| (Refer to Ph. Eur.) | | than No. 3 | | | | | | | | |
| Clarity, NTU | | ≤60.0 | 21.0 | 20.2 | 20.4 | 21.6 | 20.8 | 21.4 | 22.4 | 21.3 |
| pH | | 6.7-7.7 | 7.1 | 7.2 | 7.2 | 7.1 | 7.1 | 7.1 | 6.9 | 7.0 |
| Protein Concentration, mg/mL | | 90.0-110.0 | 96.8 | 96.0 | 95.8 | 96.4 | 96.1 | 96.2 | 98.1 | 96.2 |
| Charge | Main Peak | ≥30.0 | 74.6 | 73.4 | 73.7 | 74.0 | 74.3 | 73.9 | 73.8 | 74.6 |
| Variants | Acidic Peaks | ≤60.0 | 21.3 | 20.3 | 21.7 | 21.0 | 20.2 | 20.6 | 20.5 | 20.1 |
| Purity by | Basic Peaks | ≤20.0 | 4.0 | 6.3 | 4.6 | 5.0 | 5.5 | 5.6 | 5.7 | 5.3 |
| iCIEF, % | | | | | | | | | | |
| Purity by CE- | LC + HC | ≥90.0 | 98.3 | 98.9 | 99.0 | 98.6 | 98.6 | 98.7 | 98.8 | 98.6 |
| SDS | Total Minor | ≤10.0 | 1.7 | 1.1 | 1.0 | 1.4 | 1.4 | 1.3 | 1.2 | 1.4 |
| (Reduced), % | Species | | | | | | | | | |
| Purity by CE- | Main Peak | ≥90.0 | 98.6 | 99.0 | 98.9 | 98.8 | 98.7 | 98.8 | 98.8 | 98.6 |
| SDS (Non- | Total LMWS | ≤10.0 | 1.2 | 0.9 | 1.0 | 1.0 | 1.1 | 1.1 | 1.0 | 1.2 |
| reduced), % | | | | | | | | | | |
| Purity by | Monomer | ≥95.0 | 98.7 | 98.7 | 98.6 | 98.7 | 98.6 | 98.6 | 98.6 | 98.6 |
| SEC, % | (Main Peak) | | | | | | | | | |
| | HMWS | ≤5.0 | 1.3 | 1.3 | 1.4 | 1.3 | 1.4 | 1.4 | 1.4 | 1.4 |

| | | | | | | | | | |
|---|---|---|---|---|---|---|---|---|---|
| Binding Bioassay (ELISA), | 60-140 | 100 | 102 | 104 | 108 | 93 | 103 | 94 | 102 |
| % Relative potency | | | | | | | | | |

TABLE 46
Long-term Stability Data for 150 mg/mL Clinical Batch 24412202 at −70° C. + 10° C.

| | | Acceptance | Timepoints (months) | | | | | | | |
|---|---|---|---|---|---|---|---|---|---|---|
| Test/Attribute | | Criteria | 0 | 1 | 3 | 6 | 9 | 12 | 18 | 24 |
| Color, compared to | | Not more | B6 | B6 | B6 | B6 | B6 | B6 | B6 | BY5 |
| Standard Solution | | colored | | | | | | | | |
| (Refer to Ph. Eur.) | | than No. 2 | | | | | | | | |
| Clarity, NTU | | ≤60.0 | 20.8 | 19.8 | 20.2 | 19.3 | 21.6 | 25.3 | 22.4 | 20.8 |
| pH | | 6.7-7.7 | 7.1 | 7.2 | 7.1 | 7.0 | 7.0 | 7.1 | 7.0 | 7.0 |
| Protein Concentration, | | 135.0-165.0 | 154.6 | 153.8 | 153.8 | 153.4 | 155.0 | 153.1 | 160.2 | 154.0 |
| mg/mL | | | | | | | | | | |
| Charge | Main Peak | ≥30.0 | 71.1 | 73.4 | 73.2 | 73.4 | 72.4 | 73.4 | 73.0 | 72.9 |
| Variants | Acidic Peaks | ≤60.0 | 23.1 | 21.8 | 21.7 | 21.9 | 22.6 | 22.1 | 21.8 | 21.9 |
| Purity by | Basic Peaks | ≤20.0 | 5.9 | 4.7 | 5.1 | 4.6 | 5.1 | 4.5 | 5.2 | 5.2 |
| iCIEF, % | | | | | | | | | | |
| Purity by | LC + HC | ≥90.0 | 98.9 | 98.2 | 98.5 | 98.5 | 98.5 | 98.5 | 98.5 | 98.5 |
| CE-SDS | Total Minor | ≤10.0 | 1.1 | 1.8 | 1.5 | 1.5 | 1.5 | 1.5 | 1.5 | 1.5 |
| (Reduced), % | Species | | | | | | | | | |
| Purity by CE- | Main Peak | ≥90.0 | 99.0 | 98.9 | 98.7 | 98.8 | 98.8 | 98.7 | 98.6 | 98.9 |
| SDS (Non- | Total LMWS | ≤10.0 | 0.9 | 1.0 | 1.1 | 1.1 | 1.0 | 1.1 | 1.1 | 1.0 |
| reduced), % | | | | | | | | | | |
| Purity by | Monomer | ≥95.0 | 98.5 | 98.6 | 98.4 | 98.5 | 98.4 | 98.4 | 98.4 | 98.3 |
| SEC, % | (Main | | | | | | | | | |
| | Peak) | | | | | | | | | |
| | HMWS | ≤5.0 | 1.5 | 1.4 | 1.6 | 1.5 | 1.6 | 1.6 | 1.6 | 1.7 |

| | | | | | | | | | |
|---|---|---|---|---|---|---|---|---|---|
| Binding Bioassay (ELISA), | 60-140 | 100 | 99 | 103 | 100 | 108 | 104 | 102 | 103 |
| % Relative potency | | | | | | | | | |

TABLE 47
Long-term Stability Data for 150 mg/mL
Clinical Batch 0000097275 at −70° C. + 10° C.

| | | Acceptance | Timepoints (months) | | | | | |
|---|---|---|---|---|---|---|---|---|
| Test/Attribute | | Criteria | 0 | 1 | 3 | 6 | 9 | 12 |
| Color, compared to Standard | | Not more | B6 | B6 | B6 | B6 | B6 | B6 |
| Solution (Refer to Ph. Eur.) | | colored than | | | | | | |
| | | No. 2 | | | | | | |
| Clarity, NTU | | ≤60.0 | 20.2 | 24.5 | 20.5 | 22.8 | 20.3 | 20.4 |
| pH | | 6.7-7.7 | 7.2 | 7.2 | 7.2 | 7.2 | 7.2 | 7.1 |
| Protein Concentration, | | 135.0-165.0 | 156.3 | 157.9 | 148.0 | 154.3 | 152.1 | 151.3 |
| mg/mL | | | | | | | | |
| Charge Variants | Main Peak | ≥30.0 | 74.8 | 75.7 | 74.7 | 75.0 | 75.7 | 75.4 |
| Purity by | Acidic Peaks | ≤60.0 | 19.8 | 19.9 | 20.1 | 20.1 | 19.2 | 19.8 |
| iCIEF, % | Basic Peaks | ≤20.0 | 5.4 | 4.3 | 5.3 | 4.9 | 5.2 | 4.8 |
| Purity by CE- | LC + HC | ≥90.0 | 98.7 | 98.5 | 98.7 | 98.5 | 98.6 | 98.6 |
| SDS | Total Minor | ≤10.0 | 1.3 | 1.5 | 1.3 | 1.5 | 1.4 | 1.4 |
| (Reduced), % | Species | | | | | | | |
| Purity by CE- | Main Peak | ≥90.0 | 98.8 | 98.9 | 98.9 | 98.7 | 98.8 | 98.7 |
| SDS (Non- | Total | ≤10.0 | 1.1 | 1.1 | 1.0 | 1.1 | 1.1 | 1.1 |
| reduced), % | LMWS | | | | | | | |
| Purity by | Monomer | ≥95.0 | 98.5 | 98.5 | 98.5 | 98.5 | 98.5 | 98.6 |
| SEC, % | (Main Peak) | | | | | | | |
| | HMWS | ≤5.0 | 1.5 | 1.5 | 1.5 | 1.5 | 1.5 | 1.4 |

| | | | | | | | |
|---|---|---|---|---|---|---|---|
| Binding Bioassay (ELISA), | 60-140 | 100 | 94 | 109 | 106 | 103 | 97 |
| % Relative potency | | | | | | | |

TABLE 48
Long-term Stability Data for 150 mg/mL Clinical
Batch 0000114279 at −70° C. + 10° C.

| | | Acceptance | Timepoints (months) | | | | |
|---|---|---|---|---|---|---|---|
| Test/Attribute | | Criteria | 0 | 1 | 3 | 6 | 9 |
| Color, compared to | | Not more | B6 | B6 | B6 | B6 | B6 |
| Standard Solution | | colored | | | | | |
| (Refer to Ph. Eur.) | | than No. 2 | | | | | |
| Clarity, NTU | | ≤60.0 | 20.9 | 20.8 | 19.5 | 20.3 | 21 |
| pH | | 6.7-7.7 | 7.1 | 7.2 | 7.2 | 7.0 | 7.0 |
| Protein Concentration, | | 135.0- | 151.8 | 152.6 | 152.5 | 152.7 | 150.8 |
| mg/mL | | 165.0 | | | | | |
| Charge | Main Peak | ≥30.0 | 72.9 | 75.0 | 73.8 | 74.1 | 74.7 |
| Variants | Acidic Peaks | ≤60.0 | 21.9 | 20.9 | 21.5 | 21.2 | 20.8 |
| Purity by | Basic Peaks | ≤20.0 | 5.1 | 4.2 | 4.8 | 4.7 | 4.5 |
| iCIEF, % | | | | | | | |
| Purity by | LC + HC | ≥90.0 | 98.9 | 98.3 | 98.5 | 98.4 | 98.6 |
| CE-SDS | Total Minor | ≤10.0 | 1.1 | 1.7 | 1.5 | 1.6 | 1.4 |
| (Reduced), | Species | | | | | | |
| % | | | | | | | |
| Purity by | Main Peak | ≥90.0 | 98.8 | 98.7 | 98.7 | 98.6 | 98.7 |
| CE-SDS | Total | ≤10.0 | 1.0 | 1.2 | 1.1 | 1.3 | 1.2 |
| (Non- | LMWS | | | | | | |
| reduced), | | | | | | | |
| % | | | | | | | |
| Purity by | Monomer | ≥95.0 | 98.6 | 98.5 | 98.5 | 98.5 | 98.5 |
| SEC, % | (Main | | | | | | |
| | Peak) | | | | | | |
| | HMWS | ≤5.0 | 1.4 | 1.5 | 1.5 | 1.5 | 1.5 |

| | | | | | | |
|---|---|---|---|---|---|---|
| Binding Bioassay | 60-140 | 104 | 86 | 100 | 101 | 102 |
| (ELISA), | | | | | | |
| % Relative potency | | | | | | |

TABLE 49
Long-term Stability Data for 150 mg/mL Clinical Batch
0000124360 at −70° C. + 10° C.

| | | Acceptance | Timepoints (months) | | | |
|---|---|---|---|---|---|---|
| Test/Attribute | | Criteria | 0 | 1 | 3 | 6 |
| Color, compared to | | Not more | BY5 | BY5 | BY5 | B5 |
| Standard Solution | | colored | | | | |
| (Refer to Ph. Eur.) | | than No. 2 | | | | |
| Clarity, NTU | | ≤60.0 | 20.3 | 19.8 | 19.9 | 20.8 |
| pH | | 6.7-7.7 | 7.2 | 7.2 | 7.1 | 7.1 |
| Protein Concentration, | | 135.0- | 154.7 | 156.0 | 153.3 | 161.0 |
| mg/mL | | 165.0 | | | | |
| Charge | Main Peak | ≥30.0 | 74.0 | 72.1 | 72.5 | 72.0 |
| Variants | Acidic Peaks | ≤60.0 | 20.2 | 22.0 | 21.7 | 21.7 |
| Purity by | Basic Peaks | ≤20.0 | 5.8 | 6.0 | 5.8 | 6.4 |
| iCIEF | | | | | | |
| Purity by | LC + HC | ≥90.0 | 98.5 | 98.6 | 98.5 | 98.5 |
| CE-SDS | Total Minor | ≤10.0 | 1.5 | 1.4 | 1.5 | 1.5 |
| (Reduced), % | Species | | | | | |
| Purity by CE- | Main Peak | ≥90.0 | 98.6 | 98.7 | 98.7 | 98.5 |
| SDS (Non- | Total LMWS | ≤10.0 | 1.2 | 1.1 | 1.1 | 1.3 |
| reduced), % | | | | | | |
| Purity by | Monomer | ≥95.0 | 98.4 | 98.4 | 98.4 | 98.4 |
| SEC, % | (Main | | | | | |
| | Peak) | | | | | |
| | HMWS | ≤5.0 | 1.6 | 1.6 | 1.6 | 1.6 |

| | | | | | |
|---|---|---|---|---|---|
| Binding Bioassay (ELISA), | 60-140 | 104 | 101 | 102 | 102 |
| % Relative potency | | | | | |

TABLE 50
Accelerated Stability Data for 100 mg/mL Clinical Batch
24412201 at 5° C. = 3° C.

| | | Acceptance | Timepoints (months) | | | |
|---|---|---|---|---|---|---|
| Test/Attribute | | Criteria | 0 | 1 | 3 | 6 |
| Color, compared to | | Not more | B6 | B6 | B6 | B6 |
| Standard Solution | | colored | | | | |
| (Refer to Ph. Eur.) | | than No. 3 | | | | |
| Clarity, NTU | | ≤60.0 | 21.0 | 20.9 | 20.7 | 20.5 |
| pH | | 6.7-7.7 | 7.1 | 7.2 | 7.2 | 7.2 |
| Protein Concentration, mg/mL | | 90.0-110.0 | 96.8 | 97.2 | 99.4 | 100.2 |
| Charge Variants | Main Peak | ≥30.0 | 74.6 | 72.4 | 72.8 | 72.3 |
| Purity by iCIEF, | Acidic Peaks | ≤60.0 | 21.3 | 20.7 | 22.4 | \|23.0 |
| % | Basic Peaks | ≤20.0 | 4.0 | 6.9 | 4.7 | 4.7 |
| Purity by CE-SDS | LC + HC | ≥90.0 | 98.3 | 98.7 | 98.9 | 98.2 |
| (Reduced), % | Total | ≤10.0 | 1.7 | 1.3 | 1.1 | 1.8 |
| | Minor | | | | | |
| | Species | | | | | |
| Purity by CE- | Main Peak | ≥90.0 | 98.6 | 98.8 | 98.7 | 98.5 |
| SDS (Non- | Total LMWS | ≤10.0 | 1.2 | 1.0 | 1.1 | 1.2 |
| reduced), % | | | | | | |
| Purity by | Monomer | ≥95.0 | 98.7 | 98.7 | 198.4 | 98.4 |
| SEC, % | (Main | | | | | |
| | Peak) | | | | | |
| | HMWS | ≤5.0 | 1.3 | 1.3 | 1.6 | 1.5 |

| | | | | | |
|---|---|---|---|---|---|
| Binding Bioassay (ELISA), | 60-140 | 100 | 107 | 100 | 110 |
| % Relative potency | | | | | |

TABLE 51
Accelerated Stability Data for 150 mg/mL Clinical Batch
24412202 at 5° C. = 3° C.

| | | Acceptance | Timepoints (months) | | | |
|---|---|---|---|---|---|---|
| Test/Attribute | | Criteria | 0 | 1 | 3 | 6 |
| Color, compared to | | Not more | B6 | B6 | B6 | B5 |
| Standard Solution | | colored than | | | | |
| (Refer to Ph. Eur.) | | No. 2 | | | | |
| Clarity, NTU | | ≤60.0 | 20.8 | 20.2 | 19.6 | 18.3 |
| pH | | 6.7-7.7 | 7.1 | 7.1 | 7.2 | 7.2 |
| Protein Concentration, | | 135.0- | 154.6 | 153.1 | 156.1 | 158.4 |
| mg/ml | | 165.0 | | | | |
| Charge | Main Peak | ≥30.0% | 71.1 | 71.9 | 71.7 | 70.6 |
| Variants | Acidic Peaks | ≤60.0 | 23.1 | 22.3 | 23.1 | 24.4 |
| Purity by | Basic Peaks | ≤20.0 | 5.9 | 5.8 | 5.1 | 5.0 |
| iCIEF, % | | | | | | |
| Purity by | LC + HC | ≥90.0 | 98.9 | 98.6 | 98.3 | 98.4 |
| CE-SDS | Total Minor | ≤10.0 | 1.1 | 1.4 | 1.7 | 1.6 |
| (Reduced), % | Species | | | | | |
| Purity by CE- | Main Peak | ≥90.0 | 99.0 | 98.7 | 98.6 | 198.3 |
| SDS (Non- | Total LMWS | ≤10.0 | 0.9 | 1.0 | 1.1 | 1.3 |
| reduced), % | | | | | | |
| Purity by | Monomer | ≥95.0 | 98.5 | 98.5 | 98.1 | 98.0 |
| SEC, % | (Main Peak) | | | | | |
| | HMWS | ≤5.0 | 1.5 | 1.5 | 1.9 | 2.0 |

| | | | | | |
|---|---|---|---|---|---|
| Binding Bioassay | 60-140 | 100 | 101 | 111 | 99 |
| (ELISA), % | | | | | |
| Relative potency | | | | | |

TABLE 52
Accelerated Stability Data for 150 mg/mL Clinical
Batch 0000097275 at 5° C. = 3° C.

| | | Acceptance | Timepoints (months) | | | |
|---|---|---|---|---|---|---|
| Test/Attribute | | Criteria | 0 | 1 | 3 | 6 |
| Color, compared to | | Not more | B6 | B6 | B6 | B6 |
| Standard Solution | | colored than | | | | |
| (Refer to Ph. Eur.) | | No. 2 | | | | |
| Clarity, NTU | | ≤60.0 | 20.2 | 24.0 | 20.0 | 21.4 |
| pH | | 6.7-7.7 | 7.2 | 7.3 | 7.2 | 7.2 |
| Protein Concentration, | | 135.0- | 156.3 | 159.5 | 151.0 | 160.1 |
| mg/ml | | 165.0 | | | | |
| Charge | Main Peak | ≥30.0 | 74.8 | 75.7 | 74.2 | 73.0 |
| Variants | Acidic Peaks | ≤60.0 | 19.8 | 20.8 | 21.1 | 22.4 |
| Purity by | Basic Peaks | ≤20.0 | 5.4 | 3.6 | 4.7 | 4.6 |
| iCIEF, % | | | | | | |
| Purity | LC + HC | ≥90.0 | 98.7 | 98.4 | 98.4 | 98.6 |
| by CE- | Total Minor | ≤10.0 | 1.3 | 1.6 | 1.6 | 1.4 |
| SDS | Species | | | | | |
| (Reduced), % | | | | | | |
| Purity by | Main Peak | ≥90.0 | 98.8 | 98.7 | 98.6 | 98.5 |
| CE-SDS (Non- | Total | ≤10.0 | 1.1 | 1.2 | 1.1 | 1.2 |
| reduced), % | LMWS | | | | | |
| Purity by | Monomer | ≥95.0 | 98.5 | 98.3 | 98.3 | 98.2 |
| SEC, % | (Main Peak) | | | | | |
| | HMWS | ≤5.0 | 1.5 | 1.7 | 1.7 | 1.8 |

| | | | | | |
|---|---|---|---|---|---|
| Binding Bioassay | 60-140 | 100 | 92 | 103 | 112 |
| (ELISA), % Relative | | | | | |
| potency | | | | | |

TABLE 53
Accelerated Stability Data for 150 mg/mL Clinical Batch
0000114279 at 5° C. = 3° C.

| | | Acceptance | Timepoints (months) | | | |
|---|---|---|---|---|---|---|
| Test/Attribute | | Criteria | 0 | 1 | 3 | 6 |
| Color, compared to | | Not more | B6 | B6 | BY5 | B5 |
| Standard Solution | | colored | | | | |
| (Refer to Ph. Eur.) | | than No. 2 | | | | |
| Clarity, NTU | | ≤60.0 | 20.9 | 20.5 | 18.9 | 18.9 |
| pH | | 6.7-7.7 | 7.2 | 7.2 | 7.2 | 7.2 |
| Protein Concentration, | | 135.0-165.0 | 151.8 | 154.1 | 152.9 | 158.9 |
| mg/mL | | | | | | |
| Charge | Main Peak | ≥30.0 | 72.9 | 73.9 | 73.3 | 72.2 |
| Variants | Acidic Peaks | ≤60.0 | 21.9 | 21.4 | 22.4 | 22.9 |
| Purity by | Basic Peaks | ≤20.0 | 5.1 | 4.7 | 4.3 | 4.9 |
| iCIEF, % | | | | | | |
| Purity by | LC + HC | ≥90.0 | 98.9 | 98.5 | 98.3 | 98.3 |
| CE-SDS | Total Minor | ≤10.0 | 1.1 | 1.5 | 1.7 | 1.7 |
| (Reduced), | Species | | | | | |
| % | | | | | | |
| Purity by | Main Peak | ≥90.0 | 98.8 | 98.7 | 98.6 | 98.4 |
| CE-SDS | Total | ≤10.0 | 1.0 | 1.2 | 1.2 | 1.2 |
| (Non- | LMWS | | | | | |
| reduced), % | | | | | | |
| Purity | Monomer | ≥95.0 | 98.6 | 98.3 | 98.2 | 98.1 |
| by | (Main Peak) | | | | | |
| SEC, % | HMWS | ≤5.0 | 1.4 | 1.7 | 1.8 | 1.9 |

| | | | | | |
|---|---|---|---|---|---|
| Binding Bioassay | 60-140 | 104 | 101 | 110 | 89 |
| (ELISA), % Relative | | | | | |
| Potency | | | | | |

TABLE 54
Accelerated Stability Data for 150 mg/mL Clinical Batch
0000124360 at 5° C. + 3° C.

| | | Acceptance | Timepoints (months) | | | |
|---|---|---|---|---|---|---|
| Test/Attribute | | Criteria | 0 | 1 | 3 | 6 |
| Color, compared to | | Not more | BY5 | BY5 | B5 | B5 |
| Standard Solution | | colored than | | | | |
| (Refer to Ph. Eur.) | | No. 2 | | | | |
| Clarity, NTU | | ≤60.0 | 20.3 | 20.0 | 19.6 | 18.9 |
| pH | | 6.7-7.7 | 7.2 | 7.2 | 7.2 | 7.2 |
| Protein Concentration, | | 135.0-165.0 | 154.7 | 156.9 | 156.1 | 170.2 |
| mg/mL | | | | | | |
| Charge | Main Peak | ≥30.0 | 74.0 | 71.8 | 70.9 | 70.1 |
| Variants | Acidic Peaks | ≤60.0 | 20.2 | 22.2 | 23.4 | 23.5 |
| Purity by | Basic Peaks | ≤20.0 | 5.8 | 6.0 | 5.7 | 6.3 |
| iCIEF, % | | | | | | |
| Purity | LC + HC | ≥90.0 | 98.5 | 98.4 | 98.4 | 98.3 |
| by CE- | Total Minor | ≤10.0 | 1.5 | 1.6 | 1.6 | 1.7 |
| SDS | Species | | | | | |
| (Reduced), % | | | | | | |
| Purity by | Main Peak | ≥90.0 | 98.6 | 98.6 | 98.6 | 98.4 |
| CE-SDS (Non- | Total | ≤10.0 | 1.2 | 1.1 | 1.1 | 1.3 |
| reduced), % | LMWS | | | | | |
| Purity by | Monomer | ≥95.0 | 98.4 | 98.3 | 98.1 | 197.9 |
| SEC, % | (Main Peak) | | | | | |
| | HMWS | ≤5.0 | 1.6 | 1.7 | 1.9 | 2.1 |

| | | | | | |
|---|---|---|---|---|---|
| Binding Bioassay | 60-140 | 104 | 103 | 105 | 95 |
| (ELISA), % Relative | | | | | |
| Potency | | | | | |

TABLE 55
Stress Stability Data for 100 mg/mL Clinical Batch 24412201 at
25° C. + 2° C./60% + 5% RH

| | | Acceptance | Timepoints (months) | | | |
|---|---|---|---|---|---|---|
| Test/Attribute | | Criteria | 0 | 1 | 2 | 3 |
| Color, compared to | | Not more | B6 | B6 | B6 | B5 |
| Standard Solution | | colored | | | | |
| (Refer to Ph. Eur.) | | than No. 3 | | | | |
| Clarity, NTU | | ≤60.0 | 21.0 | 20.2 | 20.1 | 20.3 |
| pH | | 6.7-7.7 | 7.1 | 7.2 | 7.2 | 7.2 |
| Protein Concentration, | | 90.0-110.0 | 96.8 | 99.4 | 101.7 | 103.3 |
| mg/mL | | | | | | |
| Charge Variants | Main Peak | ≥30.0 | 74.6 | 64.3 | 59.1 | 54.1 |
| Purity by | Acidic Peaks | ≤60.0 | 21.3 | 28.7 | 35.1 | 42.4 |
| iCIEF, % | Basic Peaks | ≤20.0 | 4.0 | 7.0 | 5.8 | 3.6 |
| Purity by | LC + HC | ≥90.0 | 98.3 | 98.5 | 97.9 | 97.8 |
| CE-SDS | Total Minor | ≤10.0 | 1.7 | 1.5 | 2.1 | 2.2 |
| (Reduced), % | Species | | | | | |
| Purity by CE- | Main Peak | ≥90.0 | 98.6 | 98.6 | 97.8 | 97.3 |
| SDS (Non- | Total | ≤10.0 | 1.2 | 1.2 | 1.8 | 2.2 |
| reduced), % | LMWS | | | | | |
| Purity by | Monomer | ≥95.0 | 98.7 | 98.3 | 98.0 | 97.3 |
| SEC, % | (Main Peak) | | | | | |
| | HMWS | ≤5.0 | 1.3 | 1.7 | 2.0 | 2.6 |

| | | | | | |
|---|---|---|---|---|---|
| Binding Bioassay | 60-140 | 100 | 93 | 94 | 97 |
| (ELISA), % | | | | | |
| Relative Potency | | | | | |

TABLE 56
Stress Stability Data for 150 mg/mL Clinical Batch 24412202 at
25° C. + 2° C./60% + 5% RH

| | | Acceptance | Timepoints (months) | | | |
|---|---|---|---|---|---|---|
| Test/Attribute | | Criteria | 0 | 1 | 2 | 3 |
| Color, compared to | | Not more | B6 | B5 | B5 | B5 |
| Standard Solution | | colored | | | | |
| (Refer to Ph. Eur.) | | than No. 2 | | | | |
| Clarity, NTU | | ≤60.0 | 20.8 | 19.4 | 18.9 | 18.3 |
| pH | | 6.7-7.7 | 7.1 | 7.2 | 7.2 | 7.2 |
| Protein Concentration, | | 135.0-165.0 | 154.6 | 154.8 | 159.1 | 161.2 |
| mg/mL | | | | | | |
| Charge Variants | Main Peak | ≥30.0 | 71.1 | 65.3 | 57.6 | 51.8 |
| Purity by | Acidic Peaks | ≤60.0 | 23.1 | 30.2 | 37.4 | 43.7 |
| iCIEF, % | Basic Peaks | ≤20.0 | 5.9 | 4.5 | 5.1 | 4.5 |
| Purity by | LC + HC | ≥90.0 | 98.9 | 98.1 | 97.6 | 96.4 |
| CE-SDS | Total Minor | ≤10.0 | 1.1 | 1.9 | 2.4 | 3.6 |
| (Reduced), % | Species | | | | | |
| Purity by CE- | Main Peak | ≥90.0 | 99.0 | 98.0 | 97.2 | 96.4 |
| SDS (Non- | Total LMWS | ≤10.0 | 0.9 | 1.5 | 2.1 | 2.6 |
| reduced), % | | | | | | |
| Purity by | Monomer | ≥95.0 | 98.5 | 97.9 | 96.9 | 96.5 |
| SEC, % | (Main Peak) | | | | | |
| | HMWS | ≤5.0 | 1.5 | 2.1 | 3.0 | 3.4 |

| | | | | | |
|---|---|---|---|---|---|
| Binding Bioassay (ELISA), | 60-140 | 100 | 102 | 102 | 105 |
| % Relative Potency | | | | | |

TABLE 57
Stress Stability Data for 150mg/mL Clinical Batch 0000097275
at 25° C. + 2° C./60% + 5% RH

| | | Acceptance | Timepoints (months) | | | | |
|---|---|---|---|---|---|---|---|
| Test/Attribute | | Criteria | 0 | 1 | 2 | 3 | 6 |
| Color, compared to | | Not more | B6 | B6 | B5 | B5 | B5 |
| Standard Solution | | colored | | | | | |
| (Refer to Ph. Eur.) | | than No. 2 | | | | | |
| Clarity, NTU | | ≤60.0 | 20.2 | 23.2 | 23.0 | 18.8 | 19.5 |
| pH | | 6.7-7.7 | 7.2 | 7.3 | 7.2 | 7.2 | 7.2 |
| Protein Concentration, | | 135.0-165.0 | 156.3 | 161.3 | 157.8 | 156.7 | 167.2 |
| mg/mL | | | | | | | |
| Charge | Main Peak | ≥30.0 | 74.8 | 68.2 | 59.2 | 53.8 | 37.5 |
| Variants | Acidic Peaks | ≤60.0 | 19.8 | \|27.9 | 35.2 | 41.0 | 56.5 |
| Purity by | Basic Peaks | ≤20.0 | 5.4 | 3.9 | 3.8 | 5.2 | 6.0 |
| iCIEF, % | | | | | | | |
| Purity by | LC + HC | ≥90.0 | 98.7 | 97.9 | 97.4 | 96.7 | 95.4 |
| CE-SDS | Total Minor | ≤10.0 | 1.3 | 2.1 | 2.6 | 3.3 | 4.6 |
| (Reduced), | Species | | | | | | |
| % | | | | | | | |
| Purity by | Main Peak | ≥90.0 | 98.8 | 98.2 | 97.5 | 96.8 | 94.4 |
| CE-SDS | Total | ≤10.0 | 1.1 | 1.5 | 2.0 | 2.4 | 4.1 |
| (Non- | LMWS | | | | | | |
| reduced), | | | | | | | |
| % | | | | | | | |
| Purity by | Monomer | ≥95.0 | 98.5 | 97.8 | 97.4 | 97.0 | 95.7 |
| SEC, % | (Main Peak) | | | | | | |
| | HMWS | ≤5.0 | 1.5 | 2.1 | 2.5 | 2.9 | 4.2 |

| | | | | | | |
|---|---|---|---|---|---|---|
| Binding Bioassay | 60-140 | 100 | 93 | 100 | 99 | 106 |
| (ELISA), % | | | | | | |
| Relative potency | | | | | | |

TABLE 58
Stress Stability Data for 150 mg/mL Clinical Batch 0000114279 at
25° C. + 2° C./60% = 5% RH

| | | Acceptance | Timepoints (months) | | | | |
|---|---|---|---|---|---|---|---|
| Test/Attribute | | Criteria | 0 | 1 | 2 | 3 | 6 |
| Color, compared to | | Not more | B6 | B5 | B5 | B5 | B5 |
| Standard Solution | | colored | | | | | |
| (Refer to Ph. Eur.) | | than No. 2 | | | | | |
| Clarity, NTU | | ≤60.0 | 20.9 | 19.5 | 19.0 | 18.4 | 16.7 |
| pH | | 6.7-7.7 | 7.1 | 7.2 | 7.2 | 7.2 | 7.2 |
| Protein Concentration, | | 135.0- | 151.8 | 158.1 | 152.6 | 159.8 | 167.8 |
| mg/mL | | 165.0 | | | | | |
| Charge | Main Peak | ≥30.0 | 72.9 | 67.1 | 60.5 | 54.3 | 38.8 |
| Variants | Acidic Peaks | ≤60.0 | 21.9 | 28.7 | 35.3 | 41.9 | 57.3 |
| Purity by | Basic Peaks | ≤20.0 | 5.1 | 4.2 | 4.2 | 3.9 | 3.9 |
| iCIEF, % | | | | | | | |
| Purity by | LC + HC | ≥90.0 | 98.9 | 98.0 | 97.4 | 96.7 | 95.0 |
| CE-SDS | Total Minor | ≤10.0 | 1.1 | 2.0 | 2.6 | 3.3 | 5.0 |
| (Reduced), | Species | | | | | | |
| % | | | | | | | |
| Purity by | Main Peak | ≥90.0 | 98.8 | 97.9 | 97.2 | 96.6 | 94.4 |
| CE-SDS | Total | ≤10.0 | 1.0 | 1.7 | 2.0 | 2.5 | 4.2 |
| (Non- | LMWS | | | | | | |
| reduced), | | | | | | | |
| % | | | | | | | |
| Purity by | Monomer | ≥95.0 | 98.6 | 97.8 | 97.3 | 96.7 | 95.5 |
| SEC, % | (Main Peak) | | | | | | |
| | HMWS | ≤5.0 | 1.4 | 2.1 | 2.6 | 3.1 | 4.4 |

| | | | | | | |
|---|---|---|---|---|---|---|
| Binding Bioassay | 60-140 | 104 | 98 | 106 | 105 | 73 |
| (ELISA), % | | | | | | |
| Relative potency | | | | | | |

TABLE 59
Stress Stability Data for 150 mg/mL Clinical Batch 0000124360 at
25° C. + 2° C./60% = 5% RH

| | | Acceptance | Timepoints (months) | | | | |
|---|---|---|---|---|---|---|---|
| Test/Attribute | | Criteria | 0 | 1 | 2 | 3 | 6 |
| Color, compared to | | Not more | BY5 | B5 | B5 | B5 | B5 |
| Standard Solution | | colored | | | | | |
| (Refer to Ph. Eur.) | | than No. 2 | | | | | |
| Clarity, NTU | | ≤60.0 | 20.3 | 19.0 | 19.0 | 18.4 | 17.2 |
| pH | | 6.7-7.7 | 7.2 | 7.2 | 7.2 | 7.2 | 7.2 |
| Protein Concentration, | | 135.0- | 154.7 | 159.7 | 157.8 | 161.1 | 180.9 |
| mg/mL | | 165.0 | | | | | |
| Charge | Main Peak | ≥30.0 | 74.0 | 64.7 | 58.3 | 52.5 | 36.2 |
| Variants | Acidic Peaks | ≤60.0 | 20.2 | 29.5 | 35.4 | 42.7 | 58.2 |
| Purity by | Basic Peaks | ≤20.0 | 5.8 | 5.8 | 6.3 | 4.8 | 5.6 |
| iCIEF, % | | | | | | | |
| Purity by | LC + HC | ≥90.0 | 98.5 | 97.8 | 96.9 | 96.4 | 94.6 |
| CE-SDS | Total Minor | ≤10.0 | 1.5 | 2.2 | 3.1 | 3.6 | 5.4 |
| (Reduced), | Species | | | | | | |
| % | | | | | | | |
| Purity by | Main Peak | ≥90.0 | 98.6 | 97.8 | 97.2 | 96.6 | 194.4 |
| CE-SDS | Total | ≤10.0 | 1.2 | 1.6 | 2.0 | 2.5 | 4.0 |
| (Non- | LMWS | | | | | | |
| reduced), | | | | | | | |
| % | | | | | | | |
| Purity by | Monomer | ≥95.0 | 98.4 | 97.7 | 97.1 | 96.7 | 95.0 |
| SEC, % | (Main Peak) | | | | | | |
| | HMWS | ≤5.0 | 1.6 | 2.3 | 2.7 | 3.2 | 4.8 |

| | | | | | | |
|---|---|---|---|---|---|---|
| Binding Bioassay | 60-140 | 104 | 91 | 98 | 98 | 91 |
| (ELISA), % | | | | | | |
| Relative potency | | | | | | |

TABLE 60
Accelerated Stability Data for Clinical Lot 20240603 at 25° C.
± 2° C. / 60% RH ± 5% RH, Inverted

| | | | Timepoints | |
|---|---|---|---|---|
| | | Acceptance | (months) | |
| Test/ Attribute | | Criteria | T0 | 1 Month |
| Visible particles (Refer to USP/Ph. Eur.) | | Liquid | L | L |
| | | EFOVP | EFOVP | EFOVP |
| Color, compared to Standard Solution | | Not more colored | BY5 | B5 |
| (Refer to Ph. Eur.) | | than No. 2 | | |
| Clarity, NTU | | ≤60.0 | 20.0 | 19.5 |
| pH | | 6.7-7.7 | 7.2 | 7.2 |
| Subvisible | ≥25 μm | ≤600 | 1 | 0 |
| Particulate | ≥10 μm | ≤6000 | 2 | 16 |
| Matter, | ≥5 μm | Report Result | 24 | 78 |
| p/c | ≥2 μm | Report Result | 189 | 454 |
| Protein Concentration, mg/mL | | 135.0-165.0 | 151.3 | 153.9 |
| Charge Variants | Main Peak | ≥30.0 | 71.8 | 63.2 |
| Purity by iCIEF, | Acidic Peaks | ≤60.0 | 21.8 | 30.4 |
| % | Basic Peaks | ≤20.0 | 6.4 | 6.4 |
| Purity | LC + HC | ≥90.0 | 98.4 | 97.9 |
| by CE- | Total Minor Species | Report Result | 1.6 | 2.1 |
| SDS | | | | |
| (Reduced), % | | | | |
| Purity by | Main Peak | ≥90.0 | 98.7 | 97.6 |
| CE-SDS (Non- | | | | |
| reduced), % | Total LMWS | Report Result | 1.1 | 1.8 |
| Purity by SEC, | Monomer (Main | ≥95.0 | 98.2 | 97.4 |
| | Peak) | | | |
| % | HMWS | ≤5.0 | 1.8 | 2.4 |
| | LMWS | Report Result | ND | 0.2 |

| | | | |
|---|---|---|---|
| Poloxamer 188 Content %(w/v) | Report Result | 0.10 | NT |
| (ELISA Binding) Bioassay, % Relative | 60-140 | 104 | 107 |
| Potency | | | |

TABLE 61
Stress Stability Data for Clinical Lot 20240603 at
40° C. + 2° C./75% RH + 5% RH, Inverted

| | | | Timepoints | | | |
|---|---|---|---|---|---|---|
| | | Acceptance | | 1 | 2 | 1 |
| Test/Attribute | | Criteria | T0 | Week | Weeks | Month |
| Visible particles | | Liquid | L | L | L | L |
| (Refer to USP/ | | EFOVP | EFOVP | EFOVP | EFOVP | EFOVP |
| Ph. Eur.) | | | | | | |
| Color, compared to | | Not more | BY5 | B5 | B5 | B5 |
| Standard Solution | | colored | | | | |
| (Refer to Ph. Eur.) | | than No. 2 | | | | |
| Clarity, NTU | | ≤60.0 | 20.0 | 19.7 | 19.4 | 20.3 |
| pH | | 6.7-7.7 | 7.2 | 7.2 | 7.2 | 7.2 |
| Subvisible | ≥25 μm | ≤600 | 1 | 0 | 0 | 0 |
| Particulate | ≥10 μm | ≤6000 | 2 | 16 | 6 | 2 |
| Matter, | ≥5 μm | Report | 24 | 111 | 30 | 22 |
| p/c | | Result | | | | |
| | ≥2 μm | Report | 189 | 430 | 174 | 158 |
| | | Result | | | | |
| Protein Concentration, | | 135.0- | 151.3 | 152.7 | 154.3 | 154.8 |
| mg/mL | | 165.0 | | | | |
| Charge | Main Peak | ≥30.0 | 71.8 | 59.8 | 49.3 | 25.8 |
| Variants | Acidic | ≤60.0 | 21.8 | 34.9 | 45.1 | 70.0 |
| | Peaks | | | | | |
| Purity by | Basic | ≤20.0 | 6.4 | 5.3 | 5.6 | 4.3 |
| iCIEF, % | Peaks | | | | | |
| Purity by | LC + HC | ≥90.0 | 98.4 | 97.0 | 96.7 | 92.9 |
| CE-SDS | Total | Report | 1.6 | 3.0 | 3.3 | 7.1 |
| (Reduced), | Minor | Result | | | | |
| % | Species | | | | | |
| Purity by | Main Peak | ≥90.0 | 98.7 | 96.9 | 96.2 | 91.9 |
| CE-SDS | Total | Report | 1.1 | 2.0 | 3.0 | 6.3 |
| (Non- | LMWS | Result | | | | |
| reduced), | | | | | | |
| % | | | | | | |
| Purity by | Monomer | ≥95.0 | 98.2 | 96.5 | 95.1 | 90.4 |
| SEC, % | (Main | | | | | |
| | Peak) | | | | | |
| | HMWS | ≤5.0 | 1.8 | 3.3 | 4.7 | 9.1 |
| | LMWS | Report | ND | 0.2 | 0.3 | 0.5 |
| | | Result | | | | |

| | | | | | |
|---|---|---|---|---|---|
| (ELISA Binding) | 60-140 | 104 | 102 | 99 | 98 |
| Bioassay, | | | | | |
| % Relative Potency | | | | | |
| Polyxmaer 188 | Report | 0.10 | NT | NT | NT |
| Content | Result | | | | |

TABLE 62
Long Term Stability Data for Clinical Lot 20240603 at
5° C. + 3° C., Inverted

| | | | Timepoints | |
|---|---|---|---|---|
| | | Acceptance | (months) | |
| Test/ Attribute | | Criteria | 0 | 1 |
| Visible particles (Refer to | | Liquid | L | L |
| USP/Ph. Eur.) | | EFOVP | EFOVP | EFOVP |
| Color, compared to | | Not more | BY5 | B5 |
| Standard | | colored | | |
| Solution (Refer to Ph. Eur.) | | than No. | | |
| | | 2 | | |
| Clarity, NTU | | ≤60.0 | 20.0 | 19.8 |
| pH | | 6.7-7.7 | 7.2 | 7.2 |
| Subvisible | ≥25 μm | ≤600 | 1 | 0 |
| Particulate | ≥10 μm | ≤6000 | 2 | 2 |
| Matter, | ≥5 μm | Report Result | 24 | 29 |
| p/c | ≥2 μm | Report Result | 189 | 269 |
| Protein Concentration, | | 135.0-165.0 | 151.3 | 152.1 |
| mg/mL | | | | |
| Charge | Main Peak | ≥30.0 | 71.8 | 71.3 |
| Variants | Acidic Peaks | ≤60.0 | 21.8 | 22.8 |
| Purity | Basic Peaks | ≤20.0 | 6.4 | 5.9 |
| by | | | | |
| iCIEF, % | | | | |
| Purity | LC + HC | ≥90.0 | 98.4 | 98.5 |
| by CE- | Total | Report Result | 1.6 | 1.5 |
| SDS | Minor | | | |
| (Reduced), % | Species | | | |
| Purity | Main Peak | ≥85.0 | 98.7 | 98.4 |
| by CE- | Total LMWS | Report Result | 1.1 | 1.3 |
| SDS | | | | |
| (Non- | | | | |
| reduced), % | | | | |
| Purity by | Monomer | ≥90.0 | 98.2 | 98.1 |
| SEC, % | (Main Peak) | | | |
| | HMWS | 5.0 | 1.8 | 1.9 |
| | LMWS | Report Result | ND | ND |

| | | | |
|---|---|---|---|
| Poloxamer 188 Content, % | Report Result | 0.10 | NT |
| (w/v) | | | |
| (ELISA Binding) Bioassay, % | 60-140 | 104 | 104 |
| Relative Potency | | | |
| Sterility | No Growth | NG | NT |
| CCIT | Pass | NT | NT |

TABLE 63
Long Term Stability Data for Clinical Lot 20240603
at 5° C. ± 3° C., Upright

| | | | Timepoints | |
|---|---|---|---|---|
| | | Acceptance | (months) | |
| Test/ Attribute | | Criteria | 0 | 1 |
| Visible particles (Refer to | | Liquid | L | L |
| USP/Ph. Eur.) | | EFOVP | EFOVP | EFOVP |
| Color, compared to Standard | | Not more | BY5 | B5 |
| Solution (Refer to Ph. Eur.) | | colored | | |
| | | than No. | | |
| | | 2 | | |
| Clarity, NTU | | ≤60.0 | 20.0 | 20.0 |
| pH | | 6.7-7.7 | 7.2 | 7.2 |
| Subvisible | ≥25 μm | ≤600 | 1 | NT |
| Particulate | ≥10 μm | ≤6000 | 2 | NT |
| Matter, p/c | ≥5 μm | Report | 24 | NT |
| | | Result | | |
| | ≥2 μm | Report | 189 | NT |
| | | Result | | |
| Protein Concentration, | | 135.0-165.0 | 151.3 | 152.8 |
| mg/mL | | | | |
| Charge | Main Peak | ≥30.0 | 71.8 | 71.4 |
| Variants | Acidic Peaks | ≤60.0 | 21.8 | 22.7 |
| Purity | Basic Peaks | ≤20.0 | 6.4 | 5.9 |
| by iCIEF, % | | | | |
| Purity | LC + HC | ≥90.0 | 98.4 | 98.5 |
| by CE- | Total | Report | 1.6 | 1.5 |
| SDS | Minor | Result | | |
| (Reduced), % | Species | | | |
| Purity | Main Peak | ≥85.0 | 98.7 | 98.4 |
| by CE- | Total LMWS | Report | 1.1 | 1.3 |
| SDS | | Result | | |
| (Non- | | | | |
| reduced), % | | | | |
| Purity by | Monomer | ≥90.0 | 98.2 | 98.1 |
| SEC, % | (Main Peak) | | | |
| | HMWS | ≤5.0 | 1.8 | 1.9 |
| | LMWS | Report | ND | ND |
| | | Result | | |

| | | | |
|---|---|---|---|
| Poloxamer 188 Content, % | Report | 0.10 | NT |
| (w/v) | Result | | |
| (ELISA Binding) Bioassay, % | 60-140 | 104 | 104 |
| Relative Potency | | | |
| Sterility | No Growth | NG | NT |
| CCIT | Pass | NT | NT |

The stability data demonstrate no significant change over time and no significant variability in stability measures. Stability results meet all acceptance criteria.

Thus the embodiments and examples provided for herein demonstrate the surprising and/or unexpected results that a high concentration formulation comprising the antibodies provided for herein can be sufficiently stable for sufficient periods of time to be used, for example, in the methods provided for herein. The formulations prevent sufficient aggregation as determined by the absence of a significant percentage of HMWS as well as prevents degradation, which would be measured by the presence of LMWS. The purity of the composition is also demonstrated the high percentage of the main peaks provided for in the examples.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications in addition to those described herein will become apparent to those skilled in the art from the foregoing description. Such modifications are intended to fall within the scope of the embodiments and any appended claims.

The present specification is considered to be sufficient to enable one skilled in the art to practice the embodiments. Various modifications in addition to those shown and described herein will become apparent to those skilled in the art from the foregoing description and fall within the scope of the present disclosure and any appended claims.

1. A pharmaceutical composition comprising:
about 25 mg/mL to about 300 mg/mL of an antibody having a heavy chain and a light chain, wherein the heavy chain comprises a HCDR1 of SEQ ID NO: 61, a HCDR2 of SEQ ID NO: 62, and a HCDR3 of SEQ ID NO: 78, and wherein the light chain comprises a LCDR1 of SEQ ID NO: 64, a LCDR2 of SEQ ID NO: 65, and a LCDR3 of SEQ ID NO: 79;
a buffer at a concentration of about 5 mM to about 50 mM;
an antioxidant at a concentration of about 5 mM to about 15 mM;
a sugar at a concentration of about 1% w/v to about 14% w/v;
a viscosity modifying agent at a concentration of about 20 mM to about 180 mM; and
a surfactant present in an amount of about 0.001% w/v to about 0.4% w/v, and
wherein the pharmaceutical composition has a pH of about 6.5 to about 8.0.

2-5. (canceled)

6. The pharmaceutical composition of claim 1, wherein the buffer is selected from tris buffer, histidine buffer, HEPES, phosphate buffer, acetate buffer, citrate buffer, succinate buffer, ascorbate buffer, glutamate buffer, lactate buffer, maleate buffer, trometamol buffer, gluconate buffer, or any combination thereof.

7-12. (canceled)

13. The pharmaceutical composition of claim 1, wherein the antioxidant is selected from L-methionine, ascorbic acid, EDTA, or any combination thereof.

14-19. (canceled)

20. The pharmaceutical composition of claim 1, wherein the sugar is selected from sucrose, trehalose, sorbitol, mannitol, or any combination thereof.

21-23. (canceled)

24. The pharmaceutical composition of claim 1, wherein the viscosity modifying agent is L-arginine hydrochloride (L-Arg-HCL) or sodium chloride (NaCl).

25-27. (canceled)

28. The pharmaceutical composition of claim 1, wherein the surfactant is Polysorbate 80 (PS80), Polysorbate 20 (PS20), polyethylene glycol 3350 (PEG3350), or Poloxamer 188 (P188).

29-34. (canceled)

35. The pharmaceutical composition of claim 1, wherein:
the antibody comprises a heavy chain variable region with an amino acid sequence of SEQ ID NO: 17; and a light chain variable region with an amino acid sequence of SEQ ID NO: 18; or
the antibody comprises a heavy chain variable region with an amino acid sequence of SEQ ID NO: 342; and a light chain variable region with an amino acid sequence of SEQ ID NO: 18.

36-37. (canceled)

38. The pharmaceutical composition of claim 1, wherein the pharmaceutical composition is stable at about −25° C., about −20° C., about −15° C., about −5° C., about 0° C., about 5° C., or about 10° C. for at least 1 month.

39-44. (canceled)

45. A syringe comprising the pharmaceutical composition of claim 1.

46-47. (canceled)

48. The pharmaceutical composition of claim 1, wherein the pharmaceutical composition is suitable for intravenous or subcutaneous injection.

49. The pharmaceutical composition of claim 1, wherein:
the antibody is at a concentration of about 100 mg/mL;
the buffer is phosphate buffer at a concentration of about 20 mM;
the antioxidant is L-methionine a concentration of at about 10 mM;
the sugar is sucrose at a concentration of about 4% w/v;
the viscosity modifying agent is L-arginine hydrochloride at a concentration of about 70 mM; and
the surfactant is P188 present in an amount of about 0.1% w/v,
wherein the pharmaceutical composition has a pH of about 7.2.

50. The pharmaceutical composition of claim 1, wherein:
the antibody is at a concentration of about 150 mg/mL;
the buffer is phosphate buffer at a concentration of about 20 mM;
the antioxidant is L-methionine at a concentration of about 10 mM;
the sugar is sucrose at a concentration of about 3.5% w/v;
the viscosity modifying agent is L-arginine hydrochloride at a concentration of about 70 mM; and
the surfactant is P188 present in an amount of about 0.1% w/v,
wherein the pharmaceutical composition has a pH of about 7.2.

51. The pharmaceutical composition of claim 1, wherein:
the pharmaceutical composition main peak, measured by SEC, decreases in area percent by less than 0.2% after 24 months at a temperature of about −70° C., relative to the initial value,
the pharmaceutical composition main peak, measured by SEC, decreases in area percent by less than 0.4% after 24 months at a temperature of about −20° C., relative to the initial value;
the pharmaceutical composition main peak, measured by SEC, decreases in area percent by less than 0.5% after 6 months at a temperature of about 5° C., relative to the initial value;
the pharmaceutical composition main peak, measured by SEC, decreases in area percent by less than 3% after 6 months at a temperature of about 25° C., relative to the initial value; or
the pharmaceutical composition main peak, measured by SEC, decreases in area percent by less than 7.8% after 1 month at a temperature of about 40° C., relative to the initial value.

52-55. (canceled)

56. The pharmaceutical composition of claim 1, wherein:
the main peak as measured by CE-SDS under non-reducing conditions is greater than 98% when stored at −20 C for up to 1, 3, 6, 9, 18, or 24 months,
the main peak as measured by CE-SDS under non-reducing conditions is greater than 97% when stored at 5 C for up to 1, 3, 6, 9, 18, or 24 months; or
the main peak as measured by CE-SDS under non-reducing conditions is greater than 93% when stored at 25 C for up to 1, 3, 6, 9, 18, or 24 months.

57. The pharmaceutical composition of claim 1, wherein:
the percentage of low molecular weight species (LMWS) as measured by CE-SDS under non-reducing conditions is less than, or about, 1.5, 1.1, or 1.0% when stored at −20 C for up to 1, 3, 6, 9, 18, or 24 months;
the percentage of low molecular weight species (LMWS) as measured by CE-SDS under non-reducing conditions is less than, or about, 2.5%, 2.0%, or 1.7% when stored at 5 C for up to 1, 3, 6, 9, 18, or 24 months; or
the percentage of low molecular weight species (LMWS) as measured by CE-SDS under non-reducing conditions is less than, or about, 5%, 3.5%, or 1.5% when stored at 25 C for up to 1, 3, 6, 9, 18, or 24 months.

58. The pharmaceutical composition of claim 1, wherein:
the percentage of high molecular weight species (HMWS) as measured by SEC is less than 2.0% when stored at −20 C for up to 1, 3, 6, 9, 18, or 24 months;
the percentage of high molecular weight species (HMWS) as measured by SEC is less than 2.0% when stored at 5 C for up to 1, 3, 6, 9, 18, or 24 months; or
the percentage of high molecular weight species (HMWS) as measured by SEC is less than 5%, 3.5%, or 1.5% when stored at 25 C for up to 1, 3, 6, 9, 18, or 24 months.

59-112. (canceled)

113. A method of treating a subject with a C1s mediated disorder, the method comprising administering to the subject the pharmaceutical composition of claim 1.

114. The method of claim 113, wherein the C1s mediated disorder is Myasthenia Gravis, hemolysis, Cold Agglutinin Disease, Immune Thrombocytopenia (ITP), Glomerulopathies, Atypical Hemolytic uremic syndrome, antiphospholipid antibody syndrome, transplant rejection, chronic inflammatory demyelinating polyneuropathy (CIDP), multifocal motor neuropathy (MMN), dermatomyositis, anti MAG neuropathy, due to stroke, or due to spinal cord injury.

115. The method of claim 113, wherein the volume of the pharmaceutical composition administered is about 1 mL or about 2 mL.

116. A kit comprising container a container comprising the pharmaceutical composition of claim 1.

117. (canceled)
